(19) United States
(12) Patent Application Publication
(10) Pub. No.: US 2025/0093370 A1
(43) Pub. Date: Mar. 20, 2025

(54) BIOMARKERS AND THEIR USE IN DIAGNOSING PARKINSON'S DISEASE, VITREORETINAL DISEASES, AND AGE-RELATED PATHOLOGIES

(71) Applicant: The Board of Trustees of the Leland Standord Junior University, Stanford, CA (US)

(72) Inventors: Vinit Mahajan, Los Altos, CA (US); Julian Wolf, Redwood City, CA (US)

(21) Appl. No.: 18/887,791

(22) Filed: Sep. 17, 2024

Related U.S. Application Data
(60) Provisional application No. 63/583,412, filed on Sep. 18, 2023.

(51) Int. Cl. G01N 33/68; G16H 50/30

(52) U.S. Cl. CPC G01N 33/6896; G16H 50/30; G01N 2333/4704; G01N 2333/4727; G01N 2333/475; G01N 2333/50; G01N 2333/70571; G01N 2333/7158; G01N 2333/78; G01N 2333/91142; G01N 2333/912; G01N 2333/916; G01N 2333/96419; G01N 2800/164; G01N 2800/2835; G01N 2800/52

(57) ABSTRACT

Compositions, methods, and kits are provided for diagnosing Parkinson's disease, vitreoretinal diseases, and age-related pathologies. In particular, aqueous humor biomarkers have been identified that correlate with biological aging and age-related pathologies and morbidity. The use of such biomarkers may allow earlier intervention in treatment of aging-related diseases. In addition, methods of using aqueous humor biomarkers for prognosis, diagnosis, and monitoring treatment of Parkinson's disease and vitreoretinal diseases are also provided.

### CROSS REFERENCE TO RELATED APPLICATIONS

This application claims benefit of U.S. Provisional Patent Application No. 63/583,412, filed Sep. 18, 2023, which application is incorporated herein by reference in its entirety.

### BACKGROUND OF THE INVENTION

In human clinical trials, ninety percent of all drug candidates fail despite having shown efficacy in an animal or cell model (Dowden and Munro, J. (2019) Nat Rev Drug Discov 18, 495-496). The failure rate implies most models do not provide sufficient insight into the disease mechanisms of living humans (Seok et al. (2013) Proc Natl Acad Sci USA 110, 3507-3512). The incomplete understanding of which specific cells are driving disease reflects the difficulty of interrogating single cells and their molecules in vivo within highly complex dynamic human tissues. In particular, cell-level analysis has been severely limited in non-regenerative organs and tissues such as the brain and the retina because direct biopsies would cause serious, irreversible functional damage. Liquid biopsies that capture locally enriched fluid may be the only benign way to obtain proteomic information. However, current analyses cannot resolve proteins at refined cellular resolution. Conversely, while single-cell RNA sequencing (scRNA-seq) and spatial transcriptomics yield a description of gene expression at cellular resolution, it either requires direct tissue biopsy or is limited to postmortem tissues. Moreover, one cannot infer if any given mRNA is translated into protein, making it challenging to assess proteome-wide relevance to human disease.

There remains a need for better methods of monitoring molecular pathophysiology and human health.

### SUMMARY OF THE INVENTION

Compositions, methods, and kits are provided for diagnosing Parkinson's disease, vitreoretinal diseases and age-related pathologies. In particular, aqueous humor biomarkers have been identified that correlate with biological aging and age-related pathologies and morbidity. The use of such biomarkers may allow earlier intervention in treatment of aging-related diseases. In addition, the inventors have discovered that protein exchange occurs between the vitreous and aqueous humor of the eye, which enables monitoring of biomarkers of various vitreoretinal diseases by measuring levels of biomarkers in the aqueous humor.

Biomarkers that can be used in the practice of the subject methods include, but are not limited to, biomarkers for diagnosing retinitis pigmentosa such as retinoschisin 1 (RS1), S-antigen visual arrestin (SAG), serine peptidase inhibitor Kazal type 4 (SPINK4), fucosyltransferase 3 (Lewis blood group) (FUT3), growth differentiation factor 10 (GDF10), leucine rich repeat, Ig-like and transmembrane domains 2 (LRIT2), Fas apoptotic inhibitory molecule (FAIM), cadherin related family member 1 (CDHR1), recoverin (RCVRN), GSK3B interacting protein (GSKIP), eyes shut homolog (EYS), natriuretic peptide C (NPPC), hes family bHLH transcription factor 6 (HES6), fibroblast growth factor 23 (FGF23), adaptor related protein complex 4 subunit mu 1 (AP4M1), guanylate cyclase activator 1A (GUCA1A), spermatogenesis associated 33 (SPATA33), metallophosphoesterase domain containing 2 (MPPED2), acid sensing ion channel subunit family member 4 (ASIC4), junctophilin 4 (JPH4), copine 7 (CPNE7), teneurin transmembrane protein 4 (TENM4), glutamate metabotropic receptor 4 (GRM4), tankyrase (TNKS), glutamate decarboxylase 1 (GAD1), synaptic vesicle glycoprotein 2A (SV2A), synaptotagmin 5 (SYT5), RIC3 acetylcholine receptor chaperone (RIC3), calcium/calmodulin dependent protein kinase II alpha (CAMK2A), leucine rich repeat transmembrane neuronal (4LRRTM4), potassium voltage-gated channel modifier subfamily G member 4 (KCNG4), dipeptidyl peptidase like 10 (DPP10), protein phosphatase 1 regulatory subunit 27 (PPP1R27), regulator of G protein signaling 5 (RGS5), GRB2 related adaptor protein (GRAP), receptor activity modifying protein 3 (RAMP3), mesenchyme homeobox 1 (MEOX1), actin filament associated protein 1 like 1 (AFAP1L1), opioid binding protein/cell adhesion molecule like (OPCML), leucine rich repeat, Ig-like and transmembrane domains 3 (LRIT3), phospholipase A2 group V (PLA2G5), polypeptide N-acetylgalactosaminyltransferase 11 (GALNT11), and potassium channel tetramerization domain containing 4 (KCTD4); biomarkers for diagnosing diabetic retinopathy such as lipopolysaccharide binding protein (LBP), C-reactive protein (CRP), inter-alpha-trypsin inhibitor heavy chain 3 (ITIH3), apolipoprotein A1 (APOA1), carboxypeptidase N subunit 2 (CPN2), complement component 4 binding protein alpha (C4BPA), hyaluronan binding protein 2 (HABP2), coagulation factor XIII B chain (F13B), serpin family A member 10 (SERPINA10), insulin like growth factor binding protein 1 (IGFBP1), fibronectin 1 (FN1), matrix metallopeptidase 19 (MMP19), angiopoietin 2 (ANGPT2), roundabout guidance receptor 4 (ROBO4), vascular endothelial growth factor D (VEGFD), angiopoietin like 4 (ANGPTL4), fms related receptor tyrosine kinase 4 (FLT4), filamin A (FLNA), EPH receptor A1 (EPHA1), endoglin (ENG), thrombospondin 1 (THBS1), chloride intracellular channel 4 (CLIC4), neuropilin 2 (NRP2), vascular endothelial growth factor A (VEGFA), tryptophanyl-tRNA synthetase 1 (WARS1), C-X-C motif chemokine ligand 8 (CXCL8), thymidine phosphorylase (TYMP), C-C motif chemokine ligand 2 (CCL2), mitogen-activated protein kinase 14 (MAPK14), and AKT serine/threonine kinase 1 (AKT1); biomarkers for distinguishing between a diagnosis of non-proliferative diabetic retinopathy and proliferative diabetic retinopathy such as fibronectin 1 (FN1), matrix metallopeptidase 19 (MMP19), angiopoietin 2 (ANGPT2), roundabout guidance receptor 4 (ROBO4), vascular endothelial growth factor D (VEGFD), angiopoietin like 4 (ANGPTL4), fms related receptor tyrosine kinase 4 (FLT4), filamin A (FLNA), EPH receptor A1 (EPHA1), and endoglin (ENG); biomarkers for diagnosing Parkinson's disease such as trio Rho guanine nucleotide exchange factor (TRIO), junctophilin 4 (JPH4), copine 7 (CPNE7), glutamate metabotropic receptor 4 (GRM4), tankyrase (TNKS), serine active site containing 1 (SERAC1), activin A receptor type 2B (ACVR2B), synaptic vesicle glycoprotein 2A (SV2A), RIC3 acetylcholine receptor chaperone (RIC3), calcium/calmodulin dependent protein kinase II alpha (CAMK2A), BCL2 like 2 (BCL2L2), dysbindin domain containing 1 (DBNDD1), sulfotransferase family 4A member 1 (SULT4A1), hyaluronan binding protein 4 (HABP4), syntaphilin (SNPH), Rac family small GTPase 3 (RAC3), synaptotagmin 13 (SYT13), p21 (RAC1) activated kinase 5 (PAK5), neurotrophic receptor tyrosine kinase 1 (NTRK1), complement C1q like 4 (C1QL4), potassium voltage-gated channel modifier subfamily G member 4 (KCNG4), dipeptidyl peptidase like 10 (DPP10), protein phosphatase 1 regulatory subunit 27 (PPP1R27), PIH1 domain containing 2 (PIH1D2), cadherin related family member 1 (CDHR1), chromosome 17 open reading frame 67 (C17orf67), GSK3B interacting protein (GSKIP), centrosomal protein 112 (CEP112), hes family bHLH transcription factor 6 (HES6), fibroblast growth factor 23 (FGF23), adaptor related protein complex 4 subunit mu 1 (AP4M1), spermatogenesis associated 33 (SPATA33), MPPED2 (MPPED2), cholinergic receptor nicotinic alpha 5 subunit (CHRNA5), and acid sensing ion channel subunit family member 4 (ASIC4); biomarkers for diagnosing uveitis such as immunoglobulin heavy constant mu (IGHM), thioredoxin domain containing 5 (TXNDC5), SLAM family member 7 (SLAMF7), marginal zone B and B1 cell specific protein (MZB1), immunoglobulin heavy constant delta (IGHD), complement C3d receptor 2 (CR2), immunoglobulin heavy constant gamma 4 (IGHG4), lymphocyte antigen 9 (LY9), Fc receptor like 5 (FCRL5), TNF receptor superfamily member 13B (TNFRSF13B), matrix metallopeptidase 9 (MMP9), interleukin 1 receptor type 2 (IL1R2), GLI pathogenesis related 2 (GLIPR2), myeloperoxidase (MPO), resistin (RETN), CD177 molecule (CD177), proline-serine-threonine phosphatase interacting protein 1 (PSTPIP1), C-type lectin domain family 12 member A (CLEC12A), high mobility group box 2 (HMGB2), leukotriene A4 hydrolase (LTA4H), von Willebrand factor (VWF), tyrosine kinase with immunoglobulin like and EGF like domains 1 (TIE1), selectin E (SELE), cadherin 5 (CDH5), angiopoietin 2 (ANGPT2), fms related receptor tyrosine kinase 4 (FLT4), adhesion G protein-coupled receptor F5 (ADGRF5), vascular endothelial growth factor C (VEGFC), TEK receptor tyrosine kinase (TEK), MYC target 1 (MYCT1), C-X-C motif chemokine ligand 10 (CXCL10), interleukin 18 binding protein (IL18BP), C-C motif chemokine ligand 22 (CCL22), TNF receptor superfamily member 8 (TNFRSF8), C-C motif chemokine ligand 7 (CCL7), matrix metallopeptidase 12 (MMP12), interleukin 10 (IL10), C-X-C motif chemokine ligand 11 (CXCL11), chymotrypsin like elastase 1 (CELA1), granulysin (GNLY), cystatin F (CST7), CD8 subunit alpha (CD8A), CD5 molecule (CD5), granzyme K (GZMK), granzyme A (GZMA), C-C motif chemokine ligand 5 (CCL5), CD7 molecule (CD7), LCK proto-oncogene, Src family tyrosine kinase (LCK), and SH2 domain containing 1A (SH2D1A); biomarkers for determining biological age of an eye such as leukocyte cell derived chemotaxin 2 (LECT2), discoidin, CUB and LCCL domain containing 1 (DCBLD1), secretagogin, EF-hand calcium binding protein (SCGN), aggrecan (ACAN), amine oxidase copper containing 2 (AOC2), calcyphosine (CAPS), trefoil factor 3 (TFF3), amnion associated transmembrane protein (AMN), ABO, alpha 1-3-N-acetylgalactosaminyltransferase and alpha 1-3-galactosyltransferase (ABO), neuropilin and tolloid like 1 (NETO1), CD274 molecule (CD274), pro-platelet basic protein (PPBP), ABL proto-oncogene 1, non-receptor tyrosine kinase (ABL1), hepcidin antimicrobial peptide (HAMP), insulin like growth factor binding protein 1 (IGFBP1), adipogenesis associated Mth938 domain containing (AAMDC), alpha-1-B glycoprotein (A1 BG), adenosine deaminase 2 (ADA2), major vault protein (MVP), crystallin gamma C (CRYGC), alpha 1,4-galactosyltransferase (P1 PK blood group) (A4GALT), alpha-1,4-N-acetylglucosaminyltransferase (A4GNT), aminoadipate aminotransferase (AADAT), alpha-2-macroglobulin (A2M), and ABI family member 3 (ABI3); and biomarkers for estimating age of a subject such as leukocyte cell derived chemotaxin 2 (LECT2), discoidin, CUB and LCCL domain containing 1 (DCBLD1), secretagogin, EF-hand calcium binding protein (SCGN), aggrecan (ACAN), amine oxidase copper containing 2 (AOC2), calcyphosine (CAPS), trefoil factor 3 (TFF3), amnion associated transmembrane protein (AMN), ABO, alpha 1-3-N-acetylgalactosaminyltransferase and alpha 1-3-galactosyltransferase (ABO), neuropilin and tolloid like 1 (NETO1), CD274 molecule (CD274), pro-platelet basic protein (PPBP), ABL proto-oncogene 1, non-receptor tyrosine kinase (ABL1), hepcidin antimicrobial peptide (HAMP), insulin like growth factor binding protein 1 (IGFBP1), adipogenesis associated Mth938 domain containing (AAMDC), alpha-1-B glycoprotein (A1BG), adenosine deaminase 2 (ADA2), major vault protein (MVP), crystallin gamma C (CRYGC), alpha 1,4-galactosyltransferase (P1 PK blood group) (A4GALT), alpha-1,4-N-acetylglucosaminyltransferase (A4GNT), aminoadipate aminotransferase (AADAT), alpha-2-macroglobulin (A2M), and ABI family member 3 (ABI3).

In one aspect, a method of diagnosing and treating retinitis pigmentosa in a patient is provided, the method comprising: obtaining an aqueous humor sample from an eye of the patient; measuring levels of one or more biomarkers selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LRIT3, PLA2G5, GALNT11, and KCTD4 in the aqueous humor sample, wherein decreased levels of the one or more biomarkers selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LRIT3, PLA2G5, GALNT11, and KCTD4 compared to reference value ranges for the levels of the one or more biomarkers in a control aqueous humor sample indicate that the patient has retinitis pigmentosa; and treating the patient for the retinitis pigmentosa if the patient has a positive diagnosis for retinitis pigmentosa.

In certain embodiments, the levels of at least two, at least three, or at least four biomarkers selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LRIT3, PLA2G5, GALNT11, and KCTD4 are measured in the aqueous humor sample.

In certain embodiments, the levels of RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LRIT3, PLA2G5, GALNT11, and KCTD4 are measured in the aqueous humor sample.

In certain embodiments, the levels of RS1, SAG, SPINK4, FUT3, GDF10, LRIT2, FAIM, CDHR1, RCVRN, and GSKIP are measured in the aqueous humor sample.

In certain embodiments, the EYS, NPPC, HES6, FGF23, AP4M1, GUCA1A, SPATA33, MPPED2, and ASIC4 are measured in the aqueous humor sample.

In certain embodiments, the levels of JPH4, CPNE7, TENM4, GRM4, TNKS, GAD1, SV2A, SYT5, RIC3, and CAMK2A are measured in the aqueous humor sample.

In certain embodiments, the levels of LRRTM4, KCNG4, DPP10, and PPP1R27 are measured in the aqueous humor sample.

In certain embodiments, the levels of RGS5, GRAP, RAMP3, and MEOX1, AFAP1L1 are measured in the aqueous humor sample.

In certain embodiments, the levels of OPCML, LRIT3, PLA2G5, GALNT11, and KCTD4 are measured in the aqueous humor sample.

In certain embodiments, the treating the patient for the retinitis pigmentosa comprises retinal sheet transplantation, RPE65 gene therapy, implanting a retinal prosthesis, or administering vitamin A, docosahexaenoic acid (DHA), N-acetylcysteine (NAC), and lutein, or a combination thereof.

In another aspect, a method of monitoring retinitis pigmentosa in a patient is provided, the method comprising: obtaining a first aqueous humor sample from an eye of the patient at a first time point and a second aqueous humor sample from the eye of the patient later at a second time point; and measuring levels of one or more biomarkers in the first aqueous humor sample and the second aqueous humor sample, wherein the biomarkers are selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LRIT3, PLA2G5, GALNT11, and KCTD4 in the aqueous humor sample, wherein detection of decreased levels of the one or more biomarkers selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LRIT3, PLA2G5, GALNT11, and KCTD4 in the second aqueous humor sample compared to the first aqueous humor sample indicate that the patient is worsening, and wherein detection of increased levels of the one or more biomarkers selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LRIT3, PLA2G5, GALNT11, and KCTD4 in the second aqueous humor sample compared to the first aqueous humor sample indicate that the patient is improving.

In another aspect, a method of monitoring efficacy of a treatment of a patient for retinitis pigmentosa is provided, the method comprising: obtaining a first aqueous humor sample from the patient before the patient undergoes the treatment and a second aqueous humor sample from the patient after the patient undergoes the treatment; measuring levels of one or more biomarkers in the first aqueous humor sample and the second aqueous humor sample, wherein the one or more biomarkers are selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LRIT3, PLA2G5, GALNT11, and KCTD4; and evaluating the efficacy of the treatment, wherein detection of decreased levels of the one or more biomarkers selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LRIT3, PLA2G5, GALNT11, and KCTD4 in the second aqueous humor sample compared to the first aqueous humor sample indicate that the patient is worsening or not responding to the treatment, and detection of increased levels of the one or more biomarkers selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LRIT3, PLA2G5, GALNT11, and KCTD4 in the second aqueous humor sample compared to the first aqueous humor sample indicate that the patient is improving. In some embodiments, the method further comprises altering the treatment if the patient is worsening or not responding to the treatment.

In another aspect, a method of diagnosing and treating diabetic retinopathy in a patient is provided, the method comprising: obtaining an aqueous humor sample from an eye of the patient; measuring levels of one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 in the aqueous humor sample, wherein increased levels of the one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 compared to reference value ranges for the levels of the one or more biomarkers in a control aqueous humor sample indicate that the patient has diabetic retinopathy; and treating the patient for the diabetic retinopathy if the patient has a positive diagnosis for diabetic retinopathy.

In certain embodiments, the levels of at least two, at least three, or at least four biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 are measured in the aqueous humor sample.

In certain embodiments, the levels of LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 are measured in the aqueous humor sample.

In certain embodiments, treating the patient for the diabetic retinopathy comprises administering an anti-vascular endothelial growth factor (VEGF) agent or a steroid, or performing panretinal laser photocoagulation or a vitrectomy, or a combination thereof. In some embodiments, the anti-VEGF agent is bevacizumab, ranibizumab, sunitinib, sorafenib, axitinib, aflibercept, brolucizuma, faricimab, or pazopanib. In some embodiments, the steroid is triamcinolone acetonide, fluocinolone acetonide, or dexamethasone.

In certain embodiments, the levels of FN1, MMP19, ANGPT2, ROBO4, VEGFD, ANGPTL4, FLT4, FLNA, EPHA1, and ENG are measured in the aqueous humor sample, wherein increased levels of the one or more biomarkers selected from FN1, MMP19, ANGPT2, ROBO4, VEGFD, ANGPTL4, FLT4, FLNA, EPHA1, and ENG compared to reference value ranges for the levels of the one or more biomarkers in a control aqueous humor sample indicate that the patient has non-proliferative diabetic retinopathy.

In certain embodiments, the method further comprises treating the patient for the non-proliferative diabetic retinopathy if the patient has a positive diagnosis for non-proliferative diabetic retinopathy.

In certain embodiments, the treating the patient for the non-proliferative diabetic retinopathy comprises administering an anti-VEGF agent or a steroid, performing vitrectomy or photocoagulation, or increasing frequency of retinal exams or a combination thereof.

In certain embodiments, the levels of THBS1, CLIC4, NRP2, VEGFA, WARS1, CXCL8, TYMP, CCL2, MAPK14, AKT1 are measured in the aqueous humor sample, wherein increased levels of the one or more biomarkers selected from THBS1, CLIC4, NRP2, VEGFA, WARS1, CXCL8, TYMP, CCL2, MAPK14, AKT1 compared to reference value ranges for the levels of the one or more biomarkers in a control aqueous humor sample indicate that the patient has proliferative diabetic retinopathy.

In certain embodiments, the method further comprises treating the patient for the proliferative diabetic retinopathy if the patient has a positive diagnosis for proliferative diabetic retinopathy.

In certain embodiments, treating the patient for the proliferative diabetic retinopathy comprises administering an anti-VEGF agent or performing panretinal laser photocoagulation, or a combination thereof.

In another aspect, a method of monitoring diabetic retinopathy in a patient is provided, the method comprising: obtaining a first aqueous humor sample from an eye of the patient at a first time point and a second aqueous humor sample from the eye of the patient later at a second time point; and measuring levels of one or more biomarkers in the first aqueous humor sample and the second aqueous humor sample, wherein the biomarkers are selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1, wherein detection of increased levels of the one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 in the second aqueous humor sample compared to the first aqueous humor sample indicate that the patient is worsening, and wherein detection of decreased levels of the one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 in the second aqueous humor sample compared to the first aqueous humor sample indicate that the patient is improving.

In another aspect, a method of monitoring efficacy of a treatment of a patient for diabetic retinopathy is provided, the method comprising: obtaining a first aqueous humor sample from the patient before the patient undergoes the treatment and a second aqueous humor sample from the patient after the patient undergoes the treatment; measuring levels of one or more biomarkers in the first aqueous humor sample and the second aqueous humor sample, wherein the one or more biomarkers are selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1; and evaluating the efficacy of the treatment, wherein detection of increased levels of the one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 in the second aqueous humor sample compared to the first aqueous humor sample indicate that the patient is worsening or not responding to the treatment, and detection of decreased levels of the one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 in the second aqueous humor sample compared to the first aqueous humor sample indicate that the patient is improving. In some embodiments, the method further comprises altering the treatment if the patient is worsening or not responding to the treatment.

In another aspect, a method of monitoring a patient having non-proliferative diabetic retinopathy for progression to proliferative diabetic retinopathy is provided, the method comprising: obtaining an aqueous humor sample from an eye of the patient; measuring levels of one or more biomarkers selected from THBS1, CLIC4, NRP2, VEGFA, WARS1, CXCL8, TYMP, CCL2, MAPK14, and AKT1 in the aqueous humor sample, wherein increased levels of the one or more biomarkers selected from THBS1, CLIC4, NRP2, VEGFA, WARS1, CXCL8, TYMP, CCL2, MAPK14, and AKT1 compared to reference value ranges for the levels of the one or more biomarkers in a control aqueous humor sample indicate that the patient has proliferative diabetic retinopathy.

In certain embodiments, the method further comprises treating the patient for the proliferative diabetic retinopathy if the patient has a positive diagnosis for proliferative diabetic retinopathy.

In certain embodiments, treating the patient for the proliferative diabetic retinopathy comprises administering an anti-VEGF agent or performing panretinal laser photocoagulation, or a combination thereof.

In another aspect, a method of distinguishing between a diagnosis of non-proliferative diabetic retinopathy and proliferative diabetic retinopathy for a patient is provided, the method comprising: obtaining an aqueous humor sample from an eye of the patient; measuring levels of one or more biomarkers selected from FN1, MMP19, ANGPT2, ROBO4, VEGFD, ANGPTL4, FLT4, FLNA, EPHA1, and ENG in the aqueous humor sample, wherein increased levels of the one or more biomarkers selected from FN1, MMP19, ANGPT2, ROBO4, VEGFD, ANGPTL4, FLT4, FLNA, EPHA1, and ENG compared to reference value ranges for the levels of the one or more biomarkers in a control aqueous humor sample indicate that the patient has non-proliferative diabetic retinopathy; and measuring levels of one or more biomarkers selected from THBS1, CLIC4, NRP2, VEGFA, WARS1, CXCL8, TYMP, CCL2, MAPK14, and AKT1 in the aqueous humor sample, wherein increased levels of the one or more biomarkers selected from THBS1, CLIC4, NRP2, VEGFA, WARS1, CXCL8, TYMP, CCL2, MAPK14, and AKT1 compared to reference value ranges for the levels of the one or more biomarkers in a control aqueous humor sample indicate that the patient has proliferative diabetic retinopathy.

In certain embodiments, the levels of at least two, at least three, or at least four biomarkers selected from FN1, MMP19, ANGPT2, ROBO4, VEGFD, ANGPTL4, FLT4, FLNA, EPHA1, and ENG, and the levels of at least two, at least three, or at least four biomarkers selected from THBS1, CLIC4, NRP2, VEGFA, WARS1, CXCL8, TYMP, CCL2, MAPK14, and AKT1 are measured in the aqueous humor sample.

In certain embodiments, the levels of FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 are measured in the aqueous humor sample.

In certain embodiments, the method further comprises treating the patient for the non-proliferative diabetic retinopathy if the patient has a positive diagnosis for non-proliferative diabetic retinopathy, or treating the patient for the proliferative diabetic retinopathy if the patient has a positive diagnosis for proliferative diabetic retinopathy.

In certain embodiments, treating the patient for the non-proliferative diabetic retinopathy comprises administering an anti-VEGF agent or a steroid, performing vitrectomy or photocoagulation, or increasing frequency of retinal exams or a combination thereof.

In certain embodiments, treating the patient for the proliferative diabetic retinopathy comprises administering an anti-VEGF agent or performing panretinal laser photocoagulation, or a combination thereof.

In another aspect, a method of diagnosing and treating Parkinson's disease in a patient is provided, the method comprising: obtaining an aqueous humor sample from an eye of the patient; measuring levels of one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 in the aqueous humor sample, wherein decreased levels of the one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 compared to reference value ranges for the levels of the one or more biomarkers in a control aqueous humor sample indicate that the patient has Parkinson's disease; and treating the patient for the Parkinson's disease if the patient has Parkinson's disease.

In certain embodiments, the levels of at least two, at least three, or at least four biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 are measured in the aqueous humor sample.

In certain embodiments, the levels of TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 are measured in the aqueous humor sample.

In certain embodiments, the levels of TRIO, JPH4, CPNE7, GRM4, TNKS, SERAC1, ACVR2B, SV2A, RIC3, and CAMK2A are measured in the aqueous humor sample.

In certain embodiments, the levels of BCL2L2, DBNDD1, SULT4A1, HABP4, SNPH, RAC3, and SYT13 are measured in the aqueous humor sample.

In certain embodiments, the levels of PAK5, NTRK1, and C1QL4 are measured in the aqueous humor sample.

In certain embodiments, the levels of KCNG4, DPP10, and PPP1R27 are measured in the aqueous humor sample.

In certain embodiments, the levels of PIH1D2, CDHR1, C17orf67, GSKIP, and CEP112 are measured in the aqueous humor sample.

In certain embodiments, the levels of HES6, FGF23, AP4M1, SPATA33, MPPED2, CHRNA5, and ASIC4 are measured in the aqueous humor sample.

In certain embodiments, treating the patient for the Parkinson's disease comprises administering 1-3,4-dihydroxyphenylalanine (L-DOPA), an aromatic L-amino acid decarboxylase inhibitor, a catechol-O-methyltransferase (COMT) inhibitor, a dopamine agonist, a monoamine oxidase inhibitor, an anticholinergic, or a combination thereof. In some embodiments, the aromatic L-amino acid decarboxylase inhibitor is carbidopa, benserazide, methyldopa, alpha-difluoromethyl-DOPA, 3′,4′,5,7-tetrahydroxy-8-methoxyisoflavone, epigallocatechin gallate, or epigallocatechin. In some embodiments, the COMT inhibitor is entacapone, nebicapone, nitecapone, opicapone, or tolcapone. In some embodiments, the dopamine agonist is pramipexole, ropinirole, bromocriptine, pergolide, rotigotine, apomorphine, aripiprazole, phencyclidine, quinpirole, cabergoline, ciladopa, dihydrexidine, dinapsoline, doxanthrine, lisuride, piribedil, propylnorapomorphine, quinagolide, roxindole, or sumanirole. In some embodiments, the monoamine oxidase inhibitor is rasagiline, or selegiline, safinamide. In some embodiments, the anticholinergic is procyclidine or trihexyphenidyl.

In another aspect, a method of monitoring Parkinson's disease in a patient is provided, the method comprising: obtaining a first aqueous humor sample from an eye of the patient at a first time point and a second aqueous humor sample from the eye of the patient later at a second time point; and measuring levels of one or more biomarkers in the first aqueous humor sample and the second aqueous humor sample, wherein the biomarkers are selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 in the aqueous humor sample, wherein detection of decreased levels of the one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 in the second aqueous humor sample compared to the first aqueous humor sample indicate that the patient is worsening, and wherein detection of increased levels of the one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 in the second aqueous humor sample compared to the first aqueous humor sample indicate that the patient is improving.

In another aspect, a method of monitoring efficacy of a treatment of a patient for Parkinson's disease is provided, the method comprising: obtaining a first aqueous humor sample from the patient before the patient undergoes the treatment and a second aqueous humor sample from the patient after the patient undergoes the treatment; measuring levels of one or more biomarkers in the first aqueous humor sample and the second aqueous humor sample, wherein the one or more biomarkers are selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4; and evaluating the efficacy of the treatment, wherein detection of decreased levels of the one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4, and KCTD4 in the second aqueous humor sample compared to the first aqueous humor sample indicate that the patient is worsening or not responding to the treatment, and detection of increased levels of the one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 in the second aqueous humor sample compared to the first aqueous humor sample indicate that the patient is improving. In some embodiments, the method further comprises altering the treatment if the patient is worsening or not responding to the treatment.

In another aspect, a method of diagnosing and treating uveitis in a patient is provided, the method comprising: obtaining an aqueous humor sample from an eye of the patient; measuring levels of one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A in the aqueous humor sample, wherein increased levels of the one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A compared to reference value ranges for the levels of the one or more biomarkers in a control aqueous humor sample indicate that the patient has uveitis; and treating the patient for the uveitis if the patient has a positive diagnosis for uveitis.

In certain embodiments, the levels of at least two, at least three, or at least four biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A are measured in the aqueous humor sample.

In certain embodiments, the levels of IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A are measured in the aqueous humor sample.

In certain embodiments, the levels of IGHM, TXNDC5, SLAMF7, MZB1, IGHD, CR2, IGHG4, LY9, FCRL5, TNFRSF13B are measured in the aqueous humor sample.

In certain embodiments, the levels of MMP9, IL1R2, GLIPR2, MPO, RETN, CD177, PSTPIP1, CLEC12A, HMGB2, LTA4H are measured in the aqueous humor sample.

In certain embodiments, the levels of VWF, TIE1, SELE, CDH5, ANGPT2, FLT4, ADGRF5, VEGFC, TEK, MYCT1 are measured in the aqueous humor sample.

In certain embodiments, the levels of CXCL10, IL18BP, CCL22, TNFRSF8, CCL7, MMP12, IL110, CXCL11, CELA1 are measured in the aqueous humor sample.

In certain embodiments, the levels of GNLY, CST7, CD8A, CD5, GZMK, GZMA, CCL5, CD7, LCK, SH2D1A are measured in the aqueous humor sample.

In certain embodiments, the method further comprises measuring levels of one or more biomarkers selected from BICDL1, NPPC, HES6, FGF23, AP4M1, SPATA33, MPPED2, and CHRNA5, GPC2 in the aqueous humor sample, wherein decreased levels of the one or more biomarkers selected from BICDL1, NPPC, HES6, FGF23, AP4M1, SPATA33, MPPED2, CHRNA5, and GPC2 compared to reference value ranges for the levels of the one or more biomarkers in a control aqueous humor sample indicate that the uveitis is causing damage to cone cells.

In certain embodiments, the method further comprises measuring levels of one or more biomarkers selected from OPCML, GAP43, RIPPLY3, LRIT3, PLA2G5, FGF18, PLD5, and KCTD4 in the aqueous humor sample, wherein decreased levels of the one or more biomarkers selected OPCML, GAP43, RIPPLY3, LRIT3, PLA2G5, FGF18, PLD5, and KCTD4 compared to reference value ranges for the levels of the one or more biomarkers in a control aqueous humor sample indicate that the uveitis is causing damage to retinal pigment epithelium (RPE) cells.

In certain embodiments, the method further comprises measuring levels of one or more biomarkers selected from CPNE7, TYRO3, TENM4, MDM4, RDH13, SV2A, SYT5, OLFM3, RIC3, and SEMA6C in the aqueous humor sample, wherein decreased levels of the one or more biomarkers selected CPNE7, TYRO3, TENM4, MDM4, RDH13, SV2A, SYT5, OLFM3, RIC3, and SEMA6C compared to reference value ranges for the levels of the one or more biomarkers in a control aqueous humor sample indicate that the uveitis is causing damage to amacrine cells.

In certain embodiments, treating the patient for the uveitis comprises administering a glucocorticoid steroid, a cycloplegic agent, an antimetabolite, a T-cell inhibitor, an anti-tumor necrosis factor (TNF) agent, a biologic agent, an alkylating agent, an antibiotic for bacterial uveitis, an antiviral agent for viral uveitis, or an antifungal agent for fungal uveitis, or performing a vitrectomy, or a combination thereof. In some embodiments, the glucocorticoid steroid is selected from the group consisting of prednisolone, methylprednisolone, iluvien, ozurdex, retisert, and triamcinolone. In some embodiments, the T-cell inhibitor is a calcineurin inhibitor or a mTOR inhibitor. In some embodiments, the calcineurin inhibitor is selected from the group consisting of cyclosporine, tacrolimus and voclosporin. In some embodiments, the mTOR inhibitor is selected from the group consisting of everolimus and sirolimus. In some embodiments, the antimetabolite is a purine antagonist, a dihydrofolate reductase (DHFR) inhibitor, or an inosine monophosphate dehydrogenase (IMPDH) inhibitor. In some embodiments, the antimetabolite is selected from the group consisting of azathioprine, methotrexate, and mycophenolate mofetil. In some embodiments, the anti-TNF agent is selected from the group consisting of adalimumab, certolizumab, golimumab, infliximab, and etanercept. In some embodiments, the biologic agent is selected from the group consisting of efalizumab, rituximab, abatacept, alemtuzumab, anakinra, canakinumab, gevokizumab, daclizumab, tocilizumab, secukinumab, interferon α/β, fingolimod, aflibercept, bevacizumab, ranibizumab, and intravenous immunoglobulin (IVIG). In some embodiments, the alkylating agent is chlorambucil or cyclophosphamide. In some embodiments, the cycloplegic agent is atropine or homatropine. In some embodiments, the antibiotic is selected from the group consisting of cephalosporins, vancomycin, ceftazidime, amikacin, gentamycin, and moxifloxacin. In some embodiments, the antiviral agent is selected from the group consisting of ganciclovir, acyclovir, foscarnet, valacyclovir, and cidofivir. In some embodiments, the antifungal agent is selected from the group consisting of amphotericin B, voriconazole, caspofungin, and fluconazole.

In another aspect, a method of monitoring uveitis in a patient is provided, the method comprising: obtaining a first aqueous humor sample from an eye of the patient at a first time point and a second aqueous humor sample from the eye of the patient later at a second time point; and measuring levels of one or more biomarkers in the first aqueous humor sample and the second aqueous humor sample, wherein the biomarkers are selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A in the aqueous humor sample, wherein detection of increased levels of the one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A in the second aqueous humor sample compared to the first aqueous humor sample indicate that the patient is worsening, and wherein detection of decreased levels of the one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A in the second aqueous humor sample compared to the first aqueous humor sample indicate that the patient is improving.

In another aspect, a method of monitoring efficacy of a treatment of a patient for uveitis is provided, the method comprising: obtaining a first aqueous humor sample from the patient before the patient undergoes the treatment and a second aqueous humor sample from the patient after the patient undergoes the treatment; measuring levels of one or more biomarkers in the first aqueous humor sample and the second aqueous humor sample, wherein the one or more biomarkers are selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL110, CXCL11, CELA1, GNLY, CST7, CD8A, CD5, GZMK, GZMA, CCL5, CD7, LCK, and SH2D1A; and evaluating the efficacy of the treatment, wherein detection of increased levels of the one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A in the second aqueous humor sample compared to the first aqueous humor sample indicate that the patient is worsening or not responding to the treatment, and detection of decreased levels of the one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL110, CXCL11, CELA1, GNLY, CST7, CD8A, CD5, GZMK, GZMA, CCL5, CD7, LCK, and SH2D1A in the second aqueous humor sample compared to the first aqueous humor sample indicate that the patient is improving. In some embodiments, the method further comprises altering the treatment if the patient is worsening or not responding to the treatment.

In another aspect, a method of determining biological age of an eye in a patient is provided, the method comprising: obtaining an aqueous humor sample from an eye of the patient; and measuring levels of one or more biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 in the aqueous humor sample; and using a machine learning aging model to determine the biological age of the eye based on the levels of the one or more biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3.

In certain embodiments, the levels of at least two, at least three, or at least four biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 are measured in the aqueous humor sample.

In certain embodiments, the levels of LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 are measured in the aqueous humor sample.

In certain embodiments, the method further comprises screening the patient for an aging-related disease if the biological age of the eye indicates a risk of age-related pathology and morbidity.

In certain embodiments, the method further comprises screening the patient for an age-related eye disease if the biological age of the eye indicates a risk of age-related pathology and morbidity. In some embodiments, the method further comprises administering a treatment for the age-related eye disease to the patient if the patient is identified as having the age-related eye disease.

In another aspect, a method of estimating age of a subject is provided, the method comprising: obtaining an aqueous humor sample from an eye of the patient; and measuring levels of one or more biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 in the aqueous humor sample; and using a machine learning aging model to estimate age of a subject based on the levels of the one or more biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3.

Exemplary methods for measuring biomarkers include, without limitation, performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot.

### BRIEF DESCRIPTION OF THE DRAWINGS

FIGS. 1A-1D. TEMPO multi-omics approach determines the cellular origin of liquid biopsy proteins. (FIG. 1A) Aqueous humor (AH) and vitreous liquid biopsies were obtained from patients undergoing eye surgery. (FIG. 1B) The proteomic profile was analyzed using an aptamer-based assay. (FIG. 1C) To determine which cells specifically expressed the corresponding genes of the identified proteins, scRNA-seq data from healthy ocular and extra-ocular cell types were integrated with the proteomics data. (FIG. 1D) AH and vitreous proteins are represented by dots in the anterior or posterior chamber of the eye; dot color represents each protein's cellular origin. RPE: retinal pigment epithelium. See also Table 5.

FIGS. 2A-2C: Cell type-specific marker proteins in liquid biopsies. (FIG. 2A) Number of detected proteins and corresponding genes expressed in ocular and extra-ocular tissues. (FIG. 2B) Unsupervised clustering of all cell types based on the gene expression profile encoding for all detected proteins (left panel). Each dot represents one cell type. The color encodes for tissue types. Clusters of cell types are labeled. The right panel shows the number of proteins from each cluster and the association to tissue types. Specific expression was defined as: highest z-score (>3) and >10^{th} percentile of all expressed genes in this cell type. For spleen and liver, only genes encoding for secreted and extracellular proteins were considered. (FIG. 2C) Cell type-specific markers in liquid biopsies (criteria from (FIG. 2B) plus: mean gene expression>maximal gene expression in any other cell type and >2*maximal mean gene expression in any cell cluster, and protein level >10^{th} percentile in AH and vitreous). The number of cell type-specific proteins is shown in parenthesis. Bile duct tissue is the negative control (neg. ctrl). The cartoon on the right illustrates that retinal cell type-specific proteins can be detected in AH liquid biopsies. Proteins are shown as dots, the color corresponding to their retinal cell type. C: corneal, CB: ciliary body, RPE: retinal pigment epithelium, TM: trabecular meshwork, t-SNE: t-distributed stochastic neighbor embedding. See also FIG. 8 and Table 1.

FIGS. 3A-3B: Tissue disease phenotypes can be molecularly monitored by locally enriched fluid biopsy. (FIG. 3A) Clinical phenotype of retinitis pigmentosa (RP). Fundus photography, retinal cross sectioning using optical coherence tomography (OCT), and electroretinogram (ERG) from a healthy patient and from a patient with RP. (FIG. 3B) Aqueous humor (AH) liquid biopsies were obtained from 7 patients with RP and 19 control patients. The bar graph shows the cell type-specific marker proteins which were significantly decreased in RP compared to control AH (log 2FC<−2 and adjusted p-value<0.05) for the top 10 affected cell types. The absolute numbers are shown in the graph. The boxplots visualize protein levels of the top 10 marker proteins for each of the indicated cell types. Each point represents one sample. RFU: relative fluorescence unit, RPE: retinal pigment epithelium. See also Table 2.

FIGS. 4A-4C: The molecular and cellular phenotypes differ between disease stages. (FIG. 4A) Clinical phenotype of diabetic retinopathy (DR). (FIG. 4B) Aqueous humor (AH) liquid biopsies were obtained from 10 patients with proliferative diabetic retinopathy (PDR), 5 patients with non-proliferative diabetic retinopathy (NPDR), 10 patients with diabetes mellitus without DR (DM w/o DR), and 19 patients with cataracts serving as controls. The bar graph shows the cell type-specific marker proteins which were significantly increased in DR vs. control AH (log 2FC >2 and adjusted p-value<0.05) for the top 10 changed cell types. The absolute numbers are shown in the graph. The boxplots below visualize protein levels of the top 10 liver proteins in AH. Each point represents one sample. (FIG. 4C): The levels of significantly increased angiogenic proteins in DR AH vs. control are visualized in the heatmap. Each row represents one protein, each column represents one sample. The boxplots in the middle column visualize the levels of the top 10 angiogenic proteins in each group. Each point represents one sample. Sankey plots on the right show the cellular origin of the top 10 proteins for each group. A cell type was considered as the origin of a protein if the corresponding gene expression was at least 2 standard deviations above the mean expression in all cell types. RFU: relative fluorescence unit. See also Table 3.

FIGS. 5A-5C: Eye fluids reflect phenotypic molecules of Parkinson's disease. (FIG. 5A) Aqueous humor (AH) liquid biopsies were obtained from 5 patients with Parkinson's disease (PD) and 19 control patients. Heatmap visualizing the AH protein level of PD-relevant proteins in PD vs. control patients. Each row represents one protein, and each column represents one sample. (FIG. 5B) Sankey plot showing the cellular origin of the increased PD-relevant proteins. A cell type was considered as origin of a protein if the corresponding gene expression was at least 2 standard deviations above the mean expression in all cell types. (FIG. 5C) Bar graph showing the cell type-specific marker proteins which were significantly decreased in PD AH vs. control AH (log 2FC <−2 and adjusted p-value<0.05) for the top 10 changed cell types. The absolute numbers are shown in the graph. The boxplots visualize AH protein levels of the top 10 proteins from the indicated cell types. Each point represents one sample. RFU: relative fluorescence unit. See also FIG. 10.

FIGS. 6A-6P: The molecular age of the eye follows predominantly non-linear trajectories. (FIG. 6A) 46 aqueous humor (AH) liquid biopsies were obtained from patients undergoing cataract surgery with otherwise healthy eyes. AH protein levels were z-scored, and age trajectories of 6,313 AH proteins were estimated by locally estimated scatterplot smoothing (LOESS). Each line represents one protein. (FIG. 6B) Unsupervised clustering grouped AH proteins with similar trajectories. (FIGS. 6C-6L) Protein trajectories of the 10 identified clusters. Each line represents one protein; the thicker lines represent the average trajectory for each cluster. The most significantly enriched pathways and the top cell type origins are shown for each cluster. (FIG. 6M-6P) Protein trajectories for molecules known to be involved in aging associated processes. See also FIG. 11.

FIGS. 7A-7K: AI models predict the global and cellular molecular age of the eye. (FIG. 7A) Artificial intelligence (AI) model prediction of the patient's age based on aqueous humor (AH) liquid biopsy proteomics in control patients (training cohort). Each point represents one sample. (FIG. 7B) Prediction of the patient's age in an independent validation cohort of 19 control patients. The final model was built on the 26 features (“age proteins”) which were identified in at least 3 of the 10 cross validation models in the training cohort. (FIGS. 7C-7D) Prediction of the molecular age in patients with diabetic retinopathy (DR) (FIGS. 7C-7D), uveitis (FIG. 7D), and retinitis pigmentosa (RP) (FIG. 7D). The boxplots in (FIG. 7D) visualize the molecular age difference in years relative to control samples. A positive value means that the predicted molecular age was higher than the chronological age. (FIG. 7E) Sankey plot showing the cellular origin of the age proteins. A cell type was considered as origin of a protein if the corresponding gene expression was at least 2 standard deviations above the mean expression in all cell types. Proteins are ordered according to the feature importance in the final AI model. (FIGS. 7F-7H) Three AI models were developed to predict the vascular, immune, and retinal age of the eye. These models were based on proteins specific to the respective cell types (FIG. 12). (FIGS. 7I-7K) Bar plots showing the cellular origin of the proteins identified by the cell type-specific AI models. DM w/o DR: diabetes mellitus without DR, NPDR: non-proliferative DR, PDR: proliferative DR, p-values: *<0.05, **<0.01, ***<0.001, ns: not significant, RMSE: root-mean-square error. See also FIG. 12 and Table 4.

FIG. 8: Proteins from eye-specific cell types are strongly enriched in aqueous humor compared to plasma, related to FIG. 2. The protein level in healthy aqueous humor (AH) was compared to published proteomics data from the blood, which was generated using the same proteomics platform.⁶⁷ The percentile of protein level was determined for each protein in AH and blood and the difference between the AH percentile and the blood percentile was calculated for each protein. The bar graph shows the difference for all identified cell type-specific marker proteins, with the height of the bars corresponding to the mean difference and the error bar to the standard error of the mean. Significance was calculated using the Mann-Whitney U test (p<0.05 was considered significant).

FIGS. 9A-9D: Liquid biopsies to assess the immune cell profile and to monitor retinal damage in intraocular inflammation, related to FIG. 3. (FIGS. 9A-9B) Clinical phenotype of intraocular inflammation in uveitis. (FIG. 9C) Aqueous humor (AH) liquid biopsies were obtained from 6 patients with uveitis and 19 patients with cataracts (but otherwise healthy eyes) serving as controls. The bar graph shows the cell type-specific marker proteins which were significantly increased in uveitis AH compared to control AH (log 2FC>2 and adjusted p-value<0.05) for the top 10 changed cell types (in percent of all marker proteins for each cell type). The numbers in the graph represent the absolute numbers. The boxplots visualize protein levels of the top 10 proteins from the indicated cell types in AH. Each point represents one sample. (FIG. 9D) AH liquid biopsy can detect a decrease of retinal cell type-specific marker proteins in uveitis, an indicator of retinal damage. The bar graph shows the cell type-specific marker proteins which were significantly decreased in uveitis AH compared to control AH (log 2FC<−2 and adjusted p-value<0.05) for the top 10 changed cell types (in percent of all marker proteins for each cell type). The absolute numbers are shown in the graph. The boxplots visualize protein levels of the top 10 proteins from the indicated cell types in AH. Each point represents one sample. Abbreviations: ERG: electroretinogram, OCT: optical coherence tomography, RFU: relative fluorescence unit, RPE: retinal pigment epithelium.

FIG. 10: Distinct alterations of the aqueous humor proteomic profile in Parkinson's disease, related to FIG. 5. Aqueous humor (AH) liquid biopsies were obtained from 5 patients with Parkinson's disease and 19 patients with cataracts serving as controls. The protein level of all detected 5,692 proteins is visualized in the heatmap. Each row represents one protein, and each column represents one sample. The dendrogram shows clustering of the proteins (rows). The z-score represents a protein's abundance level in relation to its mean abundance in all samples by standard deviation units.

FIG. 11: Protein trajectories of targets of serum response factor (SRF) with age, related to FIG. 6. In mice, SRF has been shown to mediate, at least in part, the rejuvenating power of young cerebrospinal fluid.⁴¹ Age trajectories were estimated by locally estimated scatterplot smoothing (LOESS). Each line represents one protein whereas the thicker line represents the average trajectory. The top cell type origins are shown.

FIGS. 12A-12I: Cell type-specific AI aging models predict the vascular, immune, and retinal age. To predict the vascular, immune, and retinal age of the eye, artificial intelligence (AI) models were built based on proteins originating specifically from vascular, immune, and retinal cells, respectively (training cohort, 46 samples, 10-fold cross validation) (FIGS. 12A, 12D, 12G). Each point represents one sample. The final models were built based on 24 (vascular aging), 21 (immune aging), and 27 proteins (retinal aging) which were selected by the AI models in the training cohort. The validity of these models was then prospectively evaluated in an independent cohort of 19 healthy patients (FIGS. 12B, 12E, 12H). The vascular (FIG. 12C), immune (FIG. 12F), and retinal age (FIG. 12I) was predicted in patients with diabetes without retinopathy (DM w/o DR), non-proliferative diabetic retinopathy (NPDR), proliferative diabetic retinopathy (PDR), retinitis pigmentosa (RP), and uveitis.

### DETAILED DESCRIPTION OF THE INVENTION

Compositions, methods, and kits are provided for diagnosing Parkinson's disease, vitreoretinal diseases and age-related pathologies. In particular, aqueous humor biomarkers have been identified that correlate with biological aging and age-related pathologies and morbidity. The use of such biomarkers may allow earlier intervention in treatment of aging-related diseases. In addition, the inventors have discovered that protein exchange occurs between the vitreous and aqueous humor of the eye, which enables monitoring of biomarkers of various vitreoretinal diseases by measuring levels of biomarkers in the aqueous humor.

Before the present compositions, methods, and kits are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a biomarker” includes a plurality of such biomarkers and reference to “the polypeptide” includes reference to one or more polypeptides and equivalents thereof, e.g., peptides or proteins known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

### Definitions

Biomarkers. The term “biomarker” as used herein refers to a compound, such as a protein, a polypeptide, a peptide, a mRNA, a metabolite, or a metabolic byproduct which is differentially expressed or present at different concentrations, levels or frequencies in one sample compared to another, such as an aqueous humor sample from patients who have a vitreoretinal disease, Parkinson's disease, or risk of age-related pathology and morbidity compared to an aqueous humor sample from healthy control subjects (i.e., subjects not having a vitreoretinal disease or Parkinson's disease, or younger healthy subjects not at risk of age-related pathology and morbidity).

Biomarkers include, but are not limited to, biomarkers for diagnosing retinitis pigmentosa such as retinoschisin 1 (RS1), S-antigen visual arrestin (SAG), serine peptidase inhibitor Kazal type 4 (SPINK4), fucosyltransferase 3 (Lewis blood group) (FUT3), growth differentiation factor 10 (GDF10), leucine rich repeat, Ig-like and transmembrane domains 2 (LRIT2), Fas apoptotic inhibitory molecule (FAIM), cadherin related family member 1 (CDHR1), recoverin (RCVRN), GSK3B interacting protein (GSKIP), eyes shut homolog (EYS), natriuretic peptide C (NPPC), hes family bHLH transcription factor 6 (HES6), fibroblast growth factor 23 (FGF23), adaptor related protein complex 4 subunit mu 1 (AP4M1), guanylate cyclase activator 1A (GUCA1A), spermatogenesis associated 33 (SPATA33), metallophosphoesterase domain containing 2 (MPPED2), acid sensing ion channel subunit family member 4 (ASIC4), junctophilin 4 (JPH4), copine 7 (CPNE7), teneurin transmembrane protein 4 (TENM4), glutamate metabotropic receptor 4 (GRM4), tankyrase (TNKS), glutamate decarboxylase 1 (GAD1), synaptic vesicle glycoprotein 2A (SV2A), synaptotagmin 5 (SYT5), RIC3 acetylcholine receptor chaperone (RIC3), calcium/calmodulin dependent protein kinase II alpha (CAMK2A), leucine rich repeat transmembrane neuronal (4LRRTM4), potassium voltage-gated channel modifier subfamily G member 4 (KCNG4), dipeptidyl peptidase like 10 (DPP10), protein phosphatase 1 regulatory subunit 27 (PPP1R27), regulator of G protein signaling 5 (RGS5), GRB2 related adaptor protein (GRAP), receptor activity modifying protein 3 (RAMP3), mesenchyme homeobox 1 (MEOX1), actin filament associated protein 1 like 1 (AFAP1 L1), opioid binding protein/cell adhesion molecule like (OPCML), leucine rich repeat, Ig-like and transmembrane domains 3 (LRIT3), phospholipase A2 group V (PLA2G5), polypeptide N-acetylgalactosaminyltransferase 11 (GALNT11), and potassium channel tetramerization domain containing 4 (KCTD4); biomarkers for diagnosing diabetic retinopathy such as lipopolysaccharide binding protein (LBP), C-reactive protein (CRP), inter-alpha-trypsin inhibitor heavy chain 3 (ITIH3), apolipoprotein A1 (APOA1), carboxypeptidase N subunit 2 (CPN2), complement component 4 binding protein alpha (C4BPA), hyaluronan binding protein 2 (HABP2), coagulation factor XIII B chain (F13B), serpin family A member 10 (SERPINA10), insulin like growth factor binding protein 1 (IGFBP1), fibronectin 1 (FN1), matrix metallopeptidase 19 (MMP19), angiopoietin 2 (ANGPT2), roundabout guidance receptor 4 (ROBO4), vascular endothelial growth factor D (VEGFD), angiopoietin like 4 (ANGPTL4), fms related receptor tyrosine kinase 4 (FLT4), filamin A (FLNA), EPH receptor A1 (EPHA1), endoglin (ENG), thrombospondin 1 (THBS1), chloride intracellular channel 4 (CLIC4), neuropilin 2 (NRP2), vascular endothelial growth factor A (VEGFA), tryptophanyl-tRNA synthetase 1 (WARS1), C-X-C motif chemokine ligand 8 (CXCL8), thymidine phosphorylase (TYMP), C-C motif chemokine ligand 2 (CCL2), mitogen-activated protein kinase 14 (MAPK14), and AKT serine/threonine kinase 1 (AKT1); biomarkers for distinguishing between a diagnosis of non-proliferative diabetic retinopathy and proliferative diabetic retinopathy such as fibronectin 1 (FN1), matrix metallopeptidase 19 (MMP19), angiopoietin 2 (ANGPT2), roundabout guidance receptor 4 (ROBO4), vascular endothelial growth factor D (VEGFD), angiopoietin like 4 (ANGPTL4), fms related receptor tyrosine kinase 4 (FLT4), filamin A (FLNA), EPH receptor A1 (EPHA1), and endoglin (ENG); biomarkers for diagnosing Parkinson's disease such as trio Rho guanine nucleotide exchange factor (TRIO), junctophilin 4 (JPH4), copine 7 (CPNE7), glutamate metabotropic receptor 4 (GRM4), tankyrase (TNKS), serine active site containing 1 (SERAC1), activin A receptor type 2B (ACVR2B), synaptic vesicle glycoprotein 2A (SV2A), RIC3 acetylcholine receptor chaperone (RIC3), calcium/calmodulin dependent protein kinase II alpha (CAMK2A), BCL2 like 2 (BCL2L2), dysbindin domain containing 1 (DBNDD1), sulfotransferase family 4A member 1 (SULT4A1), hyaluronan binding protein 4 (HABP4), syntaphilin (SNPH), Rac family small GTPase 3 (RAC3), synaptotagmin 13 (SYT13), p21 (RAC1) activated kinase 5 (PAK5), neurotrophic receptor tyrosine kinase 1 (NTRK1), complement C1q like 4 (C1QL4), potassium voltage-gated channel modifier subfamily G member 4 (KCNG4), dipeptidyl peptidase like 10 (DPP10), protein phosphatase 1 regulatory subunit 27 (PPP1R27), PIH1 domain containing 2 (PIH1D2), cadherin related family member 1 (CDHR1), chromosome 17 open reading frame 67 (C17orf67), GSK3B interacting protein (GSKIP), centrosomal protein 112 (CEP112), hes family bHLH transcription factor 6 (HES6), fibroblast growth factor 23 (FGF23), adaptor related protein complex 4 subunit mu 1 (AP4M1), spermatogenesis associated 33 (SPATA33), MPPED2 (MPPED2), cholinergic receptor nicotinic alpha 5 subunit (CHRNA5), and acid sensing ion channel subunit family member 4 (ASIC4); biomarkers for diagnosing uveitis such as immunoglobulin heavy constant mu (IGHM), thioredoxin domain containing 5 (TXNDC5), SLAM family member 7 (SLAMF7), marginal zone B and B1 cell specific protein (MZB1), immunoglobulin heavy constant delta (IGHD), complement C3d receptor 2 (CR2), immunoglobulin heavy constant gamma 4 (IGHG4), lymphocyte antigen 9 (LY9), Fc receptor like 5 (FCRL5), TNF receptor superfamily member 13B (TNFRSF13B), matrix metallopeptidase 9 (MMP9), interleukin 1 receptor type 2 (IL1R2), GLI pathogenesis related 2 (GLIPR2), myeloperoxidase (MPO), resistin (RETN), CD177 molecule (CD177), proline-serine-threonine phosphatase interacting protein 1 (PSTPIP1), C-type lectin domain family 12 member A (CLEC12A), high mobility group box 2 (HMGB2), leukotriene A4 hydrolase (LTA4H), von Willebrand factor (VWF), tyrosine kinase with immunoglobulin like and EGF like domains 1 (TIE1), selectin E (SELE), cadherin 5 (CDH5), angiopoietin 2 (ANGPT2), fms related receptor tyrosine kinase 4 (FLT4), adhesion G protein-coupled receptor F5 (ADGRF5), vascular endothelial growth factor C (VEGFC), TEK receptor tyrosine kinase (TEK), MYC target 1 (MYCT1), C-X-C motif chemokine ligand 10 (CXCL10), interleukin 18 binding protein (IL18BP), C-C motif chemokine ligand 22 (CCL22), TNF receptor superfamily member 8 (TNFRSF8), C-C motif chemokine ligand 7 (CCL7), matrix metallopeptidase 12 (MMP12), interleukin 10 (IL10), C-X-C motif chemokine ligand 11 (CXCL11), chymotrypsin like elastase 1 (CELA1), granulysin (GNLY), cystatin F (CST7), CD8 subunit alpha (CD8A), CD5 molecule (CD5), granzyme K (GZMK), granzyme A (GZMA), C-C motif chemokine ligand 5 (CCL5), CD7 molecule (CD7), LCK proto-oncogene, Src family tyrosine kinase (LCK), and SH2 domain containing 1A (SH2D1A); biomarkers for determining biological age of an eye such as leukocyte cell derived chemotaxin 2 (LECT2), discoidin, CUB and LCCL domain containing 1 (DCBLD1), secretagogin, EF-hand calcium binding protein (SCGN), aggrecan (ACAN), amine oxidase copper containing 2 (AOC2), calcyphosine (CAPS), trefoil factor 3 (TFF3), amnion associated transmembrane protein (AMN), ABO, alpha 1-3-N-acetylgalactosaminyltransferase and alpha 1-3-galactosyltransferase (ABO), neuropilin and tolloid like 1 (NETO1), CD274 molecule (CD274), pro-platelet basic protein (PPBP), ABL proto-oncogene 1, non-receptor tyrosine kinase (ABL1), hepcidin antimicrobial peptide (HAMP), insulin like growth factor binding protein 1 (IGFBP1), adipogenesis associated Mth938 domain containing (AAMDC), alpha-1-B glycoprotein (A1BG), adenosine deaminase 2 (ADA2), major vault protein (MVP), crystallin gamma C (CRYGC), alpha 1,4-galactosyltransferase (P1 PK blood group) (A4GALT), alpha-1,4-N-acetylglucosaminyltransferase (A4GNT), aminoadipate aminotransferase (AADAT), alpha-2-macroglobulin (A2M), and ABI family member 3 (ABI3); and biomarkers for estimating age of a subject such as leukocyte cell derived chemotaxin 2 (LECT2), discoidin, CUB and LCCL domain containing 1 (DCBLD1), secretagogin, EF-hand calcium binding protein (SCGN), aggrecan (ACAN), amine oxidase copper containing 2 (AOC2), calcyphosine (CAPS), trefoil factor 3 (TFF3), amnion associated transmembrane protein (AMN), ABO, alpha 1-3-N-acetylgalactosaminyltransferase and alpha 1-3-galactosyltransferase (ABO), neuropilin and tolloid like 1 (NETO1), CD274 molecule (CD274), pro-platelet basic protein (PPBP), ABL proto-oncogene 1, non-receptor tyrosine kinase (ABL1), hepcidin antimicrobial peptide (HAMP), insulin like growth factor binding protein 1 (IGFBP1), adipogenesis associated Mth938 domain containing (AAMDC), alpha-1-B glycoprotein (A1 BG), adenosine deaminase 2 (ADA2), major vault protein (MVP), crystallin gamma C (CRYGC), alpha 1,4-galactosyltransferase (P1 PK blood group) (A4GALT), alpha-1,4-N-acetylglucosaminyltransferase (A4GNT), aminoadipate aminotransferase (AADAT), alpha-2-macroglobulin (A2M), and ABI family member 3 (ABI3).

In some embodiments, the concentration or level of a biomarker is determined before and after the administration of a treatment to a patient. The treatment may comprise, for example, without limitation, administering a corticosteroid, a vascular endothelial growth factor inhibitor, vitamin A, docosahexaenoic acid (DHA), N-acetylcysteine (NAC), lutein, I-3,4-dihydroxyphenylalanine (L-DOPA), an aromatic L-amino acid decarboxylase inhibitor, a catechol-O-methyltransferase (COMT) inhibitor, a dopamine agonist, a monoamine oxidase inhibitor, or an anticholinergic agent, implanting a retinal prosthesis, or performing RPE65 gene therapy, retinal sheet transplantation, laser surgery, photocoagulation, or a vitrectomy. The degree of change in the concentration or level of a biomarker, or lack thereof, is interpreted as an indication of whether the treatment has the desired effect (e.g., preventing or reducing damage to the retina and loss of vision). In other words, the concentration or level of a biomarker is determined before and after the administration of the treatment to an individual, and the degree of change, or lack thereof, in the level is interpreted as an indication of whether the individual is “responsive” to the treatment.

A “reference level” or “reference value” of a biomarker means a level of the biomarker that is indicative of a particular biological age, disease state (e.g., Parkinson's disease or a vitreoretinal disease such as retinitis pigmentosa, diabetic retinopathy (e.g., non-proliferative or proliferative), or uveitis), phenotype, or predisposition to developing a particular disease state or phenotype, or lack thereof, as well as combinations of disease states, phenotypes, or predisposition to developing a particular disease state or phenotype, or lack thereof. A “positive” reference level of a biomarker means a level that is indicative of a particular biological age or disease state or phenotype. A “negative” reference level of a biomarker means a level that is indicative of a lack of a particular biological age or disease state or phenotype. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a particular disease state, phenotype, or lack thereof in a certain age or gender group). Such reference levels may also be tailored to specific techniques that are used to measure levels of biomarkers in aqueous humor samples (e.g., aptamer-based assays, immunoassays (e.g., ELISA), mass spectrometry (e.g., LC-MS, GC-MS), tandem mass spectrometry, NMR, biochemical or enzymatic assays, PCR, microarray analysis, etc.), where the levels of biomarkers may differ based on the specific technique that is used.

A “similarity value” is a number that represents the degree of similarity between two things being compared. For example, a similarity value may be a number that indicates the overall similarity between a patient's biomarker profile using specific phenotype-related biomarkers and reference value ranges for the biomarkers in one or more control samples or a reference profile (e.g., the similarity to a “biological age” biomarker expression profile, a “retinitis pigmentosa” biomarker expression profile, a “diabetic retinopathy” biomarker expression profile, a “proliferative diabetic retinopathy” biomarker expression profile, a “non-proliferative diabetic retinopathy” biomarker expression profile, a “uveitis” biomarker expression profile, or a “Parkinson's disease” biomarker expression profile). The similarity value may be expressed as a similarity metric, such as a correlation coefficient, or may simply be expressed as the expression level difference, or the aggregate of the expression level differences, between levels of biomarkers in a patient sample and a control sample or reference expression profile.

The terms “quantity”, “amount”, and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

Aqueous humor sample. The term “aqueous humor sample” with respect to an individual encompasses samples of ocular fluid secreted from the ciliary body of the eye. Aqueous humor is located in the anterior and posterior chambers of the eye. Aqueous humor samples can be obtained by any suitable method such as by liquid biopsy or surgically. The definition also includes samples that have been manipulated in any way after their procurement, such as by treatment with reagents; washed; or enriched for particular types of molecules, e.g., proteins, peptides, etc.

Obtaining and assaying a sample. The term “assaying” is used herein to include the physical steps of manipulating an aqueous humor sample to generate data related to the aqueous humor sample. As will be readily understood by one of ordinary skill in the art, an aqueous humor sample must be “obtained” prior to assaying the sample. Thus, the term “assaying” implies that the sample has been obtained. The terms “obtained” or “obtaining” as used herein encompass the act of receiving an extracted or isolated aqueous humor sample. For example, a testing facility can “obtain” an aqueous humor sample in the mail (or via delivery, etc.) prior to assaying the sample. In some such cases, the aqueous humor sample was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample.

The terms “obtained” or “obtaining” as used herein can also include the physical extraction or isolation of an aqueous humor sample from a subject. Accordingly, an aqueous humor sample can be isolated from a subject (and thus “obtained”) by the same person or same entity that subsequently assays the sample. When an aqueous humor sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating an aqueous humor sample.

In some embodiments, the step of obtaining comprises the step of isolating an aqueous humor sample (e.g., a pre-treatment aqueous humor sample, a post-treatment aqueous humor sample, etc.). Methods and protocols for isolating various aqueous humor samples will be known to one of ordinary skill in the art and any convenient method may be used to isolate an aqueous humor sample.

It will be understood by one of ordinary skill in the art that in some cases, it is convenient to wait until multiple samples have been obtained prior to assaying the samples. Accordingly, in some cases an isolated aqueous humor sample is stored until all appropriate samples have been obtained. One of ordinary skill in the art will understand how to appropriately store a variety of different types of aqueous humor samples and any convenient method of storage may be used (e.g., refrigeration) that is appropriate for the particular aqueous humor sample. In some embodiments, a pre-treatment aqueous humor sample is assayed prior to obtaining a post-treatment aqueous humor sample. In some cases, a pre-treatment aqueous humor sample and a post-treatment aqueous humor sample are assayed in parallel. In some cases, multiple different post-treatment aqueous humor samples and/or a pre-treatment aqueous humor sample are assayed in parallel. In some cases, aqueous humor samples are processed immediately or as soon as possible after they are obtained.

In some embodiments, the concentration (i.e., “level”), or expression level of a gene product, which may be a protein, peptide, etc., (which will be referenced herein as a biomarker), in an aqueous humor sample is measured (i.e., “determined”). By “expression level” (or “level”) it is meant the level of gene product (e.g., the absolute and/or normalized value determined for the RNA expression level of a biomarker or for the expression level of the encoded polypeptide, or the concentration of the protein in an aqueous humor sample). The term “gene product” or “expression product” are used herein to refer to the RNA transcription products (RNA transcripts, e.g., mRNA, an unspliced RNA, a splice variant mRNA, and/or a fragmented RNA) of the gene, including mRNA, and the polypeptide translation products of such RNA transcripts. A gene product can be, for example, an unspliced RNA, an mRNA, a splice variant mRNA, a microRNA, a fragmented RNA, a polypeptide, a post-translationally modified polypeptide, a splice variant polypeptide, etc.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assaying may be relative or absolute. For example, “assaying” can be determining whether the expression level is less than or “greater than or equal to” a particular threshold, (the threshold can be pre-determined or can be determined by assaying a control sample). On the other hand, “assaying to determine the expression level” can mean determining a quantitative value (using any convenient metric) that represents the level of expression (i.e., expression level, e.g., the amount of protein and/or RNA, e.g., mRNA) of a particular biomarker. The level of expression can be expressed in arbitrary units associated with a particular assay (e.g., fluorescence units, e.g., mean fluorescence intensity (MFI)), or can be expressed as an absolute value with defined units (e.g., number of mRNA transcripts, number of protein molecules, concentration of protein, etc.). Additionally, the level of expression of a biomarker can be compared to the expression level of one or more additional genes (e.g., nucleic acids and/or their encoded proteins) to derive a normalized value that represents a normalized expression level. The specific metric (or units) chosen is not crucial as long as the same units are used (or conversion to the same units is performed) when evaluating multiple aqueous humor samples from the same individual (e.g., aqueous humor samples taken at different points in time from the same individual). This is because the units cancel when calculating a fold-change (i.e., determining a ratio) in the expression level from one aqueous humor sample to the next (e.g., aqueous humor samples taken at different points in time from the same individual).

For measuring RNA levels, the amount or level of an RNA in the sample is determined, e.g., the level of an mRNA. In some instances, the expression level of one or more additional RNAs may also be measured, and the level of biomarker expression compared to the level of the one or more additional RNAs to provide a normalized value for the biomarker expression level. Any convenient protocol for evaluating RNA levels may be employed wherein the level of one or more RNAs in the assayed sample is determined.

For measuring protein levels, the amount or level of a protein in the aqueous humor sample is determined. In some cases, the protein comprises a post-translational modification (e.g., phosphorylation, glycosylation) associated with regulation of activity of the protein such as by a signaling cascade, wherein the modified protein is the biomarker, and the amount of the modified protein is therefore measured. In some embodiments, an extracellular protein level is measured. For example, in some cases, the protein (i.e., polypeptide) being measured is a secreted protein, and the concentration can be measured in aqueous humor. In some embodiments, concentration is a relative value measured by comparing the level of one protein relative to another protein. In other embodiments the concentration is an absolute measurement of weight/volume or weight/weight.

In some instances, the concentration of one or more additional proteins may also be measured, and biomarker concentration compared to the level of the one or more additional proteins to provide a normalized value for the biomarker concentration. Any convenient protocol for evaluating protein levels may be employed wherein the level of one or more proteins in the assayed sample is determined.

While a variety of different methods of assaying protein levels are known to one of ordinary skill in the art, and any convenient method may be used, two representative and convenient techniques for assaying protein levels include aptamer-based assays and antibody-based methods such as the enzyme-linked immunosorbent assay (ELISA). Aptamer-based assays use aptamers comprising single-stranded oligonucleotides that bind specifically to biomarker proteins of interest. Either high affinity RNA aptamers or DNA aptamers with specificity for a protein of interest may be used. Functional groups that mimic amino acid side-chains may be added to aptamers to confer protein-like properties to improve binding affinity to a protein of interest. Aptamers that bind specifically and with high affinity to a biomarker protein of interest can be selected from large libraries of aptamers having randomized sequences using Systematic Evolution of Ligands by EXponential enrichment (SELEX). The aptamers may be designed with unique nucleotide sequences recognizable by specific hybridization probes for capture on a hybridization array for multiplexed detection of biomarkers (see, e.g., Gold et al. (2010) Aptamer-Based Multiplexed Proteomic Technology for Biomarker Discovery. PLoS ONE 5(12):e15004; herein incorporated by reference in its entirety.

In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Following incubation, the antisera-contacted surface is washed so as to remove non-immunocomplexed material. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody. The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Alternatively, other methods for measuring the levels of one or more proteins in a sample may be employed. Representative exemplary methods include but are not limited to antibody-based methods (e.g., immunofluorescence assay, radioimmunoassay, immunoprecipitation, Western blotting, proteomic arrays, xMAP microsphere technology (e.g., Luminex technology), immunohistochemistry, flow cytometry, and the like) as well as non-antibody-based methods (e.g., mass spectrometry or tandem mass spectrometry).

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

### Additional Terms

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with Parkinson's disease or a vitreoretinal disease) as well as those in which prevention is desired, those with a genetic predisposition to developing Parkinson's disease or a vitreoretinal disease, those with increased susceptibility to Parkinson's disease or a vitreoretinal disease, those suspected of having Parkinson's disease or a vitreoretinal disease, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

The term “about,” particularly in reference to a given quantity, is meant to encompass deviations of plus or minus five percent.

The terms “individual”, “subject”, and “patient” are used interchangeably herein and refer to any mammalian subject for whom diagnosis, treatment, or therapy is desired, particularly humans. Mammals include human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows. In some cases, the methods of the invention find use in experimental animals, in veterinary application, and in the development of animal models for disease, including, but not limited to, rodents including mice, rats, and hamsters; primates, and transgenic animals.

“Substantially purified” generally refers to isolation of a component such as a substance (compound, drug, inhibitor, metabolite, nucleic acid, polynucleotide, protein, or polypeptide) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography, gel filtration, and sedimentation according to density.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer. Both full-length proteins and fragments thereof are encompassed by the definition. The terms also include postexpression modifications of the polypeptide, for example, phosphorylation, glycosylation, acetylation, hydroxylation, oxidation, and the like.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), and any other type of polynucleotide which is an N- or C-glycoside of a purine or pyrimidine base. There is no intended distinction in length between the terms “polynucleotide,” “oligonucleotide,” “nucleic acid” and “nucleic acid molecule,” and these terms are used interchangeably.

By “isolated” is meant, when referring to a protein, polypeptide, or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The term “antibody” encompasses monoclonal antibodies, polyclonal antibodies, as well as hybrid antibodies, altered antibodies, chimeric antibodies, and humanized antibodies. The term antibody includes: hybrid (chimeric) antibody molecules (see, for example, Winter et al. (1991) Nature 349:293-299; and U.S. Pat. No. 4,816,567); bispecific antibodies, bispecific T cell engager antibodies (BiTE), trispecific antibodies, and other multispecific antibodies (see, e.g., Fan et al. (2015) J. Hematol. Oncol. 8:130, Krishnamurthy et al. (2018) Pharmacol Ther. 185:122-134), F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

The phrase “specifically (or selectively) binds” with reference to binding of an antibody to an antigen (e.g., biomarker) refers to a binding reaction that is determinative of the presence of the antigen in a heterogeneous population of proteins and other biologics. Thus, under designated immunoassay conditions, the specified antibodies bind to a particular antigen at least two times over the background and do not substantially bind in a significant amount to other antigens present in the sample. Specific binding to an antigen under such conditions may require an antibody that is selected for its specificity for a particular antigen. For example, antibodies raised to an antigen from specific species such as rat, mouse, or human can be selected to obtain only those antibodies that are specifically immunoreactive with the antigen and not with other proteins, except for polymorphic variants and alleles. This selection may be achieved by subtracting out antibodies that cross-react with molecules from other species. A variety of immunoassay formats may be used to select antibodies specifically immunoreactive with a particular antigen. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background.

“Providing an analysis” is used herein to refer to the delivery of an oral or written analysis (i.e., a document, a report, etc.). A written analysis can be a printed or electronic document. A suitable analysis (e.g., an oral or written report) provides any or all of the following information: identifying information of the subject (name, age, etc.), a description of what type of aqueous humor sample(s) was used and/or how it was used, the technique used to assay the sample, the results of the assay (e.g., the level of the biomarker as measured, and/or the fold-change of a biomarker level over time, or in a post-treatment assay compared to a pre-treatment assay), the assessment as to whether the individual is determined to have Parkinson's disease or a vitreoretinal disease, the predicted biological age of the individual and risk of age-related pathology and morbidity, a recommendation for additional screening for pathology, a recommendation for treatment, and/or to continue or alter therapy, a recommended strategy for additional therapy, etc. The report can be in any format including, but not limited to printed information on a suitable medium or substrate (e.g., paper); or electronic format. If in electronic format, the report can be in any computer readable medium, e.g., diskette, compact disk (CD), flash drive, and the like, on which the information has been recorded. In addition, the report may be present as a website address which may be used via the internet to access the information at a remote site.

### Biomarkers and Diagnostic Methods

Biomarkers that can be used in the practice of the subject methods include, without limitation, biomarkers for diagnosing retinitis pigmentosa such as retinoschisin 1 (RS1), S-antigen visual arrestin (SAG), serine peptidase inhibitor Kazal type 4 (SPINK4), fucosyltransferase 3 (Lewis blood group) (FUT3), growth differentiation factor 10 (GDF10), leucine rich repeat, Ig-like and transmembrane domains 2 (LRIT2), Fas apoptotic inhibitory molecule (FAIM), cadherin related family member 1 (CDHR1), recoverin (RCVRN), GSK3B interacting protein (GSKIP), eyes shut homolog (EYS), natriuretic peptide C (NPPC), hes family bHLH transcription factor 6 (HES6), fibroblast growth factor 23 (FGF23), adaptor related protein complex 4 subunit mu 1 (AP4M1), guanylate cyclase activator 1A (GUCA1A), spermatogenesis associated 33 (SPATA33), metallophosphoesterase domain containing 2 (MPPED2), acid sensing ion channel subunit family member 4 (ASIC4), junctophilin 4 (JPH4), copine 7 (CPNE7), teneurin transmembrane protein 4 (TENM4), glutamate metabotropic receptor 4 (GRM4), tankyrase (TNKS), glutamate decarboxylase 1 (GAD1), synaptic vesicle glycoprotein 2A (SV2A), synaptotagmin 5 (SYT5), RIC3 acetylcholine receptor chaperone (RIC3), calcium/calmodulin dependent protein kinase II alpha (CAMK2A), leucine rich repeat transmembrane neuronal (4LRRTM4), potassium voltage-gated channel modifier subfamily G member 4 (KCNG4), dipeptidyl peptidase like 10 (DPP10), protein phosphatase 1 regulatory subunit 27 (PPP1R27), regulator of G protein signaling 5 (RGS5), GRB2 related adaptor protein (GRAP), receptor activity modifying protein 3 (RAMP3), mesenchyme homeobox 1 (MEOX1), actin filament associated protein 1 like 1 (AFAP1L1), opioid binding protein/cell adhesion molecule like (OPCML), leucine rich repeat, Ig-like and transmembrane domains 3 (LRIT3), phospholipase A2 group V (PLA2G5), polypeptide N-acetylgalactosaminyltransferase 11 (GALNT11), and potassium channel tetramerization domain containing 4 (KCTD4); biomarkers for diagnosing diabetic retinopathy such as lipopolysaccharide binding protein (LBP), C-reactive protein (CRP), inter-alpha-trypsin inhibitor heavy chain 3 (ITIH3), apolipoprotein A1 (APOA1), carboxypeptidase N subunit 2 (CPN2), complement component 4 binding protein alpha (C4BPA), hyaluronan binding protein 2 (HABP2), coagulation factor XIII B chain (F13B), serpin family A member 10 (SERPINA10), insulin like growth factor binding protein 1 (IGFBP1), fibronectin 1 (FN1), matrix metallopeptidase 19 (MMP19), angiopoietin 2 (ANGPT2), roundabout guidance receptor 4 (ROBO4), vascular endothelial growth factor D (VEGFD), angiopoietin like 4 (ANGPTL4), fms related receptor tyrosine kinase 4 (FLT4), filamin A (FLNA), EPH receptor A1 (EPHA1), endoglin (ENG), thrombospondin 1 (THBS1), chloride intracellular channel 4 (CLIC4), neuropilin 2 (NRP2), vascular endothelial growth factor A (VEGFA), tryptophanyl-tRNA synthetase 1 (WARS1), C-X-C motif chemokine ligand 8 (CXCL8), thymidine phosphorylase (TYMP), C-C motif chemokine ligand 2 (CCL2), mitogen-activated protein kinase 14 (MAPK14), and AKT serine/threonine kinase 1 (AKT1); biomarkers for distinguishing between a diagnosis of non-proliferative diabetic retinopathy and proliferative diabetic retinopathy such as fibronectin 1 (FN1), matrix metallopeptidase 19 (MMP19), angiopoietin 2 (ANGPT2), roundabout guidance receptor 4 (ROBO4), vascular endothelial growth factor D (VEGFD), angiopoietin like 4 (ANGPTL4), fms related receptor tyrosine kinase 4 (FLT4), filamin A (FLNA), EPH receptor A1 (EPHA1), and endoglin (ENG); biomarkers for diagnosing Parkinson's disease such as trio Rho guanine nucleotide exchange factor (TRIO), junctophilin 4 (JPH4), copine 7 (CPNE7), glutamate metabotropic receptor 4 (GRM4), tankyrase (TNKS), serine active site containing 1 (SERAC1), activin A receptor type 2B (ACVR2B), synaptic vesicle glycoprotein 2A (SV2A), RIC3 acetylcholine receptor chaperone (RIC3), calcium/calmodulin dependent protein kinase II alpha (CAMK2A), BCL2 like 2 (BCL2L2), dysbindin domain containing 1 (DBNDD1), sulfotransferase family 4A member 1 (SULT4A1), hyaluronan binding protein 4 (HABP4), syntaphilin (SNPH), Rac family small GTPase 3 (RAC3), synaptotagmin 13 (SYT13), p21 (RAC1) activated kinase 5 (PAK5), neurotrophic receptor tyrosine kinase 1 (NTRK1), complement C1q like 4 (C1QL4), potassium voltage-gated channel modifier subfamily G member 4 (KCNG4), dipeptidyl peptidase like 10 (DPP10), protein phosphatase 1 regulatory subunit 27 (PPP1R27), PIH1 domain containing 2 (PIH1D2), cadherin related family member 1 (CDHR1), chromosome 17 open reading frame 67 (C17orf67), GSK3B interacting protein (GSKIP), centrosomal protein 112 (CEP112), hes family bHLH transcription factor 6 (HES6), fibroblast growth factor 23 (FGF23), adaptor related protein complex 4 subunit mu 1 (AP4M1), spermatogenesis associated 33 (SPATA33), MPPED2 (MPPED2), cholinergic receptor nicotinic alpha 5 subunit (CHRNA5), and acid sensing ion channel subunit family member 4 (ASIC4); biomarkers for diagnosing uveitis such as immunoglobulin heavy constant mu (IGHM), thioredoxin domain containing 5 (TXNDC5), SLAM family member 7 (SLAMF7), marginal zone B and B1 cell specific protein (MZB1), immunoglobulin heavy constant delta (IGHD), complement C3d receptor 2 (CR2), immunoglobulin heavy constant gamma 4 (IGHG4), lymphocyte antigen 9 (LY9), Fc receptor like 5 (FCRL5), TNF receptor superfamily member 13B (TNFRSF13B), matrix metallopeptidase 9 (MMP9), interleukin 1 receptor type 2 (IL1R2), GLI pathogenesis related 2 (GLIPR2), myeloperoxidase (MPO), resistin (RETN), CD177 molecule (CD177), proline-serine-threonine phosphatase interacting protein 1 (PSTPIP1), C-type lectin domain family 12 member A (CLEC12A), high mobility group box 2 (HMGB2), leukotriene A4 hydrolase (LTA4H), von Willebrand factor (VWF), tyrosine kinase with immunoglobulin like and EGF like domains 1 (TIE1), selectin E (SELE), cadherin 5 (CDH5), angiopoietin 2 (ANGPT2), fms related receptor tyrosine kinase 4 (FLT4), adhesion G protein-coupled receptor F5 (ADGRF5), vascular endothelial growth factor C (VEGFC), TEK receptor tyrosine kinase (TEK), MYC target 1 (MYCT1), C-X-C motif chemokine ligand 10 (CXCL10), interleukin 18 binding protein (IL18BP), C-C motif chemokine ligand 22 (CCL22), TNF receptor superfamily member 8 (TNFRSF8), C-C motif chemokine ligand 7 (CCL7), matrix metallopeptidase 12 (MMP12), interleukin 10 (IL10), C-X-C motif chemokine ligand 11 (CXCL11), chymotrypsin like elastase 1 (CELA1), granulysin (GNLY), cystatin F (CST7), CD8 subunit alpha (CD8A), CD5 molecule (CD5), granzyme K (GZMK), granzyme A (GZMA), C-C motif chemokine ligand 5 (CCL5), CD7 molecule (CD7), LCK proto-oncogene, Src family tyrosine kinase (LCK), and SH2 domain containing 1A (SH2D1A); biomarkers for determining biological age of an eye such as leukocyte cell derived chemotaxin 2 (LECT2), discoidin, CUB and LCCL domain containing 1 (DCBLD1), secretagogin, EF-hand calcium binding protein (SCGN), aggrecan (ACAN), amine oxidase copper containing 2 (AOC2), calcyphosine (CAPS), trefoil factor 3 (TFF3), amnion associated transmembrane protein (AMN), ABO, alpha 1-3-N-acetylgalactosaminyltransferase and alpha 1-3-galactosyltransferase (ABO), neuropilin and tolloid like 1 (NETO1), CD274 molecule (CD274), pro-platelet basic protein (PPBP), ABL proto-oncogene 1, non-receptor tyrosine kinase (ABL1), hepcidin antimicrobial peptide (HAMP), insulin like growth factor binding protein 1 (IGFBP1), adipogenesis associated Mth938 domain containing (AAMDC), alpha-1-B glycoprotein (A1 BG), adenosine deaminase 2 (ADA2), major vault protein (MVP), crystallin gamma C (CRYGC), alpha 1,4-galactosyltransferase (P1 PK blood group) (A4GALT), alpha-1,4-N-acetylglucosaminyltransferase (A4GNT), aminoadipate aminotransferase (AADAT), alpha-2-macroglobulin (A2M), and ABI family member 3 (ABI3); and biomarkers for estimating age of a subject such as leukocyte cell derived chemotaxin 2 (LECT2), discoidin, CUB and LCCL domain containing 1 (DCBLD1), secretagogin, EF-hand calcium binding protein (SCGN), aggrecan (ACAN), amine oxidase copper containing 2 (AOC2), calcyphosine (CAPS), trefoil factor 3 (TFF3), amnion associated transmembrane protein (AMN), ABO, alpha 1-3-N-acetylgalactosaminyltransferase and alpha 1-3-galactosyltransferase (ABO), neuropilin and tolloid like 1 (NETO1), CD274 molecule (CD274), pro-platelet basic protein (PPBP), ABL proto-oncogene 1, non-receptor tyrosine kinase (ABL1), hepcidin antimicrobial peptide (HAMP), insulin like growth factor binding protein 1 (IGFBP1), adipogenesis associated Mth938 domain containing (AAMDC), alpha-1-B glycoprotein (A1BG), adenosine deaminase 2 (ADA2), major vault protein (MVP), crystallin gamma C (CRYGC), alpha 1,4-galactosyltransferase (P1 PK blood group) (A4GALT), alpha-1,4-N-acetylglucosaminyltransferase (A4GNT), aminoadipate aminotransferase (AADAT), alpha-2-macroglobulin (A2M), and ABI family member 3 (ABI3).

In certain embodiments, a panel of biomarkers is provided. Biomarker panels of any size can be used in the practice of the subject methods. Biomarker panels typically comprise at least 3 biomarkers and up to 50 biomarkers, including any number of biomarkers in between, such as 3, 4, 5, 6, 7, 8, 9, 10, 11, 12, 13, 14, 15, 16, 17, 18, 19, 20, 25, 30, 35, 40, 45, or 50 biomarkers. In certain embodiments, a biomarker panel comprising at least 3, or at least 4, or at least 5, or at least 6, or at least 7, or at least 8, or at least 9, or at least 10, or at least 11, or at least 12, or at least 13, or at least 14, or at least 5, or at least 16, or at least 17, or at least 18, or at least 19, or at least 20, or more biomarkers. Although smaller biomarker panels are usually more economical, larger biomarker panels (i.e., greater than 20 biomarkers) have the advantage of providing more detailed information and can also be used in the practice of the subject methods.

In some embodiments, a biomarker panel for diagnosing retinitis pigmentosa comprises or consists of at least two, at least three, or at least four biomarkers selected from RS1, SAG, SPINK4, FUT3, GDF10, LRIT2, FAIM, CDHR1, RCVRN, GSKIP, EYS, NPPC, HES6, FGF23, APAM1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LPIT3, PLA2G5, GALNT11, and KCTD4. In certain embodiments, the biomarker panel comprises or consists of RS1, SAG, SPINK4, FUT3, GDF10, LRIT2, FAIM, CDHR1, RCVRN, GSKIP, EYS, NPPC, HES6, FGF23, APAM1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LPIT3, PLA2G5, GALNT11, and KCTD4. In certain embodiments, the biomarker panel comprises or consists of RS1, SAG, SPINK4, FUT3, GDF10, LRIT2, FAIM, CDHR1, RCVRN, and GSKIP. In certain embodiments, the biomarker panel comprises or consists of EYS, NPPC, HES6, FGF23, APAM1, GUCA1A, SPATA33, MPPED2, and ASIC4. In certain embodiments, the biomarker panel comprises or consists of JPH4, CPNE7, TENM4, GRM4, TNKS, GAD1, SV2A, SYT5, RIC3, and CAMK2A. In certain embodiments, the biomarker panel comprises or consists of LRRTM4, KCNG4, DPP10, and PPP1R27. In certain embodiments, the biomarker panel comprises or consists of RGS5, GRAP, RAMP3, and MEOX1, AFAP1 L1. In certain embodiments, the biomarker panel comprises or consists of OPCML, LPIT3, PLA2G5, GALNT11, and KCTD4.

In some embodiments, a biomarker panel for diagnosing diabetic retinopathy comprises or consists of at least two, at least three, or at least four biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1. In certain embodiments, the biomarker panel comprises or consists of LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT.

In some embodiments, a biomarker panel for distinguishing between a diagnosis of non-proliferative diabetic retinopathy and proliferative diabetic retinopathy comprises or consists of at least two, at least three, or at least four biomarkers selected from FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1. In some embodiments, a biomarker panel for diagnosing non-proliferative diabetic retinopathy comprises or consists of FN1, MMP19, ANGPT2, ROBO4, VEGFD, ANGPTL4, FLT4, FLNA, EPHA1, and ENG. In some embodiments, a biomarker panel for diagnosing proliferative diabetic retinopathy comprises or consists of THBS1, CLIC4, NRP2, VEGFA, WARS1, CXCL8, TYMP, CCL2, MAPK14, and AKT1.

In some embodiments, a biomarker panel for diagnosing Parkinson's disease comprises or consists of at least two, at least three, or at least four biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, APAM1, SPATA33, MPPED2, CHRNA5, and ASIC4. In some embodiments, a biomarker panel for diagnosing Parkinson's disease comprises or consists of TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, APAM1, SPATA33, MPPED2, CHRNA5, and ASIC4. In some embodiments, a biomarker panel for diagnosing Parkinson's disease comprises or consists of TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, APAM1, SPATA33, MPPED2, CHRNA5, and ASIC4. In some embodiments, a biomarker panel for diagnosing Parkinson's disease comprises or consists of TRIO, JPH4, CPNE7, GRM4, TNKS, SERAC1, ACVR2B, SV2A, RIC3, and CAMK2A. In some embodiments, a biomarker panel for diagnosing Parkinson's disease comprises or consists of BCL2L2, DBNDD1, SULT4A1, HABP4, SNPH, RAC3, and SYT13. In some embodiments, a biomarker panel for diagnosing Parkinson's disease comprises or consists of PAK5, NTRK1, and C1QL4. In some embodiments, a biomarker panel for diagnosing Parkinson's disease comprises or consists of KCNG4, DPP10, and PPP1R27. In some embodiments, a biomarker panel for diagnosing Parkinson's disease comprises or consists of PIH1 D2, CDHR1, C17orf67, GSKIP, and CEP112 are measured in the aqueous humor sample. In some embodiments, a biomarker panel for diagnosing Parkinson's disease comprises or consists of HES6, FGF23, APAM1, SPATA33, MPPED2, CHRNA5, and ASIC4.

In some embodiments, a biomarker panel for diagnosing uveitis comprises or consists of at least two, at least three, or at least four biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A. In some embodiments, a biomarker panel for diagnosing uveitis comprises or consists of IGHM, TXNDC5, SLAMF7, MZB1, IGHD, CR2, IGHG4, LY9, FCRL5, TNFRSF13B, MMP9, IL1R2, GLIPR2, MPO, RETIN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A. In some embodiments, a biomarker panel for diagnosing uveitis comprises or consists of IGHM, TXNDC5, SLAMF7, MZB1, IGHD, CR2, IGHG4, LY9, FCRL5, TNFRSF13B. In some embodiments, a biomarker panel for diagnosing uveitis comprises or consists of MMP9, IL1R2, GLIPR2, MPO, RETIN, CD177, PSTPIP1, CLEC12A, HMGB2, LTA4H. In some embodiments, a biomarker panel for diagnosing uveitis comprises or consists of VWF, TIE1, SELE, CDH5, ANGPT2, FLT4, ADGRF5, VEGFC, TEK, MYCT1. In some embodiments, a biomarker panel for diagnosing uveitis comprises or consists of CXCL10, IL18BP, CCL22, TNFRSF8, CCL7, MMP12, IL10, CXCL11, CELA1. In some embodiments, a biomarker panel for diagnosing uveitis comprises or consists of GNLY, CST7, CD8A, CD5, GZMK, GZMA, CCL5, CD7, LCK, SH2D1A are measured in the aqueous humor sample.

In some embodiments, a biomarker panel for diagnosing damage to cone cells comprises or consists of at least two, at least three, or at least four biomarkers selected from BICDL1, NPPC, HES6, FGF23, APAM1, SPATA33, MPPED2, CHRNA5, and GPC2. In some embodiments, a biomarker panel for diagnosing damage to cone cells comprises or consists of BICDL1, NPPC, HES6, FGF23, APAM1, SPATA33, MPPED2, CHRNA5, and GPC2.

In some embodiments, a biomarker panel for diagnosing damage to retinal pigment epithelium (RPE) cells. comprises or consists of at least two, at least three, or at least four biomarkers selected from OPCML, GAP43, RIPPLY3, LRIT3, PLA2G5, FGF18, PLD5, and KCTD4. In some embodiments, a biomarker panel for diagnosing damage to RPE comprises or consists of OPCML, GAP43, RIPPLY3, LRIT3, PLA2G5, FGF18, PLD5, and KCTD4.

In some embodiments, a biomarker panel for diagnosing damage to amacrine cells comprises or consists of at least two, at least three, or at least four biomarkers selected from CPNE7, TYRO3, TENM4, MDM4, RDH13, SV2A, SYT5, OLFM3, RIC3, and SEMA6C. In some embodiments, a biomarker panel for diagnosing damage to amacrine cells comprises or consists of CPNE7, TYRO3, TENM4, MDM4, RDH13, SV2A, SYT5, OLFM3, RIC3, and SEMA6C.

In some embodiments, a biomarker panel for determining biological age of an eye comprises or consists of at least two, at least three, or at least four biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3.

In some embodiments, a biomarker panel for estimating age of a subject comprises or consists of at least two, at least three, or at least four biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3.

An aqueous humor sample comprising the expressed biomarkers is obtained from the subject. The sample is taken from the aqueous humor (i.e., ocular fluid secreted from the ciliary body found in the anterior and posterior chambers of the eye). A “control” sample, as used herein, refers to an aqueous humor sample obtained from a normal or healthy subject (i.e., subject not having a vitreoretinal disease or Parkinson's disease, or younger healthy subject not at risk of age-related pathology and morbidity). An aqueous humor sample can be obtained from a subject by conventional techniques. For example, aqueous humor samples can be obtained by any suitable method such as liquid biopsy or surgically according to methods well known in the art.

When analyzing the levels of biomarkers in an aqueous humor sample from a subject, the reference value ranges used for comparison can represent the levels of one or more biomarkers in an aqueous humor sample from one or more subjects without disease (i.e., normal or healthy control). In some embodiments, the reference value ranges used for comparison can represent the levels of one or more biomarkers in aqueous humor samples from one or more subjects at certain biological ages to allow the biological age of an individual to be determined. Alternatively, the reference values can represent the levels of one or more biomarkers from one or more subjects with a disease (e.g., vitreoretinal disease or melanoma), wherein similarity to the reference value ranges indicates the subject has the disease. More specifically, the reference value ranges can represent the levels of one or more biomarkers from one or more subjects with retinitis pigmentosa (a “retinitis pigmentosa” biomarker expression profile), diabetic retinopathy (a “diabetic retinopathy” biomarker expression profile), non-proliferative diabetic retinopathy (a “non-proliferative diabetic retinopathy” biomarker expression profile), proliferative diabetic retinopathy (a “proliferative diabetic retinopathy” biomarker expression profile), uveitis (a “uveitis” biomarker expression profile), Parkinson's disease (a “Parkinson's disease” biomarker expression profile), or an eye biological age (an “eye biological age” biomarker expression profile).

Accordingly, in one aspect, a method of diagnosing retinitis pigmentosa in a patient is provided, the method comprising: measuring levels of one or more biomarkers selected from RS1, SAG, SPINK4, FUT3, GDF10, LRIT2, FAIM, CDHR1, RCVRN, GSKIP, EYS, NPPC, HES6, FGF23, APAM1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LPIT3, PLA2G5, GALNT11, and KCTD4 in the aqueous humor sample, wherein decreased levels of the one or more biomarkers selected from RS1, SAG, SPINK4, FUT3, GDF10, LRIT2, FAIM, CDHR1, RCVRN, GSKIP, EYS, NPPC, HES6, FGF23, APAM1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LPIT3, PLA2G5, GALNT11, and KCTD4 compared to reference value ranges for the levels of the one or more biomarkers in a control aqueous humor sample indicate that the patient has retinitis pigmentosa. In certain embodiments, the levels of at least two, at least three, or at least four biomarkers selected from RS1, SAG, SPINK4, FUT3, GDF10, LRIT2, FAIM, CDHR1, RCVRN, GSKIP, EYS, NPPC, HES6, FGF23, APAM1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LPIT3, PLA2G5, GALNT11, and KCTD4 are measured in the aqueous humor sample. In certain embodiments, the levels of RS1, SAG, SPINK4, FUT3, GDF10, LRIT2, FAIM, CDHR1, RCVRN, GSKIP, EYS, NPPC, HES6, FGF23, APAM1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LPIT3, PLA2G5, GALNT11, and KCTD4 are measured in the aqueous humor sample. In certain embodiments, the levels of RS1, SAG, SPINK4, FUT3, GDF10, LRIT2, FAIM, CDHR1, RCVRN, and GSKIP are measured in the aqueous humor sample. In certain embodiments, the EYS, NPPC, HES6, FGF23, APAM1, GUCA1A, SPATA33, MPPED2, and ASIC4 are measured in the aqueous humor sample. In certain embodiments, the levels of JPH4, CPNE7, TENM4, GRM4, TNKS, GAD1, SV2A, SYT5, RIC3, and CAMK2A are measured in the aqueous humor sample. In certain embodiments, the levels of LRRTM4, KCNG4, DPP10, and PPP1R27 are measured in the aqueous humor sample. In certain embodiments, the levels of RGS5, GRAP, RAMP3, and MEOX1, AFAP1L1 are measured in the aqueous humor sample. In certain embodiments, the levels of OPCML, LPIT3, PLA2G5, GALNT11, and KCTD4 are measured in the aqueous humor sample.

The method may further comprise determining an appropriate treatment regimen for a patient and, in particular, whether a patient should be treated for retinitis pigmentosa. For example, a patient is selected for treatment for retinitis pigmentosa if the patient has a positive diagnosis for retinitis pigmentosa based on a biomarker expression profile, as described herein. The treatment for retinitis pigmentosa may comprise, for example, retinal sheet transplantation, RPE65 gene therapy, implanting a retinal prosthesis, or administering vitamin A, docosahexaenoic acid (DHA), N-acetylcysteine (NAC), and lutein, or a combination thereof.

In some embodiments, the methods described herein are used for monitoring retinitis pigmentosa in a subject. For example, a first aqueous humor sample can be obtained from the patient at a first time point and one or more aqueous humor samples can be obtained from the subject at later time points and levels of the biomarkers can be compared at different time points. In one embodiment, retinitis pigmentosa is monitored in a patient by a method comprising: obtaining a first aqueous humor sample from an eye of the patient at a first time point and a second aqueous humor sample from the eye of the patient later at a second time point; and measuring levels of one or more biomarkers in the first aqueous humor sample and the second aqueous humor sample, wherein the biomarkers are selected from RS1, SAG, SPINK4, FUT3, GDF10, LRIT2, FAIM, CDHR1, RCVRN, GSKIP, EYS, NPPC, HES6, FGF23, APAM1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LPIT3, PLA2G5, GALNT11, and KCTD4 in the aqueous humor sample, wherein detection of decreased levels of the one or more biomarkers selected from RS1, SAG, SPINK4, FUT3, GDF10, LRIT2, FAIM, CDHR1, RCVRN, GSKIP, EYS, NPPC, HES6, FGF23, APAM1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LPIT3, PLA2G5, GALNT11, and KCTD4 in the second aqueous humor sample compared to the first aqueous humor sample indicate that the patient is worsening, and wherein detection of increased levels of the one or more biomarkers selected from RS1, SAG, SPINK4, FUT3, GDF10, LRIT2, FAIM, CDHR1, RCVRN, GSKIP, EYS, NPPC, HES6, FGF23, APAM1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LPIT3, PLA2G5, GALNT11, and KCTD4 in the second aqueous humor sample compared to the first aqueous humor sample indicate that the patient is improving.

The subject methods may also be used for assaying pre-treatment and post-treatment aqueous humor samples obtained from an individual to determine whether the individual is responsive or not responsive to a treatment. For example, a first aqueous humor sample can be obtained from a subject before the subject undergoes the therapy, and a second aqueous humor sample can be obtained from the subject after the subject undergoes the therapy. In one embodiment, the efficacy of a treatment of a patient for retinitis pigmentosa is monitored by obtaining a first aqueous humor sample from the patient before the patient undergoes the treatment and a second aqueous humor sample from the patient after the patient undergoes the treatment; measuring levels of one or more biomarkers in the first aqueous humor sample and the second aqueous humor sample, wherein the one or more biomarkers are selected from RS1, SAG, SPINK4, FUT3, GDF10, LRIT2, FAIM, CDHR1, RCVRN, GSKIP, EYS, NPPC, HES6, FGF23, APAM1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LPIT3, PLA2G5, GALNT11, and KCTD4; and evaluating the efficacy of the treatment, wherein detection of decreased levels of the one or more biomarkers selected from RS1, SAG, SPINK4, FUT3, GDF10, LRIT2, FAIM, CDHR1, RCVRN, GSKIP, EYS, NPPC, HES6, FGF23, APAM1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LPIT3, PLA2G5, GALNT11, and KCTD4 in the second aqueous humor sample compared to the first aqueous humor sample indicate that the patient is worsening or not responding to the treatment, and detection of increased levels of the one or more biomarkers selected from RS1, SAG, SPINK4, FUT3, GDF10, LRIT2, FAIM, CDHR1, RCVRN, GSKIP, EYS, NPPC, HES6, FGF23, APAM1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LPIT3, PLA2G5, GALNT11, and KCTD4 in the second aqueous humor sample compared to the first aqueous humor sample indicate that the patient is improving. In certain embodiments, the method further comprises altering the treatment if the patient is worsening or not responding to the treatment.

In another aspect, a method of diagnosing diabetic retinopathy in a patient is provided, the method comprising: obtaining an aqueous humor sample from an eye of the patient; measuring levels of one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 in the aqueous humor sample, wherein increased levels of the one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 compared to reference value ranges for the levels of the one or more biomarkers in a control aqueous humor sample indicate that the patient has diabetic retinopathy; and treating the patient for the diabetic retinopathy if the patient has a positive diagnosis for diabetic retinopathy. In certain embodiments, the levels of at least two, at least three, or at least four biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 are measured in the aqueous humor sample. In certain embodiments, the levels of LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 are measured in the aqueous humor sample.

The method may further comprise determining an appropriate treatment regimen for a patient and, in particular, whether a patient should be treated for diabetic retinopathy. For example, a patient is selected for treatment for diabetic retinopathy if the patient has a positive diagnosis for diabetic retinopathy based on a biomarker expression profile, as described herein. The treatment for diabetic retinopathy may comprise, for example, administering an anti-vascular endothelial growth factor (VEGF) agent or a steroid, or performing panretinal laser photocoagulation or a vitrectomy, or a combination thereof. In some embodiments, the anti-VEGF agent is bevacizumab, ranibizumab, sunitinib, sorafenib, axitinib, aflibercept, brolucizuma, faricimab, or pazopanib. In some embodiments, the steroid is triamcinolone acetonide, fluocinolone acetonide, or dexamethasone.

In certain embodiments, the levels of FN1, MMP19, ANGPT2, ROBO4, VEGFD, ANGPTL4, FLT4, FLNA, EPHA1, and ENG are measured in the aqueous humor sample, wherein increased levels of the one or more biomarkers selected from FN1, MMP19, ANGPT2, ROBO4, VEGFD, ANGPTL4, FLT4, FLNA, EPHA1, and ENG compared to reference value ranges for the levels of the one or more biomarkers in a control aqueous humor sample indicate that the patient has non-proliferative diabetic retinopathy. In certain embodiments, the method further comprises treating the patient for the non-proliferative diabetic retinopathy if the patient has a positive diagnosis for non-proliferative diabetic retinopathy. The treatment for non-proliferative diabetic retinopathy comprises administering an anti-VEGF agent or a steroid, performing vitrectomy or photocoagulation, or increasing frequency of retinal exams or a combination thereof.

In certain embodiments, the levels of THBS1, CLIC4, NRP2, VEGFA, WARS1, CXCL8, TYMP, CCL2, MAPK14, AKT1 are measured in the aqueous humor sample, wherein increased levels of the one or more biomarkers selected from THBS1, CLIC4, NRP2, VEGFA, WARS1, CXCL8, TYMP, CCL2, MAPK14, AKT1 compared to reference value ranges for the levels of the one or more biomarkers in a control aqueous humor sample indicate that the patient has proliferative diabetic retinopathy. In certain embodiments, the method further comprises treating the patient for the proliferative diabetic retinopathy if the patient has a positive diagnosis for proliferative diabetic retinopathy. The treatment for proliferative diabetic retinopathy may comprise administering an anti-VEGF agent or performing panretinal laser photocoagulation, or a combination thereof.

In another aspect, a method of monitoring diabetic retinopathy in a patient is provided, the method comprising: obtaining a first aqueous humor sample from an eye of the patient at a first time point and a second aqueous humor sample from the eye of the patient later at a second time point; and measuring levels of one or more biomarkers in the first aqueous humor sample and the second aqueous humor sample, wherein the biomarkers are selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1, wherein detection of increased levels of the one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 in the second aqueous humor sample compared to the first aqueous humor sample indicate that the patient is worsening, and wherein detection of decreased levels of the one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 in the second aqueous humor sample compared to the first aqueous humor sample indicate that the patient is improving.

In another aspect, a method of monitoring efficacy of a treatment of a patient for diabetic retinopathy is provided, the method comprising: obtaining a first aqueous humor sample from the patient before the patient undergoes the treatment and a second aqueous humor sample from the patient after the patient undergoes the treatment; measuring levels of one or more biomarkers in the first aqueous humor sample and the second aqueous humor sample, wherein the one or more biomarkers are selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1; and evaluating the efficacy of the treatment, wherein detection of increased levels of the one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 in the second aqueous humor sample compared to the first aqueous humor sample indicate that the patient is worsening or not responding to the treatment, and detection of decreased levels of the one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 in the second aqueous humor sample compared to the first aqueous humor sample indicate that the patient is improving. In some embodiments, the method further comprises altering the treatment if the patient is worsening or not responding to the treatment.

In another aspect, a method of monitoring a patient having non-proliferative diabetic retinopathy for progression to proliferative diabetic retinopathy is provided, the method comprising: obtaining an aqueous humor sample from an eye of the patient; measuring levels of one or more biomarkers selected from THBS1, CLIC4, NRP2, VEGFA, WARS1, CXCL8, TYMP, CCL2, MAPK14, and AKT1 in the aqueous humor sample, wherein increased levels of the one or more biomarkers selected from THBS1, CLIC4, NRP2, VEGFA, WARS1, CXCL8, TYMP, CCL2, MAPK14, and AKT1 compared to reference value ranges for the levels of the one or more biomarkers in a control aqueous humor sample indicate that the patient has proliferative diabetic retinopathy. In certain embodiments, the method further comprises treating the patient for the proliferative diabetic retinopathy if the patient has a positive diagnosis for proliferative diabetic retinopathy. In certain embodiments, treating the patient for the proliferative diabetic retinopathy comprises administering an anti-VEGF agent or performing panretinal laser photocoagulation, or a combination thereof.

In another aspect, a method of distinguishing between a diagnosis of non-proliferative diabetic retinopathy and proliferative diabetic retinopathy for a patient is provided, the method comprising: obtaining an aqueous humor sample from an eye of the patient; measuring levels of one or more biomarkers selected from FN1, MMP19, ANGPT2, ROBO4, VEGFD, ANGPTL4, FLT4, FLNA, EPHA1, and ENG in the aqueous humor sample, wherein increased levels of the one or more biomarkers selected from FN1, MMP19, ANGPT2, ROBO4, VEGFD, ANGPTL4, FLT4, FLNA, EPHA1, and ENG compared to reference value ranges for the levels of the one or more biomarkers in a control aqueous humor sample indicate that the patient has non-proliferative diabetic retinopathy; and measuring levels of one or more biomarkers selected from THBS1, CLIC4, NRP2, VEGFA, WARS1, CXCL8, TYMP, CCL2, MAPK14, and AKT1 in the aqueous humor sample, wherein increased levels of the one or more biomarkers selected from THBS1, CLIC4, NRP2, VEGFA, WARS1, CXCL8, TYMP, CCL2, MAPK14, and AKT1 compared to reference value ranges for the levels of the one or more biomarkers in a control aqueous humor sample indicate that the patient has proliferative diabetic retinopathy. In certain embodiments, the levels of at least two, at least three, or at least four biomarkers selected from FN1, MMP19, ANGPT2, ROBO4, VEGFD, ANGPTL4, FLT4, FLNA, EPHA1, and ENG, and the levels of at least two, at least three, or at least four biomarkers selected from THBS1, CLIC4, NRP2, VEGFA, WARS1, CXCL8, TYMP, CCL2, MAPK14, and AKT1 are measured in the aqueous humor sample. In certain embodiments, the levels of FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 are measured in the aqueous humor sample. In certain embodiments, the method further comprises treating the patient for the non-proliferative diabetic retinopathy if the patient has a positive diagnosis for non-proliferative diabetic retinopathy, or treating the patient for the proliferative diabetic retinopathy if the patient has a positive diagnosis for proliferative diabetic retinopathy. In certain embodiments, treating the patient for the non-proliferative diabetic retinopathy comprises administering an anti-VEGF agent or a steroid, performing vitrectomy or photocoagulation, or increasing frequency of retinal exams or a combination thereof. In certain embodiments, treating the patient for the proliferative diabetic retinopathy comprises administering an anti-VEGF agent or performing panretinal laser photocoagulation, or a combination thereof.

In another aspect, a method of diagnosing Parkinson's disease in a patient is provided, the method comprising: obtaining an aqueous humor sample from an eye of the patient; measuring levels of one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, APAM1, SPATA33, MPPED2, CHRNA5, and ASIC4 in the aqueous humor sample, wherein decreased levels of the one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, APAM1, SPATA33, MPPED2, CHRNA5, and ASIC4 compared to reference value ranges for the levels of the one or more biomarkers in a control aqueous humor sample indicate that the patient has Parkinson's disease; and treating the patient for the Parkinson's disease if the patient has Parkinson's disease. In certain embodiments, the levels of at least two, at least three, or at least four biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, APAM1, SPATA33, MPPED2, CHRNA5, and ASIC4 are measured in the aqueous humor sample. In certain embodiments, the levels of TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, APAM1, SPATA33, MPPED2, CHRNA5, and ASIC4 are measured in the aqueous humor sample. In certain embodiments, the levels of TRIO, JPH4, CPNE7, GRM4, TNKS, SERAC1, ACVR2B, SV2A, RIC3, and CAMK2A are measured in the aqueous humor sample. In certain embodiments, the levels of BCL2L2, DBNDD1, SULT4A1, HABP4, SNPH, RAC3, and SYT13 are measured in the aqueous humor sample. In certain embodiments, the levels of PAK5, NTRK1, and C1QL4 are measured in the aqueous humor sample. In certain embodiments, the levels of KCNG4, DPP10, and PPP1R27 are measured in the aqueous humor sample. In certain embodiments, the levels of PIH1D2, CDHR1, C17orf67, GSKIP, and CEP112 are measured in the aqueous humor sample. In certain embodiments, the levels of HES6, FGF23, APAM1, SPATA33, MPPED2, CHRNA5, and ASIC4 are measured in the aqueous humor sample.

The method may further comprise determining an appropriate treatment regimen for a patient and, in particular, whether a patient should be treated for Parkinson's disease. For example, a patient is selected for treatment for Parkinson's disease if the patient has a positive diagnosis for Parkinson's disease based on a biomarker expression profile, as described herein. The treatment for Parkinson's disease may comprise, for example, administering 1-3,4-dihydroxyphenylalanine (L-DOPA), an aromatic L-amino acid decarboxylase inhibitor, a catechol-O-methyltransferase (COMT) inhibitor, a dopamine agonist, a monoamine oxidase inhibitor, an anticholinergic, or a combination thereof. In some embodiments, the aromatic L-amino acid decarboxylase inhibitor is carbidopa, benserazide, methyldopa, alpha-difluoromethyl-DOPA, 3′,4′,5,7-tetrahydroxy-8-methoxyisoflavone, epigallocatechin gallate, or epigallocatechin. In some embodiments, the COMT inhibitor is entacapone, nebicapone, nitecapone, opicapone, or tolcapone. In some embodiments, the dopamine agonist is pramipexole, ropinirole, bromocriptine, pergolide, rotigotine, apomorphine, aripiprazole, phencyclidine, quinpirole, cabergoline, ciladopa, dihydrexidine, dinapsoline, doxanthrine, lisuride, piribedil, propylnorapomorphine, quinagolide, roxindole, or sumanirole. In some embodiments, the monoamine oxidase inhibitor is rasagiline, or selegiline, safinamide. In some embodiments, the anticholinergic is procyclidine or trihexyphenidyl.

In another aspect, a method of monitoring Parkinson's disease in a patient is provided, the method comprising: obtaining a first aqueous humor sample from an eye of the patient at a first time point and a second aqueous humor sample from the eye of the patient later at a second time point; and measuring levels of one or more biomarkers in the first aqueous humor sample and the second aqueous humor sample, wherein the biomarkers are selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, APAM1, SPATA33, MPPED2, CHRNA5, and ASIC4 in the aqueous humor sample, wherein detection of decreased levels of the one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, APAM1, SPATA33, MPPED2, CHRNA5, and ASIC4 in the second aqueous humor sample compared to the first aqueous humor sample indicate that the patient is worsening, and wherein detection of increased levels of the one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, APAM1, SPATA33, MPPED2, CHRNA5, and ASIC4 in the second aqueous humor sample compared to the first aqueous humor sample indicate that the patient is improving.

In another aspect, a method of monitoring efficacy of a treatment of a patient for Parkinson's disease is provided, the method comprising: obtaining a first aqueous humor sample from the patient before the patient undergoes the treatment and a second aqueous humor sample from the patient after the patient undergoes the treatment; measuring levels of one or more biomarkers in the first aqueous humor sample and the second aqueous humor sample, wherein the one or more biomarkers are selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, APAM1, SPATA33, MPPED2, CHRNA5, and ASIC4; and evaluating the efficacy of the treatment, wherein detection of decreased levels of the one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, APAM1, SPATA33, MPPED2, CHRNA5, and ASIC4, and KCTD4 in the second aqueous humor sample compared to the first aqueous humor sample indicate that the patient is worsening or not responding to the treatment, and detection of increased levels of the one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, APAM1, SPATA33, MPPED2, CHRNA5, and ASIC4 in the second aqueous humor sample compared to the first aqueous humor sample indicate that the patient is improving. In some embodiments, the method further comprises altering the treatment if the patient is worsening or not responding to the treatment.

In another aspect, a method of diagnosing and treating uveitis in a patient is provided, the method comprising: obtaining an aqueous humor sample from an eye of the patient; measuring levels of one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETIN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A in the aqueous humor sample, wherein increased levels of the one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETIN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A compared to reference value ranges for the levels of the one or more biomarkers in a control aqueous humor sample indicate that the patient has uveitis; and treating the patient for the uveitis if the patient has a positive diagnosis for uveitis. In certain embodiments, the levels of at least two, at least three, or at least four biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETIN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A are measured in the aqueous humor sample. In certain embodiments, the levels of IGHM, TXNDC5, SLAMF7, MZB1, IGHD, CR2, IGHG4, LY9, FCRL5, TNFRSF13B, MMP9, IL1R2, GLIPR2, MPO, RETIN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A are measured in the aqueous humor sample. In certain embodiments, the levels of IGHM, TXNDC5, SLAMF7, MZB1, IGHD, CR2, IGHG4, LY9, FCRL5, TNFRSF13B are measured in the aqueous humor sample. In certain embodiments, the levels of MMP9, IL1R2, GLIPR2, MPO, RETIN, CD177, PSTPIP1, CLEC12A, HMGB2, LTA4H are measured in the aqueous humor sample. In certain embodiments, the levels of VWF, TIE1, SELE, CDH5, ANGPT2, FLT4, ADGRF5, VEGFC, TEK, MYCT1 are measured in the aqueous humor sample. In certain embodiments, the levels of CXCL10, IL18BP, CCL22, TNFRSF8, CCL7, MMP12, IL10, CXCL11, CELA1 are measured in the aqueous humor sample. In certain embodiments, the levels of GNLY, CST7, CD8A, CD5, GZMK, GZMA, CCL5, CD7, LCK, SH2D1A are measured in the aqueous humor sample. In certain embodiments, the method further comprises measuring levels of one or more biomarkers selected from BICDL1, NPPC, HES6, FGF23, APAM1, SPATA33, MPPED2, and CHRNA5, GPC2 in the aqueous humor sample, wherein decreased levels of the one or more biomarkers selected from BICDL1, NPPC, HES6, FGF23, APAM1, SPATA33, MPPED2, CHRNA5, and GPC2 compared to reference value ranges for the levels of the one or more biomarkers in a control aqueous humor sample indicate that the uveitis is causing damage to cone cells. In certain embodiments, the method further comprises measuring levels of one or more biomarkers selected from OPCML, GAP43, RIPPLY3, LRIT3, PLA2G5, FGF18, PLD5, and KCTD4 in the aqueous humor sample, wherein decreased levels of the one or more biomarkers selected OPCML, GAP43, RIPPLY3, LRIT3, PLA2G5, FGF18, PLD5, and KCTD4 compared to reference value ranges for the levels of the one or more biomarkers in a control aqueous humor sample indicate that the uveitis is causing damage to retinal pigment epithelium (RPE) cells. In certain embodiments, the method further comprises measuring levels of one or more biomarkers selected from CPNE7, TYRO3, TENM4, MDM4, RDH13, SV2A, SYT5, OLFM3, RIC3, and SEMA6C in the aqueous humor sample, wherein decreased levels of the one or more biomarkers selected CPNE7, TYRO3, TENM4, MDM4, RDH13, SV2A, SYT5, OLFM3, RIC3, and SEMA6C compared to reference value ranges for the levels of the one or more biomarkers in a control aqueous humor sample indicate that the uveitis is causing damage to amacrine cells.

The method may further comprise determining an appropriate treatment regimen for a patient and, in particular, whether a patient should be treated for uveitis. For example, a patient is selected for treatment for uveitis if the patient has a positive diagnosis for uveitis based on a biomarker expression profile, as described herein. The treatment for uveitis may comprise, for example, administering a glucocorticoid steroid, a cycloplegic agent, an antimetabolite, a T-cell inhibitor, an anti-tumor necrosis factor (TNF) agent, a biologic agent, an alkylating agent, an antibiotic for bacterial uveitis, an antiviral agent for viral uveitis, or an antifungal agent for fungal uveitis, or performing a vitrectomy, or a combination thereof. In some embodiments, the glucocorticoid steroid is selected from the group consisting of prednisolone, methylprednisolone, iluvien, ozurdex, retisert, and triamcinolone. In some embodiments, the T-cell inhibitor is a calcineurin inhibitor or a mTOR inhibitor. In some embodiments, the calcineurin inhibitor is selected from the group consisting of cyclosporine, tacrolimus and voclosporin. In some embodiments, the mTOR inhibitor is selected from the group consisting of everolimus and sirolimus. In some embodiments, the antimetabolite is a purine antagonist, a dihydrofolate reductase (DHFR) inhibitor, or an inosine monophosphate dehydrogenase (IMPDH) inhibitor. In some embodiments, the antimetabolite is selected from the group consisting of azathioprine, methotrexate, and mycophenolate mofetil. In some embodiments, the anti-TNF agent is selected from the group consisting of adalimumab, certolizumab, golimumab, infliximab, and etanercept. In some embodiments, the biologic agent is selected from the group consisting of efalizumab, rituximab, abatacept, alemtuzumab, anakinra, canakinumab, gevokizumab, daclizumab, tocilizumab, secukinumab, interferon α/β, fingolimod, aflibercept, bevacizumab, ranibizumab, and intravenous immunoglobulin (IVIG). In some embodiments, the alkylating agent is chlorambucil or cyclophosphamide. In some embodiments, the cycloplegic agent is atropine or homatropine. In some embodiments, the antibiotic is selected from the group consisting of cephalosporins, vancomycin, ceftazidime, amikacin, gentamycin, and moxifloxacin. In some embodiments, the antiviral agent is selected from the group consisting of ganciclovir, acyclovir, foscarnet, valacyclovir, and cidofivir. In some embodiments, the antifungal agent is selected from the group consisting of amphotericin B, voriconazole, caspofungin, and fluconazole.

In another aspect, a method of monitoring uveitis in a patient is provided, the method comprising: obtaining a first aqueous humor sample from an eye of the patient at a first time point and a second aqueous humor sample from the eye of the patient later at a second time point; and measuring levels of one or more biomarkers in the first aqueous humor sample and the second aqueous humor sample, wherein the biomarkers are selected from IGHM, TXNDC5, SLAMF7, MZB1, IGHD, CR2, IGHG4, LY9, FCRL5, TNFRSF13B, MMP9, IL1R2, GLIPR2, MPO, RETIN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A in the aqueous humor sample, wherein detection of increased levels of the one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETIN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A in the second aqueous humor sample compared to the first aqueous humor sample indicate that the patient is worsening, and wherein detection of decreased levels of the one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETIN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A in the second aqueous humor sample compared to the first aqueous humor sample indicate that the patient is improving.

In another aspect, a method of monitoring efficacy of a treatment of a patient for uveitis is provided, the method comprising: obtaining a first aqueous humor sample from the patient before the patient undergoes the treatment and a second aqueous humor sample from the patient after the patient undergoes the treatment; measuring levels of one or more biomarkers in the first aqueous humor sample and the second aqueous humor sample, wherein the one or more biomarkers are selected from IGHM, TXNDC5, SLAMF7, MZB1, IGHD, CR2, IGHG4, LY9, FCRL5, TNFRSF13B, MMP9, IL1R2, GLIPR2, MPO, RETIN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A; and evaluating the efficacy of the treatment, wherein detection of increased levels of the one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETIN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A in the second aqueous humor sample compared to the first aqueous humor sample indicate that the patient is worsening or not responding to the treatment, and detection of decreased levels of the one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETIN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL110, CXCL11, CELA1, GNLY, CST7, CD8A, CD5, GZMK, GZMA, CCL5, CD7, LCK, and SH2D1A in the second aqueous humor sample compared to the first aqueous humor sample indicate that the patient is improving. In some embodiments, the method further comprises altering the treatment if the patient is worsening or not responding to the treatment.

In another aspect, a method of determining biological age of an eye in a patient is provided, the method comprising: obtaining an aqueous humor sample from an eye of the patient; and measuring levels of one or more biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 in the aqueous humor sample; and using a machine learning aging model to determine the biological age of the eye based on the levels of the one or more biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3. In certain embodiments, the levels of at least two, at least three, or at least four biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 are measured in the aqueous humor sample. In certain embodiments, the levels of LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 are measured in the aqueous humor sample.

In certain embodiments, the method further comprises increasing screening of the patient for an aging-related disease if the biological age of the eye indicates a risk of age-related pathology and morbidity. The subject methods may allow earlier treatment of aging-related diseases to delay disease progression and earlier medical intervention to prolong the life and/or improve the health of the patient. In certain embodiments, the method further comprises administering a treatment for an age-related eye disease to the patient if the patient is identified as having the age-related eye disease. Age-related eye diseases include, but are not limited to, age-related macular degeneration, cataracts, retinitis pigmentosa, diabetic retinopathy (e.g., proliferative or non-proliferative), glaucoma, uveitis, neovascular inflammatory vitreoretinopathy, eye melanoma (e.g., choroidal melanoma), Parkinson's disease-associated eye disorders, dry eye, retinal detachment, presbyopia, keratoconjunctivitis sicca, and epiphora.

In another aspect, a method of estimating age of a subject is provided, the method comprising: obtaining an aqueous humor sample from an eye of the patient; and measuring levels of one or more biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 in the aqueous humor sample; and using a machine learning aging model to estimate age of a subject based on the levels of the one or more biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3.

In some cases, the diagnostic methods described herein may be used by themselves or combined with medical imaging or other ophthalmology techniques for detecting ocular lesions to confirm the diagnosis and further evaluate the severity and extent of disease (e.g., detect retina damage, leaking, macular degeneration, inflammation, neovascularization, or other abnormalities of retinal arteries and veins, and/or macular edema) to aid in determining prognosis and evaluating optimal strategies for treatment (e.g., laser surgery, laser coagulation, vitrectomy, or injection of corticosteroids (e.g., triamcinolone) or vascular endothelial growth factor inhibitors (e.g., ranibizumab) into the eye, etc.). Exemplary medical imaging and ophthalmology techniques include, without limitation, fundus photography, fluorescein angiography, retinal vessel analysis, ultrasonography, optical coherence tomography, autofluorescence, indocyanine green angiography, and the radioactive phosphorus uptake test on the eye.

In some cases, combinations of biomarkers or combinations of biomarker panels are used in the subject methods. In some such cases, the levels of all measured biomarkers must change (as described above) in order for the diagnosis to be made. In some embodiments, only some biomarkers are used in the methods described herein. For example, a single biomarker, 2 biomarkers, 3 biomarkers, 4 biomarkers, 5 biomarkers, 6 biomarkers, 7 biomarkers, 8 biomarkers, 9 biomarkers, 10 biomarkers, 11 biomarkers, 12 biomarkers, 13 biomarkers, 14 biomarkers, 15 biomarkers, 16 biomarkers, 17 biomarkers, 18 biomarkers, 19 biomarkers, or 20 biomarkers can be used in any combination. In other embodiments, all the biomarkers are used. The quantitative values may be combined in linear or non-linear fashion to calculate one or more risk scores for a disease such as Parkinson's disease or a vitreoretinal disease such as retinitis pigmentosa, diabetic retinopathy (e.g., proliferative or non-proliferative), or uveitis; or the biological age of an eye to estimate age of an individual and/or risk of an age-related eye disease or other age-related pathology.

The level of a biomarker in a pre-treatment aqueous humor sample can be referred to as a “pre-treatment value” because the first aqueous humor sample is isolated from the individual prior to the administration of the therapy (i.e., “pre-treatment”). The level of a biomarker in the pre-treatment aqueous humor sample can also be referred to as a “baseline value” because this value is the value to which “post-treatment” values are compared. In some cases, the baseline value (i.e., “pre-treatment value”) is determined by determining the level of a biomarker in multiple (i.e., more than one, e.g., two or more, three or more, for or more, five or more, etc.) pre-treatment aqueous humor samples. In some cases, the multiple pre-treatment aqueous humor samples are isolated from an individual at different time points in order to assess natural fluctuations in biomarker levels prior to treatment. As such, in some cases, one or more (e.g., two or more, three or more, for or more, five or more, etc.) pre-treatment aqueous humor samples are isolated from the individual. In some embodiments, all of the pre-treatment aqueous humor samples will be the same type of aqueous humor sample (e.g., a biopsy sample). In some cases, two or more pre-treatment aqueous humor samples are pooled prior to determining the level of the biomarker in the aqueous humor samples. In some cases, the level of the biomarker is determined separately for two or more pre-treatment aqueous humor samples and a “pre-treatment value” is calculated by averaging the separate measurements.

A post-treatment aqueous humor sample is isolated from an individual after the administration of a therapy. Thus, the level of a biomarker in a post-treatment sample can be referred to as a “post-treatment value”. In some embodiments, the level of a biomarker is measured in additional post-treatment aqueous humor samples (e.g., a second, third, fourth, fifth, etc. post-treatment aqueous humor sample). Because additional post-treatment aqueous humor samples are isolated from the individual after the administration of a treatment, the levels of a biomarker in the additional aqueous humor samples can also be referred to as “post-treatment values.”

The term “responsive” as used herein means that the treatment is having the desired effect such as improving vision, preventing, reducing or delaying vision loss, preventing or reducing retina damage, preventing or reducing neovascularization, and/or preventing or reducing macular edema. When the individual does not improve in response to the treatment, it may be desirable to seek a different therapy or treatment regime for the individual.

The determination that an individual has a certain biological age, is at risk of an age-related eye disease or other age-related pathology, or has a disease by expression profiling is an active clinical application of the correlation between levels of a biomarker and biological age, an age-related eye disease or other age-related pathology, or a certain disease. For example, “determining” requires the active step of reviewing the data, which is produced during the active assaying step(s), and determining biological age or resolving whether an individual does or does not have a disease or risk of a disease. Additionally, in some cases, a decision is made to proceed with a current treatment (i.e., therapy), or instead to alter the treatment. In some cases, the subject methods include the step of continuing therapy or altering therapy.

The term “continue treatment” (i.e., continue therapy) is used herein to mean that the current course of treatment (e.g., continued administration of a therapy) is to continue. If the current course of treatment is not effective, the treatment may be altered. “Altering therapy” is used herein to mean “discontinuing therapy” or “changing the therapy” (e.g., changing the type of treatment, changing the particular dose and/or frequency of administration of medication, e.g., increasing the dose and/or frequency). In some cases, therapy can be altered until the individual is deemed to be responsive. In some embodiments, altering therapy means changing which type of treatment is administered, discontinuing a particular treatment altogether, etc.

As a non-limiting illustrative example, a patient may be initially treated for proliferative diabetic retinopathy by administering a vascular endothelial growth factor inhibitor. Then to “continue treatment” would be to continue with this type of treatment. If the current course of treatment is not effective, the treatment may be altered, e.g., switching treatment to a different vascular endothelial growth factor inhibitor or increasing the dose or frequency of administration of the vascular endothelial growth factor inhibitor, or changing to a different type of treatment such as laser surgery, laser coagulation, or vitrectomy.

In other words, the level of one or more biomarkers may be monitored in order to determine when to continue therapy and/or when to alter therapy. As such, a post-treatment aqueous humor sample can be isolated after any of the administrations and the aqueous humor sample can be assayed to determine the level of a biomarker. Accordingly, the subject methods can be used to determine whether an individual being treated is responsive or is maintaining responsiveness to a treatment.

The therapy can be administered to an individual any time after a pre-treatment aqueous humor sample is isolated from the individual, but it is preferable for the therapy to be administered simultaneous with or as soon as possible (e.g., about 7 days or less, about 3 days or less, e.g., 2 days or less, 36 hours or less, 1 day or less, 20 hours or less, 18 hours or less, 12 hours or less, 9 hours or less, 6 hours or less, 3 hours or less, 2.5 hours or less, 2 hours or less, 1.5 hours or less, 1 hour or less, 45 minutes or less, 30 minutes or less, 20 minutes or less, 15 minutes or less, 10 minutes or less, 5 minutes or less, 2 minutes or less, or 1 minute or less) after a pre-treatment aqueous humor sample is isolated (or, when multiple pre-treatment aqueous humor samples are isolated, after the final pre-treatment aqueous humor sample is isolated).

In some cases, more than one type of therapy may be administered to the individual. For example, a subject who has proliferative diabetic retinopathy may be treated with a corticosteroid and a vascular endothelial growth factor inhibitor or laser surgery. A subject who has more severe disease or who is at high risk of disease progression, may be treated more aggressively. For example, treatment of a high-risk patient may include, without limitation, laser surgery, laser coagulation, or vitrectomy.

In some embodiments, the subject methods include providing an analysis (e.g., an oral or written report) having any or all of the following information: identifying information of the subject (name, age, etc.), a description of what type of aqueous humor sample(s) was used and/or how it was used, the technique used to assay the sample, the results of the assay (e.g., the level of the biomarker as measured, and/or the fold-change of a biomarker level over time, or in a post-treatment assay compared to a pre-treatment assay), the assessment as to whether the individual is determined to have Parkinson's disease or a vitreoretinal disease such as retinitis pigmentosa, diabetic retinopathy (e.g., non-proliferative or proliferative), or uveitis, the predicted biological age of an eye or the individual and risk of age-related pathology and morbidity, a recommendation for additional screening for pathology, a recommendation for treatment, and/or to continue or alter therapy, a recommended strategy for additional therapy, etc. As described above, an analysis can be an oral or written report (e.g., written or electronic document). The analysis can be provided to the subject, to the subject's physician, to a testing facility, etc. The analysis can also be accessible as a website address via the internet. In some such cases, the analysis can be accessible by multiple different entities (e.g., the subject, the subject's physician, a testing facility, etc.).

### Detecting and Measuring Biomarkers

It is understood that the biomarkers in a sample can be measured by any suitable method known in the art. Measurement of the expression level of a biomarker can be direct or indirect. For example, the abundance levels of RNAs or proteins can be directly quantitated. Alternatively, the amount of a biomarker can be determined indirectly by measuring abundance levels of cDNAs, amplified RNAs or DNAs, or by measuring quantities or activities of RNAs, proteins, or other molecules (e.g., metabolites or metabolic byproducts) that are indicative of the expression level of the biomarker. The methods for measuring biomarkers in a sample have many applications. For example, one or more biomarkers can be measured to aid in diagnosing a patient with a disease or risk of age-related pathology and determining the appropriate treatment for a subject, as well as monitoring responses of a subject to treatment.

In some embodiments, the amount or level in the sample of one or more proteins/polypeptides encoded by a gene of interest is determined. Any convenient protocol for evaluating protein levels may be employed, wherein the level of one or more proteins in the assayed sample is determined. Two representative and convenient techniques for assaying protein levels include aptamer-based assays and antibody-based methods such as the enzyme-linked immunosorbent assay (ELISA).

Aptamer-based assays use aptamers comprising single-stranded oligonucleotides that bind specifically to biomarker proteins of interest. Either high affinity RNA or DNA aptamers with specificity for a protein of interest may be used. Functional groups that mimic amino acid side-chains may be added to aptamers to confer protein-like properties to improve binding affinity to a protein of interest. Aptamers that bind specifically and with high affinity to a protein of interest can be selected from large libraries of aptamers having randomized sequences using Systematic Evolution of Ligands by EXponential enrichment (SELEX). The aptamers may be designed with unique nucleotide sequences recognizable by specific hybridization probes for capture on a hybridization array for multiplexed detection of biomarkers (see, e.g., Gold et al. (2010) Aptamer-Based Multiplexed Proteomic Technology for Biomarker Discovery. PLoS ONE 5(12):e15004; herein incorporated by reference in its entirety.

For antibody-based methods of protein level determination, any convenient antibody can be used that specifically binds to the intended biomarker. The terms “specifically binds” or “specific binding” as used herein refer to preferential binding to a molecule relative to other molecules or moieties in a solution or reaction mixture (e.g., an antibody specifically binds to a particular polypeptide or epitope relative to other available polypeptides or epitopes). In some embodiments, the affinity of one molecule for another molecule to which it specifically binds is characterized by a K_{d} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹ M or less, 10⁻¹³ M or less, 10⁻⁴ M or less, 10⁻¹⁵ M or less, or 10⁻¹⁶ M or less). By “affinity” it is meant the strength of binding, increased binding affinity being correlated with a lower K_{d}.

While a variety of different manners of assaying for protein levels are known in the art, one representative and convenient type of protocol for assaying protein levels is the enzyme-linked immunosorbent assay (ELISA). In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hours at temperatures on the order of about 25°-27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g., urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hours at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer. The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Alternatively, other methods for measuring the levels of one or more proteins in a sample may be employed, and any convenient method may be used. Representative examples known to one of ordinary skill in the art include but are not limited to other immunoassay techniques such as radioimmunoassays (RIA), sandwich immunoassays, fluorescent immunoassays, enzyme multiplied immunoassay technique (EMIT), capillary electrophoresis immunoassays (CEIA), and immunoprecipitation assays; mass spectrometry, or tandem mass spectrometry, proteomic arrays, xMAP microsphere technology, western blotting, immunohistochemistry, flow cytometry, cytometry by time-of-flight (CyTOF), multiplexed ion beam imaging (MIBI), and detection in body fluid by electrochemical sensor. In, for example, flow cytometry methods, the quantitative level of gene products of the one or more genes of interest are detected on cells in a cell suspension by lasers. As with ELISAs and immunohistochemistry, antibodies (e.g., monoclonal antibodies) that specifically bind the polypeptides encoded by the genes of interest are used in such methods.

As another example, electrochemical sensors may be employed. In such methods, a capture aptamer or an antibody that is specific for a target protein (the “analyte”) is immobilized on an electrode. A second aptamer or antibody, also specific for the target protein, is labeled with, for example, pyrroquinoline quinone glucose dehydrogenase ((PQQ)GDH). The sample of body fluid is introduced to the sensor either by submerging the electrodes in body fluid or by adding the sample fluid to a sample chamber, and the analyte allowed to interact with the labeled aptamer/antibody and the immobilized capture aptamer/antibody. Glucose is then provided to the sample, and the electric current generated by (PQQ)GDH is observed, where the amount of electric current passing through the electrochemical cell is directly related to the amount of analyte captured at the electrode.

For measuring protein activity levels, the amount or level of protein activity in the sample of one or more proteins/polypeptides encoded by the gene of interest is determined.

In other embodiments, the amount or level in the sample of one or more proteins is determined. Any convenient method for measuring protein levels in a sample may be used, e.g., antibody-based methods, e.g., aptamer-based assays, immunoassay such as enzyme-linked immunosorbent assays (ELISAs), immunohistochemistry, and mass spectrometry.

The resultant data provides information regarding expression, amount, and/or activity for each of the biomarkers that have been measured, wherein the information is in terms of whether or not the biomarker is present (e.g., expressed) and at what level, and wherein the data may be both qualitative and quantitative.

### Data Analysis

In some embodiments, one or more pattern recognition methods can be used in analyzing the data for biomarker levels. The quantitative values may be combined in linear or non-linear fashion to calculate one or more risk scores for a disease (e.g., Parkinson's disease or a vitreoretinal disease such as retinitis pigmentosa, diabetic retinopathy (e.g., proliferative or non-proliferative), or uveitis) or the biological age of an eye to estimate age of an individual and/or risk of an age-related eye disease or other age-related pathology. In some embodiments, measurements for a biomarker or combinations of biomarkers are formulated into linear or non-linear models or algorithms (e.g., a ‘biomarker signature’) and converted into a likelihood score. This likelihood score indicates the probability that an aqueous humor sample is from a patient of a particular biological age, or a patient who may exhibit no evidence of disease, or a patient who may exhibit a particular disease or a risk of age-related pathology and morbidity. The models and/or algorithms can be provided in machine readable format, and may be used to correlate biomarker levels or a biomarker profile with a disease state, and/or to designate a treatment modality for a patient or class of patients.

Analyzing the levels of a plurality of biomarkers may comprise the use of an algorithm or classifier. In some embodiments, a machine learning algorithm is used to classify a patient as having proliferative diabetic retinopathy. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting, including XGBoost or extreme gradient boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, including XGBoost or extreme gradient boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### Kits

Also provided are kits for use in the methods, disclosed herein, for diagnosing Parkinson's disease or a vitreoretinal disease such as retinitis pigmentosa, diabetic retinopathy (e.g., proliferative or non-proliferative), or uveitis; or determining the biological age of an eye to estimate the age of an individual and/or risk of an age-related eye disease or other age-related pathology. The subject kits include agents (e.g., an aptamer or antibody that specifically binds to a biomarker and/or other assay reagents, and the like) for determining the level of at least one biomarker. In some embodiments, a kit comprises agents for determining the level of a single biomarker, two or more different biomarkers, three or more different biomarkers, four or more different biomarkers, or five or more different biomarkers. In certain embodiments, the kit comprises reagents for performing an aptamer-based assay or immunoassay.

In certain embodiments, a kit for diagnosing retinitis pigmentosa is provided, the kit comprising agents for detecting at least 3 biomarkers selected from the group consisting of RS1, SAG, SPINK4, FUT3, GDF10, LRIT2, FAIM, CDHR1, RCVRN, GSKIP, EYS, NPPC, HES6, FGF23, APAM1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LPIT3, PLA2G5, GALNT11, and KCTD4. In certain embodiments, the kit comprises agents for detecting all of the RS1, SAG, SPINK4, FUT3, GDF10, LRIT2, FAIM, CDHR1, RCVRN, GSKIP, EYS, NPPC, HES6, FGF23, APAM1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LPIT3, PLA2G5, GALNT11, and KCTD4 biomarkers. In certain embodiments, kit further comprises reagents for performing an aptamer-based proteomic assay or immunoassay. In certain embodiments, the kit comprises an aptamer or antibody that specifically binds to RS1, an aptamer or antibody that specifically binds to SAG, an aptamer or antibody that specifically binds to SPINK4, an aptamer or antibody that specifically binds to FUT3, an aptamer or antibody that specifically binds to GDF10, an aptamer or antibody that specifically binds to LRIT2, an aptamer or antibody that specifically binds to FAIM, an aptamer or antibody that specifically binds to CDHR1, an aptamer or antibody that specifically binds to RCVRN, an aptamer or antibody that specifically binds to GSKIP, an aptamer or antibody that specifically binds to EYS, an aptamer or antibody that specifically binds to NPPC, an aptamer or antibody that specifically binds to HES6, an aptamer or antibody that specifically binds to FGF23, an aptamer or antibody that specifically binds to APAM1, an aptamer or antibody that specifically binds to GUCA1A, an aptamer or antibody that specifically binds to SPATA33, an aptamer or antibody that specifically binds to MPPED2, an aptamer or antibody that specifically binds to ASIC4, an aptamer or antibody that specifically binds to JPH4, an aptamer or antibody that specifically binds to CPNE7, an aptamer or antibody that specifically binds to TENM4, an aptamer or antibody that specifically binds to GRM4, an aptamer or antibody that specifically binds to TNKS, an aptamer or antibody that specifically binds to GAD1, an aptamer or antibody that specifically binds to SV2A, an aptamer or antibody that specifically binds to SYT5, an aptamer or antibody that specifically binds to RIC3, an aptamer or antibody that specifically binds to CAMK2A, an aptamer or antibody that specifically binds to LRRTM4, an aptamer or antibody that specifically binds to KCNG4, an aptamer or antibody that specifically binds to DPP10, an aptamer or antibody that specifically binds to PPP1R27, an aptamer or antibody that specifically binds to RGS5, an aptamer or antibody that specifically binds to GRAP, an aptamer or antibody that specifically binds to RAMP3, an aptamer or antibody that specifically binds to MEOX1, an aptamer or antibody that specifically binds to AFAP1 L1, an aptamer or antibody that specifically binds to OPCML, an aptamer or antibody that specifically binds to LPIT3, an aptamer or antibody that specifically binds to PLA2G5, an aptamer or antibody that specifically binds to GALNT11, and an aptamer or antibody that specifically binds to KCTD4.

In certain embodiments, a kit for diagnosing diabetic retinopathy is provided, the kit comprising agents for detecting at least 3 biomarkers selected from the group consisting LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1. In certain embodiments, the kit comprises agents for detecting all of the LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 biomarkers. In certain embodiments, the kit comprises agents for detecting at least 3 biomarkers selected from the group consisting of THBS1, CLIC4, NRP2, VEGFA, WARS1, CXCL8, TYMP, CCL2, MAPK14, and AKT1. In certain embodiments, the kit comprises agents for detecting all of the THBS1, CLIC4, NRP2, VEGFA, WARS1, CXCL8, TYMP, CCL2, MAPK14, and AKT1 biomarkers. In certain embodiments, the kit further comprises reagents for performing an aptamer-based proteomic assay or immunoassay. In certain embodiments, the kit comprises an aptamer or antibody that specifically binds to LBP, an aptamer or antibody that specifically binds to CRP, an aptamer or antibody that specifically binds to ITIH3, an aptamer or antibody that specifically binds to APOA1, an aptamer or antibody that specifically binds to CPN2, an aptamer or antibody that specifically binds to C4BPA, an aptamer or antibody that specifically binds to HABP2, an aptamer or antibody that specifically binds to F13B, an aptamer or antibody that specifically binds to SERPINA10, an aptamer or antibody that specifically binds to IGFBP1, an aptamer or antibody that specifically binds to FN1, an aptamer or antibody that specifically binds to MMP19, an aptamer or antibody that specifically binds to ANGPT2, an aptamer or antibody that specifically binds to ROBO4, an aptamer or antibody that specifically binds to VEGFD, an aptamer or antibody that specifically binds to ANGPTL4, an aptamer or antibody that specifically binds to FLT4, an aptamer or antibody that specifically binds to FLNA, an aptamer or antibody that specifically binds to EPHA1, an aptamer or antibody that specifically binds to ENG, an aptamer or antibody that specifically binds to THBS1, an aptamer or antibody that specifically binds to CLIC4, an aptamer or antibody that specifically binds to NRP2, an aptamer or antibody that specifically binds to VEGFA, an aptamer or antibody that specifically binds to WARS1, an aptamer or antibody that specifically binds to CXCL8, an aptamer or antibody that specifically binds to TYMP, an aptamer or antibody that specifically binds to CCL2, an aptamer or antibody that specifically binds to MAPK14, and an aptamer or antibody that specifically binds to AKT1. In certain embodiments, the kit comprises an aptamer or antibody that specifically binds to FN1, an aptamer or antibody that specifically binds to MMP19, an aptamer or antibody that specifically binds to ANGPT2, an aptamer or antibody that specifically binds to ROBO4, an aptamer or antibody that specifically binds to VEGFD, an aptamer or antibody that specifically binds to ANGPTL4, an aptamer or antibody that specifically binds to FLT4, an aptamer or antibody that specifically binds to FLNA, an aptamer or antibody that specifically binds to EPHA1, and an aptamer or antibody that specifically binds to ENG. In certain embodiments, the kit comprises an aptamer or antibody that specifically binds to THBS1, an aptamer or antibody that specifically binds to CLIC4, an aptamer or antibody that specifically binds to NRP2, an aptamer or antibody that specifically binds to VEGFA, an aptamer or antibody that specifically binds to WARS1, an aptamer or antibody that specifically binds to CXCL8, an aptamer or antibody that specifically binds to TYMP, an aptamer or antibody that specifically binds to CCL2, an aptamer or antibody that specifically binds to MAPK14, and an aptamer or antibody that specifically binds to AKT1.

In certain embodiments, a kit for diagnosing Parkinson's disease is provided, the kit comprising agents for detecting at least 3 biomarkers selected from the group consisting of TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, APAM1, SPATA33, MPPED2, CHRNA5, and ASIC4. In certain embodiments, the kit comprises agents for detecting all of the TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, APAM1, SPATA33, MPPED2, CHRNA5, and ASIC4 biomarkers. In certain embodiments, the kit further comprises reagents for performing an aptamer-based proteomic assay or immunoassay. In certain embodiments, the kit comprises an aptamer or antibody that specifically binds to TRIO, an aptamer or antibody that specifically binds to JPH4, an aptamer or antibody that specifically binds to CPNE7, an aptamer or antibody that specifically binds to GRM4, an aptamer or antibody that specifically binds to TNKS, an aptamer or antibody that specifically binds to SERAC1, an aptamer or antibody that specifically binds to ACVR2B, an aptamer or antibody that specifically binds to SV2A, an aptamer or antibody that specifically binds to RIC3, an aptamer or antibody that specifically binds to CAMK2A, an aptamer or antibody that specifically binds to BCL2L2, an aptamer or antibody that specifically binds to DBNDD1, an aptamer or antibody that specifically binds to SULT4A1, an aptamer or antibody that specifically binds to HABP4, an aptamer or antibody that specifically binds to SNPH, an aptamer or antibody that specifically binds to RAC3, an aptamer or antibody that specifically binds to SYT13, an aptamer or antibody that specifically binds to PAK5, an aptamer or antibody that specifically binds to NTRK1, an aptamer or antibody that specifically binds to C1 QL4, an aptamer or antibody that specifically binds to KCNG4, an aptamer or antibody that specifically binds to DPP10, an aptamer or antibody that specifically binds to PPP1R27, an aptamer or antibody that specifically binds to PIH1 D2, an aptamer or antibody that specifically binds to CDHR1, an aptamer or antibody that specifically binds to C17orf67, an aptamer or antibody that specifically binds to GSKIP, an aptamer or antibody that specifically binds to CEP112, an aptamer or antibody that specifically binds to HES6, an aptamer or antibody that specifically binds to FGF23, an aptamer or antibody that specifically binds to APAM1, an aptamer or antibody that specifically binds to SPATA33, an aptamer or antibody that specifically binds to MPPED2, an aptamer or antibody that specifically binds to CHRNA5, and an aptamer or antibody that specifically binds to ASIC4.

In certain embodiments, a kit for diagnosing uveitis is provided, the kit comprising agents for detecting at least 3 biomarkers selected from the group consisting of IGHM, TXNDC5, SLAMF7, MZB1, IGHD, CR2, IGHG4, LY9, FCRL5, TNFRSF13B, MMP9, IL1R2, GLIPR2, MPO, RETIN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A. In certain embodiments, the kit comprises agents for detecting all of the IGHM, TXNDC5, SLAMF7, MZB1, IGHD, CR2, IGHG4, LY9, FCRL5, TNFRSF13B, MMP9, IL1R2, GLIPR2, MPO, RETIN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A biomarkers. In certain embodiments, the kit further comprises reagents for performing an aptamer-based proteomic assay or immunoassay. In certain embodiments, the kit comprises an aptamer or antibody that specifically binds to IGHM, an aptamer or antibody that specifically binds to TXNDC5, an aptamer or antibody that specifically binds to SLAMF7, an aptamer or antibody that specifically binds to MZB1, an aptamer or antibody that specifically binds to IGHD, an aptamer or antibody that specifically binds to CR2, an aptamer or antibody that specifically binds to IGHG4, an aptamer or antibody that specifically binds to LY9, an aptamer or antibody that specifically binds to FCRL5, an aptamer or antibody that specifically binds to TNFRSF13B, an aptamer or antibody that specifically binds to MMP9, an aptamer or antibody that specifically binds to IL1R2, an aptamer or antibody that specifically binds to GLIPR2, an aptamer or antibody that specifically binds to MPO, an aptamer or antibody that specifically binds to RETIN, an aptamer or antibody that specifically binds to CD177, an aptamer or antibody that specifically binds to PSTPIP1, an aptamer or antibody that specifically binds to CLEC12A, an aptamer or antibody that specifically binds to HMGB2, an aptamer or antibody that specifically binds to LTA4H, an aptamer or antibody that specifically binds to VWF, an aptamer or antibody that specifically binds to TIE1, an aptamer or antibody that specifically binds to SELE, CDH5, an aptamer or antibody that specifically binds to ANGPT2, an aptamer or antibody that specifically binds to FLT4, an aptamer or antibody that specifically binds to ADGRF5, an aptamer or antibody that specifically binds to VEGFC, an aptamer or antibody that specifically binds to TEK, an aptamer or antibody that specifically binds to MYCT1, an aptamer or antibody that specifically binds to CXCL10, an aptamer or antibody that specifically binds to IL18BP, an aptamer or antibody that specifically binds to CCL22, an aptamer or antibody that specifically binds to TNFRSF8, an aptamer or antibody that specifically binds to CCL7, an aptamer or antibody that specifically binds to MMP12, an aptamer or antibody that specifically binds to IL110, an aptamer or antibody that specifically binds to CXCL11, an aptamer or antibody that specifically binds to CELA1, an aptamer or antibody that specifically binds to GNLY, an aptamer or antibody that specifically binds to CST7, an aptamer or antibody that specifically binds to CD8A, an aptamer or antibody that specifically binds to CD5, an aptamer or antibody that specifically binds to GZMK, an aptamer or antibody that specifically binds to GZMA, an aptamer or antibody that specifically binds to CCL5, an aptamer or antibody that specifically binds to CD7, an aptamer or antibody that specifically binds to LCK, and an aptamer or antibody that specifically binds to SH2D1A.

In certain embodiments, a kit for determining biological age of an eye is provided, the kit comprising agents for detecting at least 3 biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3. In certain embodiments, the kit comprises agents for detecting all of the LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 biomarkers. In certain embodiments, the kit further comprises reagents for performing an aptamer-based proteomic assay or immunoassay. In certain embodiments, the kit comprises an aptamer or antibody that specifically binds to LECT2, an aptamer or antibody that specifically binds to LECT2, an aptamer or antibody that specifically binds to DCBLD1, an aptamer or antibody that specifically binds to SCGN, an aptamer or antibody that specifically binds to ACAN, an aptamer or antibody that specifically binds to AOC2, an aptamer or antibody that specifically binds to CAPS, an aptamer or antibody that specifically binds to TFF3, an aptamer or antibody that specifically binds to AMN, an aptamer or antibody that specifically binds to ABO, an aptamer or antibody that specifically binds to NETO1, an aptamer or antibody that specifically binds to CD274, an aptamer or antibody that specifically binds to PPBP, an aptamer or antibody that specifically binds to ABL1, an aptamer or antibody that specifically binds to HAMP, an aptamer or antibody that specifically binds to IGFBP1, an aptamer or antibody that specifically binds to AAMDC, an aptamer or antibody that specifically binds to A1BG, an aptamer or antibody that specifically binds to ADA2, an aptamer or antibody that specifically binds to MVP, an aptamer or antibody that specifically binds to CRYGC, an aptamer or antibody that specifically binds to A4GALT, an aptamer or antibody that specifically binds to A4GNT, an aptamer or antibody that specifically binds to AADAT, an aptamer or antibody that specifically binds to A2M, and an aptamer or antibody that specifically binds to ABI3.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, and the like, on which the information has been recorded. Yet another form of these instructions that may be present is a website address which may be used via the internet to access the information at a removed site.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-158 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A method of diagnosing and treating retinitis pigmentosa in a patient, the method comprising:
obtaining an aqueous humor sample from an eye of the patient;
measuring levels of one or more biomarkers selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LRIT3, PLA2G5, GALNT11, and KCTD4 in the aqueous humor sample, wherein decreased levels of the one or more biomarkers selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LRIT3, PLA2G5, GALNT11, and KCTD4 compared to reference value ranges for the levels of the one or more biomarkers in a control aqueous humor sample indicate that the patient has retinitis pigmentosa; and
treating the patient for the retinitis pigmentosa if the patient has a positive diagnosis for retinitis pigmentosa.
2. The method of aspect 1, wherein the levels of at least two, at least three, or at least four biomarkers selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LRIT3, PLA2G5, GALNT11, and KCTD4 are measured in the aqueous humor sample.
3. The method of aspect 3, wherein the levels of RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LRIT3, PLA2G5, GALNT11, and KCTD4 are measured in the aqueous humor sample.
4. The method of aspect 1, wherein the levels of RS1, SAG, SPINK4, FUT3, GDF10, LRIT2, FAIM, CDHR1, RCVRN, and GSKIP are measured in the aqueous humor sample.
5. The method of aspect 1, wherein the levels of EYS, NPPC, HES6, FGF23, AP4M1, GUCA1A, SPATA33, MPPED2, and ASIC4 are measured in the aqueous humor sample.
6. The method of aspect 1, wherein the levels of JPH4, CPNE7, TENM4, GRM4, TNKS, GAD1, SV2A, SYT5, RIC3, and CAMK2A are measured in the aqueous humor sample.
7. The method of aspect 1, wherein the levels of LRRTM4, KCNG4, DPP10, and PPP1R27 are measured in the aqueous humor sample.
8. The method of aspect 1, wherein the levels of RGS5, GRAP, RAMP3, and MEOX1, AFAP1 L1 are measured in the aqueous humor sample.
9. The method of aspect 1, wherein the levels of OPCML, LRIT3, PLA2G5, GALNT11, and KCTD4 are measured in the aqueous humor sample.
10. The method of any one of aspects 1-9, wherein said treating the patient for the retinitis pigmentosa comprises retinal sheet transplantation, RPE65 gene therapy, implanting a retinal prosthesis, or administering vitamin A, docosahexaenoic acid (DHA), N-acetylcysteine (NAC), and lutein, or a combination thereof.
11. The method of any one of aspects 1-10, wherein said measuring comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot.
12. The method of aspect 11, wherein the aptamer-based proteomic assay is performed using a multiplex aptamer array.
13. The method of any one of aspects 1-12, wherein the subject has not yet developed clinical symptoms.
14. The method of any one of aspects 1-12, wherein the subject has developed clinical symptoms.
15. A method of monitoring retinitis pigmentosa in a patient, the method comprising:
obtaining a first aqueous humor sample from an eye of the patient at a first time point and a second aqueous humor sample from the eye of the patient later at a second time point; and
measuring levels of one or more biomarkers in the first aqueous humor sample and the second aqueous humor sample, wherein the biomarkers are selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LRIT3, PLA2G5, GALNT11, and KCTD4 in the aqueous humor sample, wherein detection of decreased levels of the one or more biomarkers selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LRIT3, PLA2G5, GALNT11, and KCTD4 in the second aqueous humor sample compared to the first aqueous humor sample indicate that the patient is worsening, and wherein detection of increased levels of the one or more biomarkers selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LRIT3, PLA2G5, GALNT11, and KCTD4 in the second aqueous humor sample compared to the first aqueous humor sample indicate that the patient is improving.
16. A method of monitoring efficacy of a treatment of a patient for retinitis pigmentosa, the method comprising:
obtaining a first aqueous humor sample from the patient before the patient undergoes the treatment and a second aqueous humor sample from the patient after the patient undergoes the treatment;
measuring levels of one or more biomarkers in the first aqueous humor sample and the second aqueous humor sample, wherein the one or more biomarkers are selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LRIT3, PLA2G5, GALNT11, and KCTD4; and
evaluating the efficacy of the treatment, wherein detection of decreased levels of the one or more biomarkers selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LRIT3, PLA2G5, GALNT11, and KCTD4 in the second aqueous humor sample compared to the first aqueous humor sample indicate that the patient is worsening or not responding to the treatment, and detection of increased levels of the one or more biomarkers selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LRIT3, PLA2G5, GALNT11, and KCTD4 in the second aqueous humor sample compared to the first aqueous humor sample indicate that the patient is improving.
17. The method of aspect 30, further comprising altering the treatment if the patient is worsening or not responding to the treatment.
18. A kit for diagnosing retinitis pigmentosa comprising agents for detecting at least 3 biomarkers selected from the group consisting of RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LRIT3, PLA2G5, GALNT11, and KCTD4.
19. The kit of aspect 18, wherein the kit comprises agents for detecting all of the RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LRIT3, PLA2G5, GALNT11, and KCTD4 biomarkers.
20. The kit of aspect 18 or 19, further comprising reagents for performing an aptamer-based proteomic assay or immunoassay.
21. The kit of aspect 20, wherein the kit comprises an aptamer or antibody that specifically binds to RS1, an aptamer or antibody that specifically binds to SAG, an aptamer or antibody that specifically binds to SPINK4, an aptamer or antibody that specifically binds to FUT3, an aptamer or antibody that specifically binds to GDF10, an aptamer or antibody that specifically binds to LRIT2, an aptamer or antibody that specifically binds to FAIM, an aptamer or antibody that specifically binds to CDHR1, an aptamer or antibody that specifically binds to RCVRN, an aptamer or antibody that specifically binds to GSKIP, an aptamer or antibody that specifically binds to EYS, an aptamer or antibody that specifically binds to NPPC, an aptamer or antibody that specifically binds to HES6, an aptamer or antibody that specifically binds to FGF23, an aptamer or antibody that specifically binds to AP4M1, an aptamer or antibody that specifically binds to GUCA1A, an aptamer or antibody that specifically binds to SPATA33, an aptamer or antibody that specifically binds to MPPED2, an aptamer or antibody that specifically binds to ASIC4, an aptamer or antibody that specifically binds to JPH4, an aptamer or antibody that specifically binds to CPNE7, an aptamer or antibody that specifically binds to TENM4, an aptamer or antibody that specifically binds to GRM4, an aptamer or antibody that specifically binds to TNKS, an aptamer or antibody that specifically binds to GAD1, an aptamer or antibody that specifically binds to SV2A, an aptamer or antibody that specifically binds to SYT5, an aptamer or antibody that specifically binds to RIC3, an aptamer or antibody that specifically binds to CAMK2A, an aptamer or antibody that specifically binds to LRRTM4, an aptamer or antibody that specifically binds to KCNG4, an aptamer or antibody that specifically binds to DPP10, an aptamer or antibody that specifically binds to PPP1R27, an aptamer or antibody that specifically binds to RGS5, an aptamer or antibody that specifically binds to GRAP, an aptamer or antibody that specifically binds to RAMP3, an aptamer or antibody that specifically binds to MEOX1, an aptamer or antibody that specifically binds to AFAP1 L1, an aptamer or antibody that specifically binds to OPCML, an aptamer or antibody that specifically binds to LRIT3, an aptamer or antibody that specifically binds to PLA2G5, an aptamer or antibody that specifically binds to GALNT11, and an aptamer or antibody that specifically binds to KCTD4.
22. The kit of any one of aspects 18-21, further comprising instructions for determining whether a subject has retinitis pigmentosa.
23. A protein selected from the group consisting of RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LRIT3, PLA2G5, GALNT11, and KCTD4 for use as a biomarker in diagnosing retinitis pigmentosa.
24. An in vitro method of retinitis pigmentosa, the method comprising:
obtaining an aqueous humor sample from an eye of the patient; and
measuring levels of one or more biomarkers selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LRIT3, PLA2G5, GALNT11, and KCTD4 in the aqueous humor sample, wherein decreased levels of the one or more biomarkers selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LRIT3, PLA2G5, GALNT11, and KCTD4 compared to reference value ranges for the levels of the one or more biomarkers in a control aqueous humor sample indicate that the patient has retinitis pigmentosa.
25. A method of diagnosing and treating diabetic retinopathy in a patient, the method comprising:
obtaining an aqueous humor sample from an eye of the patient;
measuring levels of one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 in the aqueous humor sample, wherein increased levels of the one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 compared to reference value ranges for the levels of the one or more biomarkers in a control aqueous humor sample indicate that the patient has diabetic retinopathy; and
treating the patient for the diabetic retinopathy if the patient has a positive diagnosis for diabetic retinopathy.
26. The method of aspect 25, wherein the levels of at least two, at least three, or at least four biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 are measured in the aqueous humor sample.
27. The method of aspect 26, wherein the levels of LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 are measured in the aqueous humor sample.
28. The method of any one of aspects 25-27, wherein said treating the patient for the diabetic retinopathy comprises administering an anti-vascular endothelial growth factor (VEGF) agent or a steroid, or performing panretinal laser photocoagulation or a vitrectomy, or a combination thereof.
29. The method of aspect 28, wherein the anti-VEGF agent is bevacizumab, ranibizumab, sunitinib, sorafenib, axitinib, aflibercept, brolucizuma, faricimab, or pazopanib.
30. The method of aspect 28, wherein the steroid is triamcinolone acetonide, fluocinolone acetonide, or dexamethasone.
31. The method of any one of aspects 25-30, wherein the levels of FN1, MMP19, ANGPT2, ROBO4, VEGFD, ANGPTL4, FLT4, FLNA, EPHA1, and ENG are measured in the aqueous humor sample, wherein increased levels of the one or more biomarkers selected from FN1, MMP19, ANGPT2, ROBO4, VEGFD, ANGPTL4, FLT4, FLNA, EPHA1, and ENG compared to reference value ranges for the levels of the one or more biomarkers in a control aqueous humor sample indicate that the patient has non-proliferative diabetic retinopathy.
32. The method of aspect 31, further comprising treating the patient for the non-proliferative diabetic retinopathy if the patient has a positive diagnosis for non-proliferative diabetic retinopathy.
33. The method of aspect 32, wherein said treating the patient for the non-proliferative diabetic retinopathy comprises administering an anti-VEGF agent or a steroid, performing vitrectomy or photocoagulation, or increasing frequency of retinal exams or a combination thereof.
34. The method of any one of aspects 25-33, wherein the levels of THBS1, CLIC4, NRP2, VEGFA, WARS1, CXCL8, TYMP, CCL2, MAPK14, AKT1 are measured in the aqueous humor sample, wherein increased levels of the one or more biomarkers selected from THBS1, CLIC4, NRP2, VEGFA, WARS1, CXCL8, TYMP, CCL2, MAPK14, AKT1 compared to reference value ranges for the levels of the one or more biomarkers in a control aqueous humor sample indicate that the patient has proliferative diabetic retinopathy.
35. The method of aspect 34, further comprising treating the patient for the proliferative diabetic retinopathy if the patient has a positive diagnosis for proliferative diabetic retinopathy.
36. The method of aspect 35, wherein said treating the patient for the proliferative diabetic retinopathy comprises administering an anti-VEGF agent or performing panretinal laser photocoagulation, or a combination thereof.
37. The method of any one of aspects 25-36, wherein said measuring comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot.
38. The method of aspect 37, wherein the aptamer-based proteomic assay is performed using a multiplex aptamer array.
39. The method of any one of aspects 25-38, wherein the subject has not yet developed clinical symptoms.
40. The method of any one of aspects 25-38, wherein the subject has developed clinical symptoms.
41. A method of monitoring diabetic retinopathy in a patient, the method comprising:
obtaining a first aqueous humor sample from an eye of the patient at a first time point and a second aqueous humor sample from the eye of the patient later at a second time point; and
measuring levels of one or more biomarkers in the first aqueous humor sample and the second aqueous humor sample, wherein the biomarkers are selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1, wherein detection of increased levels of the one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 in the second aqueous humor sample compared to the first aqueous humor sample indicate that the patient is worsening, and wherein detection of decreased levels of the one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 in the second aqueous humor sample compared to the first aqueous humor sample indicate that the patient is improving.
42. A method of monitoring efficacy of a treatment of a patient for diabetic retinopathy, the method comprising:
obtaining a first aqueous humor sample from the patient before the patient undergoes the treatment and a second aqueous humor sample from the patient after the patient undergoes the treatment;
measuring levels of one or more biomarkers in the first aqueous humor sample and the second aqueous humor sample, wherein the one or more biomarkers are selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1; and
evaluating the efficacy of the treatment, wherein detection of increased levels of the one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 in the second aqueous humor sample compared to the first aqueous humor sample indicate that the patient is worsening or not responding to the treatment, and detection of decreased levels of the one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 in the second aqueous humor sample compared to the first aqueous humor sample indicate that the patient is improving.
43. The method of aspect 42, further comprising altering the treatment if the patient is worsening or not responding to the treatment.
44. A method of monitoring a patient having non-proliferative diabetic retinopathy for progression to proliferative diabetic retinopathy, the method comprising: obtaining an aqueous humor sample from an eye of the patient;
measuring levels of one or more biomarkers selected from THBS1, CLIC4, NRP2, VEGFA, WARS1, CXCL8, TYMP, CCL2, MAPK14, and AKT1 in the aqueous humor sample, wherein increased levels of the one or more biomarkers selected from THBS1, CLIC4, NRP2, VEGFA, WARS1, CXCL8, TYMP, CCL2, MAPK14, and AKT1 compared to reference value ranges for the levels of the one or more biomarkers in a control aqueous humor sample indicate that the patient has proliferative diabetic retinopathy.
45. The method of aspect 44, further comprising treating the patient for the proliferative diabetic retinopathy if the patient has a positive diagnosis for proliferative diabetic retinopathy.
46. The method of aspect 45, wherein said treating the patient for the proliferative diabetic retinopathy comprises administering an anti-VEGF agent or performing panretinal laser photocoagulation, or a combination thereof.
47. A method of distinguishing between a diagnosis of non-proliferative diabetic retinopathy and proliferative diabetic retinopathy for a patient, the method comprising:
obtaining an aqueous humor sample from an eye of the patient;
measuring levels of one or more biomarkers selected from FN1, MMP19, ANGPT2, ROBO4, VEGFD, ANGPTL4, FLT4, FLNA, EPHA1, and ENG in the aqueous humor sample, wherein increased levels of the one or more biomarkers selected from FN1, MMP19, ANGPT2, ROBO4, VEGFD, ANGPTL4, FLT4, FLNA, EPHA1, and ENG compared to reference value ranges for the levels of the one or more biomarkers in a control aqueous humor sample indicate that the patient has non-proliferative diabetic retinopathy; and
measuring levels of one or more biomarkers selected from THBS1, CLIC4, NRP2, VEGFA, WARS1, CXCL8, TYMP, CCL2, MAPK14, and AKT1 in the aqueous humor sample, wherein increased levels of the one or more biomarkers selected from THBS1, CLIC4, NRP2, VEGFA, WARS1, CXCL8, TYMP, CCL2, MAPK14, and AKT1 compared to reference value ranges for the levels of the one or more biomarkers in a control aqueous humor sample indicate that the patient has proliferative diabetic retinopathy.
48. The method of aspect 47, wherein the levels of at least two, at least three, or at least four biomarkers selected from FN1, MMP19, ANGPT2, ROBO4, VEGFD, ANGPTL4, FLT4, FLNA, EPHA1, and ENG, and the levels of at least two, at least three, or at least four biomarkers selected from THBS1, CLIC4, NRP2, VEGFA, WARS1, CXCL8, TYMP, CCL2, MAPK14, and AKT1 are measured in the aqueous humor sample.
49. The method of aspect 48, wherein the levels of FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 are measured in the aqueous humor sample.
50. The method of any one of aspects 47-49, further comprising treating the patient for the non-proliferative diabetic retinopathy if the patient has a positive diagnosis for non-proliferative diabetic retinopathy, or treating the patient for the proliferative diabetic retinopathy if the patient has a positive diagnosis for proliferative diabetic retinopathy.
51. The method of aspect 50, wherein said treating the patient for the non-proliferative diabetic retinopathy comprises administering an anti-VEGF agent or a steroid, performing vitrectomy or photocoagulation, or increasing frequency of retinal exams or a combination thereof.
52. The method of aspect 50, wherein said treating the patient for the proliferative diabetic retinopathy comprises administering an anti-VEGF agent or performing panretinal laser photocoagulation, or a combination thereof.
53. A kit for diagnosing diabetic retinopathy comprising agents for detecting at least 3 biomarkers selected from the group consisting LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1.
54. The kit of aspect 53, wherein the kit comprises agents for detecting all of the LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 biomarkers.
55. A kit for diagnosing proliferative diabetic retinopathy comprising agents for detecting at least 3 biomarkers selected from the group consisting of THBS1, CLIC4, NRP2, VEGFA, WARS1, CXCL8, TYMP, CCL2, MAPK14, and AKT1.
56. The kit of aspect 55, wherein the kit comprises agents for detecting all of the THBS1, CLIC4, NRP2, VEGFA, WARS1, CXCL8, TYMP, CCL2, MAPK14, and AKT1 biomarkers.
57. The kit of aspect 55 or 56, further comprising reagents for performing an aptamer-based proteomic assay or immunoassay.
58. The kit of aspect 57, wherein the kit comprises an aptamer or antibody that specifically binds to LBP, an aptamer or antibody that specifically binds to CRP, an aptamer or antibody that specifically binds to ITIH3, an aptamer or antibody that specifically binds to APOA1, an aptamer or antibody that specifically binds to CPN2, an aptamer or antibody that specifically binds to C4BPA, an aptamer or antibody that specifically binds to HABP2, an aptamer or antibody that specifically binds to F13B, an aptamer or antibody that specifically binds to SERPINA10, an aptamer or antibody that specifically binds to IGFBP1, an aptamer or antibody that specifically binds to FN1, an aptamer or antibody that specifically binds to MMP19, an aptamer or antibody that specifically binds to ANGPT2, an aptamer or antibody that specifically binds to ROBO4, an aptamer or antibody that specifically binds to VEGFD, an aptamer or antibody that specifically binds to ANGPTL4, an aptamer or antibody that specifically binds to FLT4, an aptamer or antibody that specifically binds to FLNA, an aptamer or antibody that specifically binds to EPHA1, an aptamer or antibody that specifically binds to ENG, an aptamer or antibody that specifically binds to THBS1, an aptamer or antibody that specifically binds to CLIC4, an aptamer or antibody that specifically binds to NRP2, an aptamer or antibody that specifically binds to VEGFA, an aptamer or antibody that specifically binds to WARS1, an aptamer or antibody that specifically binds to CXCL8, an aptamer or antibody that specifically binds to TYMP, an aptamer or antibody that specifically binds to CCL2, an aptamer or antibody that specifically binds to MAPK14, and an aptamer or antibody that specifically binds to AKT1.
59. The kit of aspect 57, wherein the kit comprises an aptamer or antibody that specifically binds to FN1, an aptamer or antibody that specifically binds to MMP19, an aptamer or antibody that specifically binds to ANGPT2, an aptamer or antibody that specifically binds to ROBO4, an aptamer or antibody that specifically binds to VEGFD, an aptamer or antibody that specifically binds to ANGPTL4, an aptamer or antibody that specifically binds to FLT4, an aptamer or antibody that specifically binds to FLNA, an aptamer or antibody that specifically binds to EPHA1, and an aptamer or antibody that specifically binds to ENG.
60. The kit of aspect 57, wherein the kit comprises an aptamer or antibody that specifically binds to THBS1, an aptamer or antibody that specifically binds to CLIC4, an aptamer or antibody that specifically binds to NRP2, an aptamer or antibody that specifically binds to VEGFA, an aptamer or antibody that specifically binds to WARS1, an aptamer or antibody that specifically binds to CXCL8, an aptamer or antibody that specifically binds to TYMP, an aptamer or antibody that specifically binds to CCL2, an aptamer or antibody that specifically binds to MAPK14, and an aptamer or antibody that specifically binds to AKT1.
61. The kit of any one of aspects 53-60, further comprising instructions for determining whether a subject has diabetic retinopathy.
62. A protein selected from the group consisting of LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 for use as a biomarker in diagnosing diabetic retinopathy.
63. A protein selected from the group consisting of FN1, MMP19, ANGPT2, ROBO4, VEGFD, ANGPTL4, FLT4, FLNA, EPHA1, ENG for use as a biomarker in diagnosing non-proliferative diabetic retinopathy.
64. A protein selected from the group consisting of THBS1, CLIC4, NRP2, VEGFA, WARS1, CXCL8, TYMP, CCL2, MAPK14, AKT1 for use as a biomarker in diagnosing proliferative diabetic retinopathy.
65. An in vitro method of diagnosing diabetic retinopathy, the method comprising:
obtaining an aqueous humor sample from an eye of the patient; and
measuring levels of one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 in the aqueous humor sample, wherein increased levels of the one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 compared to reference value ranges for the levels of the one or more biomarkers in a control aqueous humor sample indicate that the patient has diabetic retinopathy.
66. The method of aspect 65, wherein the levels of FN1, MMP19, ANGPT2, ROBO4, VEGFD, ANGPTL4, FLT4, FLNA, EPHA1, and ENG are measured in the aqueous humor sample, wherein increased levels of the one or more biomarkers selected from FN1, MMP19, ANGPT2, ROBO4, VEGFD, ANGPTL4, FLT4, FLNA, EPHA1, and ENG compared to reference value ranges for the levels of the one or more biomarkers in a control aqueous humor sample indicate that the patient has non-proliferative diabetic retinopathy.
67. The method of aspect 65, wherein the levels of THBS1, CLIC4, NRP2, VEGFA, WARS1, CXCL8, TYMP, CCL2, MAPK14, AKT1 are measured in the aqueous humor sample, wherein increased levels of the one or more biomarkers selected from THBS1, CLIC4, NRP2, VEGFA, WARS1, CXCL8, TYMP, CCL2, MAPK14, AKT1 compared to reference value ranges for the levels of the one or more biomarkers in a control aqueous humor sample indicate that the patient has proliferative diabetic retinopathy.
68. A method of diagnosing and treating Parkinson's disease in a patient, the method comprising:
obtaining an aqueous humor sample from an eye of the patient;
measuring levels of one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 in the aqueous humor sample, wherein decreased levels of the one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 compared to reference value ranges for the levels of the one or more biomarkers in a control aqueous humor sample indicate that the patient has Parkinson's disease; and
treating the patient for the Parkinson's disease if the patient has Parkinson's disease.
69. The method of aspect 68, wherein the levels of at least two, at least three, or at least four biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 are measured in the aqueous humor sample.
70. The method of aspect 69, wherein the levels of TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 are measured in the aqueous humor sample.
71. The method of aspect 69, wherein the levels of TRIO, JPH4, CPNE7, GRM4, TNKS, SERAC1, ACVR2B, SV2A, RIC3, and CAMK2A are measured in the aqueous humor sample.
72. The method of aspect 69, wherein the levels of BCL2L2, DBNDD1, SULT4A1, HABP4, SNPH, RAC3, and SYT13 are measured in the aqueous humor sample.
73. The method of aspect 69, wherein the levels of PAK5, NTRK1, and C1QL4 are measured in the aqueous humor sample.
74. The method of aspect 69, wherein the levels of KCNG4, DPP10, and PPP1R27 are measured in the aqueous humor sample.
75. The method of aspect 69, wherein the levels of PIH1 D2, CDHR1, C17orf67, GSKIP, and CEP112 are measured in the aqueous humor sample.
76. The method of aspect 69, wherein the levels of HES6, FGF23, AP4M1, SPATA33, MPPED2, CHRNA5, and ASIC4 are measured in the aqueous humor sample.
77. The method of any one of aspects 68-76, wherein said treating the patient for the Parkinson's disease comprises administering I-3,4-dihydroxyphenylalanine (L-DOPA), an aromatic L-amino acid decarboxylase inhibitor, a catechol-O-methyltransferase (COMT) inhibitor, a dopamine agonist, a monoamine oxidase inhibitor, an anticholinergic, or a combination thereof.
78. The method of aspect 77, wherein the aromatic L-amino acid decarboxylase inhibitor is carbidopa, benserazide, methyldopa, alpha-difluoromethyl-DOPA, 3′,4′,5,7-tetrahydroxy-8-methoxyisoflavone, epigallocatechin gallate, or epigallocatechin.
79. The method of aspect 77, wherein the COMT inhibitor is entacapone, nebicapone, nitecapone, opicapone, or tolcapone.
80. The method of aspect 77, wherein the dopamine agonist is pramipexole, ropinirole, bromocriptine, pergolide, rotigotine, apomorphine, aripiprazole, phencyclidine, quinpirole, cabergoline, ciladopa, dihydrexidine, dinapsoline, doxanthrine, lisuride, piribedil, propylnorapomorphine, quinagolide, roxindole, or sumanirole.
81. The method of aspect 77, wherein the monoamine oxidase inhibitor is rasagiline, or selegiline, safinamide.
82. The method of aspect 77, wherein the anticholinergic is procyclidine or trihexyphenidyl.
83. The method of any one of aspects 68-82, wherein said measuring comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot.
84. The method of aspect 83, wherein the aptamer-based proteomic assay is performed using a multiplex aptamer array.
85. The method of any one of aspects 68-84, wherein the subject has not yet developed clinical symptoms.
86. The method of any one of aspects 68-84, wherein the subject has developed clinical symptoms.
87. A method of monitoring Parkinson's disease in a patient, the method comprising:
obtaining a first aqueous humor sample from an eye of the patient at a first time point and a second aqueous humor sample from the eye of the patient later at a second time point; and
measuring levels of one or more biomarkers in the first aqueous humor sample and the second aqueous humor sample, wherein the biomarkers are selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 in the aqueous humor sample, wherein detection of decreased levels of the one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 in the second aqueous humor sample compared to the first aqueous humor sample indicate that the patient is worsening, and wherein detection of increased levels of the one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 in the second aqueous humor sample compared to the first aqueous humor sample indicate that the patient is improving.
88. A method of monitoring efficacy of a treatment of a patient for Parkinson's disease, the method comprising:
obtaining a first aqueous humor sample from the patient before the patient undergoes the treatment and a second aqueous humor sample from the patient after the patient undergoes the treatment;
measuring levels of one or more biomarkers in the first aqueous humor sample and the second aqueous humor sample, wherein the one or more biomarkers are selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4; and
evaluating the efficacy of the treatment, wherein detection of decreased levels of the one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4, and KCTD4 in the second aqueous humor sample compared to the first aqueous humor sample indicate that the patient is worsening or not responding to the treatment, and detection of increased levels of the one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 in the second aqueous humor sample compared to the first aqueous humor sample indicate that the patient is improving.
89. The method of aspect 88, further comprising altering the treatment if the patient is worsening or not responding to the treatment.
90. A kit for diagnosing Parkinson's disease comprising agents for detecting at least 3 biomarkers selected from the group consisting of TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4.
91. The kit of aspect 90, wherein the kit comprises agents for detecting all of the TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 biomarkers.
92. The kit of aspect 90 or 91, further comprising reagents for performing an aptamer-based proteomic assay or immunoassay.
93. The kit of aspect 92, wherein the kit comprises an aptamer or antibody that specifically binds to TRIO, an aptamer or antibody that specifically binds to JPH4, an aptamer or antibody that specifically binds to CPNE7, an aptamer or antibody that specifically binds to GRM4, an aptamer or antibody that specifically binds to TNKS, an aptamer or antibody that specifically binds to SERAC1, an aptamer or antibody that specifically binds to ACVR2B, an aptamer or antibody that specifically binds to SV2A, an aptamer or antibody that specifically binds to RIC3, an aptamer or antibody that specifically binds to CAMK2A, an aptamer or antibody that specifically binds to BCL2L2, an aptamer or antibody that specifically binds to DBNDD1, an aptamer or antibody that specifically binds to SULT4A1, an aptamer or antibody that specifically binds to HABP4, an aptamer or antibody that specifically binds to SNPH, an aptamer or antibody that specifically binds to RAC3, an aptamer or antibody that specifically binds to SYT13, an aptamer or antibody that specifically binds to PAK5, an aptamer or antibody that specifically binds to NTRK1, an aptamer or antibody that specifically binds to C1 QL4, an aptamer or antibody that specifically binds to KCNG4, an aptamer or antibody that specifically binds to DPP10, an aptamer or antibody that specifically binds to PPP1R27, an aptamer or antibody that specifically binds to PIH1 D2, an aptamer or antibody that specifically binds to CDHR1, an aptamer or antibody that specifically binds to C17orf67, an aptamer or antibody that specifically binds to GSKIP, an aptamer or antibody that specifically binds to CEP112, an aptamer or antibody that specifically binds to HES6, an aptamer or antibody that specifically binds to FGF23, an aptamer or antibody that specifically binds to AP4M1, an aptamer or antibody that specifically binds to SPATA33, an aptamer or antibody that specifically binds to MPPED2, an aptamer or antibody that specifically binds to CHRNA5, and an aptamer or antibody that specifically binds to ASIC4.
94. The kit of any one of aspects 90-93, further comprising instructions for determining whether a subject has Parkinson's disease.
95. A protein selected from the group consisting of TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 for use as a biomarker in diagnosing Parkinson's disease.
96. An in vitro method of Parkinson's disease, the method comprising:
obtaining an aqueous humor sample from an eye of the patient; and measuring levels of one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 in the aqueous humor sample, wherein decreased levels of the one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 to reference value ranges for the levels of the one or more biomarkers in a control aqueous humor sample indicate that the patient has Parkinson's disease.
97. A method of diagnosing and treating uveitis in a patient, the method comprising:
obtaining an aqueous humor sample from an eye of the patient;
measuring levels of one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A in the aqueous humor sample, wherein increased levels of the one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A compared to reference value ranges for the levels of the one or more biomarkers in a control aqueous humor sample indicate that the patient has uveitis; and
treating the patient for the uveitis if the patient has a positive diagnosis for uveitis.
98. The method of aspect 97, wherein the levels of at least two, at least three, or at least four biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A are measured in the aqueous humor sample.
99. The method of aspect 98, wherein the levels of IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A are measured in the aqueous humor sample.
100. The method of aspect 98, wherein the levels of IGHM, TXNDC5, SLAMF7, MZB1, IGHD, CR2, IGHG4, LY9, FCRL5, TNFRSF13B are measured in the aqueous humor sample.
101. The method of aspect 98, wherein the levels of MMP9, IL1R2, GLIPR2, MPO, RETN, CD177, PSTPIP1, CLEC12A, HMGB2, LTA4H are measured in the aqueous humor sample.
102. The method of aspect 98, wherein the levels of VWF, TIE1, SELE, CDH5, ANGPT2, FLT4, ADGRF5, VEGFC, TEK, MYCT1 are measured in the aqueous humor sample.
103. The method of aspect 98, wherein the levels of CXCL10, IL18BP, CCL22, TNFRSF8, CCL7, MMP12, IL10, CXCL11, CELA1 are measured in the aqueous humor sample.
104. The method of aspect 98, wherein the levels of GNLY, CST7, CD8A, CD5, GZMK, GZMA, CCL5, CD7, LCK, SH2D1A are measured in the aqueous humor sample.
105. The method of any one of aspects 97-104, further comprising measuring levels of one or more biomarkers selected from BICDL1, NPPC, HES6, FGF23, AP4M1, SPATA33, MPPED2, and CHRNA5, GPC2 in the aqueous humor sample, wherein decreased levels of the one or more biomarkers selected from BICDL1, NPPC, HES6, FGF23, AP4M1, SPATA33, MPPED2, CHRNA5, and GPC2 compared to reference value ranges for the levels of the one or more biomarkers in a control aqueous humor sample indicate that the uveitis is causing damage to cone cells.
106. The method of any one of aspects 97-105, further comprising measuring levels of one or more biomarkers selected from OPCML, GAP43, RIPPLY3, LRIT3, PLA2G5, FGF18, PLD5, and KCTD4 in the aqueous humor sample, wherein decreased levels of the one or more biomarkers selected OPCML, GAP43, RIPPLY3, LRIT3, PLA2G5, FGF18, PLD5, and KCTD4 compared to reference value ranges for the levels of the one or more biomarkers in a control aqueous humor sample indicate that the uveitis is causing damage to retinal pigment epithelium (RPE) cells.
107. The method of any one of aspects 97-106, further comprising measuring levels of one or more biomarkers selected from CPNE7, TYRO3, TENM4, MDM4, RDH13, SV2A, SYT5, OLFM3, RIC3, and SEMA6C in the aqueous humor sample, wherein decreased levels of the one or more biomarkers selected CPNE7, TYRO3, TENM4, MDM4, RDH13, SV2A, SYT5, OLFM3, RIC3, and SEMA6C compared to reference value ranges for the levels of the one or more biomarkers in a control aqueous humor sample indicate that the uveitis is causing damage to amacrine cells.
108. The method of any one of aspects 97-107, wherein said treating the patient for the uveitis comprises administering a glucocorticoid steroid, a cycloplegic agent, an antimetabolite, a T-cell inhibitor, an anti-tumor necrosis factor (TNF) agent, a biologic agent, an alkylating agent, an antibiotic for bacterial uveitis, an antiviral agent for viral uveitis, or an antifungal agent for fungal uveitis, or performing a vitrectomy, or a combination thereof.
109. The method of aspect 108, wherein the glucocorticoid steroid is selected from the group consisting of prednisolone, methylprednisolone, iluvien, ozurdex, retisert, and triamcinolone.
110. The method of aspect 108, wherein the T-cell inhibitor is a calcineurin inhibitor or a mTOR inhibitor.
111. The method of aspect 108, wherein the calcineurin inhibitor is selected from the group consisting of cyclosporine, tacrolimus and voclosporin.
112. The method of aspect 108, wherein the mTOR inhibitor is selected from the group consisting of everolimus and sirolimus.
113. The method of aspect 108, wherein the antimetabolite is a purine antagonist, a dihydrofolate reductase (DHFR) inhibitor, or an inosine monophosphate dehydrogenase (IMPDH) inhibitor.
114. The method of aspect 108, wherein the antimetabolite is selected from the group consisting of azathioprine, methotrexate, and mycophenolate mofetil.
115. The method of aspect 108, wherein the anti-TNF agent is selected from the group consisting of adalimumab, certolizumab, golimumab, infliximab, and etanercept.
116. The method of aspect 108, wherein the biologic agent is selected from the group consisting of efalizumab, rituximab, abatacept, alemtuzumab, anakinra, canakinumab, gevokizumab, daclizumab, tocilizumab, secukinumab, interferon α/β, fingolimod, aflibercept, bevacizumab, ranibizumab, and intravenous immunoglobulin (IVIG).
117. The method of aspect 108, wherein the alkylating agent is chlorambucil or cyclophosphamide.
118. The method of aspect 108, wherein the cycloplegic agent is atropine or homatropine.
119. The method of aspect 108, wherein the antibiotic is selected from the group consisting of cephalosporins, vancomycin, ceftazidime, amikacin, gentamycin, and moxifloxacin.
120. The method of aspect 108, wherein the antiviral agent is selected from the group consisting of ganciclovir, acyclovir, foscarnet, valacyclovir, and cidofivir.
121. The method of aspect 108, wherein the antifungal agent is selected from the group consisting of amphotericin B, voriconazole, caspofungin, and fluconazole.
122. The method of any one of aspects 97-121, wherein said measuring comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot.
123. The method of aspect 122, wherein the aptamer-based proteomic assay is performed using a multiplex aptamer array.
124. The method of any one of aspects 97-123, wherein the subject has not yet developed clinical symptoms.
125. The method of any one of aspects 97-123, wherein the subject has developed clinical symptoms.
126. A method of monitoring uveitis in a patient, the method comprising:
obtaining a first aqueous humor sample from an eye of the patient at a first time point and a second aqueous humor sample from the eye of the patient later at a second time point; and
measuring levels of one or more biomarkers in the first aqueous humor sample and the second aqueous humor sample, wherein the biomarkers are selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A in the aqueous humor sample, wherein detection of increased levels of the one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A in the second aqueous humor sample compared to the first aqueous humor sample indicate that the patient is worsening, and wherein detection of decreased levels of the one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A in the second aqueous humor sample compared to the first aqueous humor sample indicate that the patient is improving.
127. A method of monitoring efficacy of a treatment of a patient for uveitis, the method comprising:
obtaining a first aqueous humor sample from the patient before the patient undergoes the treatment and a second aqueous humor sample from the patient after the patient undergoes the treatment;
measuring levels of one or more biomarkers in the first aqueous humor sample and the second aqueous humor sample, wherein the one or more biomarkers are selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A; and
evaluating the efficacy of the treatment, wherein detection of increased levels of the one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A in the second aqueous humor sample compared to the first aqueous humor sample indicate that the patient is worsening or not responding to the treatment, and detection of decreased levels of the one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A in the second aqueous humor sample compared to the first aqueous humor sample indicate that the patient is improving.
128. The method of aspect 127, further comprising altering the treatment if the patient is worsening or not responding to the treatment.
129. A kit for diagnosing uveitis comprising agents for detecting at least 3 biomarkers selected from the group consisting of IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A.
130. The kit of aspect 129, wherein the kit comprises agents for detecting all of the IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A biomarkers.
131. The kit of aspect 129 or 130, further comprising reagents for performing an aptamer-based proteomic assay or immunoassay.
132. The kit of aspect 131, wherein the kit comprises an aptamer or antibody that specifically binds to IGHM, an aptamer or antibody that specifically binds to TXNDC5, an aptamer or antibody that specifically binds to SLAMF7, an aptamer or antibody that specifically binds to MZB1, an aptamer or antibody that specifically binds to IGHD, an aptamer or antibody that specifically binds to CR2, an aptamer or antibody that specifically binds to IGHG4, an aptamer or antibody that specifically binds to LY9, an aptamer or antibody that specifically binds to FCRL5, an aptamer or antibody that specifically binds to TNFRSF13B, an aptamer or antibody that specifically binds to MMP9, an aptamer or antibody that specifically binds to IL1R2, an aptamer or antibody that specifically binds to GLIPR2, an aptamer or antibody that specifically binds to MPO, an aptamer or antibody that specifically binds to RETIN, an aptamer or antibody that specifically binds to CD177, an aptamer or antibody that specifically binds to PSTPIP1, an aptamer or antibody that specifically binds to CLEC12A, an aptamer or antibody that specifically binds to HMGB2, an aptamer or antibody that specifically binds to LTA4H, an aptamer or antibody that specifically binds to VWF, an aptamer or antibody that specifically binds to TIE1, an aptamer or antibody that specifically binds to SELE, CDH5, an aptamer or antibody that specifically binds to ANGPT2, an aptamer or antibody that specifically binds to FLT4, an aptamer or antibody that specifically binds to ADGRF5, an aptamer or antibody that specifically binds to VEGFC, an aptamer or antibody that specifically binds to TEK, an aptamer or antibody that specifically binds to MYCT1, an aptamer or antibody that specifically binds to CXCL10, an aptamer or antibody that specifically binds to IL18BP, an aptamer or antibody that specifically binds to CCL22, an aptamer or antibody that specifically binds to TNFRSF8, an aptamer or antibody that specifically binds to CCL7, an aptamer or antibody that specifically binds to MMP12, an aptamer or antibody that specifically binds to IL110, an aptamer or antibody that specifically binds to CXCL11, an aptamer or antibody that specifically binds to CELA1, an aptamer or antibody that specifically binds to GNLY, an aptamer or antibody that specifically binds to CST7, an aptamer or antibody that specifically binds to CD8A, an aptamer or antibody that specifically binds to CD5, an aptamer or antibody that specifically binds to GZMK, an aptamer or antibody that specifically binds to GZMA, an aptamer or antibody that specifically binds to CCL5, an aptamer or antibody that specifically binds to CD7, an aptamer or antibody that specifically binds to LCK, and an aptamer or antibody that specifically binds to SH2D1A.
133. The kit of any one of aspects 129-132, further comprising instructions for determining whether a subject has uveitis.
134. A protein selected from the group consisting of IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A for use as a biomarker in diagnosing uveitis.
135. A protein selected from BICDL1, NPPC, HES6, FGF23, AP4M1, SPATA33, MPPED2, and CHRNA5, GPC2 in the aqueous humor sample, wherein decreased levels of the one or more biomarkers selected from BICDL1, NPPC, HES6, FGF23, AP4M1, SPATA33, MPPED2, CHRNA5, and GPC2 for use as a biomarker to determine if uveitis is causing damage to cone cells.
136. A protein selected from OPCML, GAP43, RIPPLY3, LRIT3, PLA2G5, FGF18, PLD5, and KCTD4 in the aqueous humor sample, wherein decreased levels of the one or more biomarkers selected OPCML, GAP43, RIPPLY3, LRIT3, PLA2G5, FGF18, PLD5, and KCTD4 for use as a biomarker to determine if uveitis is causing damage to retinal pigment epithelium (RPE) cells.
137. A protein selected from CPNE7, TYRO3, TENM4, MDM4, RDH13, SV2A, SYT5, OLFM3, RIC3, and SEMA6C in the aqueous humor sample, wherein decreased levels of the one or more biomarkers selected CPNE7, TYRO3, TENM4, MDM4, RDH13, SV2A, SYT5, OLFM3, RIC3, and SEMA6C for use as a biomarker to determine if uveitis is causing damage to amacrine cells.
138. An in vitro method of uveitis, the method comprising:
obtaining an aqueous humor sample from an eye of the patient; and
measuring levels of one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A in the aqueous humor sample, wherein increased levels of the one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A compared to reference value ranges for the levels of the one or more biomarkers in a control aqueous humor sample indicate that the patient has uveitis.
139. The method of aspect 138, further comprising measuring levels of one or more biomarkers selected from BICDL1, NPPC, HES6, FGF23, AP4M1, SPATA33, MPPED2, and CHRNA5, GPC2 in the aqueous humor sample, wherein decreased levels of the one or more biomarkers selected from BICDL1, NPPC, HES6, FGF23, AP4M1, SPATA33, MPPED2, CHRNA5, and GPC2 compared to reference value ranges for the levels of the one or more biomarkers in a control aqueous humor sample indicate that the uveitis is causing damage to cone cells.
140. The method of aspect 138 or 139, further comprising measuring levels of one or more biomarkers selected from OPCML, GAP43, RIPPLY3, LRIT3, PLA2G5, FGF18, PLD5, and KCTD4 in the aqueous humor sample, wherein decreased levels of the one or more biomarkers selected OPCML, GAP43, RIPPLY3, LRIT3, PLA2G5, FGF18, PLD5, and KCTD4 compared to reference value ranges for the levels of the one or more biomarkers in a control aqueous humor sample indicate that the uveitis is causing damage to retinal pigment epithelium (RPE) cells.
141. The method of any one of aspects 138-140, further comprising measuring levels of one or more biomarkers selected from CPNE7, TYRO3, TENM4, MDM4, RDH13, SV2A, SYT5, OLFM3, RIC3, and SEMA6C in the aqueous humor sample, wherein decreased levels of the one or more biomarkers selected CPNE7, TYRO3, TENM4, MDM4, RDH13, SV2A, SYT5, OLFM3, RIC3, and SEMA6C compared to reference value ranges for the levels of the one or more biomarkers in a control aqueous humor sample indicate that the uveitis is causing damage to amacrine cells.
142. A method of determining biological age of an eye in a patient, the method comprising:
obtaining an aqueous humor sample from an eye of the patient; and
measuring levels of one or more biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 in the aqueous humor sample; and
using a machine learning aging model to determine the biological age of the eye based on the levels of the one or more biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3.
143. The method of aspect 142, wherein the levels of at least two, at least three, or at least four biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 are measured in the aqueous humor sample.
144. The method of aspect 143, wherein the levels of LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 are measured in the aqueous humor sample.
145. The method of any one of aspects 142-144, wherein said measuring comprises performing an aptamer-based proteomic assay, mass spectrometry, liquid chromatography-tandem mass spectrometry, tandem mass spectrometry, an enzymatic or biochemical assay, liquid chromatography, NMR, an enzyme-linked immunosorbent assay (ELISA), a radioimmunoassay (RIA), an immunofluorescent assay (IFA), immunohistochemistry, or a Western Blot.
146. The method of aspect 145, wherein the aptamer-based proteomic assay is performed using a multiplex aptamer array.
147. The method of any one of aspects 142-146, wherein the machine learning aging model uses an XGBoost algorithm.
148. A kit comprising agents for detecting at least 3 biomarkers selected from the group consisting of LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3.
149. The kit of aspect 148, wherein the kit comprises agents for detecting all of the LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 biomarkers.
150. The kit of aspect 148 or 149, further comprising reagents for performing an aptamer-based proteomic assay or immunoassay.
151. The kit of aspect 150, wherein the kit comprises an aptamer or antibody that specifically binds to LECT2, an aptamer or antibody that specifically binds to LECT2, an aptamer or antibody that specifically binds to DCBLD1, an aptamer or antibody that specifically binds to SCGN, an aptamer or antibody that specifically binds to ACAN, an aptamer or antibody that specifically binds to AOC2, an aptamer or antibody that specifically binds to CAPS, an aptamer or antibody that specifically binds to TFF3, an aptamer or antibody that specifically binds to AMN, an aptamer or antibody that specifically binds to ABO, an aptamer or antibody that specifically binds to NETO1, an aptamer or antibody that specifically binds to CD274, an aptamer or antibody that specifically binds to PPBP, an aptamer or antibody that specifically binds to ABL1, an aptamer or antibody that specifically binds to HAMP, an aptamer or antibody that specifically binds to IGFBP1, an aptamer or antibody that specifically binds to AAMDC, an aptamer or antibody that specifically binds to A1BG, an aptamer or antibody that specifically binds to ADA2, an aptamer or antibody that specifically binds to MVP, an aptamer or antibody that specifically binds to CRYGC, an aptamer or antibody that specifically binds to A4GALT, an aptamer or antibody that specifically binds to A4GNT, an aptamer or antibody that specifically binds to AADAT, an aptamer or antibody that specifically binds to A2M, and an aptamer or antibody that specifically binds to ABI3.
152. The kit of any one of aspects 148-151, further comprising instructions for predicting biological age and determining risk of age-related pathology and morbidity in a patient or determining the biological age of the eye.
153. A method of estimating age of a subject, the method comprising:
obtaining an aqueous humor sample from an eye of the patient; and
measuring levels of one or more biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 in the aqueous humor sample; and
using a machine learning aging model to estimate age of a subject based on the levels of the one or more biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3.
154. The method of aspect 153, wherein the machine learning aging model uses an XGBoost algorithm for age classification.
155. An in vitro method of determining biological age and risk of age-related pathology and morbidity of a patient, the method comprising:
obtaining an aqueous humor sample from an eye of the patient; and
measuring levels of one or more biomarkers selected from the LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 in the aqueous humor sample; and
   using a machine learning aging model to determine biological age and the risk of age-related pathology and morbidity of the patient based on the levels of the one or more biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3.
156. A protein selected from the group consisting of LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 for use as a biomarker in predicting biological age and determining risk of age-related pathology and morbidity.
157. A protein selected from the group consisting of LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 for use as a biomarker for determining biological age of an eye.
158. A protein selected from the group consisting of LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 for use as a biomarker for estimating age of a subject.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Liquid Biopsy Proteomics Combined with AI Identifies Cellular Drivers of Eye Aging and Disease In Vivo

### Introduction

In this study we demonstrate an approach for integrating microvolume liquid biopsy proteomics, single-cell transcriptomics and artificial intelligence (AI) in a way that obviates the aforementioned limitations, creating a powerful tool to examine disease mechanisms at the cell level in vivo. We refer to this approach as TEMPO (Tracing Expression of Multiple Protein Origins). As exemplified by the human eye, TEMPO yields surprising new insights into disease mechanisms and into the connection between aging and disease.

The human eye is a complex organ composed of tissues from all three germ layers and contains a variety of cell types, including neuronal, vascular, stromal, immune, and blood cells.³ Ocular tissue transparency allows direct visualization and precise clinical phenotyping of human diseases ranging from genetic conditions to metabolic, inflammatory, and neurodegenerative diseases. While some diseases like retinitis pigmentosa are specific to the eye, others reflect systemic disease. Diabetic retinopathy is a major cause of blindness, reflecting systemic microangiopathy in patients with diabetes, a major cause of mortality affecting 1 in 11 adults worldwide.⁴ Uveitis is another leading cause of blindness due to ocular autoimmunity or autoinflammation that is often a manifestation of systemic rheumatologic disease. Parkinson's disease (PD) is one of the most common neurodegenerative diseases with an estimated lifetime risk of up to 2%.⁵ Diagnostic assessment of PD is often challenging, as routine brain tissue biopsies are not feasible. Interestingly, retinal imaging shows subtle structural changes in PD patients suggesting that alterations of the eye may correlate with brain pathology.⁶ Thus, the eye not only enables human's primary sense for interaction with the environment, it also provides a broad view into a person's health.

Fifty-seven different cell types have been identified in the human eye, all of which have been characterized by RNA-sequencing at the single cell level.⁷⁻¹⁷ All of these cell types can release their proteins into the eye's two fluid-filled chambers, which contain either the vitreous fluid overlying the retina or the aqueous fluid (aqueous humor, AH) behind the cornea. The blood-ocular barrier separates eye fluid from plasma, leading to a distinct protein composition inside the eye. Fluid from both of these chambers can be safely biopsied using local anesthesia while the patient is awake. Due to the unique, chambered structure containing locally enriched fluid, the eye represents an ideal organ to combine liquid biopsy proteomics with tissue cell level transcriptomics. Here, we applied high-resolution proteomic profiling using a DNA aptamer-based assay to trace nearly six-thousand proteins within the AH or vitreous fluid back to their cells of origin. We observed cell-specific marker proteins with unexpected disease activities. Further, analysis by artificial intelligence (AI) was used to create proteomic clocks that predict chronological age in healthy subjects and to develop cell-specific aging models, which uncovered evidence of accelerated cell aging in diseases where older age per se is not a risk factor. The multi-modal TEMPO approach makes it possible to molecularly monitor individual cell types in living humans during aging and disease, and is applicable, in principle, to other organs such as the brain (using cerebrospinal fluid), lungs (fluid from bronchoalveolar lavage), and kidney (urine), breast (breast milk), along with joints (synovial fluid) and tumor cysts.

### Results

### Multi-Omics Approach Determines the Cellular Origin of Liquid Biopsy Proteins

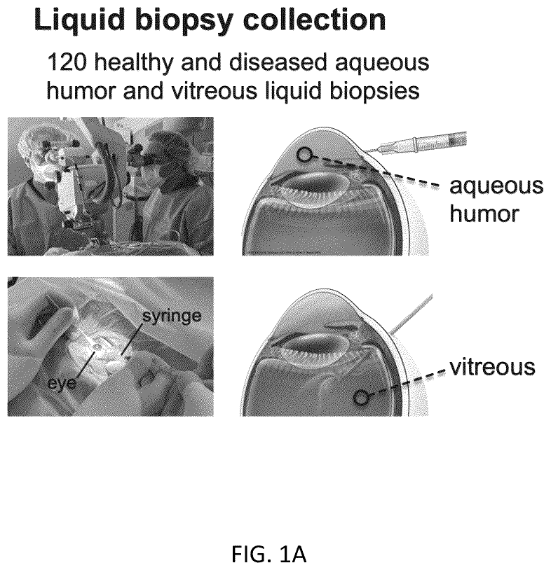

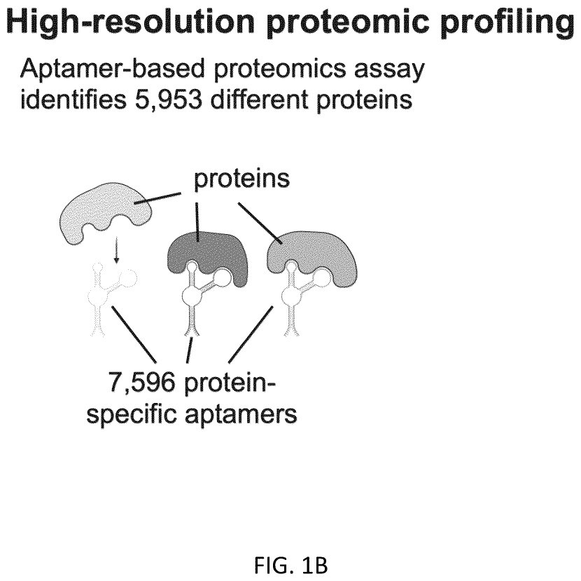

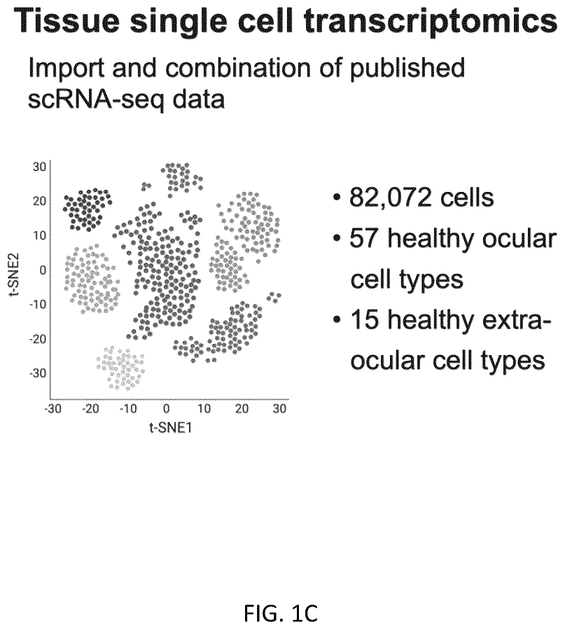

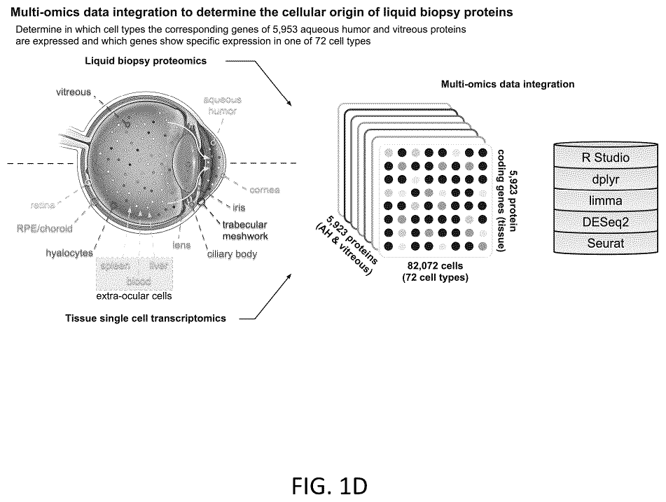

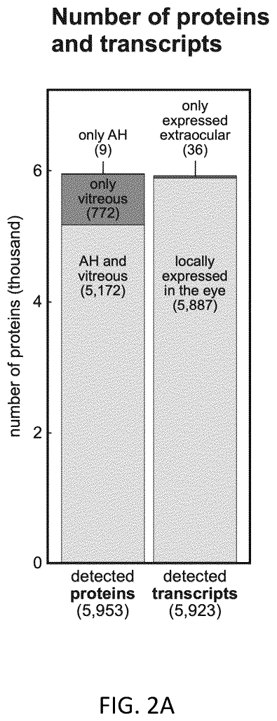

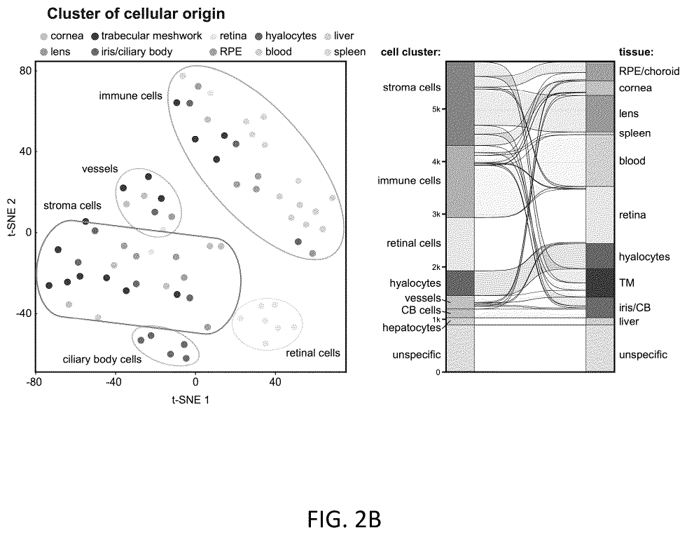

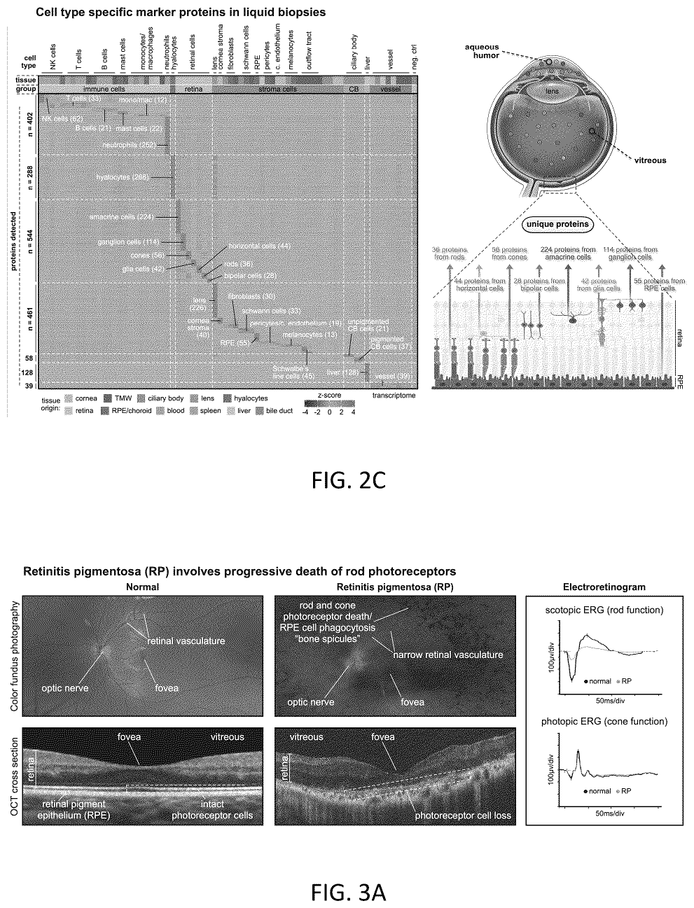

120 liquid biopsies were obtained from the AH or vitreous of patients undergoing eye surgery (FIG. 1A). High-resolution proteomic profiling was applied using an aptamer-based assay covering 6,345 human proteins,¹⁸ of which 5,953 proteins were detected in healthy samples (FIG. 1B). To determine which cell types expressed the genes encoding these proteins, scRNA-seq data covering 82,072 single cells from all 57 known healthy ocular cell types and 15 healthy extra-ocular cell types, including blood and liver cells were combined (FIG. 1C) and integrated with the liquid biopsy proteomics data (FIG. 1D). For 99.5% (5,923) of the identified proteins, the expression of the corresponding gene was confirmed within the RNA-seq data, and 99.4% (5,887) of these genes were locally expressed in the eye (FIG. 2A). AH is surgically easier to access than the vitreous. However, proteomic analysis of AH has been challenged by its low volume and low protein concentration, which is about 6 times lower than in the vitreous and about 400 times lower than in plasma.¹⁹⁻²¹ Using the aptamer-based assay, we found that 87.0% (5,172) of vitreous proteins could be detected in 50 microliters of AH, pointing towards a previously unreported substantial protein exchange between both fluid-filled compartments despite enzymatic and anatomic barriers (FIG. 2A). Unsupervised cluster analysis based on all 5,923 protein-coding genes revealed a predominantly cell-type-dependent rather than tissue-type-dependent clustering, as demonstrated by distinct clusters for immune, stroma, vascular, and retinal cells (FIG. 2B).

### Protein Biomarker Clusters Recognize Specific Cell-Types

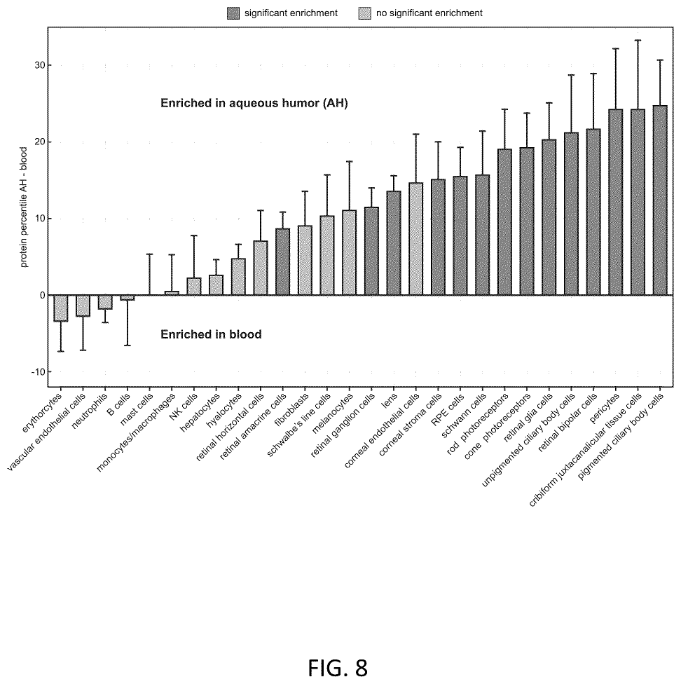

The multi-omics data integration identified 1,920 cell type marker proteins with highly specific expression in their respective cell type (FIG. 2C, Table 1). We established cell-specific markers for a) individual retinal cell types, including markers for ganglion cells (n=114 markers), amacrine cells (n=224), retinal glia cells (n=42), bipolar cells (n=28), horizontal cells (n=44), rod photoreceptors (n=36), and cone photoreceptors (n=56); b) for several immune cell types, including macrophages (n=12), hyalocytes (macrophages of the vitreous) (n=288), neutrophils (n=252), T cells (n=33), and B cells (n=21); and c) for other ocular cell types, such as retinal pigment epithelium (RPE) cells (n=55), fibroblasts (n=30), ciliary body cells (n=58), vascular endothelial cells (n=39), and pericytes (n=19). Bile duct tissue was included as a negative control and showed negative gene expression for all identified markers. The detection of cell-specific intracellular proteins from the retina demonstrates natural protein release into the vitreous and successful migration into the anterior chamber of the eye, despite proteins having to diffuse through enzymatic, proteoglycan, and connective tissue barriers (FIG. 2C). The proteins from eye-specific cell types, such as photoreceptors or ciliary body cells, were strongly enriched in AH compared to plasma, indicating a substantial local enrichment of these protein signatures (FIG. 8). Interestingly, 128 secreted proteins were expressed only in hepatocytes, implying some transport to the eye via the plasma and past the blood-ocular barrier (FIG. 2C). The cell-specific proteins in liquid biopsies provide a valuable tool to molecularly monitor individual cell types in living humans with disease in non-regenerative tissues such as the retina without the need for a direct tissue biopsy.

Cell Circuits within Complex Tissues can be Molecularly Monitored During Disease

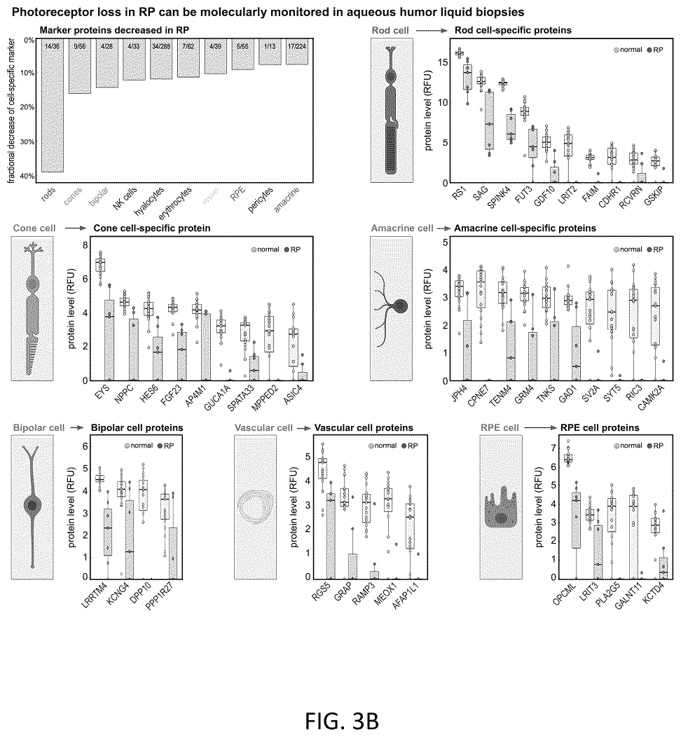

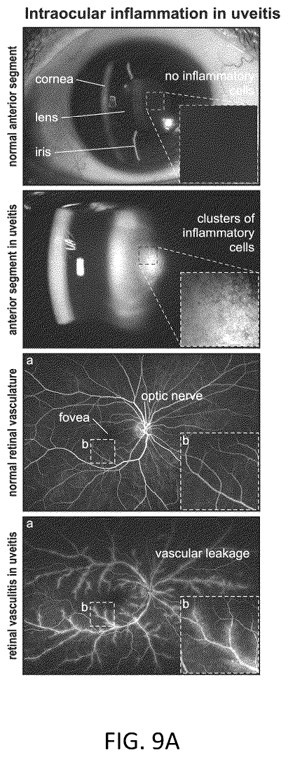

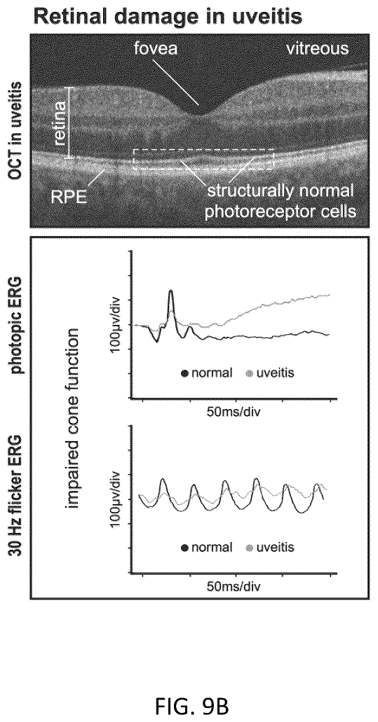

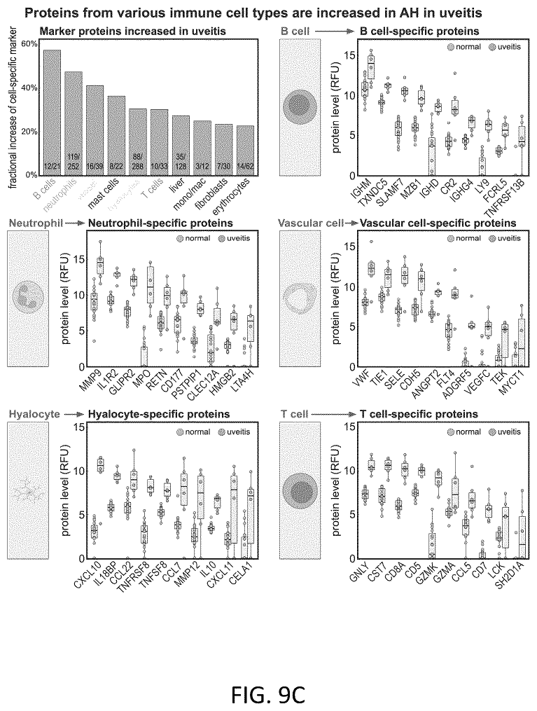

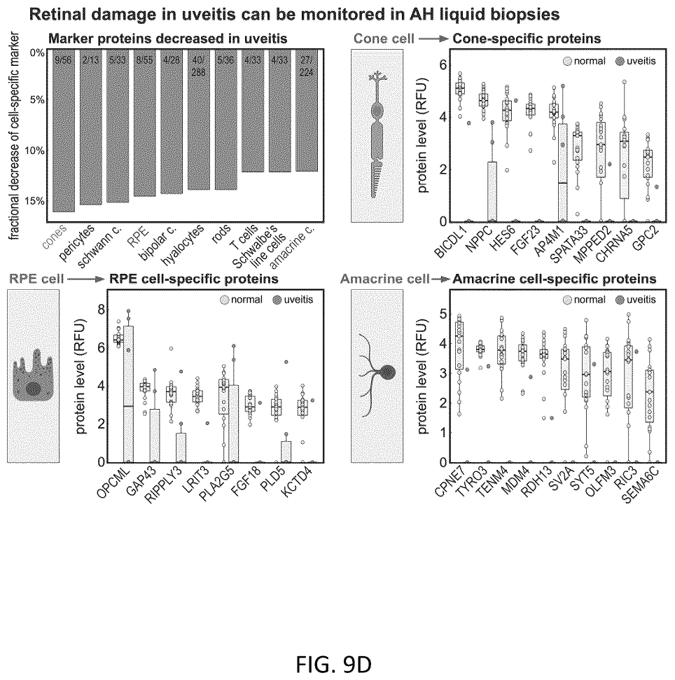

Retinitis pigmentosa (RP) is a rare genetic retinal disease characterized by the progressive death of rod photoreceptors and is a major target for human gene therapy trials (FIG. 3A). The genes linked to RP are expressed by photoreceptors or RPE, the known sites of structural damage.^{22,23} However, it is poorly understood which other cells forming a neural circuit are affected in human RP, and it has not been possible to molecularly assess RP pathology in living humans. We analyzed which of the 1,920 identified cell type-specific marker proteins (FIG. 2C) were significantly decreased in RP. As anticipated, rod photoreceptor specific proteins were the most affected (38.9% of all rod markers were decreased in RP; FIG. 3A). Although the protein encoded by any given RP-linked gene is restricted to rod photoreceptors or RPE, we also found a decrease of marker proteins from cone photoreceptors (16.1%), retinal bipolar cells (14.3%), retinal amacrine cells (7.6%), erythrocytes (11.3%), and vascular endothelial cells (10.3%) (FIG. 3B), whereas other retinal cell types, including horizontal cells and ganglion cells, remained relatively unaffected. We also tested patients with uveitis, an intraocular inflammation that can damage blood vessels and retinal cells (FIGS. 9A-9B), making uveitis one of the leading causes of visual impairment.²⁴ We found a significant increase in hundreds of marker proteins from various immune cells, including B cells, neutrophils, mast cells, hyalocytes, T cells, and macrophages (FIG. 9C). The analysis also demonstrated molecular damage from uveitis to several retinal cell types, including cone photoreceptors, bipolar cells, and amacrine cells (FIG. 9D), which correlates with observed electrophysiological changes even when cells appear anatomically normal on clinical testing (FIG. 9B). In sum, TEMPO allowed us to molecularly monitor photoreceptors and six other cell types in a retinal circuit and to detect immune-mediated molecular damage to distinct bystander neuronal cells in living humans.

Cellular Drivers of Diabetic Retinopathy Switch with Disease Stage

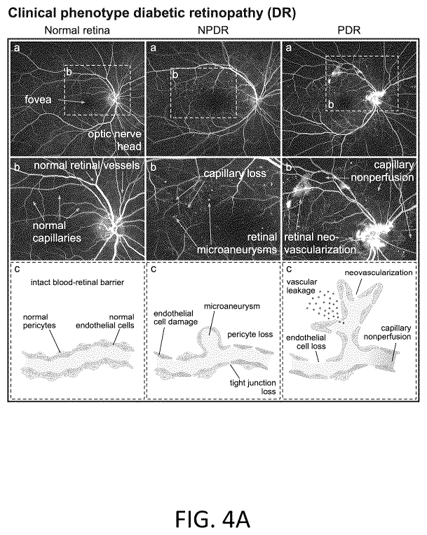

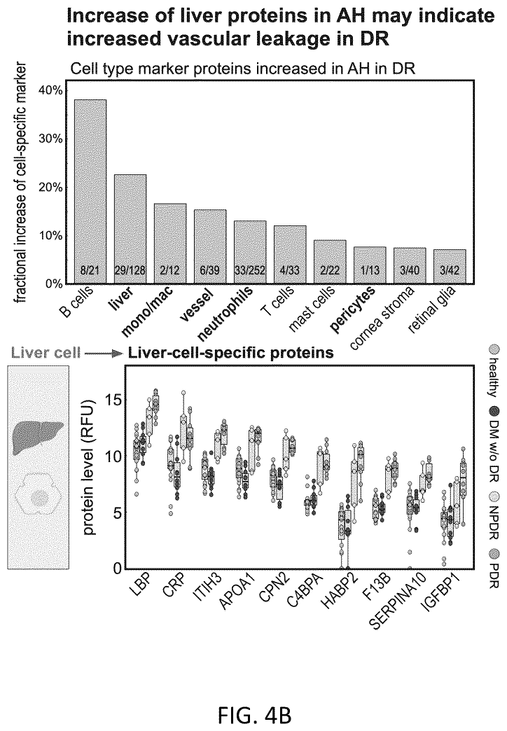

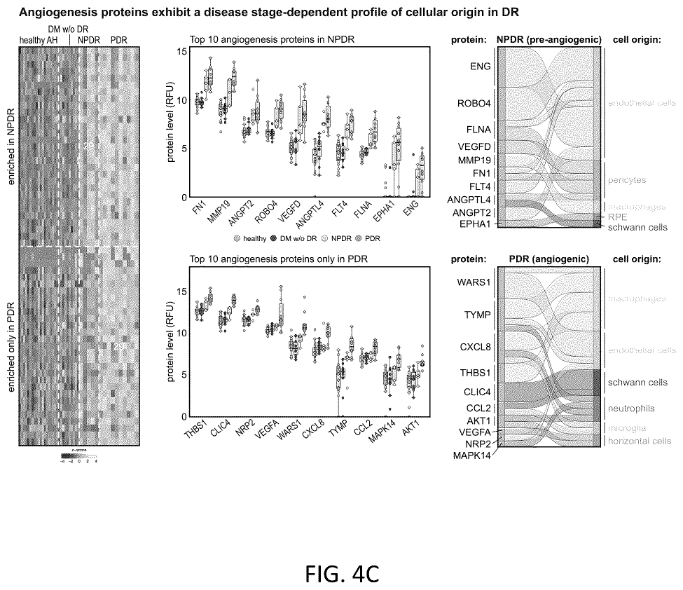

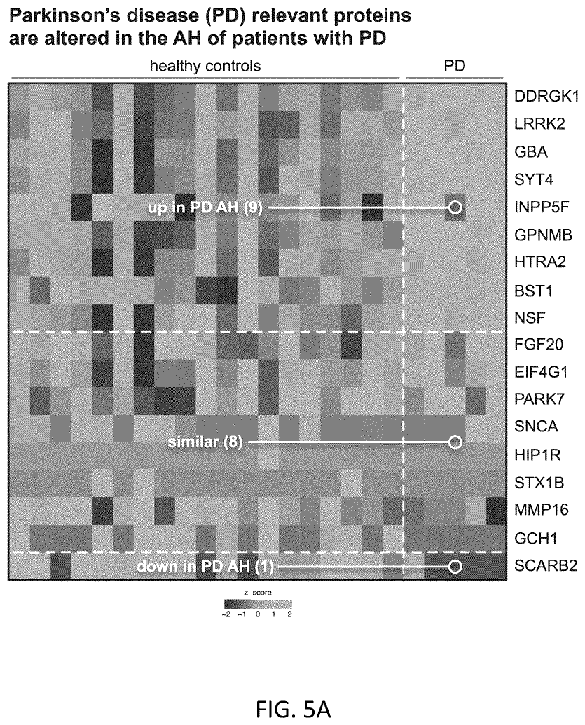

Diabetic retinopathy (DR) is a leading cause of blindness affecting 100 million people worldwide.²⁵ DR is a microangiopathy of the retina characterized by vascular damage and increased vascular permeability, leading to retinal ischemia and secondary pathological neovascularization (FIG. 4A). The disease progresses from a non-proliferative (NPDR) to proliferative stage (PDR), the latter characterized by retinal neovascularization, which can lead to severe blinding complications. Although imaging techniques are used to evaluate the retinal structural changes in DR, they cannot directly assess the molecular and cell changes in vivo, information critical for testing new drugs and optimizing therapies. scRNA-seq data has been applied to models of NPDR in animals,²⁶ but the molecular events and the cell types driving DR in humans remain poorly understood, since animal models fail to mimic most aspects of DR pathogenesis, in particular PDR.²⁷

In liquid biopsies from DR patients, we first analyzed which cell type-specific marker proteins were significantly increased. We observed marker protein expression changes from immune cells (B cells, macrophages, neutrophils, T cells, and mast cells), vascular endothelial cells, pericytes, and retinal glia cells (FIG. 4B). There was also a significant increase of liver proteins, among them CRP, APOA1, F13B, SERPINA10, and IGFBP1 (FIG. 4B), likely the result of the increased vascular permeability arising from disruption of the blood-retinal barrier in DR. Pathway analysis revealed that the liver-derived proteins in DR were involved in acute phase response, acute inflammatory response, blood coagulation, and wound healing (Table 3). This raises the possibility that liver proteins may directly contribute to inflammation in DR pathology.

We next focused on angiogenic proteins since this class of proteins is known to play a key role in DR. It is largely unknown which ocular cell types produce these factors and whether these cell types differ between disease stages. In total, 58 angiogenic proteins were differentially enriched in DR compared to controls (FIG. 4C). There were 29 proteins increased in both disease stages, including ANGPT2, MMP19, VEGFD, ANGPTL4, and ENG (FIG. 4C). Another 29 proteins were specifically increased in PDR but not in NPDR, among them VEGFA, CXCL8, TYMP, CCL2, and WARS1. Some of these proteins are known key players in DR, including VEGFA, which is a therapeutic target in PDR and NPDR when macular edema is present. The proteins involved in both disease stages were mainly derived from endothelial cells and pericytes, consistent with the microangiopathic etiology of DR.²⁵ However, it was striking that the PDR-specific angiogenic proteins predominantly originated from an entirely different class of cells, namely, immune cells, including macrophages, neutrophils, and retinal microglia (FIG. 4C). This indicates PDR is a cellularly and molecularly distinct disease compared to NPDR and is driven by a distinctive set of angiogenic cells and angiogenic factors rather than simply by the same NPDR cells expressing more of the same angiogenic proteins. These findings strongly suggest immune cells like macrophages are important molecular drivers of later-stage DR in living humans, and this disease mechanism points to new therapeutic strategies where effectiveness could also be molecularly monitored in vivo.

### Brain Disease Proteins are Detected in Eye Fluid

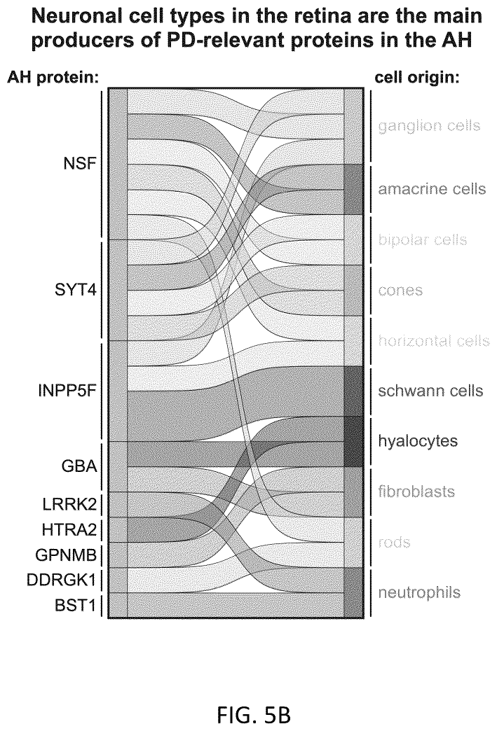

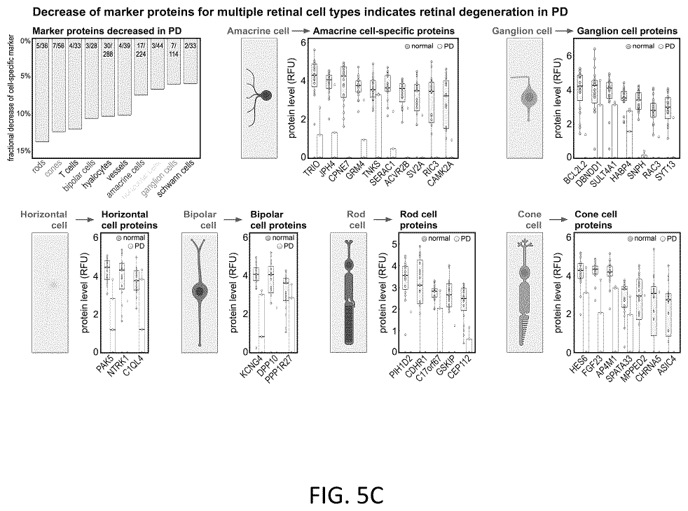

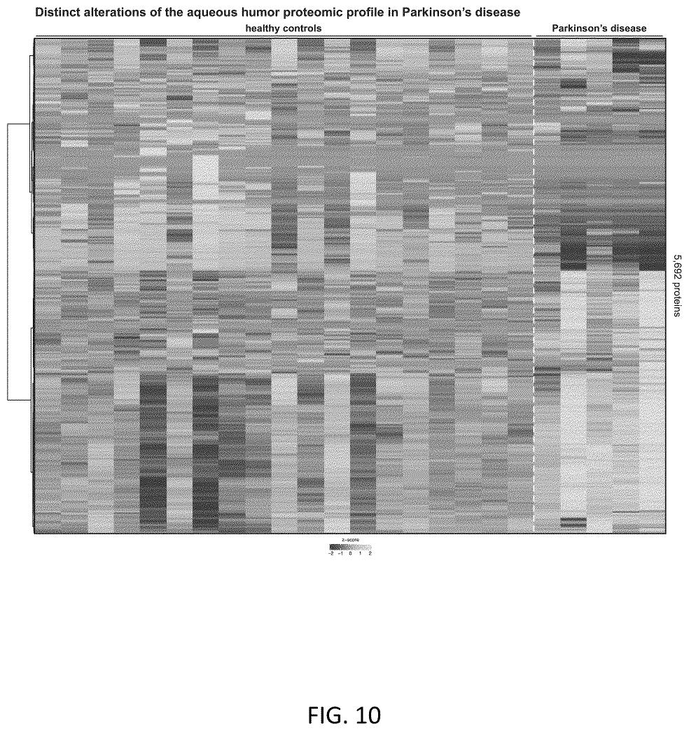

Diagnostic and prognostic assessment of neurodegenerative diseases such as Parkinson's disease (PD) is often challenging, as brain tissue biopsies are exceedingly complex. Here, we studied AH from patients with PD as a complementary, minimally-invasive, and possibly more direct way to assess neuronal cells compared to plasma. Our analysis found distinct differences between PD and control AH (FIG. 10). From a total of 48 PD-relevant proteins,²⁸ 18 proteins were detected in the AH, nine of which were significantly increased in PD, among them NSF, SYT4, and INPP5F (FIG. 5A). Neuronal cell types in the retina were the main sources, including ganglion cells, amacrine cells, bipolar cells, horizontal cells, cones, and rods (FIG. 5B). In addition, we found evidence of molecular dysfunction to several retinal cell types, including amacrine cells, ganglion cells, bipolar cells, and horizontal cells (FIG. 5C). These findings are consistent with reported anatomical changes of the inner retinal layers of PD patients.⁶ Interestingly, we also found evidence of molecular alterations in rod and cone photoreceptors (FIG. 5C), which are located in the outer retina and do not show anatomical changes, suggesting molecular biomarkers may be more sensitive than imaging/structural biomarkers. These results redefine the PD phenotype in the retina at a molecular and cell level and highlight the potential of AH liquid biopsies as a valuable tool for diagnostic assessment or even molecular monitoring of PD therapies.

### Molecular Age of the Eye Follows Non-Linear Trajectories

Chronological age is one of the most important risk factors for a variety of diseases.²⁹ Yet it is well established that tissue aging is discordant with chronological aging,^{30,31} raising questions on whether aging is a disease itself and how disease might impact aging. Based on the plasma proteome, models have been developed to estimate molecular age in humans.^{32,33} However, most models are not organ or cell type-specific, so they cannot identify the combination of cells driving the aging process in any given tissue or organ. Even though scRNA-seq data provided some insight into the molecular changes in aged postmortem human retina³⁴ and choroid,³⁵ for example, it is currently not possible to assess the molecular age of the eye and its cells in living humans under healthy and disease conditions. Understanding cellular aging is even more important now, as molecular rejuvenation therapies are making their way to clinical trials.³¹ However, it is still poorly understood which cell types are driving aging of the eye, a discovery that would open new avenues to target aging and disease.

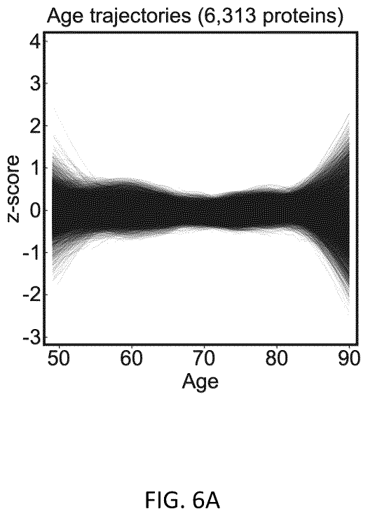

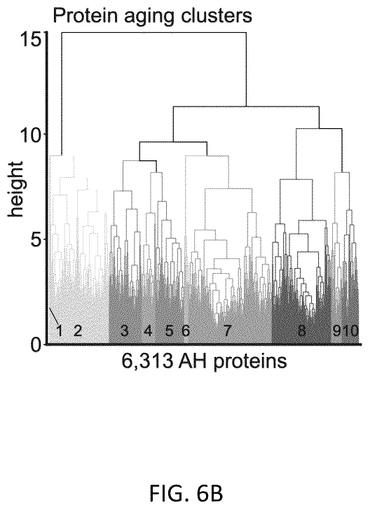

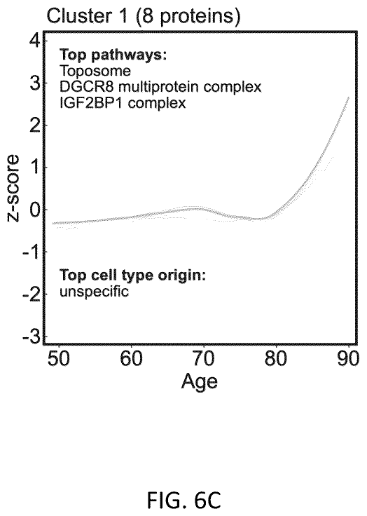

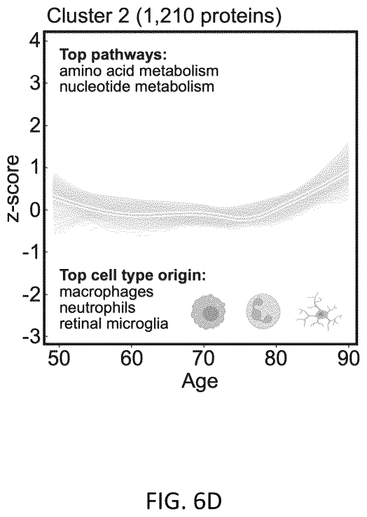

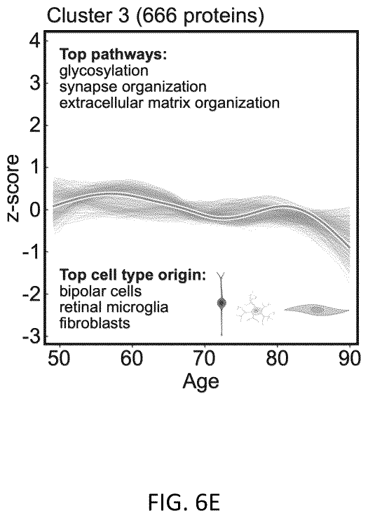

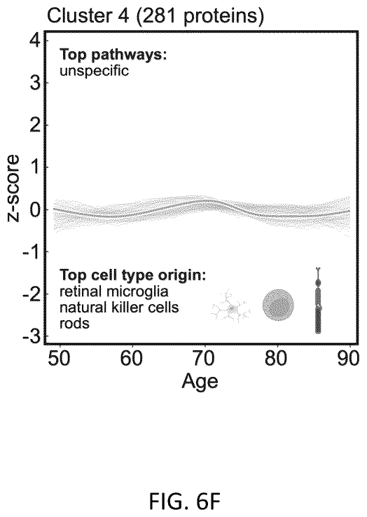

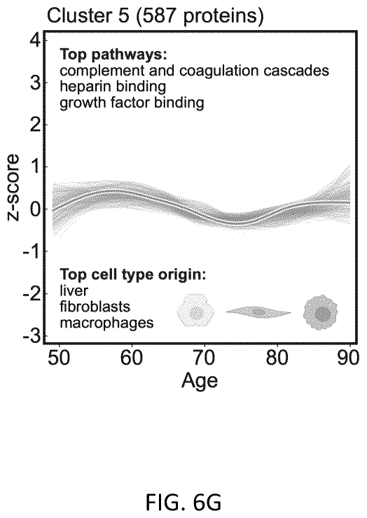

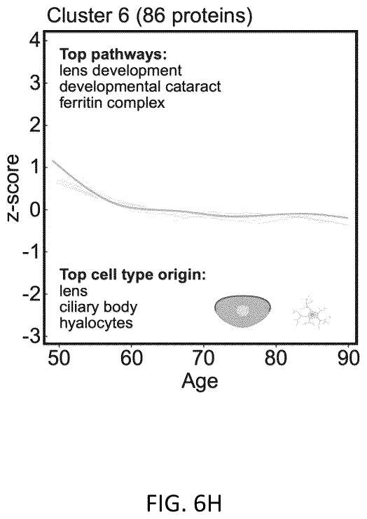

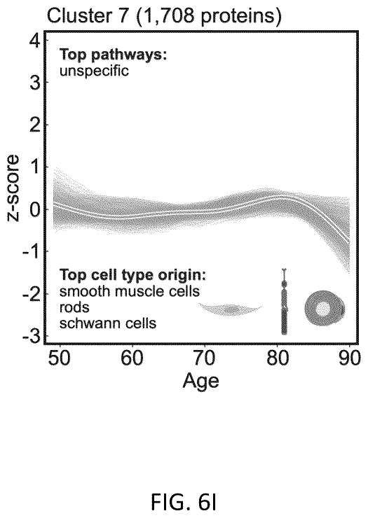

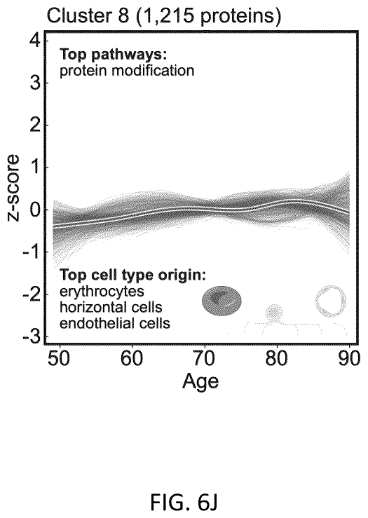

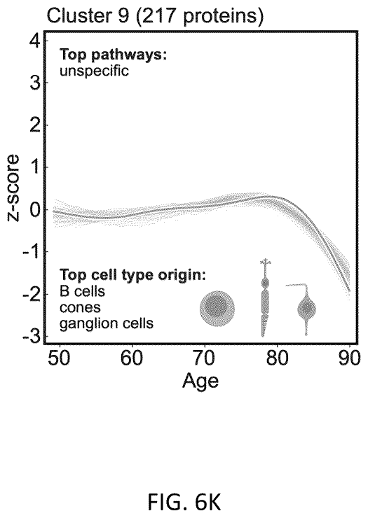

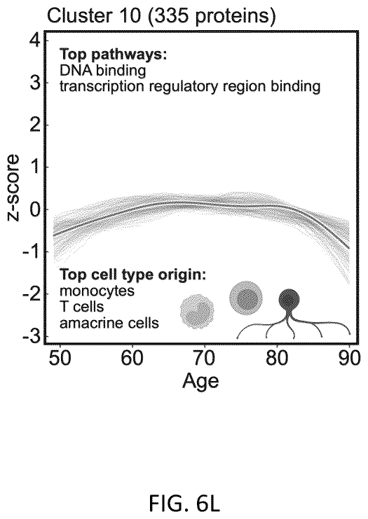

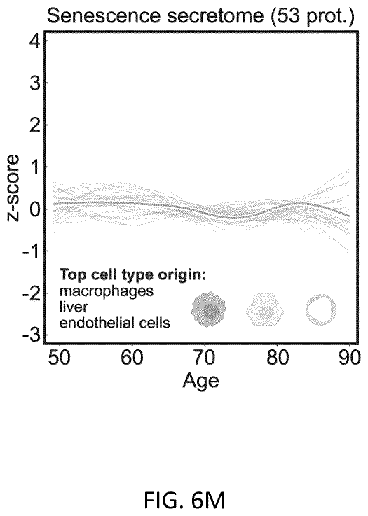

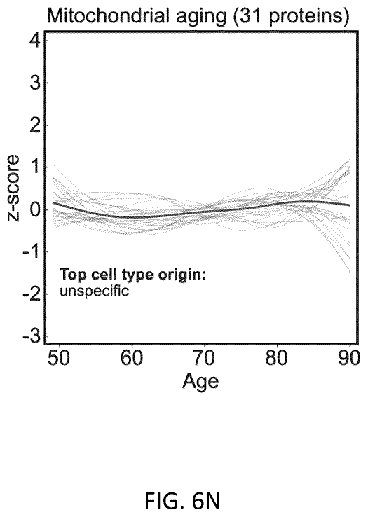

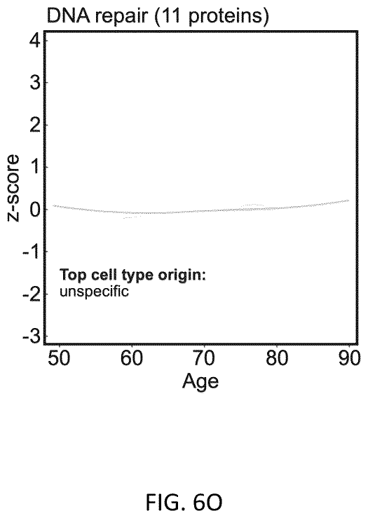

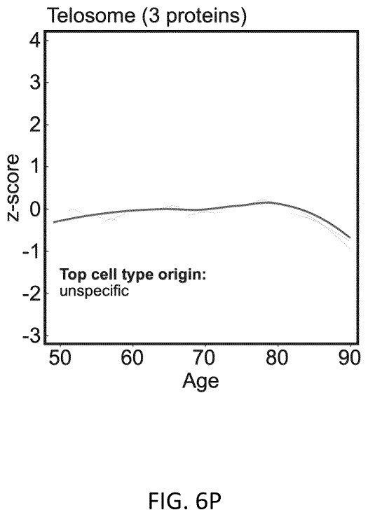

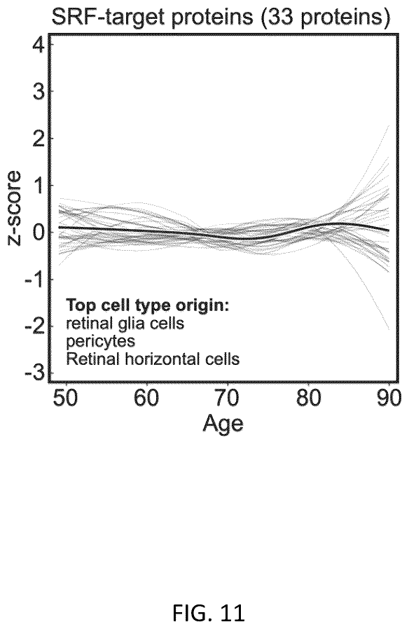

Similar to aging patterns of proteins in human and murine plasma,³² we found undulating changes of 6,313 AH proteins with aging in healthy eyes (FIG. 6A). To analyze the complexity of these proteomic changes, all 6,313 proteins were grouped in 10 clusters with similar trajectories using unsupervised hierarchical cluster analysis (FIG. 6B). Nearly all clusters revealed non-linear trajectories with aging (FIGS. 6C-6L). Seven clusters were enriched for biological pathways and specific cell types (FIGS. 6C-6L) indicating distinct changes in biological pathways and cell types during eye aging. For example, lens and ciliary body-specific proteins peaked around age 50 (Cluster 6, FIG. 6H). Proteins originating from immune cells such as macrophages, neutrophils, and retinal microglia, remained relatively stable until age 80 before increasing significantly (Cluster 2, FIG. 6D), whereas proteins from retinal cells, including cones and ganglion cells, dramatically decreased starting around age 80 (Cluster 9, FIG. 6K). The involvement of retina-specific cells during aging of the eye points towards organ-specific aging patterns which differ from age-trajectories observed in plasma. Proteins in pathways such as the complement and coagulation cascade and heparin binding, were mainly derived from the liver and demonstrated a two-wave increase with peaks around age 60 and 90 (Cluster 5, FIG. 6G) which is consistent with findings in plasma.³² We also analyzed the trajectories of known senescence associated proteins, including proteins involved in the senescence secretome,³⁷ mitochondrial aging,³¹ DNA repair,³⁹ the telosome,⁴⁰ and target genes of serum response factor,⁴¹ also indicating mainly non-linear associations with aging (FIGS. 6M-6P, FIG. 11). From these results, we draw three important conclusions, that the eye proteome changes during normal aging along mainly non-linear trajectories, that the aging process may be largely organ-specific, and that specific pathways and cell types have a critical role in the aging process of the eye.

### AI Models Predict the Global and Cellular Molecular Age

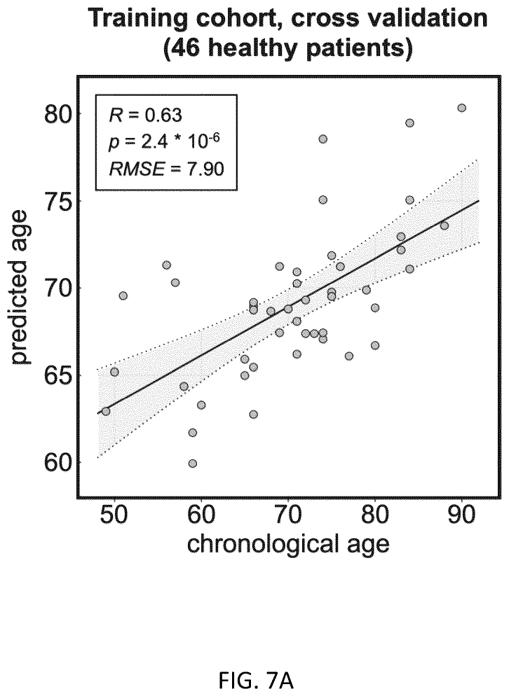

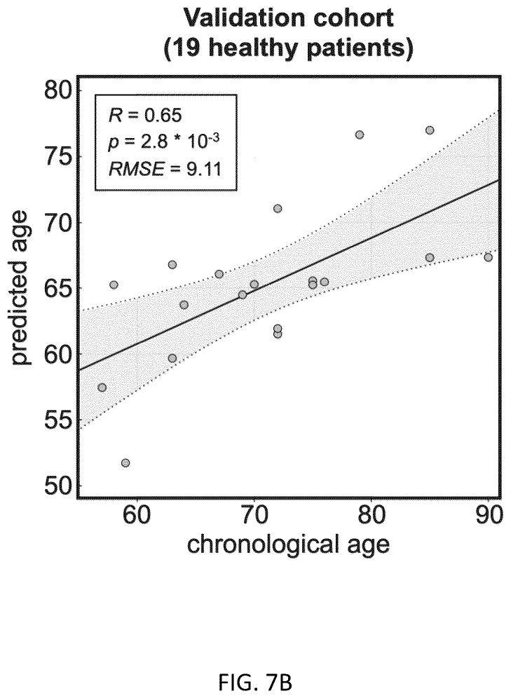

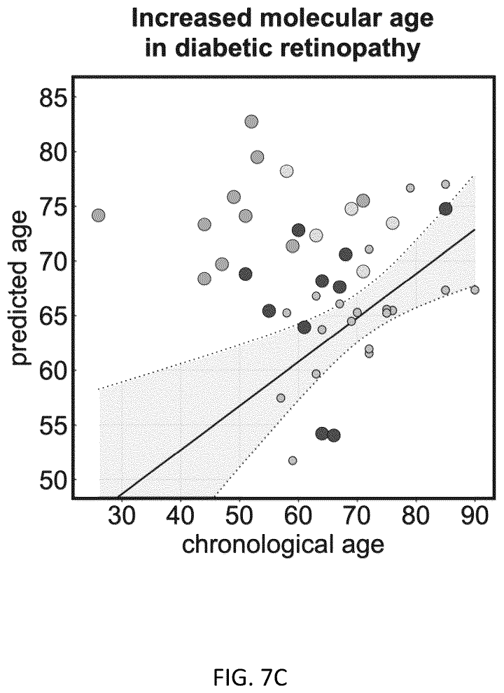

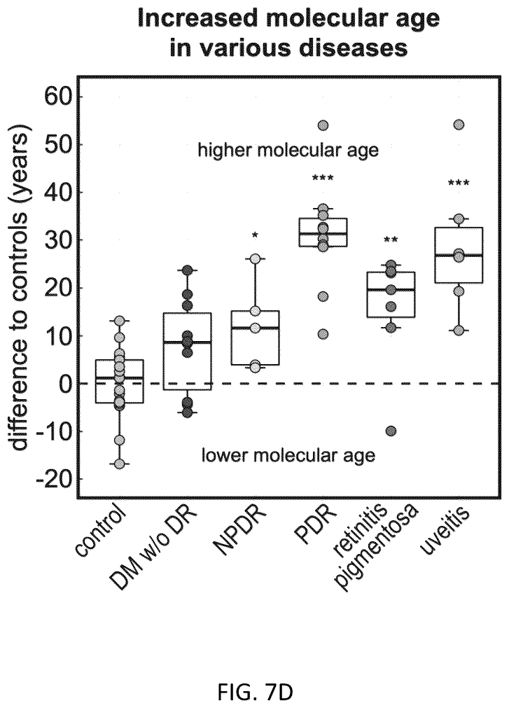

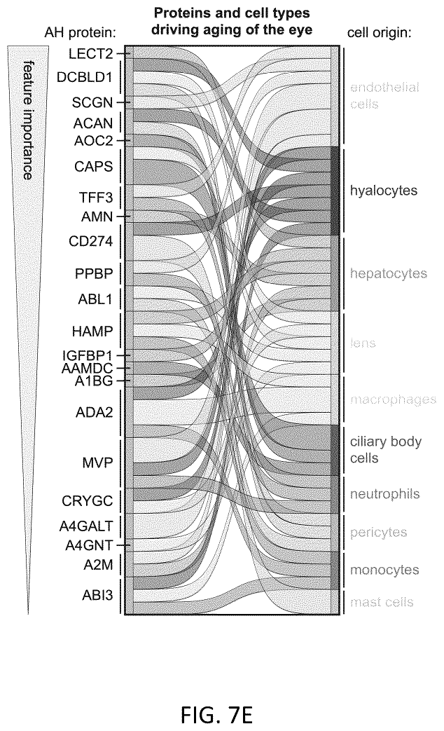

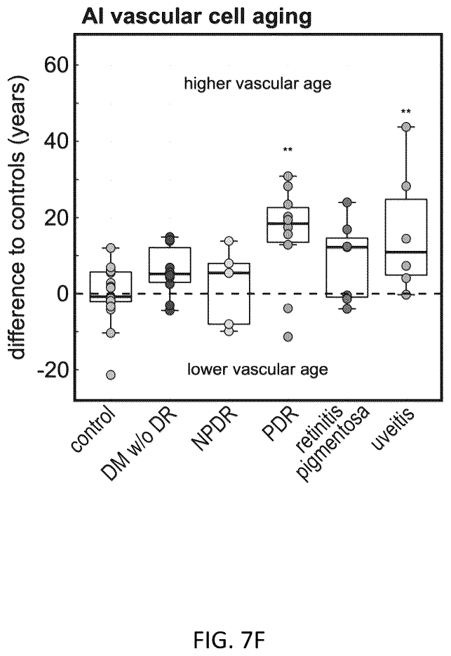

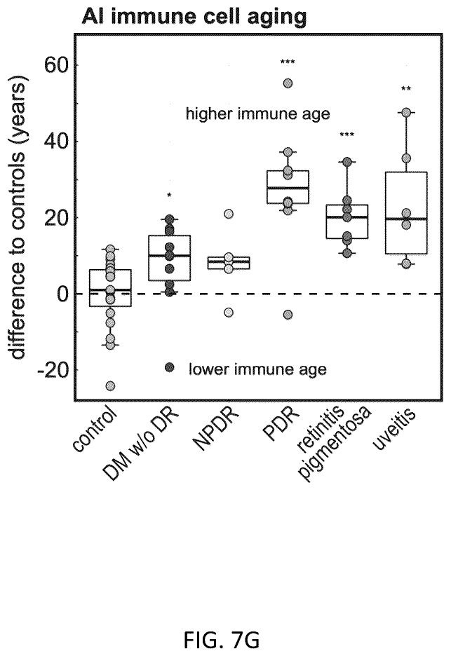

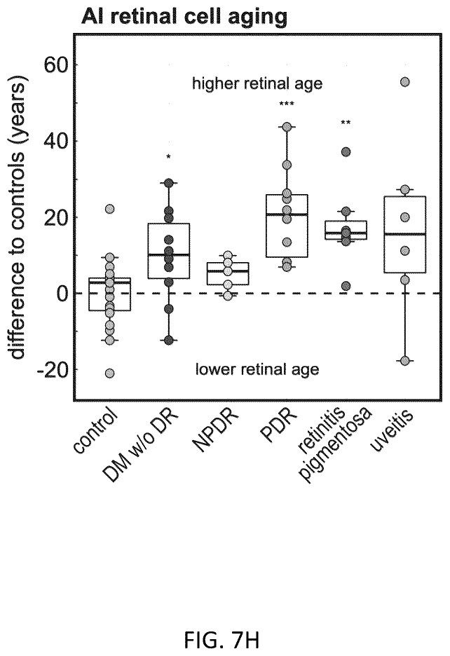

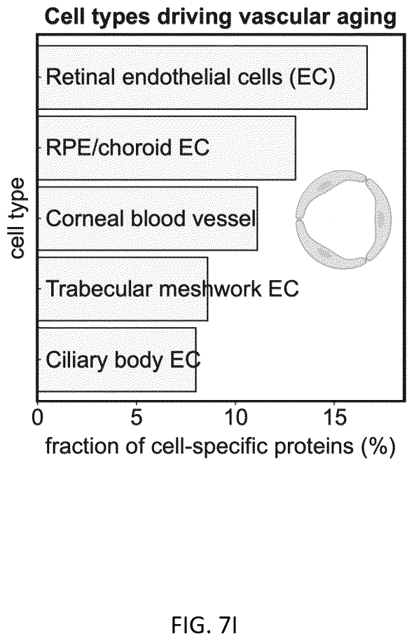

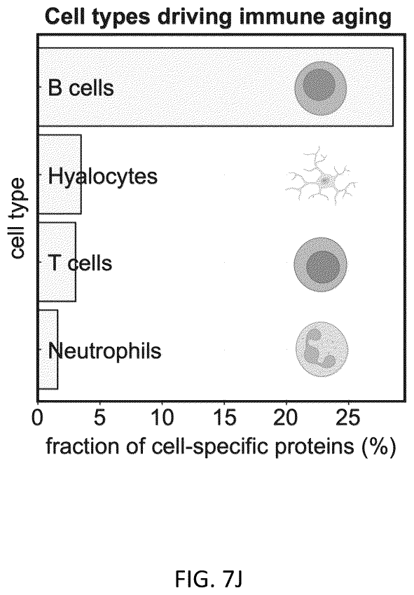

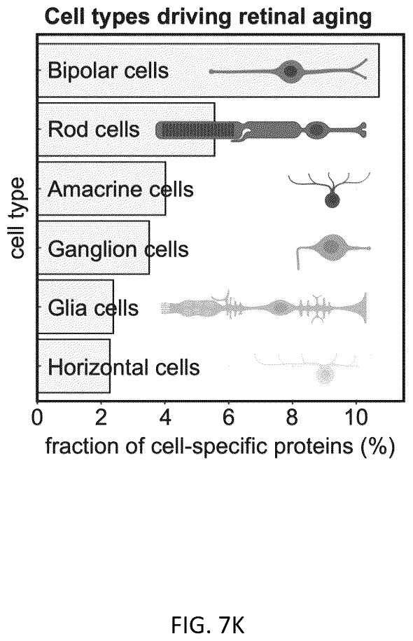

Our next goal was to develop an AI model to predict the age of the eye. A 10-fold cross-validated XGBoost model was trained in the training cohort of 46 healthy patients to predict the chronological age of the patients (R=0.63, p=2.4×10⁻⁶, RMSE=7.90; 10-fold cross-validation; FIG. 7A). A final model was built based on 26 proteins which were selected by at least 3 of the 10 folds in the training cohort. Twenty of these proteins had known associations to aging (Table 4), but, with only one exception (IGFBP1), were not among the protein biomarkers identified in the diseases we analyzed above, suggesting an independent, parallel process. The final model was prospectively validated in an independent cohort of nineteen healthy patients, demonstrating strong prediction of age in this validation cohort (R=0.65, p=2.8×10⁻³, RMSE=9.11; FIG. 7B), showing AI could accurately assess the molecular age of healthy eyes. We then applied the AI model to patients with eye diseases that are not primarily related to birth age (FIGS. 7C-7D). The molecular age of patients with early DR (NPDR) was predicted to be 12 years older compared to healthy patients (sd: 9, p=0.017) and was increased by 31 years (sd: 12, p=1.6×10⁻⁵) in patients with late DR (PDR) (FIGS. 7C-7D). Similarly, the molecular age was elevated by 16 years (sd: 12, p=5.0×10⁻³) in RP patients (FIG. 7D) and by 29 years (sd: 15, p=3.7×10⁻⁴) in uveitis patients (FIG. 7D). These results indicate diseased eyes undergo accelerated aging, providing some of the strongest molecular evidence to date of the overlap between aging and disease processes, even though protein biomarkers for aging and disease are distinct.

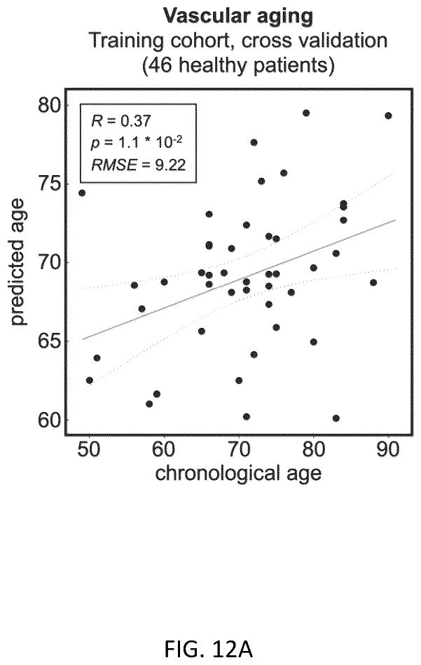

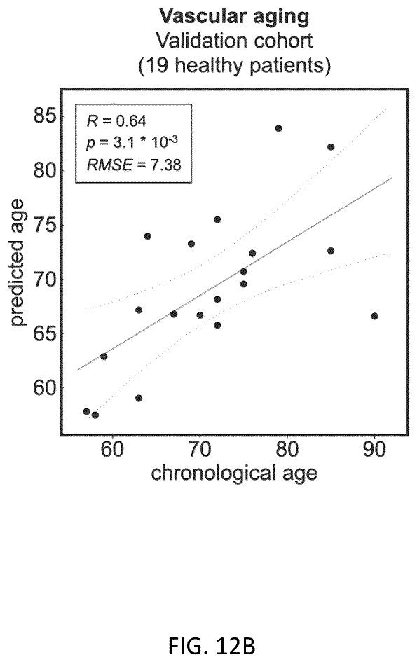

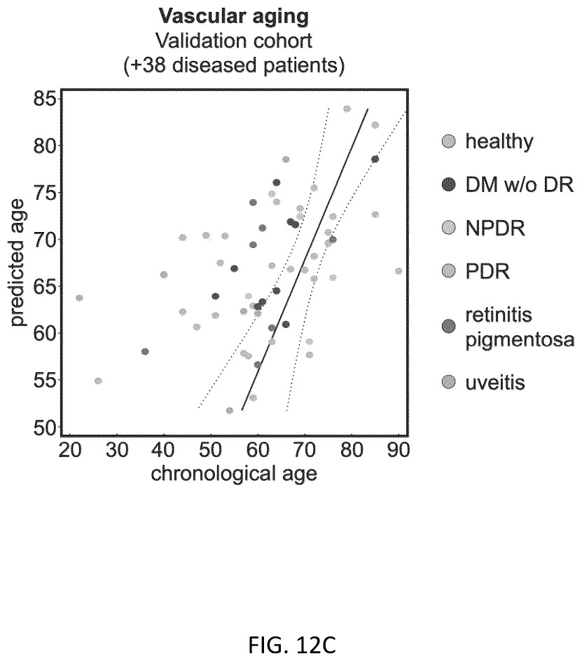

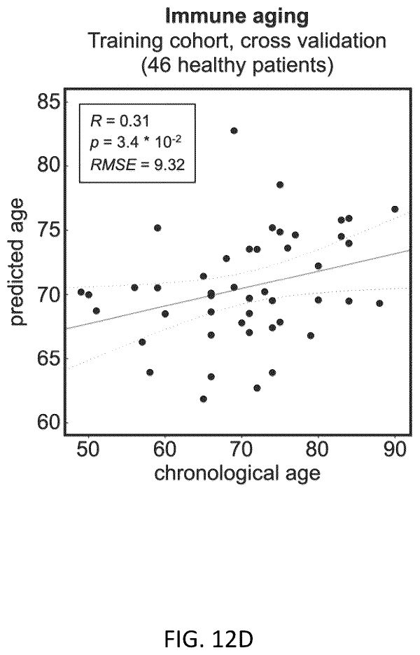

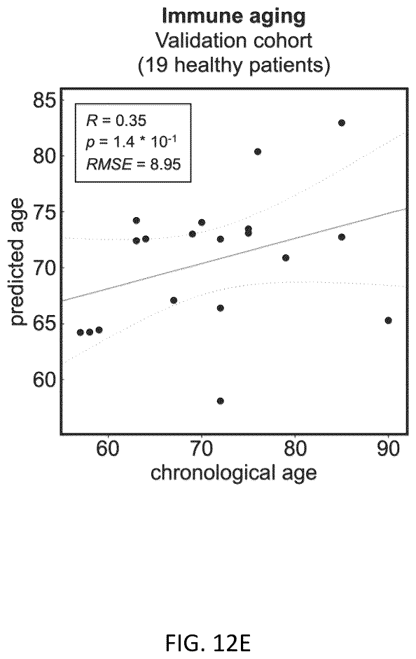

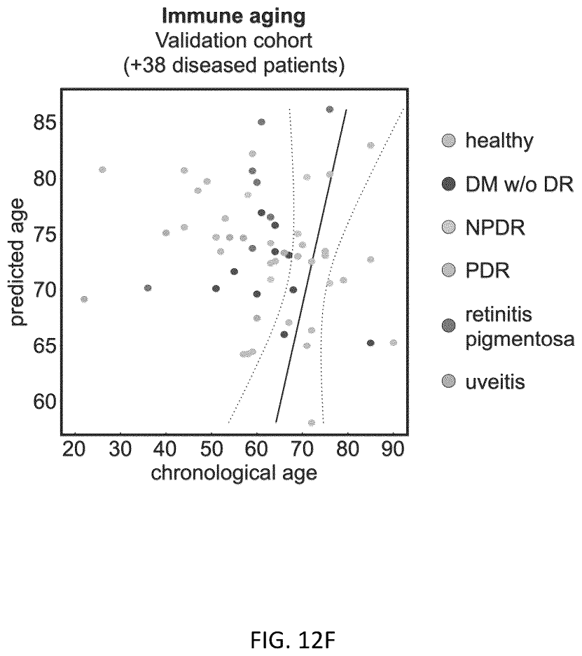

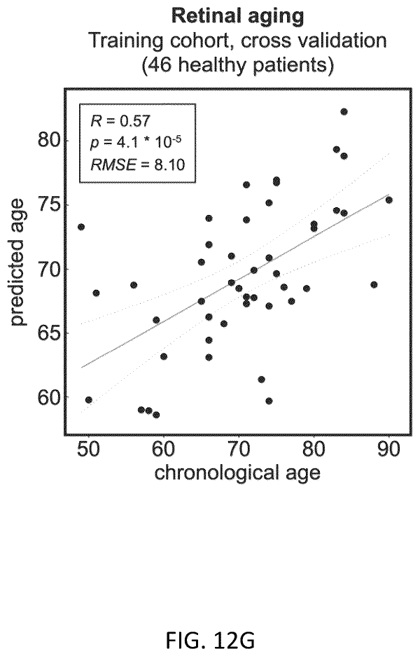

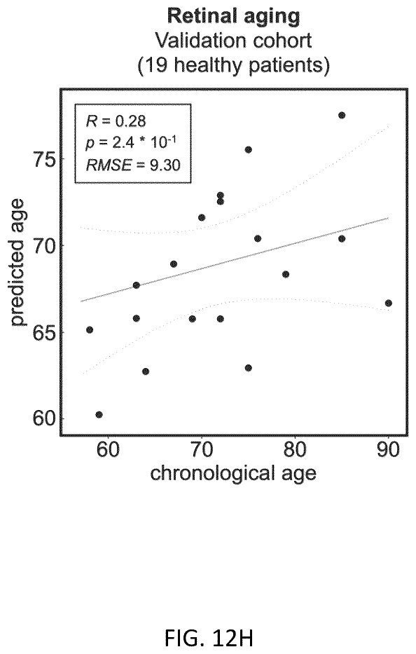

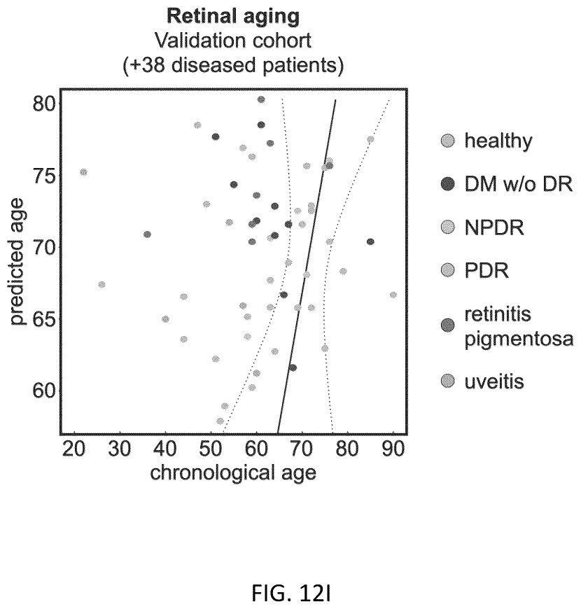

In normal eyes, we next determined which cell types were most associated with aging. We found that age-associated proteins were mainly derived from endothelial cells, pericytes, and several immune cell types, including macrophages, monocytes, neutrophils, and mast cells (FIG. 7E). Proteins including LECT2 and A1BG are specifically produced in the liver and were previously linked to aging of the liver,^{42,43} suggesting that these proteins likely enter the AH through the circulation and could be more reflective of liver age. We then explored whether diseases are associated with accelerated aging of specific cell types. We developed AI models to predict the vascular, immune, and retinal age based on proteins specific to the respective cell types. AI vascular aging (22 proteins), AI immune aging (21 proteins) and AI retinal aging (20 proteins) were all associated with chronological age in both the training and validation cohorts (FIG. 12). More than 85% of the associated proteins were not among the protein biomarkers identified in the diseases we analyzed. The three AI models revealed that eye diseases involve accelerated aging of disease-specific cell types: vascular cells in PDR (FIG. 7F), immune cells in uveitis (FIG. 7G), and retinal cells in RP (FIG. 7H). Surprisingly, we also found that accelerated aging of immune and retinal cells had already started in patients with diabetes mellitus who did not show any clinically visible signs of retinopathy (FIGS. 7G-7H). This emphasizes the potential importance of assessing cell type-specific aging rather than whole organ aging. It also demonstrates that molecular assessment may be more sensitive than current clinical imaging techniques, and further suggests that earlier preventive interventions may be beneficial to these patients. We also determined the cell types driving vascular, immune, and retinal aging of the eye. Endothelial cells in the retina and RPE/choroid contributed most to the AI vascular aging signature (FIG. 7I). B cells, hyalocytes, and T cells were the main drivers of the AI immune aging signature (FIG. 7J), and bipolar cells, rods, and amacrine cells drove the AI retinal aging model (FIG. 7K). In sum, our AI models establish that eye diseases are associated with an accelerated aging of disease-specific cell types, and that different molecules and cells may have greater impact on aging compared to disease. This raises the possibility that disease-targeted therapies may not fully address accelerated aging, which may instead require simultaneous, molecular and cellularly distinct anti-aging therapeutic strategies.

### Discussion

A fundamental question clinicians and biomedical researchers must answer is which cells are driving disease in living humans, and without a precise answer, our ability to diagnose patients, select or create new therapies, design clinical trials, and interpret the relevance of animal and cell models can be highly flawed. Proteins are the main effectors of disease and the target of most drugs, but analyzing the contribution of specific proteins at the single cell level has so far required either a tissue biopsy or postmortem tissue. This requirement has substantially limited the ability to assess disease states at cellular resolution in living patients, especially for non-regenerative organs not amenable to solid tissue biopsies. Moreover, partial tissue samples don't always reflect the entire disease burden since small biopsies can miss the key disease regions within cellularly heterogenous tissue.¹⁴ Liquid biopsies can overcome these limits, but until now, measuring large numbers of proteins within small volumes and knowing their cell origin has not been possible. Here, we developed an approach, referred to as TEMPO, that integrates microvolume liquid biopsy proteomics, single cell transcriptomics, and AI, resulting in a powerful and minimally invasive tool to examine disease and aging mechanisms at the cell level in vivo. From only 50 microliters of a biological fluid, the detection of nearly six thousand proteins that can be traced back to specific cells provides an unprecedented view into the activity of individual cells of living humans. Application of TEMPO could be transformative towards identifying cellular mechanisms, enhancing diagnosis, optimizing clinical trials, and determining the interplay between aging and disease.

There is ample evidence that aging is a risk factor that can hasten various diseases.²⁹ The reciprocal, how disease impacts aging, is less understood, specifically, whether diseased cells and neighboring “bystander” cells undergo accelerated aging in parallel with disease. Consistent with this idea, for example, clinicians and patients often note that subjective vision is not fully restored after treatment of their eye disease, even if the disease is well-controlled. The phenomenon of cellular aging in response to disease is poorly understood because there are no molecular models to measure cell-specific aging in living humans. Plasma-based DNA methylation clocks have emerged as the primary tool to assess molecular age, 45 and proteomic clocks were developed to globally estimate the molecular age on the organism level.^{32,33,46} However, these models may not assess the overall biological age of organs and cannot determine the age of individual cells within an organ. Here, we developed cell type-specific AI aging models which represent a powerful tool to assess the molecular age on the cellular level, providing critical new insights into normal aging and disease. From proteins in the aqueous humor, we found normal aging of the eye mainly follows complex non-linear trajectories, similar to aging patterns observed in plasma in humans and mice,³² although the specific proteins and cells are different. The AI model selected 26 proteins with predictive value in estimating age, 20 of which had known associations to aging in the literature, but were not among the disease biomarkers, indicating that different proteins are involved in aging and disease. One of the aging proteins (LECT2) was reported to broadly modulate known aging pathways in knockout mice.⁴⁷ Three proteins were related to cell senescence (ACAN, CD274, ABI3), 2 proteins (SCGN, ADA2) were involved in epigenetic modulation, and the expression of 9 proteins (IGFBP1, DCBLD1, TFF3, AMN, ABO, HAMP, A1BG, MVP, A2M) was found to vary with age in other studies (Table 4). The 26 aging proteins originated predominantly from immune and endothelial cells, which are cell types implicated in other aging studies.^{35,48-50} Interestingly, none of these 26 proteins overlapped with a curated list of age-associated proteins in the blood,⁵¹ further suggesting that the aging process of the eye is principally a local rather than systemic process.⁵² In addition, only 3 of the whole eye aging proteins overlapped with the 63 cell type-specific aging proteins, indicating that cell-specific aging may be different from whole organ aging.

When applied to eyes with a broad range of diseases, where chronological age is not a risk factor, the AI model found disease accelerated aging in distinctive ways in the whole eye, depending on the specific ailment. This even included some DR and uveitis patients that had been successfully treated to control inflammation or reverse retinal edema and hemorrhaging, respectively; aging protein biomarkers remained elevated in these patients, indicating eye aging was a separate process that can persist after disease control. This raises the possibility that anti-aging treatment targeting cells and molecules other than the primary diseased cells may be required to fully reverse the effects of a disease. For example, an epigenetic approach targeted mouse retinal ganglion cells in optic nerve disease to rejuvenate cell function,³⁶ but full anti-aging therapy may have to consider the acceleration of aging that is triggered by the disease in bystander cells. Most surprising in our study was that diabetic patients without any clinical evidence of retinopathy showed molecular evidence of accelerated cell-aging. This clearly supports molecular assessment of cell-type specific aging as a valuable adjunct to the currently used clinical imaging techniques, and that earlier preventive interventions may be beneficial. Future studies must consider the significance of cellular aging in the context of disease treatment.

TEMPO represents a powerful tool to identify the contribution of specific cells to human disease. We found that the cellular driver of DR changed with disease stage. The angiogenesis-related proteins common to both early and late disease stages were mainly derived from endothelial cells and pericytes, a finding consistent with the microangiopathic etiology of DR. At a later stage, unexpectedly, the PDR-specific proteins, which are also consistent with active microangiogenesis, originated mainly from immune cells, including macrophages, neutrophils, and retinal microglia. To our knowledge, this is the first in vivo human molecular data demonstrating that immune cells like macrophages are important molecular drivers of late-stage DR. Immune cells may represent a key target for PDR, and therapeutic responses could be molecularly monitored using TEMPO. We also found an increase of liver-derived proteins in the AH of DR patients, likely the result of the increased vascular permeability through disruption of the blood-retinal barrier in DR.^{53,54} The liver proteins in question are known to be involved in inflammatory processes, raising the possibility that the liver may directly contribute to inflammation in DR pathology and indicating that systemic therapeutic intervention may be beneficial.⁵⁴

By providing a minimally-invasive window into the state of individual cell types, TEMPO makes it possible to uncover new mechanisms of human disease. TEMPO, as applied to AH liquid biopsies, has uncovered a role for specific immune cells and liver proteins in DR, which could change therapeutic strategies. We also identified multiple cell types that were previously not known to be involved in human eye disease. It is well established that RP, a monogenic disease, involves a progressive loss of rod photoreceptors, and this was molecularly validated in our study. Even though other neuronal cell types appear anatomically intact in human RP, we found that dysfunction and damage to retinal neural circuits is more extensive than previously known, as other retinal cell types were molecularly affected, including cone photoreceptors, retinal bipolar cells, and retinal amacrine cells. This may partly explain the incomplete and incremental response patients have following gene therapy that only targets one cell type. While some of these findings have previously been suggested by animal models 55 and post-mortem studies,^{56,57} it has not been possible until now to molecularly confirm that these cellular changes occur in living patients. We also found evidence for molecular dysfunction of the retina in PD, especially for cell types of the inner retinal layers. This is consistent with anatomical changes observed in PD patient retinas⁶ and in a mouse model of PD.⁵⁸ But we also observed a decrease of marker proteins of the outer retina rod and cone photoreceptors, which appear anatomically intact in PD.⁶ In contrast to idiopathic PD, PD patients with LRRK2 mutations showed little or no retinal thinning in OCT,⁵⁹ suggesting that AH liquid biopsies may be of high diagnostic value in these patients.

The high failure rate of clinical trials can be improved by liquid biopsy-cell proteomics. Instead of selecting patients by anatomic phenotypes, patients could be selected by molecular markers. While this is the norm for gene therapy and some cancer therapies, many trials using a molecular therapy do not first determine if enrolled patients have the molecular target. A recently published randomized trial involving patients with early-stage diabetic retinopathy (NPDR) revealed that anti-VEGF therapy resulted in no improvement in visual acuity after 4 years.⁶⁰ Our data revealed that the protein VEGF is not increased in patients with NPDR compared to healthy controls, providing a compelling explanation for why the aforementioned human study failed to demonstrate an improved visual acuity. In contrast to VEGF, we identified several angiogenic proteins that were already elevated in NPDR, including ANGPTL4 and ANGPT2. ANGPT2 is targeted by faricimab, a bispecific antibody used in the treatment of diabetic macular edema.⁶¹ Our data points towards a potential application of faricimab for PDR as well. In PD there is evidence that LRRK2-targeted therapies could also be beneficial in PD patients without a LRRK2 mutation,⁶² which would significantly increase the target population for clinical trials. However, the selection of patients for these trials will be critical and no definitive in vivo biomarker has been identified so far to guide selection.⁶² Analyzing AH, TEMPO may serve as a valuable tool to identify appropriate PD patients for clinical trials, provide a prognostic assessment, and monitor response to therapy. This is particularly important considering the usually slow clinical progression of the disease, which is difficult to assess in a reasonable time in clinical trials. Thus, clinical trials designed with patient selection based on molecular cell biomarkers with corresponding disease stages could significantly improve their success rate.

Although collection of plasma samples is common practice, liquid biopsies from non-blood sources may provide a more sensitive and specific diagnostic method, especially in organs that are not amenable to direct tissue biopsies. Neurodegenerative diseases like PD often represent a diagnostic challenge and may not be definitively diagnosed without a post-mortem examination. Here we show the potential of using AH liquid biopsies as a valuable tool for diagnostic and prognostic assessment of brain disease. PD-associated proteins were altered in the AH of PD patients, including LRRK2,^{63,64} GBA,⁶⁵ and HTRA2,⁶⁶ and these proteins were mainly produced by neuronal cell types in the retina. For other systemic and syndromic disease studies, AH samples can be obtained during clinical trials, in an outpatient clinic, or during cataract surgery, which is one of the most frequently performed surgeries across the globe. TEMPO holds enormous potential for the analysis of these samples and, apart from the eye, it is potentially applicable to other organ systems including the brain using cerebrospinal fluid, the lungs using fluid from bronchoalveolar lavage, the joints using synovial fluid, the liver using bile, the kidneys using urine liquid biopsies, the breast using breast milk, or localized cancers and tumor cysts.⁴⁴ TEMPO could also be applied to plasma liquid biopsy proteomics in instances where there are highly specific proteins originating only from specific cell types and organs. Although challenges include the large number of different organs and cell types that can release proteins into the blood and organs that have yet to undergo extensive scRNA sequencing and data integration, TEMPO has the potential to determine the contribution of individual cell types in a variety of human diseases and generate cell type-specific proteomic clocks to assess the molecular age at the organ and cell level.

In conclusion, we developed a powerful, AI-oriented, multi-omics approach, TEMPO, combining high-resolution liquid biopsy proteomics with tissue cell level transcriptomics that overcomes the limits of each technology on its own and allowed us to identify the cellular origin of thousands of proteins in liquid biopsies. This integration of these two high resolution methodologies represents a major technical advance to examine disease and aging mechanisms at the cell level in vivo. Using the eye as a test case, we identified hundreds of cell type-specific proteins, among them markers for individual retinal cell types, an approach that can be applied to other organs and diseases. With this data, AI models can accurately assess the molecular age of the eye, and crucially, resolve this in terms of cell-type specific aging. Applying these AI models to diseases revealed independent pathways of accelerated aging in disease-specific cell types, sometimes even before the onset of clinical disease and persisting after clinical resolution. TEMPO fills a critical gap in the ability to study human disease and aging at the molecular and cellular levels in living humans.

### Data and Code Availability

All cell type-specific marker proteins identified in this study are accessible in Table 1. All proteomics data generated in this study using the aptamer-based proteomics assay (SomaScan® Assay v4.1, Somalogic) are accessible in Table 7.

### Experimental Model and Study Participant Details

### Human Participants

The study protocol was approved by the Institutional Review Board for Human Subjects Research (IRB) at Stanford University, was HIPAA compliant, and adhered to the tenets of the Declaration of Helsinki. All subjects underwent informed consent for study participation.

A total of 108 AH liquid biopsies were obtained from patients undergoing cataract surgery or during vitrectomy, and 12 vitreous samples were collected from patients undergoing vitrectomy due to epiretinal membranes or an idiopathic macular hole. An overview of all samples including basic demographic data is provided in Table 5.

### Method Details

### Aqueous Humor and Vitreous Sample Collection

The first cohort of samples, consisting of 12 healthy AH and 12 healthy vitreous samples, served to identify cell type-specific marker proteins. 34 additional healthy AH samples (second cohort) together with the 12 healthy AH samples from the first cohort served as the training cohort to build the artificial intelligence aging model. The third cohort of samples comprised 62 AH samples, including 15 samples from patients with DR, 10 samples from patients with diabetes mellitus but without DR, 6 samples from patients with uveitis, 7 samples from patients with RP, 5 samples from patients with PD, as well as 19 samples from patients with cataract, but with otherwise healthy eyes serving as controls. The third cohort also served as an independent validation cohort for the artificial intelligence aging model. After the eye was prepped and draped for surgery, anterior chamber paracentesis was performed using a 31-gauge needle, and approximately 100 μl of undiluted AH was manually aspirated with a 1 ml syringe, transferred to a tube (1.9 ml Tri-coded Tube, Azenta Life Sciences, Burlington, MA 01803, USA) and immediately frozen on dry ice in a cooling box in the operating room. To collect vitreous samples, a standard 3 port pars plana vitrectomy setup was used with a single-step transconjunctival 25-gauge trocar cannular system (Alcon Laboratories Inc, Fort Worth, TX, USA). An undiluted 0.5 to 1.0 ml vitreous sample was manually aspirated into a 3 ml syringe. Samples were transferred to a tube and immediately frozen on dry ice as described above. Samples were stored at minus 80° C. until further analysis. Apart from cataract, epiretinal membrane or idiopathic macular hole, the control patients did not suffer from other eye diseases, such as age-related macular degeneration, glaucoma, or DR. Representative clinical images and electroretinograms are shown. The same representative normal images and electroretinograms were used as comparison for several diseases.

### Aptamer-Based Proteomics Assay

For proteomic profiling, AH and vitreous samples were shipped on dry ice overnight to Somalogic (Boulder, Colorado, USA) and the samples were analyzed with an aptamer-based proteomics assay (SomaScan® Assay v4.1, Somalogic) as previously described.¹⁸ Briefly, 6,345 human protein targets were evaluated by 7,289 aptamers, which are short single-stranded, chemically modified DNA molecules which specifically bind to their protein targets and which are quantified using DNA microarray technology.

### Quantification and Statistical Analysis

### Bioinformatics

Aptamer-based assay data were normalized by Somalogic as previously described⁶⁸ Normalized data were imported to R Studio (version 2022.02.0+443, R version 4.1.2). Aptamers' target annotation and mapping to UniProt accession numbers as well as Entrez gene identifiers were provided by Somalogic. Only human protein targets were retained for subsequent analysis (7,288 out of the 7,596 aptamers). The estimated limit of detection (eLoD) was calculated for each aptamer using a ‘robust estimate’ method as previously described⁶⁸. Briefly, it was calculated as the median plus 4.9×median absolute deviation signal of buffer samples. The eLoD was then subtracted from each intensity value of each aptamer in each sample to obtain the actual protein intensity above the detection limit and values below 0 were replaced by 0. For some proteins, the assay provides more than one aptamer. In these cases (887 of expressed proteins) only the aptamer with the highest intensity was retained.

After principal component analysis, differentially expressed proteins were determined using the limma package (version 3.56.2)⁶⁹ with default parameters except using method=“robust” in ImFit. P-values were corrected for multiple testing using the Benjamini-Hochberg adjustment implemented in the limma package. Proteins with log 2 fold change (log 2FC)>2 or <−2 and adjusted p value<0.05 were considered as differentially expressed proteins (DEP). Heatmaps were created with the R package ComplexHeatmap (version 2.10.0).⁷⁰ The z-score represents a gene's or protein's expression level in relation to its mean expression in all samples by standard deviation units. Other data visualization was done using the ggplot2 package (version 3.3.5). In boxplots, the horizontal line represents the median and the height of the box represents the interquartile range.

TEMPO (Tracing Expression of Multiple Protein Origins) is an analytical method where cell type-specific expression of genes encoding for the proteins detected in AH or vitreous was investigated by integration of the proteomic data with published scRNA-seq data of human ocular and extra-ocular tissues. scRNA-seq data were obtained from the following tissues from the respective primary publications: cornea,⁷ trabecular meshwork,⁸ iris,⁹ ciliary body,⁹ lens,¹⁰ hyalocytes,^{11,12} retina,¹³ retinal pigment epithelium (RPE)/choroid,¹⁴ liver,¹⁵ spleen,¹⁶ blood,¹⁷ and bile duct¹⁶ (negative control). Data were imported to R Studio and mean expression for each gene in each cell type was calculated based on the provided cell type annotations. Gene symbols were filtered for approved symbols using the HGNC (Human Genome Organization Gene Nomenclature Committee) database.⁷¹ The full names corresponding to the symbols used in the manuscript are summarized in Table 6. Expression data were combined and normalized using DESeq2 (version 1.40.2) with default parameters.⁷²

Specific expression for each cell type was defined as follows (all criteria needed to be fulfilled): highest z-score (deviation from the mean in standard deviation units) in the respective cell type, z-score >3 (meaning that the expression in the cell type of interest had to be higher than 3 standard deviations from the mean expression of this gene in all cell types), expression of the respective gene in that cell type at least at the 10^{th} percentile of all genes expressed in that cell type. For extraocular cell types (spleen and liver) only genes encoding for extracellular proteins were considered. The subcellular localization of each protein was categorized into one of the following groups using the Uniprot database:⁷³ extracellular, membrane or intracellular. Proteins with an extracellular and another subcellular localization were categorized as extracellular. Proteins with membrane and intracellular localization were categorized as membrane. To determine cell type-specific marker proteins, the specifically expressed genes described above were further filtered using the following criteria (all criteria needed to be fulfilled): mean gene expression>maximal gene expression in any other cell type and >2*maximal mean gene expression in any cell cluster, protein detected at least at the 10^{th} percentile in both AH and vitreous.

To estimate protein trajectories during aging, AH protein levels were z-scored, and locally estimated scatterplot smoothing (LOESS) regression was fitted to model z-scores in dependence of age for each protein. Unsupervised hierarchical cluster analysis was performed based on the LOESS models for each protein using the complete method. Pathway analysis was performed using the g:Profiler web server⁷⁴ using all 6,345 human proteins measured by the SomaScan assay as the background set of proteins against which to test for over-representation. The following databases were included in the pathway enrichment analysis: GO, KEGG, Reactome, and wiki pathways.

### Artificial Intelligence Aging Model

An artificial intelligence (AI) model was built to predict the age of a patient based on an AH liquid biopsy. A 10-fold cross validation approach was applied to test the generalizability of the multivariate models to previously unseen samples. The resulting model was applied to estimate the age for the excluded patients. This procedure was repeated until the age was estimated for each patient. This cross-validation approach was applied to test a range of AI algorithms, including XGBoost,⁷⁵ randomForest,⁷⁶ and elastic net⁷⁷. For each algorithm, hyperparameter tuning was performed as described in the documentation of each software package. The XGBoost algorithm demonstrated the best performance to estimate age and was therefore applied to build the final model. For feature selection for the final model, features which were selected by at least 3 of the 10 cross validation models were selected. The cutoff 3 was chosen because it resulted in the lowest error of the model in the training cohort. The final model was then trained using all 46 samples of the training cohort using the following hyperparameters: nrounds=50, max_depth=2, eta=0.3, gamma=0.9, colsample_bytree=1, min_child_weight=1, subsample=0.7. The final model was then validated using 19 previously unseen samples of the independent validation cohort. Models to predict the vascular, immune, and retinal age were built in the same way using the XGBoost algorithm but were based solely on proteins originating specifically from vascular, immune, or retinal cells, respectively (as determined by TEMPO).

### Statistical Details for Each Figure Panel

FIG. 2A: The analysis is based on 12 control aqueous humor and 12 control vitreous liquid biopsies. The limit of detection for proteomics data was calculated as specified under bioinformatics. Single cell RNA-sequencing data of all cell types of the human eye were integrated as specified under bioinformatics.

FIG. 2B: Left panel: unsupervised cluster analysis using principal component analysis (PCA) of all cell types based on the gene expression profile encoding for all detected proteins. Right panel: the cellular origin of each protein was determined using the following criteria (all criteria needed to be fulfilled): highest z-score (deviation from the mean in standard deviation units) in the respective cell type, z-score >3 (meaning that the expression in the cell type of interest had to be higher than 3 standard deviations from the mean expression of this gene in all cell types), expression of the respective gene in that cell type at least at the 10th percentile of all genes expressed in that cell type. For extraocular cell types (spleen and liver) only genes encoding for extracellular proteins were considered.

FIG. 2C: To determine cell type-specific marker proteins, the specifically expressed genes described under FIG. 2B were further filtered using the following criteria (all criteria needed to be fulfilled): mean gene expression>maximal gene expression in any other cell type and >2*maximal mean gene expression in any cell cluster, protein detected at least at the 10th percentile in both AH and vitreous.

FIG. 3B: It was analyzed which of the 1,920 identified cell type-specific marker proteins (FIG. 2C) were significantly decreased in RP (log 2FC<−2 & adjusted p value<0.05). The analysis is based on 7 patients with RP and 19 control patients. FIG. 4B: It was analyzed which of the 1,920 identified cell type-specific marker proteins (FIG. 2C) were significantly increased in DR (log 2FC>2 & adjusted p value<0.05). The analysis is based on 10 patients with proliferative diabetic retinopathy (PDR), 5 patients with non-proliferative diabetic retinopathy (NPDR), 10 patients with diabetes mellitus without DR (DM w/o DR), and 19 control patients.

FIG. 4C: It was analyzed which known angiogenic proteins were significantly increased in PDR AH vs. control and NPDR AH vs. control (log 2FC>2 & adjusted p value<0.05). A cell type was considered as the origin of a protein if the corresponding gene expression was at least 2 standard deviations above the mean expression in all cell types.

FIG. 5A: It was analyzed which known Parkinson's disease-relevant proteins were significantly changed in Parkinson's disease AH vs. control (log 2FC>2 or <−2 & adjusted p value<0.05). The analysis is based on 5 patients with Parkinson's disease and 19 control patients.

FIG. 5B: A cell type was considered as the origin of a protein if the corresponding gene expression was at least 2 standard deviations above the mean expression in all cell types.

FIG. 5C: It was analyzed which of the 1,920 identified cell type-specific marker proteins (FIG. 2C) were significantly decreased in Parkinson's disease AH vs. control AH (log 2FC<−2 & adjusted p value<0.05). The analysis is based on 5 patients with Parkinson's disease and 19 control patients.

FIGS. 6A-6P: To estimate protein trajectories during aging, AH protein levels were z-scored, and locally estimated scatterplot smoothing (LOESS) regression was fitted to model z-scores in dependence of age for each protein. Unsupervised hierarchical cluster analysis was performed based on the LOESS models for each protein using the complete method. Protein trajectories were plotted for each cluster (6A-6L) or for proteins known to be involved in aging associated processes (6M-6P). Pathway analysis was performed using the g:Profiler web server⁷⁴ using all 6,345 human proteins measured by the SomaScan assay as the background set of proteins against which to test for over-representation. The following databases were included in the pathway enrichment analysis: GO, KEGG, Reactome, and wiki pathways. The cell type origin was determined based on the 1,920 identified cell type-specific marker proteins (FIG. 2C).

FIG. 7A-7B: An artificial intelligence (AI) model was built to predict the age of a patient based on an AH liquid biopsy. A 10-fold cross validation approach was applied to test the generalizability of the multivariate models to previously unseen samples. The resulting model was applied to estimate the age for the excluded patients. This procedure was repeated until the age was estimated for each patient. This cross-validation approach was applied to test a range of AI algorithms, including XGBoost,⁷⁵ randomForest,⁷⁶ and elastic net⁷⁷. For each algorithm, hyperparameter tuning was performed as described in the documentation of each software package. The XGBoost algorithm demonstrated the best performance to estimate age and was therefore applied to build the final model. For feature selection for the final model, features which were selected by at least 3 of the 10 cross validation models were selected. The cutoff 3 was chosen because it resulted in the lowest error of the model in the training cohort. The final model was then trained using all 46 samples of the training cohort using the following hyperparameters: nrounds=50, max_depth=2, eta=0.3, gamma=0.9, colsample_bytree=1, min_child_weight=1, subsample=0.7. The final model was then validated using 19 previously unseen samples of the independent validation cohort.

FIGS. 7C-7D: The final model was applied to predict the molecular age in diseased AH using previously unseen samples.

FIG. 7E: A cell type was considered as the origin of a protein if the corresponding gene expression was at least 2 standard deviations above the mean expression in all cell types.

FIGS. 7F-7H: Models to predict the vascular, immune, and retinal age were built in the same way as described above using the XGBoost algorithm but were based solely on proteins originating specifically from vascular, immune, or retinal cells, respectively (as determined by TEMPO).

FIGS. 7I-7K: The cellular origin of the aging-associated proteins identified in the vascular, immune, and retinal cell-specific aging models is displayed as a fraction of the number of all cell type-specific proteins for the respective cell type.

FIG. 8: The protein level in healthy aqueous humor (AH) was compared to published proteomics data from the blood, which was generated using the same proteomics platform. The percentile of protein level was determined for each protein in AH and blood and the difference between the AH percentile and the blood percentile was calculated for each protein. The bar graph shows the difference for all identified cell type-specific marker proteins, with the height of the bars corresponding to the mean difference and the error bar to the standard error of the mean. Significance was calculated using the Mann-Whitney U test (p<0.05 was considered significant).

FIG. 9C: It was analyzed which of the 1,920 identified cell type-specific marker proteins (FIG. 2C) were significantly increased in Uveitis compared to controls (log 2FC>2 & adjusted p value<0.05). The analysis is based on 6 patients with uveitis and 19 control patients.

FIG. 11: To estimate protein trajectories during aging, AH protein levels were z-scored, and locally estimated scatterplot smoothing (LOESS) regression was fitted to model z-scores in dependence of age for each protein. Protein trajectories were plotted for proteins known to be involved in aging associated processes. The cell type origin was determined based on the 1,920 identified cell type-specific marker proteins (FIG. 2C).

FIGS. 12A-12H: To predict the vascular, immune, and retinal age of the eye, artificial intelligence (AI) models were built based on proteins originating specifically from vascular, immune, and retinal cells, respectively (training cohort, 46 samples, 10-fold cross validation) (FIGS. 12A, 12D, 12G). The final models were built based on 24 (vascular aging), 21 (immune aging), and 27 proteins (retinal aging) which were selected by the AI models in the training cohort. The validity of these models was then prospectively evaluated in an independent cohort of 19 healthy patients (FIGS. 12B, 12E, 12H). The vascular (FIG. 12C), immune (FIG. 12F), and retinal age (1) was predicted in patients with diabetes without retinopathy (DM w/o DR), non-proliferative diabetic retinopathy (NPDR), proliferative diabetic retinopathy (PDR), retinitis pigmentosa (RP), and uveitis.

FIGS. 13A-13E: (FIG. 13A) Principal component analysis (PCA) based on all detected proteins from 12 cataract patients that did not suffer from any other eye disease and PCAs including cataract samples and aqueous humor samples from patients with (FIG. 13B) retinitis pigmentosa, (FIG. 13C) diabetic retinopathy, (FIG. 13D) uveitis, and (FIG. 13E) Parkinson's disease.

TABLE 1
Cell Type-Specific Markers

| Marker | Cell type |
|---|---|
| BTLA | Bcells |
| CD70 | Bcells |
| CD79A | Bcells |
| CR2 | Bcells |
| FCER2 | Bcells |
| FCRL2 | Bcells |
| FCRL5 | Bcells |
| IGHA1 | Bcells |
| IGHD | Bcells |
| IGHG2 | Bcells |
| IGHG4 | Bcells |
| IGHM | Bcells |
| IGKV1-5 | Bcells |
| IRF4 | Bcells |
| JCHAIN | Bcells |
| LY9 | Bcells |
| MZB1 | Bcells |
| SLAMF7 | Bcells |
| TCL1A | Bcells |
| TNFRSF13B | Bcells |
| TXNDC5 | Bcells |
| CA9 | CB_nonpigmented_cells |
| CADM3 | CB_nonpigmented_cells |
| CDH13 | CB_nonpigmented_cells |
| CRIM1 | CB_nonpigmented_cells |
| CRYAA | CB_nonpigmented_cells |
| CRYBB2 | CB_nonpigmented_cells |
| CRYGS | CB_nonpigmented_cells |
| CSMD2 | CB_nonpigmented_cells |
| FGFR3 | CB_nonpigmented_cells |
| GJA8 | CB_nonpigmented_cells |
| GSTM5 | CB_nonpigmented_cells |
| INHBA | CB_nonpigmented_cells |
| KMO | CB_nonpigmented_cells |
| LRP11 | CB_nonpigmented_cells |
| ME1 | CB_nonpigmented_cells |
| PDGFRA | CB_nonpigmented_cells |
| PITX3 | CB_nonpigmented_cells |
| PLOD2 | CB_nonpigmented_cells |
| RSPO1 | CB_nonpigmented_cells |
| SFRP2 | CB_nonpigmented_cells |
| STC2 | CB_nonpigmented_cells |
| AOC2 | CB_pigmented_cells |
| AOC3 | CB_pigmented_cells |
| B3GALT1 | CB_pigmented_cells |
| BOC | CB_pigmented_cells |
| CADPS2 | CB_pigmented_cells |
| COL9A1 | CB_pigmented_cells |
| COL9A3 | CB_pigmented_cells |
| CTNNBIP1 | CB_pigmented_cells |
| DLL1 | CB_pigmented_cells |
| DMKN | CB_pigmented_cells |
| EFEMP1 | CB_pigmented_cells |
| FBP2 | CB_pigmented_cells |
| FGFR1 | CB_pigmented_cells |
| GALNT10 | CB_pigmented_cells |
| GPX3 | CB_pigmented_cells |
| GULP1 | CB_pigmented_cells |
| HTR2A | CB_pigmented_cells |
| LRP2 | CB_pigmented_cells |
| NRG3 | CB_pigmented_cells |
| OPTC | CB_pigmented_cells |
| PAPLN | CB_pigmented_cells |
| PDE1A | CB_pigmented_cells |
| PDE11A | CB_pigmented_cells |
| PKP2 | CB_pigmented_cells |
| PLA2R1 | CB_pigmented_cells |
| PPARGC1A | CB_pigmented_cells |
| PRKCSH | CB_pigmented_cells |
| PTPRD | CB_pigmented_cells |
| RELN | CB_pigmented_cells |
| SCUBE1 | CB_pigmented_cells |
| SEMA3C | CB_pigmented_cells |
| SLC5A8 | CB_pigmented_cells |
| SLITRK5 | CB_pigmented_cells |
| SND1 | CB_pigmented_cells |
| TGFB2 | CB_pigmented_cells |
| TGFB3 | CB_pigmented_cells |
| TMEM132C | CB_pigmented_cells |
| ANGPTL1 | Corneal_endothel_cells |
| EPHA3 | Corneal_endothel_cells |
| RERG | Corneal_endothel_cells |
| RRAD | Corneal_endothel_cells |
| SELENOM | Corneal_endothel_cells |
| SORBS3 | Corneal_endothel_cells |
| ADGRG2 | Corneal_stroma_cells |
| ALDH3A1 | Corneal_stroma_cells |
| ANGPTL7 | Corneal_stroma_cells |
| ASIP | Corneal_stroma_cells |
| BMPER | Corneal_stroma_cells |
| C5orf38 | Corneal_stroma_cells |
| CHST6 | Corneal_stroma_cells |
| COLEC12 | Corneal_stroma_cells |
| CRABP2 | Corneal_stroma_cells |
| CYTL1 | Corneal_stroma_cells |
| DKK2 | Corneal_stroma_cells |
| ECM1 | Corneal_stroma_cells |
| FBLN7 | Corneal_stroma_cells |
| FMOD | Corneal_stroma_cells |
| GLT8D2 | Corneal_stroma_cells |
| GRP | Corneal_stroma_cells |
| HGF | Corneal_stroma_cells |
| HTRA1 | Corneal_stroma_cells |
| IGFBP6 | Corneal_stroma_cells |
| IL17RD | Corneal_stroma_cells |
| ITGA11 | Corneal_stroma_cells |
| JAM3 | Corneal_stroma_cells |
| KERA | Corneal_stroma_cells |
| LUM | Corneal_stroma_cells |
| MAMDC2 | Corneal_stroma_cells |
| MAP2K6 | Corneal_stroma_cells |
| MME | Corneal_stroma_cells |
| MMP3 | Corneal_stroma_cells |
| NDNF | Corneal_stroma_cells |
| NEFL | Corneal_stroma_cells |
| NLGN4X | Corneal_stroma_cells |
| OLFML3 | Corneal_stroma_cells |
| PCOLCE2 | Corneal_stroma_cells |
| PDGFRL | Corneal_stroma_cells |
| PRRX1 | Corneal_stroma_cells |
| SOD3 | Corneal_stroma_cells |
| SPOCK1 | Corneal_stroma_cells |
| THBS1 | Corneal_stroma_cells |
| THBS4 | Corneal_stroma_cells |
| WNT5A | Corneal_stroma_cells |
| CDH11 | CribiformJCT_cells |
| CHEK2 | CribiformJCT_cells |
| CLEC11A | CribiformJCT_cells |
| COCH | CribiformJCT_cells |
| HYKK | CribiformJCT_cells |
| MATN2 | CribiformJCT_cells |
| MSMP | CribiformJCT_cells |
| MYOC | CribiformJCT_cells |
| NELL2 | CribiformJCT_cells |
| RSPO2 | CribiformJCT_cells |
| RSPO4 | CribiformJCT_cells |
| SERPINE2 | CribiformJCT_cells |
| ADSL | erythrocytes |
| AGO2 | erythrocytes |
| AHSP | erythrocytes |
| AKR1C3 | erythrocytes |
| ARL9 | erythrocytes |
| ATP23 | erythrocytes |
| AURKB | erythrocytes |
| BIRC5 | erythrocytes |
| CA1 | erythrocytes |
| CCNB1 | erythrocytes |
| CCNB2 | erythrocytes |
| CCNE1 | erythrocytes |
| CDK1 | erythrocytes |
| CDKN3 | erythrocytes |
| CHEK1 | erythrocytes |
| CTSE | erythrocytes |
| DSCC1 | erythrocytes |
| EEF1E1 | erythrocytes |
| EIF1AY | erythrocytes |
| EPOR | erythrocytes |
| FXN | erythrocytes |
| GABPB1 | erythrocytes |
| GMNN | erythrocytes |
| HBA1 | erythrocytes |
| HBD | erythrocytes |
| HBG2 | erythrocytes |
| HBQ1 | erythrocytes |
| HMBS | erythrocytes |
| KEL | erythrocytes |
| KIF23 | erythrocytes |
| KRT1 | erythrocytes |
| MAD2L1 | erythrocytes |
| NDC80 | erythrocytes |
| NMU | erythrocytes |
| ORC6 | erythrocytes |
| PAXIP1 | erythrocytes |
| PBK | erythrocytes |
| PCLAF | erythrocytes |
| PKLR | erythrocytes |
| PLK1 | erythrocytes |
| PLPP4 | erythrocytes |
| POLE2 | erythrocytes |
| PPIH | erythrocytes |
| PRC1 | erythrocytes |
| PSMD9 | erythrocytes |
| PSMG4 | erythrocytes |
| PTTG1 | erythrocytes |
| RFESD | erythrocytes |
| SEC14L4 | erythrocytes |
| SKA1 | erythrocytes |
| SLC22A16 | erythrocytes |
| SOX6 | erythrocytes |
| SPA17 | erythrocytes |
| SPC25 | erythrocytes |
| SPDL1 | erythrocytes |
| ST6GALNAC1 | erythrocytes |
| TFRC | erythrocytes |
| TK1 | erythrocytes |
| UBE2T | erythrocytes |
| UROD | erythrocytes |
| UROS | erythrocytes |
| VRK1 | erythrocytes |
| ADAMTSL1 | fibroblasts |
| ALKAL2 | fibroblasts |
| ANTXR1 | fibroblasts |
| CD248 | fibroblasts |
| CFD | fibroblasts |
| CLMP | fibroblasts |
| COL1A1 | fibroblasts |
| COL3A1 | fibroblasts |
| CTHRC1 | fibroblasts |
| CTSK | fibroblasts |
| CXCL12 | fibroblasts |
| DPT | fibroblasts |
| EGFL6 | fibroblasts |
| FBLN1 | fibroblasts |
| FLRT2 | fibroblasts |
| FSTL1 | fibroblasts |
| GPC3 | fibroblasts |
| GSN | fibroblasts |
| LAMA2 | fibroblasts |
| LEPR | fibroblasts |
| MFAP4 | fibroblasts |
| MMP2 | fibroblasts |
| MXRA8 | fibroblasts |
| OGN | fibroblasts |
| OMD | fibroblasts |
| PAM | fibroblasts |
| SCARA5 | fibroblasts |
| SVEP1 | fibroblasts |
| THBS2 | fibroblasts |
| TNXB | fibroblasts |
| ABCF3 | hyalocytes |
| ABHD12 | hyalocytes |
| ABI3 | hyalocytes |
| ABRAXAS2 | hyalocytes |
| ACLY | hyalocytes |
| ACVR2A | hyalocytes |
| ADAMDEC1 | hyalocytes |
| ADPRH | hyalocytes |
| ADPRS | hyalocytes |
| AIM2 | hyalocytes |
| ALG2 | hyalocytes |
| ALKBH3 | hyalocytes |
| ALOX15B | hyalocytes |
| ANOS1 | hyalocytes |
| AP1AR | hyalocytes |
| APEX2 | hyalocytes |
| ARAF | hyalocytes |
| ARFIP1 | hyalocytes |
| ARNT | hyalocytes |
| ARSB | hyalocytes |
| ASCC1 | hyalocytes |
| ASPRV1 | hyalocytes |
| ATG4C | hyalocytes |
| ATIC | hyalocytes |
| B3GLCT | hyalocytes |
| B4GALT7 | hyalocytes |
| B9D2 | hyalocytes |
| BAG4 | hyalocytes |
| BATF3 | hyalocytes |
| BLMH | hyalocytes |
| BLOC1S5 | hyalocytes |
| BMF | hyalocytes |
| BMT2 | hyalocytes |
| BRMS1L | hyalocytes |
| BRPF1 | hyalocytes |
| BTN3A3 | hyalocytes |
| C1GALT1C1 | hyalocytes |
| CABLES2 | hyalocytes |
| CALCR | hyalocytes |
| CARD9 | hyalocytes |
| CARNMT1 | hyalocytes |
| CASP5 | hyalocytes |
| CASP7 | hyalocytes |
| CBFB | hyalocytes |
| CCL7 | hyalocytes |
| CCL8 | hyalocytes |
| CCL13 | hyalocytes |
| CCL17 | hyalocytes |
| CCL22 | hyalocytes |
| CD1A | hyalocytes |
| CD1D | hyalocytes |
| CD4 | hyalocytes |
| CD33 | hyalocytes |
| CD80 | hyalocytes |
| CD86 | hyalocytes |
| CD207 | hyalocytes |
| CD300C | hyalocytes |
| CDCP1 | hyalocytes |
| CDK5 | hyalocytes |
| CDYL2 | hyalocytes |
| CELA1 | hyalocytes |
| CERS5 | hyalocytes |
| CETP | hyalocytes |
| CHMP6 | hyalocytes |
| CHST10 | hyalocytes |
| CNEP1R1 | hyalocytes |
| COA7 | hyalocytes |
| COMMD5 | hyalocytes |
| COMMD8 | hyalocytes |
| COMMD10 | hyalocytes |
| CRKL | hyalocytes |
| CST6 | hyalocytes |
| CUTC | hyalocytes |
| CWF19L1 | hyalocytes |
| CXCL5 | hyalocytes |
| CXCL10 | hyalocytes |
| CXCL11 | hyalocytes |
| DARS2 | hyalocytes |
| DCAF12 | hyalocytes |
| DCP1B | hyalocytes |
| DHX58 | hyalocytes |
| DLAT | hyalocytes |
| DNASE2B | hyalocytes |
| DOK1 | hyalocytes |
| DPH2 | hyalocytes |
| DTD2 | hyalocytes |
| EBI3 | hyalocytes |
| EIF1AD | hyalocytes |
| EIF2B1 | hyalocytes |
| ELAC1 | hyalocytes |
| ENOX2 | hyalocytes |
| EPHB1 | hyalocytes |
| EPHB2 | hyalocytes |
| EPHB3 | hyalocytes |
| ETV5 | hyalocytes |
| ETV7 | hyalocytes |
| EXOSC5 | hyalocytes |
| FAM3B | hyalocytes |
| FANCF | hyalocytes |
| FANCL | hyalocytes |
| FARS2 | hyalocytes |
| FBXO28 | hyalocytes |
| FCRLB | hyalocytes |
| FGF20 | hyalocytes |
| FKBP7 | hyalocytes |
| FUCA1 | hyalocytes |
| FZD1 | hyalocytes |
| FZD9 | hyalocytes |
| GALE | hyalocytes |
| GHRL | hyalocytes |
| GM2A | hyalocytes |
| GMFB | hyalocytes |
| GOLPH3L | hyalocytes |
| GPX7 | hyalocytes |
| GSTM1 | hyalocytes |
| GTF2E1 | hyalocytes |
| GTF2E2 | hyalocytes |
| GTSF1 | hyalocytes |
| H2BU1 | hyalocytes |
| H3C1 | hyalocytes |
| HCCS | hyalocytes |
| HECTD3 | hyalocytes |
| HENMT1 | hyalocytes |
| HLA-G | hyalocytes |
| HSCB | hyalocytes |
| HTR7 | hyalocytes |
| IBSP | hyalocytes |
| IFIH1 | hyalocytes |
| IFNLR1 | hyalocytes |
| IGFLR1 | hyalocytes |
| IL2 | hyalocytes |
| IL10 | hyalocytes |
| IL12RB1 | hyalocytes |
| IL15 | hyalocytes |
| IL18BP | hyalocytes |
| IL21R | hyalocytes |
| INPP5B | hyalocytes |
| IRF5 | hyalocytes |
| ITPRIPL1 | hyalocytes |
| KCTD5 | hyalocytes |
| KDM1A | hyalocytes |
| KLHL12 | hyalocytes |
| KTI12 | hyalocytes |
| LACC1 | hyalocytes |
| LANCL2 | hyalocytes |
| LHB | hyalocytes |
| LINGO3 | hyalocytes |
| LIPN | hyalocytes |
| LOXL3 | hyalocytes |
| M1AP | hyalocytes |
| MAN2B2 | hyalocytes |
| MAP1LC3B2 | hyalocytes |
| MAPKAPK3 | hyalocytes |
| MB21D2 | hyalocytes |
| MED11 | hyalocytes |
| MED20 | hyalocytes |
| METAP1 | hyalocytes |
| METTL1 | hyalocytes |
| MILR1 | hyalocytes |
| MMGT1 | hyalocytes |
| MMP12 | hyalocytes |
| MRPS14 | hyalocytes |
| MST1R | hyalocytes |
| MTMR1 | hyalocytes |
| MVB12B | hyalocytes |
| MYBPH | hyalocytes |
| NAGLU | hyalocytes |
| NAGPA | hyalocytes |
| NANP | hyalocytes |
| NARS2 | hyalocytes |
| NAT1 | hyalocytes |
| NDUFAF1 | hyalocytes |
| NFU1 | hyalocytes |
| NFYA | hyalocytes |
| NIPSNAP3B | hyalocytes |
| NKIRAS2 | hyalocytes |
| NLRP4 | hyalocytes |
| NPPB | hyalocytes |
| NRAS | hyalocytes |
| NSDHL | hyalocytes |
| NUBP1 | hyalocytes |
| NVL | hyalocytes |
| OAS1 | hyalocytes |
| OSM | hyalocytes |
| PAPSS1 | hyalocytes |
| PARP11 | hyalocytes |
| PARP16 | hyalocytes |
| PCDHGA10 | hyalocytes |
| PDCD1LG2 | hyalocytes |
| PDGFC | hyalocytes |
| PET117 | hyalocytes |
| PF4V1 | hyalocytes |
| PIGR | hyalocytes |
| PLA2G2C | hyalocytes |
| PLA2G4A | hyalocytes |
| PLA2G7 | hyalocytes |
| PLBD2 | hyalocytes |
| PLPBP | hyalocytes |
| PNPO | hyalocytes |
| POGLUT1 | hyalocytes |
| POLB | hyalocytes |
| POMGNT2 | hyalocytes |
| PPCDC | hyalocytes |
| PPM1D | hyalocytes |
| PREP | hyalocytes |
| PRKN | hyalocytes |
| PRTFDC1 | hyalocytes |
| PSMD5 | hyalocytes |
| PSMD10 | hyalocytes |
| PTPN9 | hyalocytes |
| PUDP | hyalocytes |
| PUS7 | hyalocytes |
| PWWP2B | hyalocytes |
| RAB7B | hyalocytes |
| RAP2A | hyalocytes |
| RELL1 | hyalocytes |
| RFFL | hyalocytes |
| RFX5 | hyalocytes |
| RNF34 | hyalocytes |
| RNPEP | hyalocytes |
| RPP40 | hyalocytes |
| RTCA | hyalocytes |
| RTN4IP1 | hyalocytes |
| RTP4 | hyalocytes |
| RUFY1 | hyalocytes |
| SAYSD1 | hyalocytes |
| SCIMP | hyalocytes |
| SEC22A | hyalocytes |
| SEL1L2 | hyalocytes |
| SEPTIN6 | hyalocytes |
| SF3B4 | hyalocytes |
| SIGLEC7 | hyalocytes |
| SIGLEC9 | hyalocytes |
| SIGLEC11 | hyalocytes |
| SLAMF8 | hyalocytes |
| SLC30A5 | hyalocytes |
| SNAPIN | hyalocytes |
| SPATA9 | hyalocytes |
| SPON1 | hyalocytes |
| SPRED1 | hyalocytes |
| SPTLC1 | hyalocytes |
| STAMBPL1 | hyalocytes |
| STX6 | hyalocytes |
| STX18 | hyalocytes |
| STYX | hyalocytes |
| SUMF1 | hyalocytes |
| SUMO4 | hyalocytes |
| SVBP | hyalocytes |
| TADA1 | hyalocytes |
| TAF12 | hyalocytes |
| TAPBPL | hyalocytes |
| TATDN3 | hyalocytes |
| TBC1D13 | hyalocytes |
| TBL2 | hyalocytes |
| TCP11L1 | hyalocytes |
| TFB1M | hyalocytes |
| THAP11 | hyalocytes |
| TIMD4 | hyalocytes |
| TIPIN | hyalocytes |
| TLR3 | hyalocytes |
| TLR5 | hyalocytes |
| TMEM52B | hyalocytes |
| TMEM106A | hyalocytes |
| TMEM119 | hyalocytes |
| TNFRSF8 | hyalocytes |
| TNFRSF11A | hyalocytes |
| TNFSF8 | hyalocytes |
| TNFSF18 | hyalocytes |
| TNNI2 | hyalocytes |
| TRAPPC13 | hyalocytes |
| TREML1 | hyalocytes |
| TREML2 | hyalocytes |
| TRIM21 | hyalocytes |
| TTC27 | hyalocytes |
| UBA7 | hyalocytes |
| UBASH3B | hyalocytes |
| UTP6 | hyalocytes |
| VTA1 | hyalocytes |
| XPNPEP1 | hyalocytes |
| XRCC4 | hyalocytes |
| YAE1 | hyalocytes |
| YJU2 | hyalocytes |
| ZBTB33 | hyalocytes |
| ZKSCAN7 | hyalocytes |
| ZNF34 | hyalocytes |
| ZNF230 | hyalocytes |
| ZW10 | hyalocytes |
| ZWILCH | hyalocytes |
| ACBD7 | lens |
| ADAMTS13 | lens |
| ADAMTS15 | lens |
| ADAMTSL2 | lens |
| AMIGO1 | lens |
| AMY2A | lens |
| AMY2B | lens |
| ANKRD1 | lens |
| ARHGEF25 | lens |
| ASAP3 | lens |
| B3GALT5 | lens |
| B3GNT4 | lens |
| B3GNT8 | lens |
| B4GALT2 | lens |
| BMP8B | lens |
| C1QL3 | lens |
| C2orf66 | lens |
| C2orf73 | lens |
| C4A | lens |
| C5orf63 | lens |
| CA6 | lens |
| CA13 | lens |
| CASTOR1 | lens |
| CCDC24 | lens |
| CCDC89 | lens |
| CCNA1 | lens |
| CCNA2 | lens |
| CDC25B | lens |
| CDH20 | lens |
| CDK15 | lens |
| CDNF | lens |
| CEL | lens |
| CFAP45 | lens |
| CHAD | lens |
| CHST3 | lens |
| CILP | lens |
| CLCA2 | lens |
| CLCNKB | lens |
| CLSTN2 | lens |
| CNTN6 | lens |
| COL11A2 | lens |
| CPOX | lens |
| CRPPA | lens |
| CRYBA2 | lens |
| CRYBB3 | lens |
| CRYGD | lens |
| CSMD1 | lens |
| CYSRT1 | lens |
| DEFB118 | lens |
| DKKL1 | lens |
| DLG3 | lens |
| DLST | lens |
| DNAI1 | lens |
| DNALI1 | lens |
| DNASE1L2 | lens |
| DUSP19 | lens |
| DYNLRB2 | lens |
| EDA2R | lens |
| EDN2 | lens |
| EFNA3 | lens |
| EFNB3 | lens |
| EFS | lens |
| ELAPOR2 | lens |
| ELL3 | lens |
| EMILIN3 | lens |
| ENDOU | lens |
| EPHA5 | lens |
| ERBB2 | lens |
| FABP9 | lens |
| FAM86B1 | lens |
| FAM241B | lens |
| FCN2 | lens |
| FGF22 | lens |
| FGFBP3 | lens |
| FGFR4 | lens |
| FJX1 | lens |
| FLRT3 | lens |
| FOXO4 | lens |
| FOXP4 | lens |
| FSTL4 | lens |
| FUT2 | lens |
| GALNT13 | lens |
| GCNT4 | lens |
| GDF9 | lens |
| GFRA1 | lens |
| GNRH1 | lens |
| GP1BA | lens |
| GPC5 | lens |
| GSTT2B | lens |
| HEPACAM2 | lens |
| HEPHL1 | lens |
| HS6ST1 | lens |
| HSF2BP | lens |
| IGFBPL1 | lens |
| IGSF3 | lens |
| IL17D | lens |
| IL17RB | lens |
| IL17RE | lens |
| INHA | lens |
| INHBB | lens |
| IQCD | lens |
| ITPKA | lens |
| JAKMIP3 | lens |
| KCNF1 | lens |
| KCNN1 | lens |
| KCTD1 | lens |
| KCTD15 | lens |
| KIAA1549L | lens |
| KIRREL1 | lens |
| KLK5 | lens |
| KREMEN1 | lens |
| KRT16 | lens |
| LDHC | lens |
| LDOC1 | lens |
| LGSN | lens |
| LOXL2 | lens |
| LRATD2 | lens |
| LRFN3 | lens |
| LRFN4 | lens |
| LRP4 | lens |
| MAP3K10 | lens |
| MBLAC2 | lens |
| MEIG1 | lens |
| MFAP5 | lens |
| MGAT3 | lens |
| MMACHC | lens |
| MMEL1 | lens |
| MPL | lens |
| MREG | lens |
| MSTN | lens |
| MUC1 | lens |
| NEO1 | lens |
| NKD2 | lens |
| NMS | lens |
| NOMO2 | lens |
| NPFF | lens |
| NPTXR | lens |
| NR3C2 | lens |
| NUDT11 | lens |
| PAK4 | lens |
| PCDHB2 | lens |
| PCDHB10 | lens |
| PCDHGA2 | lens |
| PCDHGC3 | lens |
| PDZK1 | lens |
| PHEX | lens |
| PI15 | lens |
| PLCG1 | lens |
| PLXNB3 | lens |
| PRKG1 | lens |
| PRSS27 | lens |
| PXDNL | lens |
| PXYLP1 | lens |
| RAB39B | lens |
| RAB43 | lens |
| REM1 | lens |
| RGS8 | lens |
| RILPL1 | lens |
| RNF43 | lens |
| RNF122 | lens |
| RNF215 | lens |
| ROR1 | lens |
| RPS6KA6 | lens |
| RTN4R | lens |
| S100A5 | lens |
| SCGB1D2 | lens |
| SCUBE3 | lens |
| SEMA3E | lens |
| SEMA5B | lens |
| SEPTIN5 | lens |
| SFTPD | lens |
| SHBG | lens |
| SIAE | lens |
| SIRT5 | lens |
| SIRT6 | lens |
| SMN1 | lens |
| SNTA1 | lens |
| SNX12 | lens |
| SOCS7 | lens |
| SORCS3 | lens |
| SOST | lens |
| SPACA3 | lens |
| SPATC1L | lens |
| SPIN3 | lens |
| SPINK6 | lens |
| SRXN1 | lens |
| STAC | lens |
| STK16 | lens |
| STXBP4 | lens |
| SYCE1L | lens |
| SYT9 | lens |
| TCAP | lens |
| TCTN2 | lens |
| TEAD4 | lens |
| TGM1 | lens |
| THBS3 | lens |
| TLN2 | lens |
| TMEM8B | lens |
| TMEM25 | lens |
| TMEM52 | lens |
| TMEM225B | lens |
| TNC | lens |
| TNFRSF25 | lens |
| TNFSF15 | lens |
| TPPP | lens |
| TSLP | lens |
| TTC9 | lens |
| UBQLN4 | lens |
| ULBP1 | lens |
| ULBP3 | lens |
| UNC119B | lens |
| VANGL1 | lens |
| VCX | lens |
| VSTM2L | lens |
| WFIKKN1 | lens |
| WFIKKN2 | lens |
| WNT7A | lens |
| WWC1 | lens |
| YY2 | lens |
| ZNF175 | lens |
| ZNF334 | lens |
| ZNF415 | lens |
| ZNF560 | lens |
| ZNF696 | lens |
| ZNF843 | lens |
| ZNRF3 | lens |
| A1BG | hepatocytes |
| AFM | hepatocytes |
| AFP | hepatocytes |
| AGT | hepatocytes |
| AHSG | hepatocytes |
| ALB | hepatocytes |
| AMBP | hepatocytes |
| ANG | hepatocytes |
| ANGPTL3 | hepatocytes |
| ANGPTL4 | hepatocytes |
| ANGPTL8 | hepatocytes |
| APCS | hepatocytes |
| APOA1 | hepatocytes |
| APOA2 | hepatocytes |
| APOA4 | hepatocytes |
| APOA5 | hepatocytes |
| APOB | hepatocytes |
| APOC1 | hepatocytes |
| APOC2 | hepatocytes |
| APOC3 | hepatocytes |
| APOE | hepatocytes |
| APOF | hepatocytes |
| APOH | hepatocytes |
| APOM | hepatocytes |
| ASGR1 | hepatocytes |
| AZGP1 | hepatocytes |
| BCHE | hepatocytes |
| C1R | hepatocytes |
| C1RL | hepatocytes |
| C2 | hepatocytes |
| C3 | hepatocytes |
| C4BPA | hepatocytes |
| C5 | hepatocytes |
| C6 | hepatocytes |
| C8G | hepatocytes |
| C9 | hepatocytes |
| CALR | hepatocytes |
| CCL15 | hepatocytes |
| CCL16 | hepatocytes |
| CFB | hepatocytes |
| CFH | hepatocytes |
| CFHR1 | hepatocytes |
| CFHR2 | hepatocytes |
| CFHR3 | hepatocytes |
| CFHR4 | hepatocytes |
| CFHR5 | hepatocytes |
| CFI | hepatocytes |
| CHRDL2 | hepatocytes |
| COL18A1 | hepatocytes |
| CPB2 | hepatocytes |
| CPN1 | hepatocytes |
| CPN2 | hepatocytes |
| CRP | hepatocytes |
| ENTPD5 | hepatocytes |
| F2 | hepatocytes |
| F7 | hepatocytes |
| F9 | hepatocytes |
| F10 | hepatocytes |
| F11 | hepatocytes |
| F13B | hepatocytes |
| FETUB | hepatocytes |
| FGA | hepatocytes |
| FGG | hepatocytes |
| FGL1 | hepatocytes |
| FN1 | hepatocytes |
| FNDC4 | hepatocytes |
| FURIN | hepatocytes |
| GC | hepatocytes |
| GGH | hepatocytes |
| GHR | hepatocytes |
| HABP2 | hepatocytes |
| HAMP | hepatocytes |
| HGFAC | hepatocytes |
| HJV | hepatocytes |
| HP | hepatocytes |
| HPX | hepatocytes |
| HRG | hepatocytes |
| HYAL1 | hepatocytes |
| IGF1 | hepatocytes |
| IGF2 | hepatocytes |
| IGFALS | hepatocytes |
| IGFBP1 | hepatocytes |
| IGFBP2 | hepatocytes |
| IL1RAP | hepatocytes |
| ITIH1 | hepatocytes |
| ITIH2 | hepatocytes |
| ITIH3 | hepatocytes |
| ITIH4 | hepatocytes |
| KNG1 | hepatocytes |
| LBP | hepatocytes |
| LCAT | hepatocytes |
| LEAP2 | hepatocytes |
| LECT2 | hepatocytes |
| MASP1 | hepatocytes |
| MBL2 | hepatocytes |
| MST1 | hepatocytes |
| NUCB2 | hepatocytes |
| OXT | hepatocytes |
| P4HB | hepatocytes |
| PGLYRP2 | hepatocytes |
| PLA2G12B | hepatocytes |
| PLG | hepatocytes |
| PON1 | hepatocytes |
| PROC | hepatocytes |
| PROS1 | hepatocytes |
| RARRES2 | hepatocytes |
| RBP4 | hepatocytes |
| SAA1 | hepatocytes |
| SAA2 | hepatocytes |
| SAA4 | hepatocytes |
| SERPINA1 | hepatocytes |
| SERPINA4 | hepatocytes |
| SERPINA5 | hepatocytes |
| SERPINA7 | hepatocytes |
| SERPINA10 | hepatocytes |
| SERPINA11 | hepatocytes |
| SERPINC1 | hepatocytes |
| SERPINF1 | hepatocytes |
| SERPINF2 | hepatocytes |
| SERPING1 | hepatocytes |
| SMOC1 | hepatocytes |
| SORD | hepatocytes |
| SPINK1 | hepatocytes |
| ST6GAL1 | hepatocytes |
| TF | hepatocytes |
| TMPRSS6 | hepatocytes |
| TXN | hepatocytes |
| VTN | hepatocytes |
| ANXA1 | mast_cells |
| BATF | mast_cells |
| CLNK | mast_cells |
| CMA1 | mast_cells |
| COL13A1 | mast_cells |
| CSF1 | mast_cells |
| CTSG | mast_cells |
| DUSP6 | mast_cells |
| HPGDS | mast_cells |
| IL13 | mast_cells |
| IL18R1 | mast_cells |
| KIT | mast_cells |
| MAPK6 | mast_cells |
| NDEL1 | mast_cells |
| PNMT | mast_cells |
| RAB27B | mast_cells |
| RHEX | mast_cells |
| STXBP6 | mast_cells |
| TIAM2 | mast_cells |
| TPSAB1 | mast_cells |
| TPSB2 | mast_cells |
| TPSG1 | mast_cells |
| AIF1L | melanocytes |
| BCAN | melanocytes |
| CAPN3 | melanocytes |
| CDK2 | melanocytes |
| GYG2 | melanocytes |
| MET | melanocytes |
| MITF | melanocytes |
| PIR | melanocytes |
| PMEL | melanocytes |
| QPCT | melanocytes |
| RAB38 | melanocytes |
| SNAI2 | melanocytes |
| TRIB2 | melanocytes |
| AIF1 | mono_macrophages |
| C1QA | mono_macrophages |
| C1QC | mono_macrophages |
| CD5L | mono_macrophages |
| CD68 | mono_macrophages |
| CXCL3 | mono_macrophages |
| EREG | mono_macrophages |
| IL1B | mono_macrophages |
| MMP19 | mono_macrophages |
| PLAUR | mono_macrophages |
| RNASET2 | mono_macrophages |
| VCAN | mono_macrophages |
| ACAP2 | neutrophils |
| ACTN1 | neutrophils |
| ADGRE5 | neutrophils |
| ADSS2 | neutrophils |
| AGER | neutrophils |
| AGFG1 | neutrophils |
| ALDH3B1 | neutrophils |
| ALOX5 | neutrophils |
| ALPL | neutrophils |
| AMPD2 | neutrophils |
| ANXA11 | neutrophils |
| APBB1IP | neutrophils |
| ARHGAP1 | neutrophils |
| ARHGAP30 | neutrophils |
| ARHGAP45 | neutrophils |
| ARHGEF1 | neutrophils |
| ARHGEF2 | neutrophils |
| ARID1A | neutrophils |
| ARID3A | neutrophils |
| ARPC5 | neutrophils |
| ARSA | neutrophils |
| ATP2A3 | neutrophils |
| B4GALT5 | neutrophils |
| BAG6 | neutrophils |
| BCL6 | neutrophils |
| BIN2 | neutrophils |
| BMX | neutrophils |
| BPI | neutrophils |
| BST1 | neutrophils |
| BTNL8 | neutrophils |
| C16orf54 | neutrophils |
| CA4 | neutrophils |
| CAMP | neutrophils |
| CAPZA1 | neutrophils |
| CASP4 | neutrophils |
| CASP8 | neutrophils |
| CBL | neutrophils |
| CCM2 | neutrophils |
| CCPG1 | neutrophils |
| CD177 | neutrophils |
| CDA | neutrophils |
| CDKN2D | neutrophils |
| CEACAM3 | neutrophils |
| CEACAM4 | neutrophils |
| CEACAM8 | neutrophils |
| CEBPE | neutrophils |
| CERT1 | neutrophils |
| CFP | neutrophils |
| CHFR | neutrophils |
| CHI3L1 | neutrophils |
| CHIT1 | neutrophils |
| CHKB | neutrophils |
| CKAP4 | neutrophils |
| CLC | neutrophils |
| CLEC1B | neutrophils |
| CLEC4D | neutrophils |
| CLEC7A | neutrophils |
| CLEC12A | neutrophils |
| CLK2 | neutrophils |
| CPPED1 | neutrophils |
| CR1 | neutrophils |
| CRACR2A | neutrophils |
| CREBBP | neutrophils |
| CRISP2 | neutrophils |
| CRISP3 | neutrophils |
| CSF2RA | neutrophils |
| CSF3R | neutrophils |
| CSNK1A1L | neutrophils |
| CSNK1D | neutrophils |
| CTDSP1 | neutrophils |
| CXCL1 | neutrophils |
| CXCL6 | neutrophils |
| CYB5R4 | neutrophils |
| CYRIB | neutrophils |
| CYTH1 | neutrophils |
| CYTH4 | neutrophils |
| DBNL | neutrophils |
| DDX6 | neutrophils |
| DEFA1 | neutrophils |
| DEFA3 | neutrophils |
| DNM2 | neutrophils |
| DOCK2 | neutrophils |
| DPEP2 | neutrophils |
| DUSP13 | neutrophils |
| EFHD2 | neutrophils |
| EGLN2 | neutrophils |
| EHD1 | neutrophils |
| EIF4G3 | neutrophils |
| ELANE | neutrophils |
| ELAPOR1 | neutrophils |
| FAM151B | neutrophils |
| FBXL5 | neutrophils |
| FCGR3B | neutrophils |
| FCN1 | neutrophils |
| FOLR3 | neutrophils |
| G6PD | neutrophils |
| GALNS | neutrophils |
| GCA | neutrophils |
| GGA3 | neutrophils |
| GLIPR2 | neutrophils |
| GMFG | neutrophils |
| GNAQ | neutrophils |
| GPCPD1 | neutrophils |
| GRK2 | neutrophils |
| GRN | neutrophils |
| GSR | neutrophils |
| H2BC21 | neutrophils |
| HCLS1 | neutrophils |
| HDAC4 | neutrophils |
| HMGB2 | neutrophils |
| HSH2D | neutrophils |
| IFNGR2 | neutrophils |
| IGF2R | neutrophils |
| IL1R2 | neutrophils |
| IL4R | neutrophils |
| IL6R | neutrophils |
| IL10RB | neutrophils |
| IL17RA | neutrophils |
| IL18RAP | neutrophils |
| IMPA2 | neutrophils |
| IRAG2 | neutrophils |
| ITGAL | neutrophils |
| ITGAM | neutrophils |
| ITGB2 | neutrophils |
| ITPK1 | neutrophils |
| JAML | neutrophils |
| JMJD6 | neutrophils |
| KCNAB2 | neutrophils |
| KIAA0319L | neutrophils |
| KLK3 | neutrophils |
| LCN2 | neutrophils |
| LCP1 | neutrophils |
| LILRA2 | neutrophils |
| LILRA5 | neutrophils |
| LILRA6 | neutrophils |
| LILRB2 | neutrophils |
| LILRB3 | neutrophils |
| LRP10 | neutrophils |
| LRRC4 | neutrophils |
| LRRFIP2 | neutrophils |
| LRRK2 | neutrophils |
| LSP1 | neutrophils |
| LTA4H | neutrophils |
| LTB4R | neutrophils |
| LTF | neutrophils |
| LYN | neutrophils |
| MANSC1 | neutrophils |
| MAP2K3 | neutrophils |
| MAPK1 | neutrophils |
| MAPK13 | neutrophils |
| MAPK14 | neutrophils |
| MAPKAPK2 | neutrophils |
| MDM2 | neutrophils |
| MEMO1 | neutrophils |
| MICAL1 | neutrophils |
| MMP8 | neutrophils |
| MMP9 | neutrophils |
| MNDA | neutrophils |
| MPO | neutrophils |
| NADK | neutrophils |
| NATD1 | neutrophils |
| NCF2 | neutrophils |
| NLRP1 | neutrophils |
| NME8 | neutrophils |
| NOTCH1 | neutrophils |
| NPEPL1 | neutrophils |
| NUMB | neutrophils |
| OLFM4 | neutrophils |
| PELI1 | neutrophils |
| PGD | neutrophils |
| PGLYRP1 | neutrophils |
| PLEK | neutrophils |
| PLXNC1 | neutrophils |
| PNPLA2 | neutrophils |
| PPP1R9B | neutrophils |
| PPP2R5A | neutrophils |
| PRKAA1 | neutrophils |
| PRKACA | neutrophils |
| PRKCD | neutrophils |
| PROK2 | neutrophils |
| PSTPIP1 | neutrophils |
| PTEN | neutrophils |
| PTK2B | neutrophils |
| PTPN6 | neutrophils |
| PTPRJ | neutrophils |
| PYGL | neutrophils |
| QSOX1 | neutrophils |
| RAB3D | neutrophils |
| RAB5B | neutrophils |
| RAB18 | neutrophils |
| RAB24 | neutrophils |
| RAB31 | neutrophils |
| RAB37 | neutrophils |
| RAC2 | neutrophils |
| RAF1 | neutrophils |
| RASSF2 | neutrophils |
| RASSF5 | neutrophils |
| RELT | neutrophils |
| RETN | neutrophils |
| RHOG | neutrophils |
| RNF141 | neutrophils |
| S100A12 | neutrophils |
| S100P | neutrophils |
| SECTM1 | neutrophils |
| SELL | neutrophils |
| SEMA4A | neutrophils |
| SEMA4D | neutrophils |
| SERPINB1 | neutrophils |
| SETD2 | neutrophils |
| SH3BP2 | neutrophils |
| SH3GLB1 | neutrophils |
| SH3KBP1 | neutrophils |
| SIGLEC5 | neutrophils |
| SIRPA | neutrophils |
| SIRPB1 | neutrophils |
| SNX27 | neutrophils |
| SPI1 | neutrophils |
| SPN | neutrophils |
| ST3GAL2 | neutrophils |
| STAT6 | neutrophils |
| STK10 | neutrophils |
| STK17B | neutrophils |
| SULT1B1 | neutrophils |
| SVIP | neutrophils |
| SYTL1 | neutrophils |
| TACC3 | neutrophils |
| TBC1D20 | neutrophils |
| TCN1 | neutrophils |
| TMEM154 | neutrophils |
| TNFAIP8 | neutrophils |
| TP53I11 | neutrophils |
| TRABD | neutrophils |
| TREM1 | neutrophils |
| TRIM27 | neutrophils |
| TSEN34 | neutrophils |
| TTPAL | neutrophils |
| TYK2 | neutrophils |
| UNC13D | neutrophils |
| USP3 | neutrophils |
| USP15 | neutrophils |
| UTS2 | neutrophils |
| VASP | neutrophils |
| VAV1 | neutrophils |
| VNN2 | neutrophils |
| VPS53 | neutrophils |
| VSIR | neutrophils |
| VSTM1 | neutrophils |
| WAS | neutrophils |
| YTHDC1 | neutrophils |
| ZFAND2B | neutrophils |
| ZFAND3 | neutrophils |
| ZYX | neutrophils |
| CCL5 | NK_Tcells |
| CD2 | NK_Tcells |
| CD3E | NK_Tcells |
| CD3G | NK_Tcells |
| CD5 | NK_Tcells |
| CD6 | NK_Tcells |
| CD7 | NK_Tcells |
| CD8A | NK_Tcells |
| CD8B | NK_Tcells |
| CD40LG | NK_Tcells |
| CD96 | NK_Tcells |
| CD247 | NK_Tcells |
| CST7 | NK_Tcells |
| FLT3LG | NK_Tcells |
| FYN | NK_Tcells |
| GNLY | NK_Tcells |
| GZMA | NK_Tcells |
| GZMB | NK_Tcells |
| GZMH | NK_Tcells |
| GZMK | NK_Tcells |
| GZMM | NK_Tcells |
| IFNG | NK_Tcells |
| IL2RB | NK_Tcells |
| IL7R | NK_Tcells |
| IL32 | NK_Tcells |
| KLRC3 | NK_Tcells |
| KLRF1 | NK_Tcells |
| KLRK1 | NK_Tcells |
| LCK | NK_Tcells |
| SH2D1A | NK_Tcells |
| SH2D1B | NK_Tcells |
| TRAT1 | NK_Tcells |
| ZAP70 | NK_Tcells |
| ADAMTS1 | pericytes |
| BGN | pericytes |
| C1QTNF1 | pericytes |
| CHRD | pericytes |
| COX412 | pericytes |
| EPO | pericytes |
| FBLIM1 | pericytes |
| MFGE8 | pericytes |
| NGF | pericytes |
| PTP4A3 | pericytes |
| RGS5 | pericytes |
| TNFRSF12A | pericytes |
| VSTM4 | pericytes |
| AACS | retina_amacrine |
| ABCD4 | retina_amacrine |
| ACTN2 | retina_amacrine |
| ACVR2B | retina_amacrine |
| ADAM11 | retina_amacrine |
| ADGRB1 | retina_amacrine |
| ADGRB2 | retina_amacrine |
| ADGRB3 | retina_amacrine |
| ADGRL3 | retina_amacrine |
| AGAP3 | retina_amacrine |
| AKT2 | retina_amacrine |
| AKT3 | retina_amacrine |
| AMH | retina_amacrine |
| AMPH | retina_amacrine |
| ANKS3 | retina_amacrine |
| AP1G2 | retina_amacrine |
| ARHGEF7 | retina_amacrine |
| ASPSCR1 | retina_amacrine |
| ATF6B | retina_amacrine |
| B3GALT2 | retina_amacrine |
| B3GAT1 | retina_amacrine |
| B4GALNT1 | retina_amacrine |
| BAIAP2 | retina_amacrine |
| BDP1 | retina_amacrine |
| BICD1 | retina_amacrine |
| BIN1 | retina_amacrine |
| BIN3 | retina_amacrine |
| BRICD5 | retina_amacrine |
| BRSK2 | retina_amacrine |
| C1QL2 | retina_amacrine |
| C11orf87 | retina_amacrine |
| CACNB3 | retina_amacrine |
| CACNB4 | retina_amacrine |
| CAMK2A | retina_amacrine |
| CARTPT | retina_amacrine |
| CBARP | retina_amacrine |
| CBLN1 | retina_amacrine |
| CDC42BPB | retina_amacrine |
| CDH10 | retina_amacrine |
| CDKL2 | retina_amacrine |
| CEP43 | retina_amacrine |
| CLSTN3 | retina_amacrine |
| COL2A1 | retina_amacrine |
| COQ6 | retina_amacrine |
| CPNE6 | retina_amacrine |
| CPNE7 | retina_amacrine |
| CPT1B | retina_amacrine |
| CRMP1 | retina_amacrine |
| CRTC3 | retina_amacrine |
| CTSA | retina_amacrine |
| CYLD | retina_amacrine |
| DAZAP1 | retina_amacrine |
| DCLK1 | retina_amacrine |
| DDI2 | retina_amacrine |
| DDX19A | retina_amacrine |
| DNM1 | retina_amacrine |
| DNM1L | retina_amacrine |
| DOT1L | retina_amacrine |
| DPH7 | retina_amacrine |
| DPP7 | retina_amacrine |
| DPYSL4 | retina_amacrine |
| DSCAM | retina_amacrine |
| EFNA2 | retina_amacrine |
| EHMT2 | retina_amacrine |
| ELP1 | retina_amacrine |
| ENAH | retina_amacrine |
| ENTPD3 | retina_amacrine |
| ERC1 | retina_amacrine |
| EVL | retina_amacrine |
| FBXO3 | retina_amacrine |
| FIBCD1 | retina_amacrine |
| FN3K | retina_amacrine |
| FSD1 | retina_amacrine |
| GABBR2 | retina_amacrine |
| GAD1 | retina_amacrine |
| GALNT16 | retina_amacrine |
| GAS6 | retina_amacrine |
| GATAD1 | retina_amacrine |
| GDI1 | retina_amacrine |
| GGA1 | retina_amacrine |
| GPC1 | retina_amacrine |
| GPC6 | retina_amacrine |
| GPI | retina_amacrine |
| GRIA4 | retina_amacrine |
| GRID1 | retina_amacrine |
| GRIPAP1 | retina_amacrine |
| GRM4 | retina_amacrine |
| HAGHL | retina_amacrine |
| HDAC6 | retina_amacrine |
| HGS | retina_amacrine |
| HID1 | retina_amacrine |
| HIP1R | retina_amacrine |
| HMGCR | retina_amacrine |
| HOMER1 | retina_amacrine |
| ICAM5 | retina_amacrine |
| IGF1R | retina_amacrine |
| IGLON5 | retina_amacrine |
| IGSF8 | retina_amacrine |
| ITGA3 | retina_amacrine |
| JAG2 | retina_amacrine |
| JARID2 | retina_amacrine |
| JPH4 | retina_amacrine |
| KAT2A | retina_amacrine |
| KDM4C | retina_amacrine |
| KHDRBS2 | retina_amacrine |
| KMT5C | retina_amacrine |
| LNX1 | retina_amacrine |
| LPIN1 | retina_amacrine |
| LRFN5 | retina_amacrine |
| LRP1B | retina_amacrine |
| LRP2BP | retina_amacrine |
| LRRC4B | retina_amacrine |
| LRRC4C | retina_amacrine |
| LRRC37A2 | retina_amacrine |
| LRRTM2 | retina_amacrine |
| LRRTM3 | retina_amacrine |
| LRTM2 | retina_amacrine |
| LTO1 | retina_amacrine |
| LYG2 | retina_amacrine |
| LYSMD4 | retina_amacrine |
| MACROD2 | retina_amacrine |
| MADCAM1 | retina_amacrine |
| MAEA | retina_amacrine |
| MAPK10 | retina_amacrine |
| MCF2L | retina_amacrine |
| MDGA1 | retina_amacrine |
| MDM4 | retina_amacrine |
| MEIS2 | retina_amacrine |
| MPDZ | retina_amacrine |
| MTMR7 | retina_amacrine |
| MTSS2 | retina_amacrine |
| MTUS2 | retina_amacrine |
| MVD | retina_amacrine |
| MVK | retina_amacrine |
| MYSM1 | retina_amacrine |
| NAPEPLD | retina_amacrine |
| NCAN | retina_amacrine |
| NCR3LG1 | retina_amacrine |
| NECAB3 | retina_amacrine |
| NEURL4 | retina_amacrine |
| NFASC | retina_amacrine |
| NOVA1 | retina_amacrine |
| NPM2 | retina_amacrine |
| NRCAM | retina_amacrine |
| NRG2 | retina_amacrine |
| NRXN2 | retina_amacrine |
| NTRK3 | retina_amacrine |
| OLFM3 | retina_amacrine |
| OTUD3 | retina_amacrine |
| PANK3 | retina_amacrine |
| PCDH9 | retina_amacrine |
| PCSK1 | retina_amacrine |
| PCSK7 | retina_amacrine |
| PDE9A | retina_amacrine |
| PDPK1 | retina_amacrine |
| PHF3 | retina_amacrine |
| PKN2 | retina_amacrine |
| PLEKHA1 | retina_amacrine |
| PLXNA1 | retina_amacrine |
| PLXNA4 | retina_amacrine |
| PNPT1 | retina_amacrine |
| POU6F1 | retina_amacrine |
| PRKCB | retina_amacrine |
| PRKCG | retina_amacrine |
| PRRT2 | retina_amacrine |
| PSD2 | retina_amacrine |
| PSPN | retina_amacrine |
| PTPRN | retina_amacrine |
| PTPRS | retina_amacrine |
| PTPRU | retina_amacrine |
| QSOX2 | retina_amacrine |
| RAB11FIP3 | retina_amacrine |
| RAP1GAP | retina_amacrine |
| RBFOX1 | retina_amacrine |
| RBM4 | retina_amacrine |
| RDH13 | retina_amacrine |
| RFNG | retina_amacrine |
| RGS4 | retina_amacrine |
| RIC3 | retina_amacrine |
| RNPC3 | retina_amacrine |
| SAMD4B | retina_amacrine |
| SCARF2 | retina_amacrine |
| SCN3B | retina_amacrine |
| SEMA6C | retina_amacrine |
| SEMA6D | retina_amacrine |
| SERAC1 | retina_amacrine |
| SHC2 | retina_amacrine |
| SLC4A8 | retina_amacrine |
| SLCO5A1 | retina_amacrine |
| SLIT2 | retina_amacrine |
| SORCS2 | retina_amacrine |
| SSTR1 | retina_amacrine |
| ST6GAL2 | retina_amacrine |
| ST6GALNAC6 | retina_amacrine |
| STX1A | retina_amacrine |
| SV2A | retina_amacrine |
| SYNGR3 | retina_amacrine |
| SYT5 | retina_amacrine |
| SYT6 | retina_amacrine |
| SYT7 | retina_amacrine |
| TBC1D24 | retina_amacrine |
| TCEAL5 | retina_amacrine |
| TENM4 | retina_amacrine |
| TERF1 | retina_amacrine |
| THOC1 | retina_amacrine |
| TMEM59L | retina_amacrine |
| TMEM132B | retina_amacrine |
| TMEM185A | retina_amacrine |
| TNKS | retina_amacrine |
| TOM1L2 | retina_amacrine |
| TRIM9 | retina_amacrine |
| TRIO | retina_amacrine |
| TYRO3 | retina_amacrine |
| U2AF2 | retina_amacrine |
| ULK3 | retina_amacrine |
| UNC5A | retina_amacrine |
| UNC5B | retina_amacrine |
| UNC45A | retina_amacrine |
| WNT11 | retina_amacrine |
| ZC3H8 | retina_amacrine |
| ZNF10 | retina_amacrine |
| ZNF276 | retina_amacrine |
| ZNF483 | retina_amacrine |
| ZRANB1 | retina_amacrine |
| APOBEC2 | retina_bipolar |
| B4GALT6 | retina_bipolar |
| CA10 | retina_bipolar |
| CAMK2B | retina_bipolar |
| CPLX3 | retina_bipolar |
| DGKB | retina_bipolar |
| DPP10 | retina_bipolar |
| EDIL3 | retina_bipolar |
| ELFN2 | retina_bipolar |
| FAM171B | retina_bipolar |
| HS3ST4 | retina_bipolar |
| ISL1 | retina_bipolar |
| KCNG4 | retina_bipolar |
| KIRREL2 | retina_bipolar |
| KIRREL3 | retina_bipolar |
| LRRTM4 | retina_bipolar |
| LRTM1 | retina_bipolar |
| MDGA2 | retina_bipolar |
| NETO1 | retina_bipolar |
| NTNG1 | retina_bipolar |
| PCDH8 | retina_bipolar |
| PLXDC1 | retina_bipolar |
| PPP1R27 | retina_bipolar |
| SLITRK1 | retina_bipolar |
| SLITRK6 | retina_bipolar |
| TAFA4 | retina_bipolar |
| USP46 | retina_bipolar |
| VSX1 | retina_bipolar |
| ADAM12 | retina_cones |
| AP4M1 | retina_cones |
| ASAH2 | retina_cones |
| ASIC4 | retina_cones |
| BICDL1 | retina_cones |
| BMPR1A | retina_cones |
| C1orf226 | retina_cones |
| CHRNA5 | retina_cones |
| CLUAP1 | retina_cones |
| CSNK1G1 | retina_cones |
| DHRS11 | retina_cones |
| DLG4 | retina_cones |
| DPYSL3 | retina_cones |
| EGFLAM | retina_cones |
| EID3 | retina_cones |
| ENGASE | retina_cones |
| EPHA10 | retina_cones |
| EYS | retina_cones |
| FANK1 | retina_cones |
| FGF23 | retina_cones |
| FSTL5 | retina_cones |
| GAN | retina_cones |
| GJD2 | retina_cones |
| GNGT2 | retina_cones |
| GPC2 | retina_cones |
| GUCA1A | retina_cones |
| HES6 | retina_cones |
| IGDCC4 | retina_cones |
| KIN | retina_cones |
| KLHL41 | retina_cones |
| KMT2D | retina_cones |
| KREMEN2 | retina_cones |
| KYAT1 | retina_cones |
| LARGE1 | retina_cones |
| LCORL | retina_cones |
| LRFN2 | retina_cones |
| MDP1 | retina_cones |
| MPPED2 | retina_cones |
| MYL4 | retina_cones |
| NPPC | retina_cones |
| NPTX1 | retina_cones |
| PDE6H | retina_cones |
| PIWIL1 | retina_cones |
| PPM1B | retina_cones |
| PRR16 | retina_cones |
| PRTG | retina_cones |
| RABL3 | retina_cones |
| SERF1A | retina_cones |
| SPATA33 | retina_cones |
| ST8SIA1 | retina_cones |
| TAFA3 | retina_cones |
| TBCC | retina_cones |
| TBPL1 | retina_cones |
| TEAD3 | retina_cones |
| THRB | retina_cones |
| VLDLR | retina_cones |
| ACHE | retina_ganglion |
| ACYP2 | retina_ganglion |
| ADAM22 | retina_ganglion |
| ADAM23 | retina_ganglion |
| ALDOC | retina_ganglion |
| B3GNT2 | retina_ganglion |
| BCL2L2 | retina_ganglion |
| BMERB1 | retina_ganglion |
| C12orf76 | retina_ganglion |
| CALB2 | retina_ganglion |
| CALY | retina_ganglion |
| CAMK2N2 | retina_ganglion |
| CBS | retina_ganglion |
| CCDC92 | retina_ganglion |
| CGREF1 | retina_ganglion |
| CHAC1 | retina_ganglion |
| CHGA | retina_ganglion |
| CHGB | retina_ganglion |
| CKMT1A | retina_ganglion |
| CLEC2L | retina_ganglion |
| CNTN1 | retina_ganglion |
| CPLX1 | retina_ganglion |
| CPLX2 | retina_ganglion |
| CRH | retina_ganglion |
| CSDC2 | retina_ganglion |
| CYGB | retina_ganglion |
| DBNDD1 | retina_ganglion |
| DDAH1 | retina_ganglion |
| DDHD2 | retina_ganglion |
| DIRAS1 | retina_ganglion |
| DMTN | retina_ganglion |
| DNER | retina_ganglion |
| DPYSL2 | retina_ganglion |
| DPYSL5 | retina_ganglion |
| DYNLT3 | retina_ganglion |
| EIF5A2 | retina_ganglion |
| ELAVL2 | retina_ganglion |
| EPB41L1 | retina_ganglion |
| EPDR1 | retina_ganglion |
| FABP3 | retina_ganglion |
| FABP6 | retina_ganglion |
| FKBP1B | retina_ganglion |
| GABBR1 | retina_ganglion |
| GALNT9 | retina_ganglion |
| GDAP1L1 | retina_ganglion |
| HABP4 | retina_ganglion |
| HAPLN4 | retina_ganglion |
| HARS1 | retina_ganglion |
| HECW2 | retina_ganglion |
| INA | retina_ganglion |
| ISLR2 | retina_ganglion |
| JPH3 | retina_ganglion |
| KLC1 | retina_ganglion |
| KLK10 | retina_ganglion |
| LETMD1 | retina_ganglion |
| LG13 | retina_ganglion |
| LIN7A | retina_ganglion |
| MDH1 | retina_ganglion |
| NAP1L2 | retina_ganglion |
| NDRG4 | retina_ganglion |
| NEFH | retina_ganglion |
| NFE2L1 | retina_ganglion |
| NPTN | retina_ganglion |
| NUDT3 | retina_ganglion |
| OLFM1 | retina_ganglion |
| PCDH10 | retina_ganglion |
| PCMT1 | retina_ganglion |
| PCP4L1 | retina_ganglion |
| PFKM | retina_ganglion |
| PLCB1 | retina_ganglion |
| PLD3 | retina_ganglion |
| PNMA2 | retina_ganglion |
| PPP2R1A | retina_ganglion |
| PRKAR1B | retina_ganglion |
| PTH1R | retina_ganglion |
| RAB3C | retina_ganglion |
| RAB4B | retina_ganglion |
| RAB6B | retina_ganglion |
| RAC3 | retina_ganglion |
| RBFOX2 | retina_ganglion |
| RBPMS2 | retina_ganglion |
| RET | retina_ganglion |
| ROBO2 | retina_ganglion |
| RTN1 | retina_ganglion |
| SCN2B | retina_ganglion |
| SH3GL2 | retina_ganglion |
| SLC66A1L | retina_ganglion |
| SMYD2 | retina_ganglion |
| SNCG | retina_ganglion |
| SNPH | retina_ganglion |
| SNX16 | retina_ganglion |
| SPOCK2 | retina_ganglion |
| SPOCK3 | retina_ganglion |
| SPRN | retina_ganglion |
| STMN2 | retina_ganglion |
| STMN3 | retina_ganglion |
| STX1B | retina_ganglion |
| SULT4A1 | retina_ganglion |
| SYT2 | retina_ganglion |
| SYT4 | retina_ganglion |
| SYT11 | retina_ganglion |
| SYT13 | retina_ganglion |
| TAGLN3 | retina_ganglion |
| THY1 | retina_ganglion |
| TTC9B | retina_ganglion |
| UCHL1 | retina_ganglion |
| UNC5D | retina_ganglion |
| VAMP1 | retina_ganglion |
| VAPB | retina_ganglion |
| VARS1 | retina_ganglion |
| VEGFB | retina_ganglion |
| VSNL1 | retina_ganglion |
| VWC2 | retina_ganglion |
| YWHAG | retina_ganglion |
| AQP4 | retina_glia |
| ARHGEF16 | retina_glia |
| C5orf46 | retina_glia |
| CA2 | retina_glia |
| CDH23 | retina_glia |
| CNMD | retina_glia |
| COL23A1 | retina_glia |
| CTNNA2 | retina_glia |
| DDR1 | retina_glia |
| DIPK1C | retina_glia |
| DKK1 | retina_glia |
| DKK3 | retina_glia |
| EGF | retina_glia |
| EPHB6 | retina_glia |
| F3 | retina_glia |
| FAM171A2 | retina_glia |
| FAP | retina_glia |
| FGF9 | retina_glia |
| FGFRL1 | retina_glia |
| FZD5 | retina_glia |
| FZD8 | retina_glia |
| GFAP | retina_glia |
| GPR37 | retina_glia |
| HES5 | retina_glia |
| MINAR1 | retina_glia |
| NDP | retina_glia |
| NGFR | retina_glia |
| OLFM2 | retina_glia |
| OMG | retina_glia |
| PRSS35 | retina_glia |
| PTH2 | retina_glia |
| RAB3B | retina_glia |
| RCN1 | retina_glia |
| RHPN2 | retina_glia |
| SIX6 | retina_glia |
| SLITRK2 | retina_glia |
| SOX2 | retina_glia |
| TOX3 | retina_glia |
| TRH | retina_glia |
| TYMSOS | retina_glia |
| USH1C | retina_glia |
| WIF1 | retina_glia |
| ADD2 | retina_horizontal |
| APBB2 | retina_horizontal |
| APLP1 | retina_horizontal |
| ASMTL | retina_horizontal |
| BEX5 | retina_horizontal |
| BRF1 | retina_horizontal |
| C1QL1 | retina_horizontal |
| C1QL4 | retina_horizontal |
| CALB1 | retina_horizontal |
| CDC42EP4 | retina_horizontal |
| CMTM4 | retina_horizontal |
| CNTN4 | retina_horizontal |
| CNTNAP2 | retina_horizontal |
| DLK2 | retina_horizontal |
| DTX1 | retina_horizontal |
| EPHA7 | retina_horizontal |
| FCHSD1 | retina_horizontal |
| FLRT1 | retina_horizontal |
| GFRA2 | retina_horizontal |
| HECW1 | retina_horizontal |
| HK1 | retina_horizontal |
| HPCAL1 | retina_horizontal |
| HS3ST3A1 | retina_horizontal |
| LINGO1 | retina_horizontal |
| NAALAD2 | retina_horizontal |
| NRIP3 | retina_horizontal |
| NRSN1 | retina_horizontal |
| NT5C3B | retina_horizontal |
| NTRK1 | retina_horizontal |
| PAK5 | retina_horizontal |
| PRAME | retina_horizontal |
| PRKCZ | retina_horizontal |
| RELL2 | retina_horizontal |
| RETREG1 | retina_horizontal |
| SEMA5A | retina_horizontal |
| SEZ6L | retina_horizontal |
| SH3GL3 | retina_horizontal |
| SMIM13 | retina_horizontal |
| ST8SIA3 | retina_horizontal |
| STMN4 | retina_horizontal |
| TENM2 | retina_horizontal |
| TMOD1 | retina_horizontal |
| TNR | retina_horizontal |
| VAT1L | retina_horizontal |
| AIPL1 | retina_rods |
| C17orf67 | retina_rods |
| CASQ1 | retina_rods |
| CDH12 | retina_rods |
| CDHR1 | retina_rods |
| CEP112 | retina_rods |
| CLUL1 | retina_rods |
| CNGB1 | retina_rods |
| DDC | retina_rods |
| DSCAML1 | retina_rods |
| FABP12 | retina_rods |
| FAIM | retina_rods |
| FUT3 | retina_rods |
| GDF10 | retina_rods |
| GSKIP | retina_rods |
| HCN1 | retina_rods |
| HYPK | retina_rods |
| IGSF11 | retina_rods |
| LRIT2 | retina_rods |
| MTFR1 | retina_rods |
| MYL5 | retina_rods |
| NEURL1 | retina_rods |
| NTM | retina_rods |
| PIH1D2 | retina_rods |
| PLEKHB1 | retina_rods |
| PPP3R1 | retina_rods |
| PRL | retina_rods |
| RCVRN | retina_rods |
| RDH12 | retina_rods |
| RS1 | retina_rods |
| SAG | retina_rods |
| SEMA7A | retina_rods |
| SPEF1 | retina_rods |
| SPINK4 | retina_rods |
| TMEM237 | retina_rods |
| ZPBP | retina_rods |
| ABR | RPE_cells |
| ALDH1A3 | RPE_cells |
| BMP2 | RPE_cells |
| BMP4 | RPE_cells |
| BMP7 | RPE_cells |
| C1orf50 | RPE_cells |
| CALCB | RPE_cells |
| CNDP1 | RPE_cells |
| DNAJB11 | RPE_cells |
| DUSP4 | RPE_cells |
| ENPP2 | RPE_cells |
| ERMN | RPE_cells |
| FAM221A | RPE_cells |
| FGF18 | RPE_cells |
| FHIT | RPE_cells |
| GALNT11 | RPE_cells |
| GAP43 | RPE_cells |
| GDF11 | RPE_cells |
| GEM | RPE_cells |
| GRAMD1C | RPE_cells |
| HBZ | RPE_cells |
| HOMER3 | RPE_cells |
| HSD17B7 | RPE_cells |
| HYOU1 | RPE_cells |
| ITGAV | RPE_cells |
| KCTD4 | RPE_cells |
| KLK11 | RPE_cells |
| LRIT3 | RPE_cells |
| LSMEM1 | RPE_cells |
| MFAP3L | RPE_cells |
| MYRF | RPE_cells |
| NETO2 | RPE_cells |
| NOG | RPE_cells |
| NRN1L | RPE_cells |
| OPCML | RPE_cells |
| OTX2 | RPE_cells |
| PITPNA | RPE_cells |
| PLA2G5 | RPE_cells |
| PLD5 | RPE_cells |
| RBM46 | RPE_cells |
| RBP1 | RPE_cells |
| RDH10 | RPE_cells |
| RIPPLY3 | RPE_cells |
| RLBP1 | RPE_cells |
| RNASE1 | RPE_cells |
| SCGB3A1 | RPE_cells |
| SFRP5 | RPE_cells |
| SLC5A5 | RPE_cells |
| SLC16A3 | RPE_cells |
| SLC26A7 | RPE_cells |
| SOX9 | RPE_cells |
| ST6GALNAC2 | RPE_cells |
| VASN | RPE_cells |
| VILL | RPE_cells |
| WFDC1 | RPE_cells |
| AHCYL1 | schwalbes_line_cells |
| ALCAM | schwalbes_line_cells |
| ALDH7A1 | schwalbes_line_cells |
| AMIGO2 | schwalbes_line_cells |
| CA3 | schwalbes_line_cells |
| CA12 | schwalbes_line_cells |
| CCK | schwalbes_line_cells |
| CDON | schwalbes_line_cells |
| COL8A1 | schwalbes_line_cells |
| COX7A1 | schwalbes_line_cells |
| CUL4B | schwalbes_line_cells |
| CXADR | schwalbes_line_cells |
| DCC | schwalbes_line_cells |
| ENO1 | schwalbes_line_cells |
| FGF7 | schwalbes_line_cells |
| FGF10 | schwalbes_line_cells |
| GRB7 | schwalbes_line_cells |
| HMOX1 | schwalbes_line_cells |
| IER3 | schwalbes_line_cells |
| KISS1 | schwalbes_line_cells |
| LCNL1 | schwalbes_line_cells |
| LRIG1 | schwalbes_line_cells |
| NECAB1 | schwalbes_line_cells |
| NLGN4Y | schwalbes_line_cells |
| NRG1 | schwalbes_line_cells |
| NTN1 | schwalbes_line_cells |
| PAPPA | schwalbes_line_cells |
| PGR | schwalbes_line_cells |
| PRKAR1A | schwalbes_line_cells |
| SFRP1 | schwalbes_line_cells |
| SHC4 | schwalbes_line_cells |
| SYTL4 | schwalbes_line_cells |
| TAC1 | schwalbes_line_cells |
| ARHGEF10 | schwann_cells |
| ART3 | schwann_cells |
| ASPA | schwann_cells |
| CHL1 | schwann_cells |
| CNP | schwann_cells |
| CNTN2 | schwann_cells |
| COL28A1 | schwann_cells |
| DAG1 | schwann_cells |
| EHBP1 | schwann_cells |
| ERBB3 | schwann_cells |
| FOXO1 | schwann_cells |
| GDNF | schwann_cells |
| GFRA3 | schwann_cells |
| IL17B | schwann_cells |
| IRF9 | schwann_cells |
| KLK6 | schwann_cells |
| LRRTM1 | schwann_cells |
| MAG | schwann_cells |
| MIA | schwann_cells |
| MICALL2 | schwann_cells |
| MPZ | schwann_cells |
| NCAM2 | schwann_cells |
| NCMAP | schwann_cells |
| NEGR1 | schwann_cells |
| NPTX2 | schwann_cells |
| NRXN1 | schwann_cells |
| PMEPA1 | schwann_cells |
| RGMB | schwann_cells |
| SAMHD1 | schwann_cells |
| SEMA3B | schwann_cells |
| SORCS1 | schwann_cells |
| TIMP4 | schwann_cells |
| TMPRSS5 | schwann_cells |
| ACVRL1 | endothelial_cells |
| ADGRF5 | endothelial_cells |
| AFAP1L1 | endothelial_cells |
| ANGPT2 | endothelial_cells |
| BTNL9 | endothelial_cells |
| CCL14 | endothelial_cells |
| CCL21 | endothelial_cells |
| CD200 | endothelial_cells |
| CDH5 | endothelial_cells |
| DLL4 | endothelial_cells |
| ELK3 | endothelial_cells |
| ESAM | endothelial_cells |
| F8 | endothelial_cells |
| FLT1 | endothelial_cells |
| FLT4 | endothelial_cells |
| GRAP | endothelial_cells |
| IL3RA | endothelial_cells |
| ITM2A | endothelial_cells |
| JAM2 | endothelial_cells |
| LDB2 | endothelial_cells |
| LRRC32 | endothelial_cells |
| MEOX1 | endothelial_cells |
| MYCT1 | endothelial_cells |
| NOS3 | endothelial_cells |
| PALMD | endothelial_cells |
| PLVAP | endothelial_cells |
| POSTN | endothelial_cells |
| PRCP | endothelial_cells |
| RAMP3 | endothelial_cells |
| RAPGEF5 | endothelial_cells |
| RND1 | endothelial_cells |
| ROBO4 | endothelial_cells |
| SELE | endothelial_cells |
| TEK | endothelial_cells |
| TGFBR2 | endothelial_cells |
| THSD1 | endothelial_cells |
| TIE1 | endothelial_cells |
| VEGFC | endothelial_cells |
| VWF | endothelial_cells |

TABLE 2
Demographic data and associated mutations of patients with retinitis pigmentosa.

| | | Mutated | Affected | | Mutation | |
|---|---|---|---|---|---|---|
| Sex | Age | gene | alleles | Mutation | pathogenic? | Remarks |
| Female | 61 | CRB1/ | heterozygous | c. 2843G > A, | Pathogenic/ | — |
| | | CRB1 | | p. (Cys948Tyr)/ | Pathogenic | |
| | | | | c. 3997G > A, | | |
| | | | | p. (Glu1333Lys) | | |
| Male | 76 | USH2A/ | heterozygous | c2276G > T | Pathogenic/ | — |
| | | USH2A | | (pCys759Phe)/ | Pathogenic | |
| | | | | USH2A c1841 − 2A > G | | |
| Male | 63 | FAM161A | homozygous | c. 1464G > A, | Pathogenic | — |
| | | | | p. (Trp488*) | | |
| Female | 59 | USH2A/ | heterozygous | c. 2167 + 5G > A/ | Pathogenic/ | Samples from |
| | | USH2A | | c. (1328 + 1_1329 − 1)_{—} | Likely | both eyes |
| | | | | (1644 + 1_1645 − 1)del | Pathogenic | |
| Female | 36 | Not | Not | Not | Not | Phenotypically |
| | | tested | tested | tested | tested | retinitis |
| | | | | | | pigmentosa |
| Female | 60 | USH2A/ | heterozygous | c. 2299del, | Pathogenic/ | — |
| | | USH2A | | p. (Glu767Serfs*21)/ | Likely | |
| | | | | c. 14288G > A, | Pathogenic | |
| | | | | p. (Gly4763Glu) | | |

TABLE 3
Pathway analysis of enriched liver proteins in diabetic retinopathy.

| | | | Term | Query | Intersection | |
|---|---|---|---|---|---|---|
| Pathway | ID | Adjusted p | size | size | size | Proteins |
| acute-phase | GO: 0006953 | 1.96127E−09 | 39 | 29 | 9 | APCS, CRP, FN1, HAMP, ITIH4, |
| response | | | | | | LBP, MBL2, SAA1, SERPINA1 |
| acute | GO: 0002526 | 1.51118E−06 | 78 | 29 | 9 | APCS, CRP, FN1, HAMP, ITIH4, |
| inflammatory | | | | | | LBP, MBL2, SAA1, SERPINA1 |
| response | | | | | | |
| regulation of | GO: 0050878 | 1.68566E−05 | 195 | 29 | 11 | F7, F13B, FGA, FGG, FN1, HABP2, |
| body fluid | | | | | | OXT, SAA1, SERPINA1, SERPINA10, |
| levels | | | | | | VTN |
| blood | GO: 0007596 | 1.92364E−05 | 146 | 29 | 10 | F7, F13B, FGA, FGG, FN1, HABP2, |
| coagulation | | | | | | SAA1, SERPINA1, SERPINA10, VTN |
| wound healing | GO: 0042060 | 0.000580169 | 272 | 29 | 11 | APCS, F7, F13B, FGA, FGG, FN1, |
| | | | | | | HABP2, SAA1, SERPINA1, |
| | | | | | | SERPINA10, VTN |
| response to | GO: 0006950 | 0.002162239 | 1900 | 29 | 23 | ANGPTL4, APCS, APOA1, C1RL, C2, |
| stress | | | | | | C4BPA, CFHR5, CRP, F7, F13B, |
| | | | | | | FGA, FGG, FN1, HABP2, HAMP, |
| | | | | | | ITIH4, LBP, MBL2, OXT, SAA1, |
| | | | | | | SERPINA1, SERPINA10, VTN |
| response to | GO: 0009611 | 0.007442452 | 348 | 29 | 11 | APCS, F7, F13B, FGA, FGG, FN1, |
| wounding | | | | | | HABP2, SAA1, SERPINA1, |
| | | | | | | SERPINA10, VTN |
| negative | GO: 0051346 | 0.01438768 | 218 | 29 | 9 | ANGPTL4, APCS, APOA1, ITIH1, |
| regulation of | | | | | | ITIH3, ITIH4, SERPINA1, |
| hydrolase | | | | | | SERPINA10, VTN |
| activity | | | | | | |
| opsonization | GO: 0008228 | 0.016445111 | 14 | 29 | 4 | C4BPA, CRP, LBP, MBL2 |
| blood | GO: 0072378 | 0.016445111 | 14 | 29 | 4 | F13B, FGA, FGG, FN1 |
| coagulation, | | | | | | |
| fibrin clot | | | | | | |
| formation | | | | | | |
| protein | GO: 0072376 | 0.02971365 | 16 | 29 | 4 | F13B, FGA, FGG, FN1 |
| activation | | | | | | |
| cascade | | | | | | |

TABLE 4
Overview of known aging associations of the identified aging proteins.

| | | Associated | | |
|---|---|---|---|---|
| Protein | Function | diseases | Direct aging association | Mendelian diseases |
| LECT2 | Chemotactic factor to | Rheumatoid arhritis, | Decrease in survival rate of LECT2 | — |
| | neurophils and stimulates | Amyloidosis, | knockout mice (PMID: 22251704) | |
| | growth of chondrocytes and | Diabetes mellitus | | |
| | osteoblasts (PMID: | | | |
| | 9524238) | | | |
| DCBLD1 | — | Breast cancer, lungs | Higher expression in older breast cancer | — |
| | | (small cell | patients compared to younger (PMID: | |
| | | carcinoma) | 28673354) | |
| SCGN | Calcium-binding protein | — | Associated to differentiation of neuronal | — |
| | involved in KCL-stinulated | | cells (PMID: 21982882). Methylation | |
| | calcium flux and cell | | level is associated with age (PMID: | |
| | proliferation (PMID: | | 28854399) | |
| | 10811645) | | | |
| ACAN | Proteoglycan in articular | Short stature, disc | Associated with cell senescence in the | short stature, early- |
| | cartilage | herniation | nucleus pulposes (PMID: 35286985) | onset osteoarthritis |
| | | | | (MIM165800) |
| AOC2 | Copper amine oxidase | — | Upregulated with senescense in leafs | — |
| | | | (PMID: 11891244) | |
| CAPS | Calcium binding protein | Glioma, CRC | Regulates the G2/M phase transition of | — |
| | | | the cell cycle (genecards) | |
| TFF3 | Expressed in mucosa | Meningioma, | Expression lowered with age in mice | — |
| | | trigeminal nerve | (PMID: 18223102). Downregulated in | |
| | | neoplasm | senescence-accelerated mouse model | |
| | | | (PMID: 20622466) | |
| AMN | Multiligand endocytic | — | Impaired expression in elderly (PMID: | Imerslund-Grasbeck |
| | receptor | | 29159972). Modulates longevity (PMID: | (MIM618882) |
| | | | 34437681) | |
| ABO | Relevant in transfusion | — | Age-associated increase (PMID: | — |
| | medicine | | 26875505, PMID: 25689219) Different | |
| | | | variants found in centenarians (PMID: | |
| | | | 33103040) | |
| NETO1 | Transmembrane protein | Alzheimers (PMID: | Identified in GWAS study of aging | — |
| | | 30448613) | (PMID: 21782286) | |
| CD274 | Immune inhibitory receptor | — | Accumulates in scenescent cells (PMID: | — |
| | ligand | | 36323784) | |
| PPBP | Platelet-derived growth | Erythromelalgia, | — | — |
| | factor | thrombocytosis | | |
| ABL1 | Protooncogene | leukemia | Identified as an aging factor in neural | — |
| | | | stem cells (PMID: 33848469) | |
| HAMP | Maintains iron balance | Anemia (PMID: | Expression increased in cortex of aging | — |
| | | 28110585) | mice (PMID: 35014607). | |
| IGFBP1 | Regulates metabolic and | Diabetes | Expression associated with all-cause | — |
| | vacular homeostasis | | mortality (PMID: PMID: 33080140) and | |
| | | | aging in B-cells (PMID: 34297797) | |
| AAMDC | Involved in positive | — | — | — |
| | regulation of fat cell | | | |
| | differentiation | | | |
| A1BG | Plasma glycoprotein | — | Liver expression varies with age in mice | — |
| | | | (PMID: 16723264) | |
| ADA2 | Regulates adenosine levels | Vasculitis, | Involved in histone acetylation (PMID: | Sneddon syndrome |
| | | autoinflammation, | 32890768). Deficiency causes | (MIM182410); |
| | | early onset of stroke | accelerated T-cell senescence (PMID: | vasculitis, |
| | | (PMID: 24552284). | 34657246) | autoinflammation, |
| | | | | immunodeficiency |
| | | | | (MIM: 615688) |
| MVP | Involved in nucleo- | Convulsions, | Higher levels with age (PMID: | — |
| | cytoplasmic transport | leukemia | 19472297). Involved in resistense to | |
| | | | apoptosis in senescent cells (PMID: | |
| | | | 18600231). Major signalling pathway in | |
| | | | the Senescence-associated secretory | |
| | | | phenotype (PMID: 27288264) | |
| CRYGC | Maintains transparency and | Cataract, congenital | Other crystallin structures change with | cataract 2 |
| | refractive index of the lens | cataract (PMID: | aging (PMID: 28234481, PMID: | |
| | | 36246175). | 21552534). | |
| A4GALT | Blood antigen in golgi | Osteoporosis (PMID: | — | polyagglutination |
| | membrane | 35748775), cataract | | syndrom |
| | | (PMID: 34954695) | | (MIM111400) |
| A4GNT | Paralog to A4GALT | Gastric ulcer | — | — |
| AADAT | L-lysine metabolism | — | — | — |
| A2M | Protease inhibitor and | Mastitis, Alzheimers | Increases with age in rat kidney (PMID: | — |
| | cytokine transporter | (PMID: 29154276). | 29464020). Hablotype depletion in | |
| | | | individuals over age 95 (PMID: | |
| | | | 19639019) | |
| ABI3 | Regulates actin | Alzheimer's (PMID: | Induces senescence in carcinoma cells | — |
| | polymerization | 35275543) and | in vitro (PMID: 21223585) | |
| | | Parkinson's (PMID: | | |
| | | 33092647). | | |

TABLE 5
Demographic data of all included patients.

| | | | Samples | | | | |
|---|---|---|---|---|---|---|---|
| Sample | | | per | Use of sample | | | |
| number | Group | Cohort | group | for | Sample type | Age | Sex |
| 1 | Control | First cohort | 12 | Identify cell | Aqueous humor | 49 | Female |
| 2 | Control | First cohort | | type specific | Aqueous humor | 74 | Male |
| 3 | Control | First cohort | | marker | Aqueous humor | 84 | Male |
| 4 | Control | First cohort | | proteins & part | Aqueous humor | 71 | Female |
| 5 | Control | First cohort | | of training | Aqueous humor | 59 | Male |
| 6 | Control | First cohort | | cohort of the | Aqueous humor | 50 | Male |
| 7 | Control | First cohort | | machine | Aqueous humor | 77 | Female |
| 8 | Control | First cohort | | learning aging | Aqueous humor | 73 | Female |
| 9 | Control | First cohort | | model | Aqueous humor | 80 | Female |
| 10 | Control | First cohort | | | Aqueous humor | 83 | Female |
| 11 | Control | First cohort | | | Aqueous humor | 75 | Female |
| 12 | Control | First cohort | | | Aqueous humor | 72 | Male |
| 13 | Control | First cohort | 12 | Identify cell | Vitreous | 66 | Female |
| 14 | Control | First cohort | | type specific | Vitreous | 73 | Male |
| 15 | Control | First cohort | | marker | Vitreous | 71 | Female |
| 16 | Control | First cohort | | proteins | Vitreous | 61 | Female |
| 17 | Control | First cohort | | | Vitreous | 68 | Female |
| 18 | Control | First cohort | | | Vitreous | 69 | Female |
| 19 | Control | First cohort | | | Vitreous | 66 | Female |
| 20 | Control | First cohort | | | Vitreous | 71 | Male |
| 21 | Control | First cohort | | | Vitreous | 73 | Male |
| 22 | Control | First cohort | | | Vitreous | 62 | Female |
| 23 | Control | First cohort | | | Vitreous | 80 | Female |
| 24 | Control | First cohort | | | Vitreous | 70 | Female |
| 25 | Control | Second cohort | 34 | part of training | Aqueous humor | 72 | Female |
| 26 | Control | Second cohort | | cohort of the | Aqueous humor | 74 | Female |
| 27 | Control | Second cohort | | machine | Aqueous humor | 66 | Female |
| 28 | Control | Second cohort | | learning aging | Aqueous humor | 65 | Female |
| 29 | Control | Second cohort | | model | Aqueous humor | 58 | Male |
| 30 | Control | Second cohort | | | Aqueous humor | 74 | Male |
| 31 | Control | Second cohort | | | Aqueous humor | 88 | Male |
| 32 | Control | Second cohort | | | Aqueous humor | 51 | Female |
| 33 | Control | Second cohort | | | Aqueous humor | 75 | Female |
| 34 | Control | Second cohort | | | Aqueous humor | 69 | Female |
| 35 | Control | Second cohort | | | Aqueous humor | 83 | Male |
| 36 | Control | Second cohort | | | Aqueous humor | 59 | Female |
| 37 | Control | Second cohort | | | Aqueous humor | 74 | Male |
| 38 | Control | Second cohort | | | Aqueous humor | 66 | Female |
| 39 | Control | Second cohort | | | Aqueous humor | 66 | Female |
| 40 | Control | Second cohort | | | Aqueous humor | 71 | Female |
| 41 | Control | Second cohort | | | Aqueous humor | 80 | Male |
| 42 | Control | Second cohort | | | Aqueous humor | 76 | Female |
| 43 | Control | Second cohort | | | Aqueous humor | 84 | Female |
| 44 | Control | Second cohort | | | Aqueous humor | 65 | Male |
| 45 | Control | Second cohort | | | Aqueous humor | 66 | Male |
| 46 | Control | Second cohort | | | Aqueous humor | 84 | Male |
| 47 | Control | Second cohort | | | Aqueous humor | 75 | Male |
| 48 | Control | Second cohort | | | Aqueous humor | 70 | Male |
| 49 | Control | Second cohort | | | Aqueous humor | 71 | Female |
| 50 | Control | Second cohort | | | Aqueous humor | 71 | Female |
| 51 | Control | Second cohort | | | Aqueous humor | 69 | Male |
| 52 | Control | Second cohort | | | Aqueous humor | 66 | Male |
| 53 | Control | Second cohort | | | Aqueous humor | 60 | Female |
| 54 | Control | Second cohort | | | Aqueous humor | 56 | Female |
| 55 | Control | Second cohort | | | Aqueous humor | 57 | Male |
| 56 | Control | Second cohort | | | Aqueous humor | 90 | Female |
| 57 | Control | Second cohort | | | Aqueous humor | 79 | Female |
| 58 | Control | Second cohort | | | Aqueous humor | 68 | Female |
| 59 | Control | Third cohort | 19 | validation | Aqueous humor | 64 | Female |
| 60 | Control | Third cohort | | cohort of the | Aqueous humor | 57 | Male |
| 61 | Control | Third cohort | | machine | Aqueous humor | 69 | Male |
| 62 | Control | Third cohort | | learning aging | Aqueous humor | 76 | Female |
| 63 | Control | Third cohort | | model | Aqueous humor | 67 | Male |
| 64 | Control | Third cohort | | | Aqueous humor | 72 | Male |
| 65 | Control | Third cohort | | | Aqueous humor | 90 | Male |
| 66 | Control | Third cohort | | | Aqueous humor | 58 | Female |
| 67 | Control | Third cohort | | | Aqueous humor | 72 | Female |
| 68 | Control | Third cohort | | | Aqueous humor | 85 | Female |
| 69 | Control | Third cohort | | | Aqueous humor | 75 | Female |
| 70 | Control | Third cohort | | | Aqueous humor | 72 | Female |
| 71 | Control | Third cohort | | | Aqueous humor | 63 | Female |
| 72 | Control | Third cohort | | | Aqueous humor | 59 | Female |
| 73 | Control | Third cohort | | | Aqueous humor | 70 | Female |
| 74 | Control | Third cohort | | | Aqueous humor | 63 | Male |
| 75 | Control | Third cohort | | | Aqueous humor | 85 | Male |
| 76 | Control | Third cohort | | | Aqueous humor | 75 | Female |
| 77 | Control | Third cohort | | | Aqueous humor | 79 | Female |
| 78 | NPDR | Third cohort | 15 | Diseased | Aqueous humor | 63 | Male |
| 79 | NPDR | Third cohort | | samples, | Aqueous humor | 69 | Male |
| 80 | NPDR | Third cohort | | validation of | Aqueous humor | 71 | Female |
| 81 | NPDR | Third cohort | | cell type | Aqueous humor | 76 | Male |
| 82 | NPDR | Third cohort | | specific marker | Aqueous humor | 58 | Male |
| 83 | PDR | Third cohort | | proteins and | Aqueous humor | 26 | Male |
| 84 | PDR | Third cohort | | validation of | Aqueous humor | 52 | Male |
| 85 | PDR | Third cohort | | age model | Aqueous humor | 44 | Male |
| 86 | PDR | Third cohort | | | Aqueous humor | 49 | Female |
| 87 | PDR | Third cohort | | | Aqueous humor | 51 | Female |
| 88 | PDR | Third cohort | | | Aqueous humor | 44 | Male |
| 89 | PDR | Third cohort | | | Aqueous humor | 47 | Female |
| 90 | PDR | Third cohort | | | Aqueous humor | 59 | Female |
| 91 | PDR | Third cohort | | | Aqueous humor | 53 | Male |
| 92 | PDR | Third cohort | | | Aqueous humor | 71 | Male |
| 93 | DM wo DR | Third cohort | 10 | | Aqueous humor | 67 | Female |
| 94 | DM wo DR | Third cohort | | | Aqueous humor | 85 | Female |
| 95 | DM wo DR | Third cohort | | | Aqueous humor | 51 | Male |
| 96 | DM wo DR | Third cohort | | | Aqueous humor | 64 | Female |
| 97 | DM wo DR | Third cohort | | | Aqueous humor | 66 | Female |
| 98 | DM wo DR | Third cohort | | | Aqueous humor | 68 | Female |
| 99 | DM wo DR | Third cohort | | | Aqueous humor | 55 | Female |
| 100 | DM wo DR | Third cohort | | | Aqueous humor | 64 | Female |
| 101 | DM wo DR | Third cohort | | | Aqueous humor | 61 | Female |
| 102 | DM wo DR | Third cohort | | | Aqueous humor | 60 | Male |
| 103 | Uveitis | Third cohort | 6 | | Aqueous humor | 66 | Male |
| 104 | Uveitis | Third cohort | | | Aqueous humor | 57 | Female |
| 105 | Uveitis | Third cohort | | | Aqueous humor | 54 | Female |
| 106 | Uveitis | Third cohort | | | Aqueous humor | 22 | Male |
| 107 | Uveitis | Third cohort | | | Aqueous humor | 40 | Female |
| 108 | Uveitis | Third cohort | | | Aqueous humor | 60 | Female |
| 109 | Retinitis | Third cohort | 7 | | Aqueous humor | 61 | Female |
| | pigmentosa | | | | | | |
| 110 | Retinitis | Third cohort | | | Aqueous humor | 76 | Male |
| | pigmentosa | | | | | | |
| 111 | Retinitis | Third cohort | | | Aqueous humor | 63 | Male |
| | pigmentosa | | | | | | |
| 112 | Retinitis | Third cohort | | | Aqueous humor | 59 | Female |
| | pigmentosa | | | | | | |
| 113 | Retinitis | Third cohort | | | Aqueous humor | 36 | Female |
| | pigmentosa | | | | | | |
| 114 | Retinitis | Third cohort | | | Aqueous humor | 59 | Female |
| | pigmentosa | | | | | | |
| 115 | Retinitis | Third cohort | | | Aqueous humor | 60 | Female |
| | pigmentosa | | | | | | |
| 116 | Parkinson's | Third cohort | 5 | | Aqueous humor | 82 | Male |
| | disease | | | | | | |
| 117 | Parkinson's | Third cohort | | | Aqueous humor | 51 | Male |
| | disease | | | | | | |
| 118 | Parkinson's | Third cohort | | | Aqueous humor | 86 | Female |
| | disease | | | | | | |
| 119 | Parkinson's | Third cohort | | | Aqueous humor | 71 | Female |
| | disease | | | | | | |
| 120 | Parkinson's | Third cohort | | | Aqueous humor | 88 | Male |
| | disease | | | | | | |

NPDR: non-proliferative diabetic retinopathy, PDR: proliferative diabetic retinopathy, DM wo DR: diabetes mellitus without diabetic retinopathy.

TABLE 6
Abbreviations of protein names (HGNC nomenclature).

| Abbreviation | Protein name |
|---|---|
| A1BG | Alpha-1B-glycoprotein |
| A2M | Alpha-2-Macroglobulin |
| ABI3 | ABI Family Member 3 |
| ABO | ABO, Alpha 1-3-N-Acetylgalactosaminyltransferase And Alpha 1-3-Galactosyltransferase |
| ACAN | Aggrecan |
| ADA2 | Adenosine Deaminase 2 |
| AMN | Amnionless |
| ANGPT2 | Angiopoietin-2 |
| ANGPTL-4 | Angiopoietin-related protein 4 |
| ANGPTL4 | Angiopoietin-related protein 4 |
| APOA1 | Apolipoprotein A-I |
| CCL2 | C-C motif chemokine 2 |
| CD274 | CD274 Molecule |
| CDHR1 | Cadherin-related family member 1 |
| CDHR1 | Cadherin Related Family Member 1 |
| CRP | C-reactive protein |
| CXCL8 | Interleukin 8 |
| DCBLD | Discoidin, CUB And LCCL Domain Containing 2 |
| ENG | Endoglin |
| F13B | Coagulation factor XIII B chain |
| FLT4 | Vascular endothelial growth factor receptor 3 |
| GBA | Glucosylceramidase Beta |
| HAMP | Hepcidin Antimicrobial Peptide |
| HTRA2 | High Temperature Requirement Protein A2 |
| IGF | Insulin-like growth factor |
| IGFBP1 | Insulin-like growth factor-binding protein 1 |
| INPP5F | Phosphatidylinositide phosphatase SAC2 |
| LECT2 | Leukocyte cell-derived chemotaxin-2 |
| LRRK2 | Leukcine-rich repeat kinase 2 |
| MMP19 | Matrix metalloproteinase-19 |
| MVP | Major Vault Protein |
| NSF | Vesicle-fusing ATPase |
| SAG | S-arrestin |
| SCGN | Secretagogin |
| SERPINA10 | Protein Z-dependent protease inhibitor |
| SYT4 | Synaptotagmin-4 |
| TFF3 | Trefoil Factor 3 |
| THBS1 | Thrombospondin-1 |
| TYMP | Thymidine phosphorylase |
| VEGFA | Vascular endothelial growth factor A |
| VEGFC | Vascular endothelial growth factor C |
| VEGFD | Vascular endothelial growth factor D |
| WARS1 | Tryptophan-tRNA ligase, cytoplasmic). |

### REFERENCES

1. Dowden, H., and Munro, J. (2019). Trends in clinical success rates and therapeutic focus. Nat Rev Drug Discov 18, 495-496. 10.1038/d41573-019-00074-z.
2. Seok, J., Warren, H. S., Cuenca, A. G., Mindrinos, M. N., Baker, H. V., Xu, W., Richards, D. R., McDonald-Smith, G. P., Gao, H., Hennessy, L., et al. (2013). Genomic responses in mouse models poorly mimic human inflammatory diseases. Proc Natl Acad Sci USA 110, 3507-3512. 10.1073/pnas.1222878110.
3. Schwab, I. R. (2018). The evolution of eyes: major steps. The Keeler lecture 2017: centenary of Keeler Ltd. Eye (Lond) 32, 302-313. 10.1038/eye.2017.226.
4. Zheng, Y., Ley, S. H., and Hu, F. B. (2018). Global aetiology and epidemiology of type 2 diabetes mellitus and its complications. Nat Rev Endocrinol 14, 88-98. 10.1038/nrendo.2017.151.
5. Ascherio, A., and Schwarzschild, M. A. (2016). The epidemiology of Parkinson's disease: risk factors and prevention. Lancet Neurol 15, 1257-1272. 10.1016/S1474-4422(16)30230-7.
6. Lee, J. Y., Martin-Bastida, A., Murueta-Goyena, A., Gabilondo, I., Cuenca, N., Piccini, P., and Jeon, B. (2022). Multimodal brain and retinal imaging of dopaminergic degeneration in Parkinson disease. Nat Rev Neurol 18, 203-220. 10.1038/s41582-022-00618-9.
7. Collin, J., Queen, R., Zerti, D., Bojic, S., Dorgau, B., Moyse, N., Molina, M. M., Yang, C., Dey, S., Reynolds, G., et al. (2021). A single cell atlas of human cornea that defines its development, limbal progenitor cells and their interactions with the immune cells. Ocul Surf 21, 279-298. 10.1016/j.jtos.2021.03.010.
8. van Zyl, T., Yan, W., McAdams, A., Peng, Y. R., Shekhar, K., Regev, A., Juric, D., and Sanes, J. R. (2020). Cell atlas of aqueous humor outflow pathways in eyes of humans and four model species provides insight into glaucoma pathogenesis. Proc Natl Acad Sci USA 117, 10339-10349. 10.1073/pnas.2001250117.
9. Gautam, P., Hamashima, K., Chen, Y., Zeng, Y., Makovoz, B., Parikh, B. H., Lee, H. Y., Lau, K. A., Su, X., Wong, R. C. B., et al. (2021). Multi-species single-cell transcriptomic analysis of ocular compartment regulons. Nat Commun 12, 5675. 10.1038/s41467-021-25968-8.
10. Wang, Z., Su, D., Liu, S., Zheng, G., Zhang, G., Cui, T., Ma, X., Sun, Z., and Hu, S. (2021). RNA sequencing and bioinformatics analysis of human lens epithelial cells in age-related cataract. BMC Ophthalmol 21, 152. 10.1186/s12886-021-01915-5.
11. Wolf, J., Boneva, S., Rosmus, D. D., Agostini, H., Schlunck, G., Wieghofer, P., Schlecht, A., and Lange, C. (2022). Deciphering the Molecular Signature of Human Hyalocytes in Relation to Other Innate Immune Cell Populations. Invest Ophthalmol Vis Sci 63, 9. 10.1167/iovs.63.3.9.
12. Boneva, S. K., Wolf, J., Rosmus, D. D., Schlecht, A., Prinz, G., Laich, Y., Boeck, M., Zhang, P., Hilgendorf, I., Stahl, A., et al. (2020). Transcriptional Profiling Uncovers Human Hyalocytes as a Unique Innate Immune Cell Population. Front Immunol 11, 567274. 10.3389/fimmu.2020.567274.
13. Voigt, A. P., Whitmore, S. S., Flamme-Wiese, M. J., Riker, M. J., Wiley, L. A., Tucker, B. A., Stone, E. M., Mullins, R. F., and Scheetz, T. E. (2019). Molecular characterization of foveal versus peripheral human retina by single-cell RNA sequencing. Exp Eye Res 184, 234-242. 10.1016/j.exer.2019.05.001.
14. Voigt, A. P., Mulfaul, K., Mullin, N. K., Flamme-Wiese, M. J., Giacalone, J. C., Stone, E. M., Tucker, B. A., Scheetz, T. E., and Mullins, R. F. (2019). Single-cell transcriptomics of the human retinal pigment epithelium and choroid in health and macular degeneration. Proc Natl Acad Sci USA 116, 24100-24107. 10.1073/pnas.1914143116.
15. MacParland, S. A., Liu, J. C., Ma, X. Z., Innes, B. T., Bartczak, A. M., Gage, B. K., Manuel, J., Khuu, N., Echeverri, J., Linares, I., et al. (2018). Single cell RNA sequencing of human liver reveals distinct intrahepatic macrophage populations. Nat Commun 9, 4383. 10.1038/s41467-018-06318-7.
16. He, S., Wang, L. H., Liu, Y., Li, Y. Q., Chen, H. T., Xu, J. H., Peng, W., Lin, G. W., Wei, P. P., Li, B., et al. (2020). Single-cell transcriptome profiling of an adult human cell atlas of 15 major organs. Genome Biol 21, 294. 10.1186/s13059-020-02210-0.
17. Xie, X., Liu, M., Zhang, Y., Wang, B., Zhu, C., Wang, C., Li, Q., Huo, Y., Guo, J., Xu, C., et al. (2021). Single-cell transcriptomic landscape of human blood cells. Natl Sci Rev 8, nwaa180. 10.1093/nsr/nwaa180.
18. Gold, L., Ayers, D., Bertino, J., Bock, C., Bock, A., Brody, E. N., Carter, J., Dalby, A. B., Eaton, B. E., Fitzwater, T., et al. (2010). Aptamer-based multiplexed proteomic technology for biomarker discovery. PLoS One 5, e15004. 10.1371/journal.pone.0015004.
19. Chowdhury, U. R., Madden, B. J., Charlesworth, M. C., and Fautsch, M. P. (2010). Proteome analysis of human aqueous humor. Invest Ophthalmol Vis Sci 51, 4921-4931. 10.1167/iovs.10-5531.
20. Poulsen, E. T., Lumi, X., Hansen, A. K., Enghild, J. J., and Petrovski, G. (2020). Protein Composition of the Subretinal Fluid Suggests Selective Diffusion of Vitreous Proteins in Retinal Detachment. Transl Vis Sci Technol 9, 16. 10.1167/tvst.9.11.16.
21. Leeman, M., Choi, J., Hansson, S., Storm, M. U., and Nilsson, L. (2018). Proteins and antibodies in serum, plasma, and whole blood-size characterization using asymmetrical flow field-flow fractionation (AF4). Anal Bioanal Chem 410, 4867-4873. 10.1007/s00216-018-1127-2.
22. Ferrari, S., Di lorio, E., Barbaro, V., Ponzin, D., Sorrentino, F. S., and Parmeggiani, F. (2011). Retinitis pigmentosa: genes and disease mechanisms. Curr Genomics 12, 238-249. 10.2174/138920211795860107.
23. Verbakel, S. K., van Huet, R. A. C., Boon, C. J. F., den Hollander, A. I., Collin, R. W. J., Klaver, C. C. W., Hoyng, C. B., Roepman, R., and Klevering, B. J. (2018). Non-syndromic retinitis pigmentosa. Prog Retin Eye Res 66, 157-186. 10.1016/j.preteyeres.2018.03.005.
24. Burkholder, B. M., and Jabs, D. A. (2021). Uveitis for the non-ophthalmologist. BMJ 372, m4979. 10.1136/bmj.m4979.
25. Lee, R., Wong, T. Y., and Sabanayagam, C. (2015). Epidemiology of diabetic retinopathy, diabetic macular edema and related vision loss. Eye Vis (Lond) 2, 17. 10.1186/s40662-015-0026-2.
26. Sun, L., Wang, R., Hu, G., Liu, H., Lv, K., Duan, Y., Shen, N., Wu, J., Hu, J., Liu, Y., et al. (2021). Single cell RNA sequencing (scRNA-Seq) deciphering pathological alterations in streptozotocin-induced diabetic retinas. Exp Eye Res 210, 108718. 10.1016/j.exer.2021.108718.
27. Pitale, P. M., and Gorbatyuk, M. S. (2022). Diabetic Retinopathy: From Animal Models to Cellular Signaling. Int J Mol Sci 23. 10.3390/ijms23031487.
28. Foulger, R. E., Denny, P., Hardy, J., Martin, M. J., Sawford, T., and Lovering, R. C. (2016). Using the Gene Ontology to Annotate Key Players in Parkinson's Disease. Neuroinformatics 14, 297-304. 10.1007/s12021-015-9293-2.
29. Harman, D. (1991). The aging process: major risk factor for disease and death. Proc Natl Acad Sci USA 88, 5360-5363. 10.1073/pnas.88.12.5360.
30. Di Micco, R., Krizhanovsky, V., Baker, D., and d'Adda di Fagagna, F. (2021). Cellular senescence in ageing: from mechanisms to therapeutic opportunities. Nat Rev Mol Cell Biol 22, 75-95. 10.1038/s41580-020-00314-w.
31. Yang, J. H., Hayano, M., Griffin, P. T., Amorim, J. A., Bonkowski, M. S., Apostolides, J. K., Salfati, E. L., Blanchette, M., Munding, E. M., Bhakta, M., et al. (2023). Loss of epigenetic information as a cause of mammalian aging. Cell. 10.1016/j.cell.2022.12.027.
32. Lehallier, B., Gate, D., Schaum, N., Nanasi, T., Lee, S. E., Yousef, H., Moran Losada, P., Berdnik, D., Keller, A., Verghese, J., et al. (2019). Undulating changes in human plasma proteome profiles across the lifespan. Nat Med 25, 1843-1850. 10.1038/s41591-019-0673-2.
33. Tanaka, T., Biancotto, A., Moaddel, R., Moore, A. Z., Gonzalez-Freire, M., Aon, M. A., Candia, J., Zhang, P., Cheung, F., Fantoni, G., et al. (2018). Plasma proteomic signature of age in healthy humans. Aging Cell 17, e12799. 10.1111/acel.12799.
34. Yi, W., Lu, Y., Zhong, S., Zhang, M., Sun, L., Dong, H., Wang, M., Wei, M., Xie, H., Qu, H., et al. (2021). A single-cell transcriptome atlas of the aging human and macaque retina. Natl Sci Rev 8, nwaa179. 10.1093/nsr/nwaa179.
35. Voigt, A. P., Whitmore, S. S., Mulfaul, K., Chirco, K. R., Giacalone, J. C., Flamme-Wiese, M. J., Stockman, A., Stone, E. M., Tucker, B. A., Scheetz, T. E., and Mullins, R. F. (2020). Bulk and single-cell gene expression analyses reveal aging human choriocapillaris has pro-inflammatory phenotype. Microvasc Res 131, 104031. 10.1016/j.mvr.2020.104031.
36. Lu, Y., Brommer, B., Tian, X., Krishnan, A., Meer, M., Wang, C., Vera, D. L., Zeng, Q., Yu, D., Bonkowski, M. S., et al. (2020). Reprogramming to recover youthful epigenetic information and restore vision. Nature 588, 124-129. 10.1038/s41586-020-2975-4.
37. Coppe, J. P., Desprez, P. Y., Krtolica, A., and Campisi, J. (2010). The senescence-associated secretory phenotype: the dark side of tumor suppression. Annu Rev Pathol 5, 99-118. 10.1146/annurev-pathol-121808-102144.
38. Ubaida-Mohien, C., Lyashkov, A., Gonzalez-Freire, M., Tharakan, R., Shardell, M., Moaddel, R., Semba, R. D., Chia, C. W., Gorospe, M., Sen, R., and Ferrucci, L. (2019). Discovery proteomics in aging human skeletal muscle finds change in spliceosome, immunity, proteostasis and mitochondria. Elife 8. 10.7554/eLife.49874.
39. D'Amico, A. M., and Vasquez, K. M. (2021). The multifaceted roles of DNA repair and replication proteins in aging and obesity. DNA Repair (Amst) 99, 103049. 10.1016/j.dnarep.2021.103049.
40. Turner, K. J., Vasu, V., and Griffin, D. K. (2019). Telomere Biology and Human Phenotype. Cells 8. 10.3390/cells8010073.
41. Iram, T., Kern, F., Kaur, A., Myneni, S., Morningstar, A. R., Shin, H., Garcia, M. A., Yerra, L., Palovics, R., Yang, A. C., et al. (2022). Young CSF restores oligodendrogenesis and memory in aged mice via Fgf17. Nature 605, 509-515. 10.1038/s41586-022-04722-0.
42. Tiong, J. D., Gosney, E., Ding, J., Chin, E., and Kopchick, J. J. (2006). A liver specific gene that is expressed in growth hormone transgenic mice and in normal female mice as a function of age. Growth Horm IGF Res 16, 145-156. 10.1016/j.ghir.2006.03.011.
43. Ori, A., Toyama, B. H., Harris, M. S., Bock, T., Iskar, M., Bork, P., Ingolia, N. T., Hetzer, M. W., and Beck, M. (2015). Integrated Transcriptome and Proteome Analyses Reveal Organ-Specific Proteome Deterioration in Old Rats. Cell Syst 1, 224-237. 10.1016/j.cels.2015.08.012.
44. Tivey, A., Church, M., Rothwell, D., Dive, C., and Cook, N. (2022). Circulating tumour DNA—looking beyond the blood. Nat Rev Clin Oncol 19, 600-612. 10.1038/s41571-022-00660-y.
45. Poganik, J. R., Zhang, B., Baht, G. S., Tyshkovskiy, A., Deik, A., Kerepesi, C., Yim, S. H., Lu, A. T., Haghani, A., Gong, T., et al. (2023). Biological age is increased by stress and restored upon recovery. Cell Metab. 10.1016/j.cmet.2023.03.015.
46. Galkin, F., Mamoshina, P., Aliper, A., de Magalhaes, J. P., Gladyshev, V. N., and Zhavoronkov, A. (2020). Biohorology and biomarkers of aging: Current state-of-the-art, challenges and opportunities. Ageing Res Rev 60, 101050. 10.1016/j.arr.2020.101050.
47. Anson, M., Crain-Denoyelle, A. M., Baud, V., Chereau, F., Gougelet, A., Terris, B., Yamagoe, S., Colnot, S., Viguier, M., Perret, C., and Couty, J. P. (2012). Oncogenic beta-catenin triggers an inflammatory response that determines the aggressiveness of hepatocellular carcinoma in mice. J Clin Invest 122, 586-599. 10.1172/JC143937.
48. Tseng, C. W., and Liu, G. Y. (2014). Expanding roles of neutrophils in aging hosts. Curr Opin Immunol 29, 43-48. 10.1016/j.coi.2014.03.009.
49. Wallis, Z. K., and Williams, K. C. (2022). Monocytes in HIV and SIV Infection and Aging: Implications for Inflamm-Aging and Accelerated Aging. Viruses 14. 10.3390/v14020409.
50. Jia, G., Aroor, A. R., Jia, C., and Sowers, J. R. (2019). Endothelial cell senescence in aging-related vascular dysfunction. Biochim Biophys Acta Mol Basis Dis 1865, 1802-1809. 10.1016/j.bbadis.2018.08.008.
51. Moaddel, R., Ubaida-Mohien, C., Tanaka, T., Lyashkov, A., Basisty, N., Schilling, B., Semba, R. D., Franceschi, C., Gorospe, M., and Ferrucci, L. (2021). Proteomics in aging research: A roadmap to clinical, translational research. Aging Cell 20, e13325. 10.1111/acel.13325.
52. Ubaida-Mohien, C., Moaddel, R., Moore, A. Z., Kuo, P. L., Faghri, F., Tharakan, R., Tanaka, T., Nalls, M. A., and Ferrucci, L. (2021). Proteomics and Epidemiological Models of Human Aging. Front Physiol 12, 674013. 10.3389/fphys.2021.674013.
53. Song, J., Chen, S., Liu, X., Duan, H., Kong, J., and Li, Z. (2015). Relationship between C-Reactive Protein Level and Diabetic Retinopathy: A Systematic Review and Meta-Analysis. PLoS One 10, e0144406. 10.1371/journal.pone.0144406.
54. Spranger, J., Buhnen, J., Jansen, V., Krieg, M., Meyer-Schwickerath, R., Blum, W. F., Schatz, H., and Pfeiffer, A. F. (2000). Systemic levels contribute significantly to increased intraocular IGF-I, IGF-II and IGF-BP3 [correction of IFG-BP3] in proliferative diabetic retinopathy. Horm Metab Res 32, 196-200. 10.1055/s-2007-978621.
55. Puthussery, T., Gayet-Primo, J., Pandey, S., Duvoisin, R. M., and Taylor, W. R. (2009). Differential loss and preservation of glutamate receptor function in bipolar cells in the rd10 mouse model of retinitis pigmentosa. Eur J Neurosci 29, 1533-1542. 10.1111/j.1460-9568.2009.06728.x.
56. Fariss, R. N., Li, Z. Y., and Milam, A. H. (2000). Abnormalities in rod photoreceptors, amacrine cells, and horizontal cells in human retinas with retinitis pigmentosa. Am J Ophthalmol 129, 215-223. 10.1016/s0002-9394(99)00401-8.
57. Milam, A. H., Li, Z. Y., and Fariss, R. N. (1998). Histopathology of the human retina in retinitis pigmentosa. Prog Retin Eye Res 17, 175-205. 10.1016/s1350-9462(97)00012-8.
58. Mammadova, N., Summers, C. M., Kokemuller, R. D., He, Q., Ding, S., Baron, T., Yu, C., Valentine, R. J., Sakaguchi, D. S., Kanthasamy, A. G., et al. (2019). Accelerated accumulation of retinal alpha-synuclein (pSer129) and tau, neuroinflammation, and autophagic dysregulation in a seeded mouse model of Parkinson's disease. Neurobiol Dis 121, 1-16. 10.1016/j.nbd.2018.09.013.
59. Cervero, A., Sanchez-Rodriguez, A., Rivera-Sanchez, M., Martinez-Rodriguez, I., Sierra, M., Gonzalez-Aramburu, I., Gutierrez-Gonzalez, A., Andres-Pacheco, J., Sanchez-Pelaez, M. V., Casado, A., and Infante, J. (2023). Analysis of retinal nerve layers in idiopathic, LRRK2-associated Parkinson's disease and unaffected carriers of G2019S mutation. Parkinsonism Relat Disord 106, 105246. 10.1016/j.parkreldis.2022.105246.
60. Maturi, R. K., Glassman, A. R., Josic, K., Baker, C. W., Gerstenblith, A. T., Jampol, L. M., Meleth, A., Martin, D. F., Melia, M., Punjabi, O. S., et al. (2023). Four-Year Visual Outcomes in the Protocol W Randomized Trial of Intravitreous Aflibercept for Prevention of Vision-Threatening Complications of Diabetic Retinopathy. JAMA 329, 376-385. 10.1001/jama.2022.25029.
61. Wykoff, C. C., Abreu, F., Adamis, A. P., Basu, K., Eichenbaum, D. A., Haskova, Z., Lin, H., Loewenstein, A., Mohan, S., Pearce, I. A., et al. (2022). Efficacy, durability, and safety of intravitreal faricimab with extended dosing up to every 16 weeks in patients with diabetic macular oedema (YOSEMITE and RHINE): two randomised, double-masked, phase 3 trials. Lancet 399, 741-755. 10.1016/S0140-6736(22)00018-6.
62. Tolosa, E., Vila, M., Klein, C., and Rascol, O. (2020). LRRK2 in Parkinson disease: challenges of clinical trials. Nat Rev Neurol 16, 97-107. 10.1038/s41582-019-0301-2.
63. Paisan-Ruiz, C., Jain, S., Evans, E. W., Gilks, W. P., Simon, J., van der Brug, M., Lopez de Munain, A., Aparicio, S., Gil, A. M., Khan, N., et al. (2004). Cloning of the gene containing mutations that cause PARK8-linked Parkinson's disease. Neuron 44, 595-600. 10.1016/j.neuron.2004.10.023.
64. Afsari, F., Christensen, K. V., Smith, G. P., Hentzer, M., Nippe, O. M., Elliott, C. J., and Wade, A. R. (2014). Abnormal visual gain control in a Parkinson's disease model. Hum Mol Genet 23, 4465-4478. 10.1093/hmg/ddu159.
65. Aharon-Peretz, J., Rosenbaum, H., and Gershoni-Baruch, R. (2004). Mutations in the glucocerebrosidase gene and Parkinson's disease in Ashkenazi Jews. N Engl J Med 351, 1972-1977. 10.1056/NEJMoa033277.
66. Strauss, K. M., Martins, L. M., Plun-Favreau, H., Marx, F. P., Kautzmann, S., Berg, D., Gasser, T., Wszolek, Z., Muller, T., Bornemann, A., et al. (2005). Loss of function mutations in the gene encoding Omi/HtrA2 in Parkinson's disease. Hum Mol Genet 14, 2099-2111. 10.1093/hmg/ddi215.
67. Germain, A., Levine, S. M., and Hanson, M. R. (2021). In-Depth Analysis of the Plasma Proteome in ME/CFS Exposes Disrupted Ephrin-Eph and Immune System Signaling. Proteomes 9. 10.3390/proteomes9010006.
68. Pietzner, M., Wheeler, E., Carrasco-Zanini, J., Kerrison, N. D., Oerton, E., Koprulu, M., Luan, J., Hingorani, A. D., Williams, S. A., Wareham, N. J., and Langenberg, C. (2021). Synergistic insights into human health from aptamer- and antibody-based proteomic profiling. Nat Commun 12, 6822. 10.1038/s41467-021-27164-0.
69. Ritchie, M. E., Phipson, B., Wu, D., Hu, Y., Law, C. W., Shi, W., and Smyth, G. K. (2015). limma powers differential expression analyses for RNA-sequencing and microarray studies. Nucleic Acids Res 43, e47. 10.1093/nar/gkv007.
70. Gu, Z., Eils, R., and Schlesner, M. (2016). Complex heatmaps reveal patterns and correlations in multidimensional genomic data. Bioinformatics 32, 2847-2849. 10.1093/bioinformatics/btw313.
71. Tweedie, S., Braschi, B., Gray, K., Jones, T. E. M., Seal, R. L., Yates, B., and Bruford, E. A. (2021). Genenames.org: the HGNC and VGNC resources in 2021. Nucleic Acids Res 49, D939-D946. 10.1093/nar/gkaa980.
72. Love, M. I., Huber, W., and Anders, S. (2014). Moderated estimation of fold change and dispersion for RNA-seq data with DESeq2. Genome Biol 15, 550. 10.1186/s13059-014-0550-8.
73. UniProt, C. (2021). UniProt: the universal protein knowledgebase in 2021. Nucleic Acids Res 49, D480-D489. 10.1093/nar/gkaa1100.
74. Raudvere, U., Kolberg, L., Kuzmin, I., Arak, T., Adler, P., Peterson, H., and Vilo, J. (2019). g:Profiler: a web server for functional enrichment analysis and conversions of gene lists (2019 update). Nucleic Acids Res 47, W191-W198. 10.1093/nar/gkz369.
75. Guestrin, T. C. a. C. (2016). XGBoost: A Scalable Tree Boosting System. In Proceedings of the 22nd ACM SIGKDD International Conference on Knowledge Discovery and Data Mining (KDD '16). Association for Computing Machinery. New York, NY, USA, 785-794.

### SUPPLEMENTAL REFERENCES

[S1] Germain A, Levine S M, Hanson M R. In-Depth Analysis of the Plasma Proteome in ME/CFS Exposes Disrupted Ephrin-Eph and Immune System Signaling. Proteomes. 2021 Jan. 29; 9(1):6. doi: 10.3390/proteomes9010006. PMID: 33572894; PMCID: PMC7931008.
[S2] Iram T, Kern F, Kaur A, Myneni S, Morningstar A R, Shin H, Garcia M A, Yerra L, Palovics R, Yang A C, Hahn O, Lu N, Shuken S R, Haney M S, Lehallier B, lyer M, Luo J, Zetterberg H, Keller A, Zuchero J B, Wyss-Coray T. Young CSF restores oligodendrogenesis and memory in aged mice via Fgf17. Nature. 2022 May; 605(7910):509-515. doi: 10.1038/s41586-022-04722-0. Epub 2022 May 11. Erratum in: Nature. 2023 January; 613(7942):E1. PMID: 35545674; PMCID: PMC9377328.

What is claimed is:

1. A method of diagnosing and treating retinitis pigmentosa in a patient, the method comprising:
obtaining an aqueous humor sample from an eye of the patient;
measuring levels of at least two, at least three, or at least four biomarkers selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1 L1, OPCML, LRIT3, PLA2G5, GALNT11, and KCTD4 in the aqueous humor sample, wherein decreased levels of the one or more biomarkers selected from RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LRIT3, PLA2G5, GALNT11, and KCTD4 compared to reference value ranges for the levels of the one or more biomarkers in a control aqueous humor sample indicate that the patient has retinitis pigmentosa; and
treating the patient for the retinitis pigmentosa if the patient has a positive diagnosis for retinitis pigmentosa.

2. The method of claim 1, wherein:
the levels of RS1, SAG, SPINK4, FUT3, GDF10, LRIT2, FAIM, CDHR1, RCVRN, GSKIP, EYS, NPPC, HES6, FGF23, AP4M1, GUCA1A, SPATA33, MPPED2, ASIC4, JPH4, CPNE7, TENM4, GRM4, TNKS, GAD1, SV2A, SYT5, RIC3, CAMK2A, LRRTM4, KCNG4, DPP10, PPP1R27, RGS5, GRAP, RAMP3, MEOX1, AFAP1L1, OPCML, LRIT3, PLA2G5, GALNT11, and KCTD4 are measured in the aqueous humor sample;
the levels of RS1, SAG, SPINK4, FUT3, GDF10, LRIT2, FAIM, CDHR1, RCVRN, and GSKIP are measured in the aqueous humor sample;
the levels of EYS, NPPC, HES6, FGF23, AP4M1, GUCA1A, SPATA33, MPPED2, and ASIC4 are measured in the aqueous humor sample;
the levels of JPH4, CPNE7, TENM4, GRM4, TNKS, GAD1, SV2A, SYT5, RIC3, and CAMK2A are measured in the aqueous humor sample;
the levels of LRRTM4, KCNG4, DPP10, and PPP1R27 are measured in the aqueous humor sample;
the levels of RGS5, GRAP, RAMP3, and MEOX1, AFAP1L1 are measured in the aqueous humor sample; or
the levels of OPCML, LRIT3, PLA2G5, GALNT11, and KCTD4 are measured in the aqueous humor sample.

3. The method of claim 1, wherein said treating the patient for the retinitis pigmentosa comprises retinal sheet transplantation, RPE65 gene therapy, implanting a retinal prosthesis, or administering vitamin A, docosahexaenoic acid (DHA), N-acetylcysteine (NAC), and lutein, or a combination thereof.

4. A method of diagnosing and treating diabetic retinopathy in a patient, the method comprising:
obtaining an aqueous humor sample from an eye of the patient;
measuring levels of at least two, at least three, or at least four biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 in the aqueous humor sample, wherein increased levels of the one or more biomarkers selected from LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 compared to reference value ranges for the levels of the one or more biomarkers in a control aqueous humor sample indicate that the patient has diabetic retinopathy; and
treating the patient for the diabetic retinopathy if the patient has a positive diagnosis for diabetic retinopathy.

5. The method of claim 4, wherein the levels of LBP, CRP, ITIH3, APOA1, CPN2, C4BPA, HABP2, F13B, SERPINA10, IGFBP1, FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 are measured in the aqueous humor sample.

6. The method of claim 4, wherein said treating the patient for the diabetic retinopathy comprises administering an anti-vascular endothelial growth factor (VEGF) agent or a steroid, or performing panretinal laser photocoagulation or a vitrectomy, or a combination thereof.

7. The method of claim 4, wherein the levels of FN1, MMP19, ANGPT2, ROBO4, VEGFD, ANGPTL4, FLT4, FLNA, EPHA1, and ENG are measured in the aqueous humor sample, wherein increased levels of the one or more biomarkers selected from FN1, MMP19, ANGPT2, ROBO4, VEGFD, ANGPTL4, FLT4, FLNA, EPHA1, and ENG compared to reference value ranges for the levels of the one or more biomarkers in a control aqueous humor sample indicate that the patient has non-proliferative diabetic retinopathy.

8. The method of claim 7, further comprising treating the patient for the non-proliferative diabetic retinopathy if the patient has a positive diagnosis for non-proliferative diabetic retinopathy, wherein said treating the patient for the non-proliferative diabetic retinopathy comprises administering an anti-VEGF agent or a steroid, performing vitrectomy or photocoagulation, or increasing frequency of retinal exams or a combination thereof.

9. The method of claim 4, wherein the levels of THBS1, CLIC4, NRP2, VEGFA, WARS1, CXCL8, TYMP, CCL2, MAPK14, AKT1 are measured in the aqueous humor sample, wherein increased levels of the one or more biomarkers selected from THBS1, CLIC4, NRP2, VEGFA, WARS1, CXCL8, TYMP, CCL2, MAPK14, AKT1 compared to reference value ranges for the levels of the one or more biomarkers in a control aqueous humor sample indicate that the patient has proliferative diabetic retinopathy.

10. The method of claim 9, further comprising treating the patient for the proliferative diabetic retinopathy if the patient has a positive diagnosis for proliferative diabetic retinopathy, wherein said treating the patient for the proliferative diabetic retinopathy comprises administering an anti-VEGF agent or performing panretinal laser photocoagulation, or a combination thereof.

11. A method of monitoring a patient having non-proliferative diabetic retinopathy for progression to proliferative diabetic retinopathy, the method comprising:
obtaining an aqueous humor sample from an eye of the patient;
measuring levels of one or more biomarkers selected from THBS1, CLIC4, NRP2, VEGFA, WARS1, CXCL8, TYMP, CCL2, MAPK14, and AKT1 in the aqueous humor sample, wherein increased levels of the one or more biomarkers selected from THBS1, CLIC4, NRP2, VEGFA, WARS1, CXCL8, TYMP, CCL2, MAPK14, and AKT1 compared to reference value ranges for the levels of the one or more biomarkers in a control aqueous humor sample indicate that the patient has proliferative diabetic retinopathy.

12. The method of claim 11, further comprising treating the patient for the proliferative diabetic retinopathy if the patient has a positive diagnosis for proliferative diabetic retinopathy, wherein said treating the patient for the proliferative diabetic retinopathy comprises administering an anti-VEGF agent or performing panretinal laser photocoagulation, or a combination thereof.

13. A method of distinguishing between a diagnosis of non-proliferative diabetic retinopathy and proliferative diabetic retinopathy for a patient, the method comprising:
obtaining an aqueous humor sample from an eye of the patient;
measuring levels of one or more biomarkers selected from FN1, MMP19, ANGPT2, ROBO4, VEGFD, ANGPTL4, FLT4, FLNA, EPHA1, and ENG in the aqueous humor sample, wherein increased levels of the one or more biomarkers selected from FN1, MMP19, ANGPT2, ROBO4, VEGFD, ANGPTL4, FLT4, FLNA, EPHA1, and ENG compared to reference value ranges for the levels of the one or more biomarkers in a control aqueous humor sample indicate that the patient has non-proliferative diabetic retinopathy; and
measuring levels of one or more biomarkers selected from THBS1, CLIC4, NRP2, VEGFA, WARS1, CXCL8, TYMP, CCL2, MAPK14, and AKT1 in the aqueous humor sample, wherein increased levels of the one or more biomarkers selected from THBS1, CLIC4, NRP2, VEGFA, WARS1, CXCL8, TYMP, CCL2, MAPK14, and AKT1 compared to reference value ranges for the levels of the one or more biomarkers in a control aqueous humor sample indicate that the patient has proliferative diabetic retinopathy.

14. The method of claim 13, wherein the levels of at least two, at least three, or at least four biomarkers selected from FN1, MMP19, ANGPT2, ROBO4, VEGFD, ANGPTL4, FLT4, FLNA, EPHA1, and ENG, and wherein the levels of at least two, at least three, or at least four biomarkers selected from THBS1, CLIC4, NRP2, VEGFA, WARS1, CXCL8, TYMP, CCL2, MAPK14, and AKT1 are measured in the aqueous humor sample.

15. The method of claim 13, wherein the levels of FN1, MMP19, ANGPT2, ROBO4, VEGFD, ANGPTL4, FLT4, FLNA, EPHA1, ENG, THBS1, CLIC4, NRP2, VEGFA, WARS1, CXCL8, TYMP, CCL2, MAPK14, and AKT1 are measured in the aqueous humor sample.

16. The method of claim 13, further comprising treating the patient for the non-proliferative diabetic retinopathy if the patient has a positive diagnosis for non-proliferative diabetic retinopathy, or treating the patient for the proliferative diabetic retinopathy if the patient has a positive diagnosis for proliferative diabetic retinopathy, wherein said treating the patient for the non-proliferative diabetic retinopathy comprises administering an anti-VEGF agent or a steroid, performing vitrectomy or photocoagulation, or increasing frequency of retinal exams or a combination thereof, wherein said treating the patient for the proliferative diabetic retinopathy comprises administering an anti-VEGF agent or performing panretinal laser photocoagulation, or a combination thereof.

17. A method of diagnosing and treating Parkinson's disease in a patient, the method comprising:
obtaining an aqueous humor sample from an eye of the patient;
measuring levels of at least two, at least three, or at least four biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 in the aqueous humor sample, wherein decreased levels of the one or more biomarkers selected from TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 compared to reference value ranges for the levels of the one or more biomarkers in a control aqueous humor sample indicate that the patient has Parkinson's disease; and
treating the patient for the Parkinson's disease if the patient has Parkinson's disease.

18. The method of claim 17, wherein:
the levels of TRIO, JPH4, CPNE7, GRM4, TNKS, SERAC1, ACVR2B, SV2A, RIC3, CAMK2A, BCL2L2, DBNDD1, SULT4A1, HABP4, SNPH, RAC3, SYT13, PAK5, NTRK1, C1QL4, KCNG4, DPP10, PPP1R27, PIH1D2, CDHR1, C17orf67, GSKIP, CEP112, HES6, FGF23, AP4M1, SPATA33, MPPED2, CHRNA5, and ASIC4 are measured in the aqueous humor sample;
the levels of TRIO, JPH4, CPNE7, GRM4, TNKS, SERAC1, ACVR2B, SV2A, RIC3, and CAMK2A are measured in the aqueous humor sample;
the levels of BCL2L2, DBNDD1, SULT4A1, HABP4, SNPH, RAC3, and SYT13 are measured in the aqueous humor sample;
the levels of PAK5, NTRK1, and C1QL4 are measured in the aqueous humor sample;
the levels of KCNG4, DPP10, and PPP1R27 are measured in the aqueous humor sample;
the levels of PIH1 D2, CDHR1, C17orf67, GSKIP, and CEP112 are measured in the aqueous humor sample; or
the levels of HES6, FGF23, AP4M1, SPATA33, MPPED2, CHRNA5, and ASIC4 are measured in the aqueous humor sample.

19. The method of claim 17, wherein said treating the patient for the Parkinson's disease comprises administering I-3,4-dihydroxyphenylalanine (L-DOPA), an aromatic L-amino acid decarboxylase inhibitor, a catechol-O-methyltransferase (COMT) inhibitor, a dopamine agonist, a monoamine oxidase inhibitor, an anticholinergic, or a combination thereof.

20. A method of diagnosing and treating uveitis in a patient, the method comprising:
obtaining an aqueous humor sample from an eye of the patient;
measuring levels of at least two, at least three, or at least four biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A in the aqueous humor sample, wherein increased levels of the one or more biomarkers selected from IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A compared to reference value ranges for the levels of the one or more biomarkers in a control aqueous humor sample indicate that the patient has uveitis; and
treating the patient for the uveitis if the patient has a positive diagnosis for uveitis.

21. The method of claim 20, wherein:
the levels of IGHM, TXNDC5, SLAMF7, MZB1, IGHD, CR2, IGHG4, LY9, FCRL5, TNFRSF13B, MMP9, IL1R2, GLIPR2, MPO, RETN, CD177, PSTPIP1, CLEC12A, HMGB2, LTA4H, VWF, TIE1, SELE, CDH5, ANGPT2, FLT4, ADGRF5, VEGFC, TEK, MYCT1, CXCL10, IL18BP, CCL22, TNFRSF8, CCL7, MMP12, IL10, CXCL11, CELA1, GNLY, CST7, CD8A, CD5, GZMK, GZMA, CCL5, CD7, LCK, and SH2D1A are measured in the aqueous humor sample;
the levels of IGHM, TXNDC5, SLAMF7, MZB1, IGHD, CR2, IGHG4, LY9, FCRL5, TNFRSF13B are measured in the aqueous humor sample;
the levels of MMP9, IL1R2, GLIPR2, MPO, RETN, CD177, PSTPIP1, CLEC12A, HMGB2, LTA4H are measured in the aqueous humor sample;
the levels of VWF, TIE1, SELE, CDH5, ANGPT2, FLT4, ADGRF5, VEGFC, TEK, MYCT1 are measured in the aqueous humor sample;
the levels of CXCL10, IL18BP, CCL22, TNFRSF8, CCL7, MMP12, IL10, CXCL11, CELA1 are measured in the aqueous humor sample; or
the levels of GNLY, CST7, CD8A, CD5, GZMK, GZMA, CCL5, CD7, LCK, SH2D1A are measured in the aqueous humor sample.

22. The method of claim 20, further comprising measuring levels of one or more biomarkers selected from BICDL1, NPPC, HES6, FGF23, AP4M1, SPATA33, MPPED2, and CHRNA5, GPC2 in the aqueous humor sample, wherein decreased levels of the one or more biomarkers selected from BICDL1, NPPC, HES6, FGF23, AP4M1, SPATA33, MPPED2, CHRNA5, and GPC2 compared to reference value ranges for the levels of the one or more biomarkers in a control aqueous humor sample indicate that the uveitis is causing damage to cone cells.

23. The method of claim 20, further comprising measuring levels of one or more biomarkers selected from OPCML, GAP43, RIPPLY3, LRIT3, PLA2G5, FGF18, PLD5, and KCTD4 in the aqueous humor sample, wherein decreased levels of the one or more biomarkers selected OPCML, GAP43, RIPPLY3, LRIT3, PLA2G5, FGF18, PLD5, and KCTD4 compared to reference value ranges for the levels of the one or more biomarkers in a control aqueous humor sample indicate that the uveitis is causing damage to retinal pigment epithelium (RPE) cells.

24. The method of claim 20, further comprising measuring levels of one or more biomarkers selected from CPNE7, TYRO3, TENM4, MDM4, RDH13, SV2A, SYT5, OLFM3, RIC3, and SEMA6C in the aqueous humor sample, wherein decreased levels of the one or more biomarkers selected CPNE7, TYRO3, TENM4, MDM4, RDH13, SV2A, SYT5, OLFM3, RIC3, and SEMA6C compared to reference value ranges for the levels of the one or more biomarkers in a control aqueous humor sample indicate that the uveitis is causing damage to amacrine cells.

25. The method of claim 20, wherein said treating the patient for the uveitis comprises administering a glucocorticoid steroid, a cycloplegic agent, an antimetabolite, a T-cell inhibitor, an anti-tumor necrosis factor (TNF) agent, a biologic agent, an alkylating agent, an antibiotic for bacterial uveitis, an antiviral agent for viral uveitis, or an antifungal agent for fungal uveitis, or performing a vitrectomy, or a combination thereof.

26. A method of determining biological age of an eye in a patient, the method comprising:
obtaining an aqueous humor sample from an eye of the patient; and
measuring levels of at least two, at least three, or at least four biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 in the aqueous humor sample; and
using a machine learning aging model to determine the biological age of the eye based on the levels of the one or more biomarkers selected from LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3.

27. The method of claim 26, wherein the levels of LECT2, DCBLD1, SCGN, ACAN, AOC2, CAPS, TFF3, AMN, ABO, NETO1, CD274, PPBP, ABL1, HAMP, IGFBP1, AAMDC, A1BG, ADA2, MVP, CRYGC, A4GALT, A4GNT, AADAT, A2M, and ABI3 are measured in the aqueous humor sample.

28. The method of claim 26, further comprising:
screening the patient for an age-related eye disease if the biological age of the eye indicates a risk of age-related pathology and morbidity; and
administering a treatment for the age-related eye disease to the patient if the patient is identified as having the age-related eye disease.
